(19) United States
(12) Patent Application Publication
(10) Pub. No.: US 2016/0158375 A1
(43) Pub. Date: Jun. 9, 2016

(54) DIPEPTIDE LINKED MEDICINAL AGENTS

(71) Applicant: Indiana University Research and Technology Corporation, Indianapolis, IN (US)

(72) Inventor: Richard D. DiMARCHI, Carmel, IN (US)

(21) Appl. No.: 15/016,959

(22) Filed: Feb. 5, 2016

Related U.S. Application Data
(62) Division of application No. 13/697,050, filed on Jan. 28, 2013.
(60) Provisional application No. 61/358,194, filed on Jun. 24, 2010.

(51) Int. Cl. A61K 47/48; A61K 38/08

(52) U.S. Cl. CPC A61K 47/48246; A61K 38/08

(57) ABSTRACT

A non-enzymatically self cleaving dipeptide element is provided that can be linked to known medicinal agents via an amide bond. The dipeptide will spontaneously be cleaved from the medicinal agent under physiological conditions through a reaction driven by chemical instability. Accordingly, the dipeptide element provides a means of linking various compounds to known medicinal agents wherein the compounds are subsequently released from the medicinal agent after a predetermined time of exposure to physiological conditions. For example, the dipeptide can be linked to an active site of a drug to form a prodrug and/or the dipeptide may comprise a depot polymer to sequester an injectable composition comprising the complex at the point of administration.

### CROSS REFERENCE TO RELATED APPLICATIONS

This application claims priority to U.S. application Ser. No. 13/697,050, filed Jan. 28, 2013, which is a U.S. national counterpart of International Application Serial No. PCT/US2011/039755, filed Jun. 9, 2011, which claims priority to U.S. Provisional Patent Application No. 61/358,194, filed on Jun. 24, 2010. The entire disclosures of PCT/US2011/039755, U.S. Ser. No. 13/697,050 and Provisional U.S. Ser. No. 61/358,194 are hereby expressly incorporated by reference in their entirety.

### INCORPORATION BY REFERENCE OF MATERIAL SUBMITTED ELECTRONICALLY

Incorporated by reference in its entirety is a computer-readable amino acid sequence listing submitted concurrently herewith and identified as follows: One 34.8 KB ASCII (Text) file named “Sequence_Listing_248291,” created on Feb. 4, 2016.

### BACKGROUND

It is often desirable to extend the release time of an injected drug to increase its duration of action, or to reduce its toxic effects. Formulations that are readily soluble in the body are usually absorbed rapidly and provide a sudden burst of available drug as opposed to a more desirable and gradual release of the pharmacologically active product. In addition, while numerous peptide-based drugs can be used as highly effective medicines, they typically have relatively short duration of action and variable therapeutic index.

A variety of attempts have been made to provide controlled and extended release pharmaceutical compounds, but previously disclosed techniques have not succeeded in overcoming all of the problems associated with the technology, such as achieving an optimal extended release time, maximizing stability and efficacy, reducing toxicity, maximizing reproducibility in preparation, and eliminating unwanted physical, biochemical, or toxicological effects introduced by undesirable matrix materials. Accordingly, there is a need for formulations that extend the half life of existing pharmaceuticals and improve their therapeutic index.

Mechanisms for providing extended release and an enhanced therapeutic index include sequestering molecules at the injection site or the use of prodrug derivative forms of the pharmaceutical, wherein the prodrug derivative is designed to delay onset of action and extend the half life of the drug. The delayed onset of action is advantageous in that it allows systemic distribution of the prodrug prior to its activation. Accordingly, the administration of prodrugs eliminates complications caused by peak activities upon administration and increases the therapeutic index of the parent drug.

Receptor recognition and subsequent processing of peptide and protein agonists is the primary route of degradation of many peptide and protein-based drugs. Thus binding of the peptide drug to its receptor will result in biological stimulation, but will also initiate the subsequent deactivation of the peptide/protein induced pharmacology through the enzymatic degradation of the peptide or protein. In accordance with the present disclosure, existing pharmaceutical compounds can be modified to prevent their interaction with their corresponding receptor. More particularly, as disclosed herein known drugs can be modified by the linkage of a non-enzymatic self cleaving dipeptide to the drug to form a complex that functions either as a depot composition, to localize the drug at the injection site for release in a controlled manner, or as a prodrug that is distributed through out the body but incapable of interacting with its receptor.

### SUMMARY

In accordance with one embodiment a non-enzymatic self cleaving dipeptide moiety is provided that can be covalently linked to a medicinal agent, wherein the dipeptide (and any compound linked to the dipeptide) is released from the medicinal agent at a predetermined length of time after exposure to physiological conditions. Advantageously, the rate of cleavage depends on the structure and stereochemistry of the dipeptide element and also on the strength of the nucleophile present on the dipeptide that induces cleavage and diketopiperazine or diketomorpholine formation. In one embodiment a complex comprising a known drug and a dipeptide of the structure A-B is provided, wherein A is an amino acid or a hydroxyl acid and B is an N-alkylated amino acid that is linked to the drug through formation of an amide bond between B and an amine of the drug. The amino acids of the dipeptide are selected such that a non-enzymatic chemical cleavage of A-B from the drug produces a diketopiperazine or diketomorpholine and the reconstituted native drug.

In one embodiment an injectable depot composition is provided comprising a complex having the general structure of A-B-Q wherein

A is an amino acid or a hydroxyl acid;

B is an N-alkylated amino acid;

Q is a an amine bearing medicinal agent; wherein the dipeptide A-B further comprises a depot polymer linked to the side chain of A or B, and said dipeptide is linked to Q through formation of an amide bond between A-B and an amine of Q. The depot polymer is selected to be of a sufficient size that the complex A-B-Q is effectively sequestered at the site of injection or is otherwise incapable of interacting with its target (e.g., receptor). Chemical cleavage of A-B from Q produces a diketopiperazine or diketomorpholine and releases the active drug to the patient in a controlled manner over a predetermined duration of time after administration.

In another embodiment prodrug derivatives of known pharmaceutical agents are prepared to extend the peptide or protein's biological half life based on a strategy of inhibiting recognition of the prodrug by the corresponding receptor. The prodrugs disclosed herein will ultimately be chemically converted to structures that can be recognized by the receptor, wherein the speed of this chemical conversion will determine the time of onset and duration of in vivo biological action. The molecular design disclosed in this application relies upon an intramolecular chemical reaction that is not dependent upon additional chemical additives, or enzymes.

The prodrug derivative is prepared by covalently linking a dipeptide element to an active site of the medicinal agent via an amide linkage. In one embodiment the dipeptide is covalently bound to the medicinal agent at a position that interferes with the medicinal agent's ability to interact with its corresponding receptor or cofactor. In one embodiment the dipeptide element is linked to the N-terminus of a bioactive peptide. Subsequent removal of the dipeptide, under physiological conditions and in the absence of enzymatic activity, restores full activity to the polypeptide.

In one embodiment a prodrug is provided having the general structure of A-B-Q. In this embodiment Q is a medicinal agent, including for example a bioactive peptide. In one embodiment Q is selected from the group of nuclear hormones consisting of thyroid hormone, estrogen, testosterone, and glucocorticoid, as well as analogs, derivatives and conjugates of the foregoing, and A-B represents a dipeptide prodrug linked to Q through an amide bond. More particularly, in one embodiment A is an amino acid or a hydroxyl acid and B is an N-alkylated amino acid linked to Q through formation of an amide bond between A-B and an amine of Q. In accordance with one embodiment the chemical cleavage half-life (t_{1/2}) of A-B from Q is at least about 1 hour to about 1 week in PBS under physiological conditions. Furthermore, in one embodiment Q comprises an amino acid sequence, and A, B, or the amino acid of Q to which A-B is linked, is a non-coded amino acid, and chemical cleavage of A-B from Q is at least about 90% complete within about 1 to about 720 hours in PBS under physiological conditions.

In one embodiment A and B are selected to inhibit enzymatic cleavage of the A-B dipeptide from Q by enzymes found in mammalian serum. In one embodiment A and/or B are selected such that the cleavage half-life of A-B from Q in PBS under physiological conditions is not more than two fold the cleavage half-life of A-B from Q in a solution comprising a DPP-IV protease (i.e., cleavage of A-B from Q does not occur at a rate more than 2× faster in the presence of DPP-IV protease and physiological conditions relative to identical conditions in the absence of the enzyme). In one embodiment A and/or B is an amino acid in the D stereoisomer configuration. In some exemplary embodiments, A is an amino acid in the D stereoisomer configuration and B is an amino acid in the L stereoisomer configuration. In some exemplary embodiments, A is an amino acid in the L stereoisomer configuration and B is an amino acid in the D stereoisomer configuration. In some exemplary embodiments, A is an amino acid in the D stereoisomer configuration and B is an amino acid in the D stereoisomer configuration.

In one embodiment the dipeptide element linked to the medicinal agent comprises a compound having the general structure of Formula I:

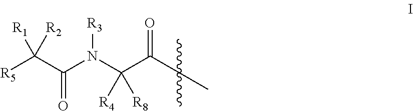

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH.

In another embodiment the dipeptide element linked to the medicinal agent comprises a compound having the general structure of Formula I:

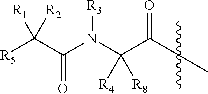

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to limit any value or range of values to only this broader definition. Each value or range of values preceded by the term “about” is also intended to encompass the embodiment of the stated absolute value or range of values.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. An amino acid can be designated by its three letter code, one letter code, or in some cases by the name of its side chain. For example, an unnatural amino acid comprising a cyclohexane group attached to the alpha carbon is termed “cyclohexane” or “cyclohexyl.” For the purposes of the present disclosure designation of an amino acid without specifying its stereochemistry is intended to encompass either the L or D form of the amino acid, or a racemic mixture. However, in the instance where an amino acid is designated by its three letter code and includes a superscript number (i.e., Lys⁻¹), such a designation is intended to specify the native L form of the amino acid, whereas the D form will be specified by inclusion of a lower case d before the three letter code and superscript number (i.e., dLys⁻¹).

As used herein the term “hydroxyl acid” refers to amino acids that have been modified to replace the alpha carbon amino group with a hydroxyl group.

As used herein the term “non-coded amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “dipeptide” is the result of the linkage of an alpha amino acid or an alpha hydroxyl acid to another amino acid, through a peptide bond.

As used herein the term “chemical cleavage” absent any further designation encompasses a non-enzymatic reaction that results in the breakage of a covalent chemical bond.

A “bioactive peptide” refers to peptides which are capable of exerting a biological effect in vitro and/or in vivo. As used herein a general reference to a peptide is intended to encompass peptides that have modified amino and carboxy termini. For example, an amino acid sequence designating the standard amino acids is intended to encompass standard amino acids at the N- and C-terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally-occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, such as DPP-IV, and (v) increased potency at a medicinal agent peptide receptor.

As used herein, an “alkylated” amino acid is an amino acid comprising an alkyl group which is non-native to a naturally-occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide. Without being held to any particular theory, it is believed that alkylation of peptides will achieve similar, if not the same, effects as acylation of the peptides, e.g., a prolonged half-life in circulation, a delayed onset of action, an extended duration of action, an improved resistance to proteases, such as DPP-IV, and increased potency at a medicinal agent peptide receptors.

As used herein, the term “prodrug” is defined as any compound that undergoes chemical modification before exhibiting its pharmacological effects.

As used herein, the term “medicinal agents” refers to a biologically active substance or substances that mediate their effect through interacting with a receptor, and for purposes of the present disclosure medicinal agents are defined as compounds falling into one of four classes:

1. nuclear hormones and derivatives thereof;

2. non-glucagon and non-insulin peptide-based hormones and derivatives;

3. proteins within the class of 4-helix bundle proteins, including for example growth hormone, leptin, erythropoietin, colony stimulating factors (such as GCSF) and interferons; and.

4. blood clotting factors, including for example, tissue plasminogen activators (TPA), Factor VII, Factor VIII and Factor IX.

As used herein a “nuclear hormone” is a compound that when bound to its corresponding receptor, will directly interact with and control the expression of genomic DNA. Examples of nuclear hormones include thyroid hormone, glucocorticoids, estrogens, androgens, vitamin A and vitamin D.

As used herein a “receptor” is a molecule that recognizes and binds with specific molecules in a high affinity interaction, producing some effect (either directly or indirectly) in a cell, or on the cells and/or tissues of the host organism. A “cellular receptor” is a molecule on or within a cell that recognizes and binds with specific molecules, producing some effect (either directly or indirectly) in the cell.

As used herein a “non-glucagon and non-insulin peptide-based hormone” is a hormone that comprises a peptide sequence, but specifically excludes insulin, insulin derivatives and analogs that specifically bind to the insulin receptor, insulin-like growth factors (IGFs) and glucagon superfamily peptides.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “glucagon related peptide” is directed to those peptides which have biological activity (as agonists or antagonists) at any one or more of the glucagon, GLP-1, GLP-2, and GIP receptors and comprise an amino acid sequence that shares at least 40% sequence identity (e.g., 45%, 50%, 55%, 60%, 65%, 70%, 75%, 80%, 85%, 90%, 95%) with at least one of native glucagon (SEQ ID NO: 1), native oxyntomodulin (SEQ ID NO: 51), native exendin-4 (SEQ ID NO: 54), native GLP-1 (SEQ ID NO: 50), native GLP-2 (SEQ ID NO: 53), or native GIP (SEQ ID NO: 52).

The term “glucagon superfamily” refers to a group of peptides related in structure in their N-terminal and C-terminal regions (see, for example, Sherwood et al., Endocrine Reviews 21: 619-670 (2000)). Members of this group include all glucagon related peptides, as well as Growth Hormone Releasing Hormone (GHRH; SEQ ID NO: 8), vasoactive intestinal peptide (VIP; SEQ ID NO: 55), Pituitary adenylate cyclase-activating polypeptide 27 (PACAP-27; SEQ ID NO: 56), peptide histidine isoleucine (PHI), peptide histidine methionine (PHM; SEQ ID NO: 57), and Secretin (SEQ ID NO: 58), and analogs, derivatives or conjugates with up to 1, 2, 3, 4, 5, 6, 7, 8, 9 or 10 amino acid modifications relative to the native peptide.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic but sufficient amount of the drug to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as subcutaneous, intramuscular, intraspinal, or intravenous.

As used herein an amino acid “modification” refers to a substitution, addition or deletion of an amino acid, and includes substitution with, or addition of, any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid.

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine

V. Large, aromatic residues:
Phe, Tyr, Trp, acetyl phenylalanine

As used herein the general term “polyethylene glycol chain” or “PEG chain”, refers to mixtures of condensation polymers of ethylene oxide and water, in a branched or straight chain, represented by the general formula H(OCH₂CH₂)ₖOH, wherein k is at least 9. Absent any further characterization, the term is intended to include polymers of ethylene glycol with an average total molecular weight selected from the range of 500 to 60,000 Daltons. “Polyethylene glycol chain” or “PEG chain” is used in combination with a numeric suffix to indicate the approximate average molecular weight thereof. For example, PEG-5,000 (5 k PEG) refers to polyethylene glycol chain having a total molecular weight average of about 5,000 Daltons.

As used herein the term “pegylated” and like terms refers to a compound that has been modified from its native state by linking a polyethylene glycol chain to the compound. A “pegylated polypeptide” is a polypeptide that has a PEG chain covalently bound to the polypeptide.

As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups.

As used herein a “dimer” is a complex comprising two subunits covalently bound to one another via a linker. The term dimer, when used absent any qualifying language, encompasses both homodimers and heterodimers. A homodimer comprises two identical subunits, whereas a heterodimer comprises two subunits that differ, although the two subunits are substantially similar to one another.

The term “C₁-Cₙ alkyl” wherein n can be from 1 through 6, as used herein, represents a branched or linear alkyl group having from one to the specified number of carbon atoms. Typical C₁-C₆ alkyl groups include, but are not limited to, methyl, ethyl, n-propyl, iso-propyl, butyl, iso-butyl, sec-butyl, tert-butyl, pentyl, hexyl and the like.

The terms “C₂-Cₙ alkenyl” wherein n can be from 2 through 6, as used herein, represents an olefinically unsaturated branched or linear group having from 2 to the specified number of carbon atoms and at least one double bond. Examples of such groups include, but are not limited to, 1-propenyl, 2-propenyl (—CH₂—CH═CH₂), 1,3-butadienyl, (—CH═CHCH═CH₂), 1-butenyl (—CH═CHCH₂CH₃), hexenyl, pentenyl, and the like.

The term “C₂-Cₙ alkynyl” wherein n can be from 2 to 6, refers to an unsaturated branched or linear group having from 2 to n carbon atoms and at least one triple bond. Examples of such groups include, but are not limited to, 1-propynyl, 2-propynyl, 1-butynyl, 2-butynyl, 1-pentynyl, and the like.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. The size of the aryl ring and the presence of substituents or linking groups are indicated by designating the number of carbons present. For example, the term “(C₁-C₃ alkyl)(C₆-C₁₀ aryl)” refers to a 6 to 10 membered aryl that is attached to a parent moiety via a one to three membered alkyl chain.

The term “heteroaryl” as used herein refers to a mono- or bi-cyclic ring system containing one or two aromatic rings and containing at least one nitrogen, oxygen, or sulfur atom in an aromatic ring. The size of the heteroaryl ring and the presence of substituents or linking groups are indicated by designating the number of carbons present. For example, the term “(C₁-Cₙ alkyl)(C₅-C₆ heteroaryl)” refers to a 5 or 6 membered heteroaryl that is attached to a parent moiety via a one to “n” membered alkyl chain.

As used herein, the term “halo” refers to one or more members of the group consisting of fluorine, chlorine, bromine, and iodine.

As used herein the term “charged amino acid” refers to an amino acid that comprises a side chain that is negatively charged (i.e., de-protonated) or positively charged (i.e., protonated) in aqueous solution at physiological pH. For example negatively charged amino acids include aspartic acid, glutamic acid, cysteic acid, homocysteic acid, and homoglutamic acid, whereas positively charged amino acids include arginine, lysine and histidine. Charged amino acids include the charged amino acids among the 20 amino acids commonly found in human proteins, as well as atypical or non-naturally occurring amino acids.

As used herein the term “acidic amino acid” refers to an amino acid that comprises a second acidic moiety (i.e. other than the α-carboyxl group that all amino acids possess), including for example, a carboxylic acid or sulfonic acid group.

As used herein the term “patient” without further designation is intended to encompass any warm blooded vertebrate domesticated animal (including for example, but not limited to livestock, horses, cats, dogs and other pets) and humans.

### EMBODIMENTS

In accordance with one embodiment a method is provided for increasing an administered drug's duration of action and improving its therapeutic index. The method comprises linking a dipeptide element to the drug via an amide linkage to produce a dipeptide/drug complex that is either sequestered at its point of administration or is biologically inactive. In accordance with one embodiment two or more dipeptide elements are linked via an amide bond to the drug. Under physiological conditions, the dipeptide will be cleaved via a non-enzymatic degradation mechanism thus releasing the active drug for interaction with its target. Advantageously, the rate of cleavage depends on the structure and stereochemistry of the dipeptide element and also on the strength of the nucleophile present on the dipeptide that induces cleavage and diketopiperazine or diketomorpholine formation. In one embodiment, based on the selected structure of the dipeptide, the non-enzymatic half time (t½) of the dipeptide/drug complex can be selected to be between 1-720 hrs under physiological conditions. Physiological conditions as disclosed herein are intended to include a temperature of about 35 to 40° C. and a pH of about 7.0 to about 7.4, and more typically include a pH of 7.2 to 7.4 and a temperature of 36 to 38° C. Since physiological pH and temperature are tightly regulated within a highly defined range, the speed of conversion from dipeptide/drug complex to drug will exhibit high intra and interpatient reproducibility.

In accordance with one embodiment the dipeptide element is covalently bound to the drug via an amide linkage at an active site of the drug to form a prodrug derivative of the drug. Typically the prodrug will exhibit no more than 10% of the activity of the parent drug, in one embodiment the prodrug exhibits less than 10%, less than 5%, about 1%, or less than 1% activity relative to the parent drug. The prodrugs disclosed herein will ultimately be chemically converted to structures that can be recognized by the native receptor of the drug, wherein the speed of this chemical conversion will determine the time of onset and duration of in vivo biological action. In one embodiment the drug is a medicinal agent. The molecular design disclosed in this application relies upon an intramolecular chemical reaction that is not dependent upon additional chemical additives, or enzymes, wherein the speed of conversion is controlled by the chemical nature of the dipeptide substituents.

In another embodiment, the dipeptide element is covalently bound to the drug via an amide linkage, and the dipeptide further comprises a depot polymer linked to dipeptide. In one embodiment the drug is a medicinal agent. In one embodiment two or more depot polymers are linked to a single dipeptide element. The depot polymer is selected to be biocompatible and of sufficient size that the drug modified by covalent attachment of the dipeptide remains sequestered at an injection site and/or incapable of interacting with its corresponding receptor upon administration to a patient. Subsequent cleavage of the dipeptide releases the drug to interact with its intended target. Selection of different combinations of substituents on the dipeptide element will allow for the preparation of injectable compositions that comprise a mixture of dipeptide/drug complexes that release the drug over a desired time frame.

In accordance with one embodiment, any known pharmaceutical that comprises a primary or secondary amine, or that can be modified to comprise such an amine without loss of function, can be modified to comprise a dipeptide element that will cleave via an intramolecular chemical reaction that is not dependent upon additional chemical additives, or enzymes. Advantageously, such a cleavage will regenerate the structure of the original pharmaceutical, with the speed of conversion exhibiting high intra and interpatient reproducibility. In one embodiment a non-enzymatic self cleaving dipeptide/drug complex is provided that comprises a known drug and a dipeptide element covalently bound to the drug through an amide bond. In one embodiment the non-enzymatic self cleaving complex comprises the structure A-B-Q wherein Q is an amine bearing medicinal agent, A is an amino acid or a hydroxyl acid and B is an N-alkylated amino acid that is linked to the medicinal agent through formation of an amide bond between B and an amine of the medicinal agent. The amino acids of the dipeptide are selected such that an intramolecular chemical reaction cleaves A-B from the medicinal agent, producing a diketopiperazine or diketomorpholine and the reconstituted native medicinal agent. In one embodiment A and/or B are selected from non-coding amino acids to inhibit cleavage of the dipeptide from the medicinal agent via an enzymatic mechanism. In one embodiment A and/or B are amino acids in the D-stereoisomer configuration. In some exemplary embodiments, A is an amino acid in the D stereoisomer configuration and B is an amino acid in the L stereoisomer configuration. In some exemplary embodiments, A is an amino acid in the L stereoisomer configuration and B is an amino acid in the D stereoisomer configuration. In some exemplary embodiments, A is an amino acid in the D stereoisomer configuration and B is an amino acid in the D stereoisomer configuration.

In one embodiment an injectable depot composition is provided comprising a dipeptide/drug complex having the general structure of A-B-Q and a depot polymer wherein

A is an amino acid or a hydroxyl acid;

B is an N-alkylated amino acid;

Q is a known drug that comprises an amine, or a derivative of a known drug modified to comprise an amine, wherein one or more depot polymers are linked to the dipeptide/drug complex. In one embodiment the depot polymer is linked to the side chain of A or B, and the dipeptide (A-B) is linked to Q through formation of an amide bond between B and an amine of Q.

In one embodiment Q is a medicinal agent. In one embodiment Q is selected from the group of compounds consisting of nuclear hormones, non-glucagon and non-insulin peptide-based hormones, proteins within the class of 4-helix bundle proteins and blood clotting factors. In one embodiment Q is a nuclear hormone or a non-glucagon and non-insulin peptide-based hormone. Examples of non-glucagon and non-insulin peptide-based hormones include, but are not limited to, calcitonin (SEQ ID NOs 14-34), parathyroid hormone (PTH; SEQ ID NO: 49), amylin (SEQ ID NOs: 35-47) or pramlitide; (SEQ ID NO: 48), somatostatin (SEQ ID NO: 12 and 13), growth hormone releasing hormone (GHRH; SEQ ID NO: 8), vasopressin (SEQ ID NO: 6), oxytocin (SEQ ID NO: 10), atrial natriuretic factor (ANF; SEQ ID NO: 7), neuropeptide Y (NPY; SEQ ID NO: 9), and pancreatic peptide Y (PYY; SEQ ID NO: 11), or peptides sharing at least 60%, 65%, 70%, 75%, 80%, 85%, 90% or 95% sequence identity with said non-glucagon and non-insulin peptide-based hormones amino acid sequences. In one embodiment Q is a compound selected from the group consisting of thyroid hormone, glucocorticoids, estrogens, androgens, vitamin D, calcitonin, parathyroid hormone (PTH), amylin (or pramlitide), growth hormone, somatostatin, growth hormone releasing hormone (GHRH), vasopressin, oxytocin, atrial natriuretic factor (ANF), neuropeptide Y (NPY), pancreatic peptide Y (PYY), leptin, erythropoietin, colony stimulating factors (such as GCSF), interferons (e.g. alpha and beta isoforms), tissue plasminogen activators (TPA), and blood clotting factors, such as Factor VII, Factor VIII and Factor IX. In one embodiment Q is a compound selected from the group consisting of thyroid hormone, glucocorticoids, estrogens, androgens, vitamin D, calcitonin, parathyroid hormone (PTH) and amylin. In one embodiment Q is a compound selected from the group consisting of thyroid hormone, calcitonin, parathyroid hormone (PTH) and amylin. In one embodiment Q is thyroid hormone.

The depot polymer is selected to be of a sufficient size that the complex A-B-Q is effectively sequestered at the site of injection upon injection of the composition, and/or the depot polymer interferes with Q's ability to interact with its natural ligand. In one embodiment one or more depot polymers are covalently linked to A and/or B either directly or indirectly through a linker. In one embodiment one or more depot polymers are non-covalently linked through a high affinity association with A or B (either through direct interaction with A or B or through a linking moiety covalently bound to A or B). Chemical cleavage of A-B from Q produces a diketopiperazine or diketomorpholine and releases the active drug, in a controlled manner over a predetermined duration of time after administration, to distribute systemically in the patient (in those embodiment where the initial complex is initially sequestered) and allows the active drug to interact with its target ligand.

In one embodiment an injectable composition is provided wherein the composition comprises a plurality of different dipeptide/drug complexes wherein the dipeptide/drug complexes differ from each other based on the structure of the dipeptide moiety. In accordance with one embodiment the dipeptide/drug complexes comprise a compound of the general structure of A-B-Q (as defined immediately above) with a depot polymer linked to A or B, wherein the dipeptide/drug complexes differ from one another based on the substituents of A and/or B. In this manner an injectable composition can be provided wherein the medicinal agent (Q) is released in a controlled manner over an extended period of time based on the cleavage rates of the individual different complexes. In accordance with one embodiment a composition is provided wherein the composition comprises the medicinal agent (Q) in a free form as well as the medicinal agent (Q) covalently bound to the dipeptide element. In this manner the administered composition will have an immediate therapeutic effect due to the presence of the active medicinal agent. In addition there will be an extended or delayed biological effect as the dipeptide is cleaved from the A-B-Q complex and releases additional active medicinal agent (Q) at a predetermined time interval after the initial administration of the composition.

In accordance with one embodiment the depot polymer is selected from biocompatible polymers known to those skilled in the art. The depot polymers typically have a size selected from a range of about 20,000 to 120,000 Daltons. In one embodiment the depot polymer has a size selected from a range of about 40,000 to 100,000 or about 40,000 to 80,000 Daltons. In one embodiment the depot polymer has a size of about 40,000, 50,000, 60,000, 70,000 or 80,000 Daltons. Suitable depot polymers include but are not limited to dextrans, polylactides, polyglycolides, caprolactone-based polymers, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof, and biodegradable polymers and their copolymers including caprolactone-based polymers, polycaprolactones and copolymers which include polybutylene terephthalate. In one embodiment the depot polymer is selected from the group consisting of polyethylene glycol, dextran, polylactic acid, polyglycolic acid and a copolymer of lactic acid and glycolic acid, and in one specific embodiment the depot polymer is polyethylene glycol. In one embodiment the depot polymer comprises one or more polyethylene glycol chains linked to the dipeptide element wherein the combined molecular weight of depot polymer(s) is 40,000 to 80,000 Daltons.

In accordance with one embodiment the depot polymer is linked to the side chain of one of the two amino acids of the dipeptide A-B (or to the side chain of a hydroxyl acid present at position “A” of the dipeptide). In one embodiment the dipeptide A-B comprises a cysteine or lysine residue to provide a reactive group for ease of attachment of the depot polymer. In one embodiment the dipeptide A-B comprises a lysine or cysteine wherein a polyethylene glycol having a molecular weight selected from the range of 40,000 to 80,000 Daltons is covalently linked to the lysine or cysteine side chain.

In a further embodiment A and/or B are selected to resist cleavage by peptidases present in human serum, including for example dipeptidyl peptidase IV (DPP-IV). Accordingly, in one embodiment the rate of cleavage of the dipeptide element from the bioactive peptide is not substantially enhanced (e.g., greater than 2×) when the reaction is conducted using physiological conditions in the presence of serum proteases relative to conducting the reaction in the absence of the proteases. Thus the cleavage half-life of A-B from the bioactive peptide in standard PBS under physiological conditions is not more than two, three, four or five fold the cleavage half-life of A-B from the bioactive protein in a solution comprising a DPP-IV protease. In one embodiment the solution comprising a DPP-IV protease is serum, more particularly mammalian serum, including human serum.

In a further embodiment one of A or B of said A-B dipeptide represents a non-coded amino acid. Alternatively, in embodiments where Q comprises a peptide, A, B, or the amino acid comprising the amino group of Q to which A-B is linked, is a non-coded amino acid. In one embodiment amino acid “B” is N-alkylated but is not proline. In one embodiment the N-alkyl group of amino acid B is a C₁-C₁₈ alkyl, and in one embodiment is C₁-C₆ alkyl. In another embodiment the dipeptide/drug complex may be further modified to comprise a covalently bound acyl group or alkyl group. In one embodiment the acyl group or alkyl group is covalently linked to the side chain of A or B of the dipeptide A-B.

In accordance with one embodiment the dipeptide element (A-B) comprises the structure:

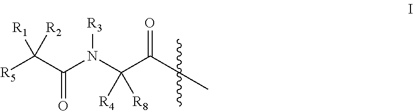

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH, with the proviso that when R₄ and R₃ together with the atoms to which they are attached form a 5 or 6 member heterocyclic ring, then at least one of R₁ and R₂ are other than hydrogen.

In another embodiment the dipeptide element (A-B) comprises the structure:

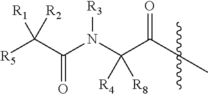

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that when R₄ and R₃ together with the atoms to which they are attached form a 5 or 6 member heterocyclic ring, then at least one of R₁ and R₂ are other than hydrogen.

In one embodiment the dipeptide A-B comprises the structure of formula I wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl or aryl;

R₅ is NHR₆; and

R₆ is H or C₁-C₈ alkyl.

In other embodiments the dipeptide prodrug element comprises the structure of Formula I, wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl;

R₃ is C₁-C₁₈ alkyl;

R₅ is NHR₆;

R₆ is H or C₁-C₈ alkyl; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo. In one embodiment when R₄ and R₃ together with the atoms to which they are attached form a 4, 5 or 6 member heterocyclic ring then at least one of R₁ and R₂ are other than hydrogen. In one embodiment when R₄ and R₃ together with the atoms to which they are attached form a 4, 5 or 6 member heterocyclic ring then both R₁ and R₂, are other than hydrogen.

In accordance with one embodiment the dipeptide element (A-B) is linked to a medicinal agent via a primary amine present on the native drug, or a primary amine introduced into the drug by chemical modification, wherein the substituents of the dipeptide element are selected to provide a dipeptide/drug complex (A-B-Q) wherein the t_{1/2} of A-B-Q is about 1 hour in standard PBS under physiological conditions. In accordance with one embodiment a dipeptide/drug complex having a t_{1/2} of about 1 hour in standard PBS under physiological conditions is provided wherein A-B comprises the structure of formula I wherein

R₁ and R₂ are independently C₁-C₁₈ alkyl or aryl; or R₁ and R₂ are linked through —(CH₂)ₚ—, wherein p is 2-9;

R₃ is C₁-C₁₈ alkyl;

R₄ and R₈ are each hydrogen; and

R₅ is an amine.

In other embodiments, prodrugs having a t_{1/2} of, e.g., about 1 hour comprise a dipeptide prodrug element with the structure of Formula I:

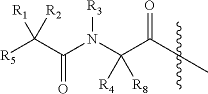

wherein

R₁ and R₂ are independently C₁-C₁₈ alkyl or (C₀-C₄ alkyl)(C₆-C₁₀ aryl)R₇; or R₁ and R₂ are linked through —(CH₂)ₚ, wherein p is 2-9;

R₃ is C₁-C₁₈ alkyl;

R₄ and R₈ are each hydrogen;

R₅ is NH₂; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In an alternative embodiment the substituents of the dipeptide element are selected to provide a complex A-B-Q, wherein the t_{1/2} of A-B-Q is about 6 to about 24 hours in standard PBS under physiological conditions. In accordance with one embodiment a dipeptide/medicinal agent complex is provided having the structure A-B-Q and a t_{1/2} of about 6 to about 24 hours in standard PBS under physiological conditions wherein A-B comprises the structure of formula I further wherein

R₁ and R₂ are independently selected from the group consisting of hydrogen, C₁-C₁₈ alkyl and aryl, or R₁ and R₂ are linked through —(CH₂)ₚ—, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and aryl; and

R₅ is an amine;

with the proviso that both R₁ and R₂ are not hydrogen and provided that one of R₄ or R₈ is hydrogen.

In some embodiments, the substituents of the dipeptide element are selected to provide a complex A-B-Q, wherein the t_{1/2} of A-B-Q is e.g., between about 12 to about 72 hours, or in some embodiments between about 12 to about 48 hours. In accordance with some embodiments, a dipeptide/medicinal agent complex is provided having the structure A-B-Q and a t_{1/2} between about 12 to about 72 hours, or in some embodiments between about 12 to about 48 hours in standard PBS under physiological conditions wherein A-B comprises the structure of formula I:

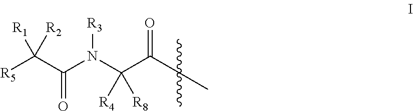

wherein R₁ and R₂ are independently selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇, or R₁ and R₂ are linked through (CH₂)ₚ, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and (C₀-C₄ alkyl)(C₆-C₁₀ aryl)R₇;

R₅ is NH₂; and

R₇ is selected from the group consisting of H, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that both R₁ and R₂ are not hydrogen and provided that at least one of R₄ or R₈ is hydrogen.

In accordance with some embodiments, a dipeptide/medicinal agent complex is provided having the structure A-B-Q and a t_{1/2} between about 12 to about 72 hours, or in some embodiments between about 12 to about 48 hours in standard PBS under physiological conditions wherein A-B comprises the structure:

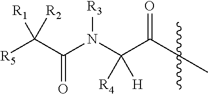

wherein R₁ and R₂ are independently selected from the group consisting of hydrogen, C₁-C₈ alkyl and (C₁-C₄ alkyl)NH₂, or R₁ and R₂ are linked through (CH₂)ₚ, wherein p is 2-9;

R₃ is C₁-C₈ alkyl or R₃ and R₄ together with the atoms to which they are attached form a 4-6 heterocyclic ring;

R₄ is selected from the group consisting of hydrogen and C₁-C₈ alkyl; and

R₅ is NH₂;

with the proviso that both R₁ and R₂ are not hydrogen.

In accordance with some embodiments, a dipeptide/medicinal agent complex is provided having the structure A-B-Q and a t_{1/2} between about 12 to about 72 hours, or in some embodiments between about 12 to about 48 hours in standard PBS under physiological conditions wherein A-B comprises the structure:

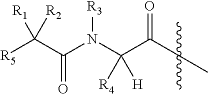

wherein

R₁ and R₂ are independently selected from the group consisting of hydrogen, C₁-C₈ alkyl and (C₁-C₄ alkyl)NH₂;

R₃ is C₁-C₆ alkyl;

R₄ is hydrogen; and

R₅ is NH₂;

with the proviso that both R₁ and R₂ are not hydrogen.

In accordance with some embodiments, a dipeptide/medicinal agent complex is provided having the structure A-B-Q and a t_{1/2} between about 12 to about 72 hours, or in some embodiments between about 12 to about 48 hours in standard PBS under physiological conditions wherein A-B comprises the structure:

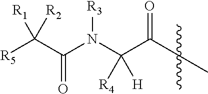

wherein

R₁ and R₂ are independently selected from the group consisting of hydrogen and C₁-C₈ alkyl, (C₁-C₄ alkyl)NH₂, or R₁ and R₂ are linked through (CH₂)ₚ, wherein p is 2-9;

R₃ is C₁-C₈ alkyl;

R₄ is (C₀-C₄ alkyl)(C₆-C₁₀ aryl)R₇;

R₅ is NH₂; and

R₇ is selected from the group consisting of hydrogen, C₁-C₈ alkyl and (C₀-C₄ alkyl)OH;

with the proviso that both R₁ and R₂ are not hydrogen.

In an alternative embodiment the substituents of the dipeptide element are selected to provide a dipeptide/medicinal agent complex (A-B-Q) wherein the t_{1/2} of A-B-Q is about 72 to about 168 hours in standard PBS under physiological conditions. In accordance with one such embodiment A-B comprises the structure of formula I wherein

R₁ is selected from the group consisting of hydrogen, C₁-C₈ alkyl and aryl;

R₃ is C₁-C₁₈ alkyl;

R₄ and R₈ are each hydrogen; and

R₅ is an amine or N-substituted amine or a hydroxyl;

with the proviso that, if R₁ is alkyl or aryl, then R₁ and R₅ together with the atoms to which they are attached form a 4-11 heterocyclic ring. In one embodiment R₁ is selected from the group consisting of hydrogen, C₁-C₈ alkyl and C₅-C₁₀ aryl, and in one embodiment R₁ is selected from the group consisting of hydrogen, C₁-C₈ alkyl and C₅-C₆ aryl.

In some embodiments, A-B comprises the structure:

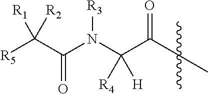

wherein

R₁ is selected from the group consisting of hydrogen, C₁-C₈ alkyl and (C₀-C₄ alkyl)(C₆-C₁₀ aryl)R₇;

R₃ is C₁-C₁₈ alkyl;

R₄ and R₈ are each hydrogen;

R₅ is NHR₆ or OH;

R₆ is H, C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that, if R₁ is alkyl or (C₀-C₄ alkyl)(C₆-C₁₀ aryl)R₇, then R₁ and R₅ together with the atoms to which they are attached form a 4-11 heterocyclic ring. In one embodiment R₁ is selected from the group consisting of hydrogen, C₁-C₈ alkyl and (C₀-C₄ alkyl)(C₅-C₁₀ aryl)R₇, and in one embodiment R₁ is selected from the group consisting of hydrogen, C₁-C₈ alkyl and (C₀-C₄ alkyl)(C₅-C₆ aryl)R₇.

The complexes comprising a depot polymer can be administered as an injectable composition to provide a sustained and controlled delivery of a beneficial agent to a subject over a prolonged duration of time. Accordingly, the dipeptide elements disclosed herein can be linked to any medicinal agent via an amide bond linkage and used to treat any disease or condition in accordance with known uses for the parent medicinal agent. The dipeptide/medicinal agent/depot polymer complexes of the present invention can provide a prolonged controlled delivery that is regulated by selection of the dipeptide substituents. In one embodiment the release is controlled over a period from about 6 to about 24 hours, about 48 to about 72 hours, 72 to about 168 hours, or about two weeks to one month after administration.

The present disclosure also encompasses the formulation of prodrug derivatives of known medicinal agent useful for treating patients. More particularly, the prodrugs disclosed herein are formulated to enhance the half life of the parent medicinal agent, while allowing for subsequent activation of the prodrug via a non-enzymatic degradation mechanism. The ideal prodrug should be soluble in water at physiological conditions (for example, a pH of 7.2 and 37° C.), and it should be stable in the powder form for long term storage. It should also be immunologically silent and exhibit a low activity relative to the parent drug. Typically the prodrug will exhibit no more than 10% of the activity of the parent drug, in one embodiment the prodrug exhibits less than 10%, less than 5%, about 1%, or less than 1% activity relative to the parent drug. Furthermore, the prodrug, when injected in the body, should be quantitatively converted to the active drug within a defined period of time. As disclosed herein, applicants have provided a general technique for producing prodrugs of a known medicinal agents, including bioactive peptides and non-peptide drugs such as thyroid hormone, estrogen, testosterone, and glucocorticoids, as well as analogs, derivatives and conjugates of the foregoing.

More particularly, in one embodiment a chemoreversible prodrug derivative of a known drug is provided, wherein the drug is modified to have a dipeptide element covalently bound to an active site of the drug via an amide linkage. Covalent attachment of the dipeptide element to an active site of the drug inhibits the activity of the drug until cleavage of the dipeptide element. In one embodiment a prodrug is provided having a non-enzymatic activation half time (t½) between 1-720 hrs under physiological conditions. Physiological conditions as disclosed herein are intended to include a temperature of about 35 to 40° C. and a pH of about 7.0 to about 7.4 and more typically include a pH of 7.2 to 7.4 and a temperature of 36 to 38° C.

Advantageously, the rate of cleavage, and thus activation of the prodrug, depends on the structure and stereochemistry of the dipeptide element and also on the strength of the dipeptide nucleophile. The prodrugs disclosed herein will ultimately be chemically converted to structures that can be recognized by the native receptor/substrate of the drug or medicinal agent, wherein the speed of this chemical conversion will determine the time of onset and duration of in vivo biological action. The molecular design disclosed in this application relies upon an intramolecular chemical reaction that is not dependent upon additional chemical additives, or enzymes. The speed of conversion is controlled by the chemical nature of the dipeptide substituent and its cleavage under physiological conditions. Since physiological pH and temperature are tightly regulated within a highly defined range, the speed of conversion from prodrug to drug will exhibit high intra and interpatient reproducibility.

As disclosed herein prodrugs are provided having half lives of at least 1 hour, and more typically greater than 20 hours. In one embodiment the half life of the prodrug is about 1, 6, 8, 12, 20, 24, 48 or 72 hours. In one embodiment the half life of the prodrug is 100 hours or greater including half lives of up to 168, 336, 504, 672 or 720 hours, wherein the prodrug is converted to the active form at physiological conditions through a non-enzymatic reaction driven by inherent chemical instability. In one embodiment the non-enzymatic activation t½ time of the prodrug is between 1-100 hrs, and more typically between 12 and 72 hours, for example, between 12 and 48 hours and between 48 and 72 hours, and in one embodiment the t½ is between 24-48 hrs as measured by incubating the prodrug in a phosphate buffer solution (e.g., PBS) at 37° C. and pH of 7.2. In another embodiment the non-enzymatic activation t_{1/2} time of the prodrug is between 1 and 6 hours, for example, about 1 hour, about 2 hours, about 3 hours, about 4 hours, about 5 hours, or about 6 hours. In another embodiment the non-enzymatic activation t_{1/2} time of the prodrug is between 6 and 24 hours. The half lives of the various prodrugs are calculated by using the formula t_{1/2}=0.693/k, where ‘k’ is the first order rate constant for the degradation of the prodrug. In one embodiment, activation of the prodrug occurs after cleavage of an amide bond linked dipeptide, and formation of a diketopiperazine or diketomorpholine, and the active medicinal agent. Specific dipeptides composed of natural, non-coding and/or synthetic amino acids have been identified that facilitate intramolecular decomposition under physiological conditions to release bioactive peptides.

In accordance with one embodiment a prodrug derivative of a known drug is provided wherein the prodrug has the structure:

A-B-Q;

wherein Q is a medicinal agent;

A is an amino acid or a hydroxyl acid;

B is an N-alkylated amino acid; and A-B is a dipeptide that is linked to Q through formation of an amide bond between B and an amine of Q, at an active site of Q. Furthermore, the amino acids of the dipeptide A-B are selected such that chemical cleavage of A-B from Q is more than 90% complete within 720 hours after solubilization in a standard PBS solution under physiological conditions. In one embodiment, one of A or B represents a non-coded amino acid, or when the dipeptide A-B is linked to Q through an amino acid, the dipeptide A-B is linked to Q through a non-coded amino acid. In an alternative embodiment the dipeptide A-B is linked to Q through an amide bond that does not constitute a peptide bond. In one embodiment the prodrug comprises the dipeptide A-B linked to the active site of a bioactive peptide wherein A, B, or the amino acid comprising the amino group of Q to which A-B is linked is a non-coded amino acid.

In one embodiment the prodrug comprises the structure A-B-Q wherein Q is a known drug that comprises an amine, or a derivative of a know drug modified to comprise an amine. In one embodiment Q is selected from the group of compounds consisting of nuclear hormones, non-glucagon and non-insulin peptide-based hormones, proteins within the class of 4-helix bundle proteins and blood clotting factors. In one embodiment Q is a nuclear hormone or a non-glucagon and non-insulin peptide-based hormone. In one embodiment Q is a compound selected from the group consisting of thyroid hormone, glucocorticoids, estrogens, androgens, vitamin D, calcitonin, parathyroid hormone (PTH), amylin, growth hormone, leptin, erythropoietin, colony stimulating factors (such as GCSF), interferons (e.g. alpha and beta isoforms), tissue plasminogen activitors (TPA), and blood clotting factors, such as Factor VII, Factor VIII and Factor IX. In one embodiment Q is a compound selected from the group consisting of thyroid hormone, glucocorticoids, estrogens, androgens, vitamin D, calcitonin, parathyroid hormone (PTH) and amylin. In one embodiment Q is a compound selected from the group consisting of thyroid hormone, calcitonin, parathyroid hormone (PTH) and amylin. In one embodiment Q is thyroid hormone.

The dipeptide element (A-B) is designed to cleave based upon an intramolecular chemical reaction that is not dependent upon additional chemical additives, or enzymes. More particularly, in one embodiment the dipeptide structure is selected to resist cleavage by peptidases present in mammalian sera, including for example dipeptidyl peptidase IV (DPP-IV). Accordingly, in one embodiment the rate of cleavage of the dipeptide element from the bioactive peptide is not substantially enhanced (e.g., greater than 2×) when the reaction is conducted using physiological conditions in the presence of serum proteases relative to conducting the reaction in the absence of the proteases. Thus the cleavage half-life of A-B from the bioactive peptide in PBS under physiological conditions is not more than two, three, four or five fold the cleavage half-life of A-B from the bioactive protein in a solution comprising a DPP-IV protease. In one embodiment the solution comprising a DPP-IV protease is serum, more particularly mammalian serum, including human serum.

In accordance with one embodiment A or B of the dipeptide element, or in the case of a bioactive peptide, the amino acid of the bioactive peptide to which A-B is linked is a non-coded amino acid. In one embodiment amino acid “B” is N-alkylated, but is not proline. In one embodiment the N-alkylated group of amino acid B is a C₁-C₁₈ alkyl, and in one embodiment is C₁-C₆ alkyl. In accordance with one embodiment the cleavage half-life of A-B from Q in standard PBS under physiological conditions is not more than two fold the cleavage half-life of A-B from Q in a solution comprising a DPP-IV protease. In one embodiment the solution comprising the DPP-IV protease is serum.

In accordance with one embodiment an aliphatic amino group of Q (i.e., a primary amine), including for example the N-terminal amine or the amino group of an amino acid side chain of a bioactive peptide, is modified by the covalent linkage of the dipeptide element via an amide bond. In one embodiment the dipeptide element is linked to an amino group present in Q, either directly or through a linking moiety. In one embodiment the linking moiety comprises a primary amine bearing acyl group or alkyl group.

Alternatively, the dipeptide element can be linked to an amino substituent present on an aryl ring of the peptide, including for example an aromatic amino acid of a bioactive peptide selected from the group consisting of amino-Phe, amino-napthyl alanine, amino tryptophan, amino-phenyl-glycine, amino-homo-Phe, and amino tyrosine. In one embodiment the dipeptide element is linked to the side chain amino group of a lysine amino acid or the aromatic amino group of a 4-aminophenylalanine (substituted for a native phenylalanine or tyrosine residue of the bioactive peptide). In one embodiment the dipeptide element is linked to an amine present on an internal amino acid of a bioactive peptide. In one embodiment is the dipeptide element is linked to a primary amine.

In accordance with one embodiment the dipeptide element can be further modified to comprise a hydrophilic moiety. In one embodiment the hydrophilic moiety is a polyethylene glycol chain. In accordance with one embodiment a polyethylene glycol chain of 40 k or higher is covalently bound to the side chain of the A or B amino acid of the dipeptide element. In another embodiment the dipeptide element is acylated or alkylated with a fatty acid or bile acid, or salt thereof, e.g. a C4 to C30 fatty acid, a C8 to C24 fatty acid, cholic acid, a C4 to C30 alkyl, a C8 to C24 alkyl, or an alkyl comprising a steroid moiety of a bile acid. Alternatively, the dipeptide element can be linked to a depot polymer such as dextran or a polyethylene glycol molecule (e.g. having a size of approximately 40,000 to 80,000 daltons) that serves to sequester the prodrug at an injection site until cleavage of the dipeptide releases the active bioactive peptide.

In one embodiment the dipeptide element has the general structure of Formula I:

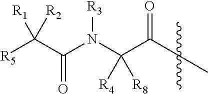

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and R₇ is selected from the group consisting of H and OH.

In some embodiments the dipeptide element has the general structure of Formula I:

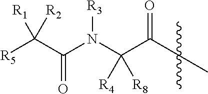

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In one embodiment R₈ is H and R₅ is NHR₆

In one embodiment the dipeptide element has the structure of Formula I, wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl or aryl;

R₅ is NHR₆; and

R₆ is H or C₁-C₈ alkyl.

In other embodiments the dipeptide prodrug element has the structure of Formula I, wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl;

R₃ is C₁-C₁₈ alkyl;

R₅ is NHR₆;

R₆ is H or C₁-C₈ alkyl; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

The half life of the prodrug formed in accordance with the present disclosure is determined by the substituents of the dipeptide element and the site on the drug to which it is attached. For example, the prodrug may comprise a dipeptide element linked through an aliphatic amino group of the drug. In this embodiment prodrugs having a t_{1/2} of 1 hour comprise a dipeptide element with the structure:

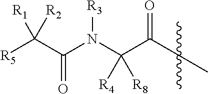

wherein

R₁ and R₂ are independently C₁-C₁₈ alkyl or aryl; or R₁ and R₂ are linked through —(CH₂)ₚ, wherein p is 2-9;

R₃ is C₁-C₁₈ alkyl;

R₄ and R₈ are each hydrogen; and

R₅ is an amine.

In some embodiments, prodrugs comprising a dipeptide element linked through an aliphatic amino group of the drug and having a t_{1/2}, e.g., of about 1 hour have the structure:

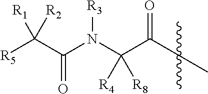

wherein

R₁ and R₂ are independently C₁-C₈ alkyl or (C₀-C₄ alkyl)(C₆-C₁₀ aryl)R₇; or R₁ and R₂ are linked through —(CH₂)ₚ—, wherein p is 2-9;

R₃ is C₁-C₁₈ alkyl;

R₄ and R₈ are each hydrogen;

R₅ is NH₂; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

Furthermore, in one embodiment prodrugs having the dipeptide element linked through an aliphatic amino group of the drug and having a t½ between about 6 to about 24 hours comprise a dipeptide element with the structure:

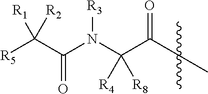

wherein R₁ and R₂ are independently selected from the group consisting of hydrogen, C₁-C₁₈ alkyl and aryl, or R₁ and R₂ are linked through (CH₂)ₚ, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and aryl; and R₅ is an amine;

with the proviso that both R₁ and R₂ are not hydrogen and provided that one of R₄ or R₈ is hydrogen.

In some embodiments prodrugs having the dipeptide element linked through an aliphatic amino group of the drug and having a t½ between about 12 to about 72 hours, or in some embodiments between about 12 to about 48 hours comprise a dipeptide element with the structure:

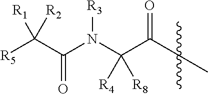

wherein R₁ and R₂ are independently selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇, or R₁ and R₂ are linked through (CH₂)ₚ, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and (C₀-C₄ alkyl)(C₆-C₁₀ aryl)R₇;

R₅ is NH₂; and

R₇ is selected from the group consisting of H, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that both R₁ and R₂ are not hydrogen and provided that at least one of R₄ or R₈ is hydrogen.

In some embodiments prodrugs having the dipeptide element linked through an aliphatic amino group of the drug and having a t½ between about 12 to about 72 hours, or in some embodiments between about 12 to about 48 hours comprise a dipeptide element with the structure:

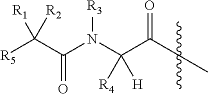

wherein R₁ and R₂ are independently selected from the group consisting of hydrogen, C₁-C₈ alkyl and (C₁-C₄ alkyl)NH₂, or R₁ and R₂ are linked through (CH₂)ₚ, wherein p is 2-9;

R₃ is C₁-C₈ alkyl or R₃ and R₄ together with the atoms to which they are attached form a 4-6 heterocyclic ring;

R₄ is selected from the group consisting of hydrogen and C₁-C₈ alkyl; and

R₅ is NH₂;

with the proviso that both R₁ and R₂ are not hydrogen.

In other embodiments prodrugs having the dipeptide element linked through an aliphatic amino group of the drug and having a t½ between about 12 to about 72 hours, or in some embodiments between about 12 to about 48 hours comprise a dipeptide element with the structure:

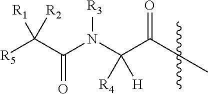

wherein

R₁ and R₂ are independently selected from the group consisting of hydrogen, C₁-C₈ alkyl and (C₁-C₄ alkyl)NH₂;

R₃ is C₁-C₆ alkyl;

R₄ is hydrogen; and

R₅ is NH₂;

with the proviso that both R₁ and R₂ are not hydrogen.

In some embodiments prodrugs having the dipeptide element linked through an aliphatic amino group of the drug and having a t½ between about 12 to about 72 hours, or in some embodiments between about 12 to about 48 hours comprise a dipeptide element with the structure:

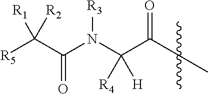

wherein

R₁ and R₂ are independently selected from the group consisting of hydrogen and C₁-C₈ alkyl, (C₁-C₄ alkyl)NH₂, or R₁ and R₂ are linked through (CH₂)ₚ, wherein p is 2-9;

R₃ is C₁-C₈ alkyl;

R₄ is (C₀-C₄ alkyl)(C₆-C₁₀ aryl)R₇;

R₅ is NH₂; and

R₇ is selected from the group consisting of hydrogen, C₁-C₈ alkyl and (C₀-C₄ alkyl)OH;

with the proviso that both R₁ and R₂ are not hydrogen.

In addition a prodrug having the dipeptide element linked through an aliphatic amino group of the drug and having a t½ of about 72 to about 168 hours is provided wherein the dipeptide element has the structure:

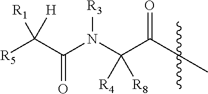

wherein R₁ is selected from the group consisting of hydrogen, C₁-C₈ alkyl and aryl;

R₃ is C₁-C₁₈ alkyl;

R₄ and R₈ are each hydrogen; and

R₅ is an amine or N-substituted amine or a hydroxyl;

with the proviso that, if R₁ is alkyl or aryl, then R₁ and R₅ together with the atoms to which they are attached form a 4-11 heterocyclic ring.

In some embodiments a prodrug having the dipeptide element linked through an aliphatic amino group of the drug and having a t½ of about 72 to about 168 hours is provided wherein the dipeptide element has the structure:

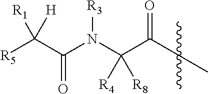

wherein R₁ is selected from the group consisting of hydrogen, C₁-C₁₈ alkyl and (C₀-C₄ alkyl)(C₆-C₁₀ aryl)R₇;

R₃ is C₁-C₁₈ alkyl;

R₄ and R₈ are each hydrogen;

R₅ is NHR₆ or OH;

R₆ is H or C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that, if R₁ and R₂ are both independently an alkyl or (C₀-C₄ alkyl)(C₆-C₁₀ aryl)R₇, either R₁ or R₂ is linked through (CH₂)ₚ to R₅, wherein p is 2-9.

In one embodiment the dipeptide element is linked to a side chain amine of an internal amino acid of a bioactive peptide. In this embodiment prodrugs having a t_{1/2} of about 1 hour have the structure:

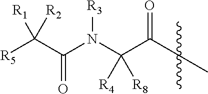

wherein

R₁ and R₂ are independently C₁-C₈ alkyl or aryl; or R₁ and R₂ are linked through (CH₂)ₚ, wherein p is 2-9;

R₃ is C₁-C₁₈ alkyl;

R₄ and R₈ are each hydrogen; and R₅ is an amine.

In some embodiments, the dipeptide element linked to a side chain amine of an internal amino acid of a bioactive peptide and having a t_{1/2}, e.g., of about 1 hour has the structure:

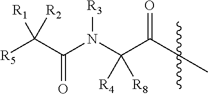

wherein

R₁ and R₂ are independently C₁-C₈ alkyl or (C₀-C₄ alkyl)(C₆-C₁₀ aryl)R₇; or R₁ and R₂ are linked through —(CH₂)ₚ—, wherein p is 2-9;

R₃ is C₁-C₁₈ alkyl;

R₄ and R₈ are each hydrogen;

R₅ is NH₂; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

Furthermore, in one embodiment prodrugs having a t½ between about 6 to about 24 hours and having the dipeptide element linked to an internal amino acid side chain comprise a dipeptide element with the structure:

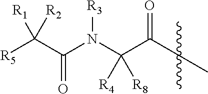

wherein R₁ and R₂ are independently selected from the group consisting of hydrogen, C₁-C₈ alkyl and aryl, or R₁ and R₂ are linked through —(CH₂)ₚ, wherein p is 2-9;

R₃ is C₁-C₁₈ alkyl or R₃ and R₄ together with the atoms to which they are attached form a 4-12 heterocyclic ring;

R₄ and R₈ are independently C₁-C₁₈ alkyl or aryl; and

R₅ is an amine or N-substituted amine;

with the proviso that both R₁ and R₂ are not hydrogen and provided that one of R₄ or R₈ is hydrogen.

In some embodiments, prodrugs having a t_{1/2}, e.g., between about 12 to about 72 hours, or in some embodiments between about 12 to about 48 hours, and having the dipeptide prodrug element linked to a internal amino acid side chain of a bioactive peptide comprises a dipeptide prodrug element with the structure:

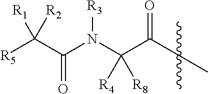

wherein R₁ and R₂ are independently selected from the group consisting of hydrogen, C₁-C₈ alkyl, and (C₀-C₄ alkyl)(C₆-C₁₀ aryl)R₇, or R₁ and R₂ are linked through —(CH₂)ₚ—, wherein p is 2-9;

R₃ is C₁-C₁₈ alkyl or R₃ and R₄ together with the atoms to which they are attached form a 4-12 heterocyclic ring;

R₄ and R₈ are independently hydrogen, C₁-C₁₈ alkyl or (C₀-C₄ alkyl)(C₆-C₁₀ aryl)R₇;

R₅ is NHR₆;

R₆ is H or C₁-C₈ alkyl, or R₆ and R₂ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that both R₁ and R₂ are not hydrogen and provided that at least one of R₄ or R₈ is hydrogen.

In some embodiments, prodrugs having a t_{1/2}, e.g., between about 12 to about 72 hours, or in some embodiments between about 12 to about 48 hours, and having the dipeptide prodrug element linked to a internal amino acid side chain of a bioactive peptide comprises a dipeptide prodrug element with the structure:

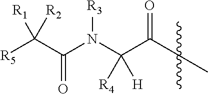

wherein R₁ and R₂ are independently selected from the group consisting of hydrogen, C₁-C₈ alkyl and (C₁-C₄ alkyl)NH₂, or R₁ and R₂ are linked through (CH₂)ₚ, wherein p is 2-9;

R₃ is C₁-C₈ alkyl or R₃ and R₄ together with the atoms to which they are attached form a 4-6 heterocyclic ring;

R₄ is selected from the group consisting of hydrogen and C₁-C₈ alkyl; and

R₅ is NH₂;

with the proviso that both R₁ and R₂ are not hydrogen.

In some embodiments, prodrugs having a t_{1/2}, e.g., between about 12 to about 72 hours, or in some embodiments between about 12 to about 48 hours, and having the dipeptide prodrug element linked to a internal amino acid side chain of a bioactive peptide comprises a dipeptide prodrug element with the structure:

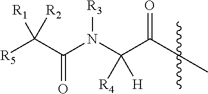

wherein

R₁ and R₂ are independently selected from the group consisting of hydrogen, C₁-C₈ alkyl and (C₁-C₄ alkyl)NH₂;

R₃ is C₁-C₆ alkyl;

R₄ is hydrogen; and

R₅ is NH₂;

with the proviso that both R₁ and R₂ are not hydrogen.

In some embodiments, prodrugs having a t_{1/2}, e.g., between about 12 to about 72 hours, or in some embodiments between about 12 to about 48 hours, and having the dipeptide prodrug element linked to a internal amino acid side chain of a bioactive peptide comprises a dipeptide prodrug element with the structure:

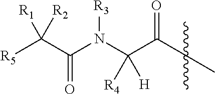

wherein

R₁ and R₂ are independently selected from the group consisting of hydrogen and C₁-C₈ alkyl, (C₁-C₄ alkyl)NH₂, or R₁ and R₂ are linked through (CH₂)ₚ, wherein p is 2-9;

R₃ is C₁-C₈ alkyl;

R₄ is (C₀-C₄ alkyl)(C₆-C₁₀ aryl)R₇;

R₅ is NH₂; and

R₇ is selected from the group consisting of hydrogen, C₁-C₈ alkyl and (C₀-C₄ alkyl)OH;

with the proviso that both R₁ and R₂ are not hydrogen.

In addition a prodrug having a t½ of about 72 to about 168 hours and having the dipeptide element linked to an internal amino acid side chain is provided wherein the dipeptide element has the structure:

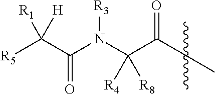

wherein R₁ and R₂ are independently selected from the group consisting of hydrogen, C₁-Cᵢₛ alkyl and aryl;

R₃ is C₁-C₁₈ alkyl;

R₄ and R₈ are each hydrogen; and

R₅ is an amine or N-substituted amine or a hydroxyl;

with the proviso that, if R₁ and R₂ are both independently an alkyl or aryl, either R₁ or R₂ is linked through (CH2)ₚ to R₅, wherein p is 2-9.

In some embodiments, a prodrug having a t_{1/2}, e.g., of about 72 to about 168 hours and having the dipeptide prodrug element linked to an internal amino acid side chain is provided wherein the dipeptide prodrug element has the structure:

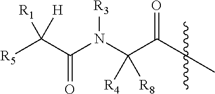

wherein R₁ and R₂ are independently selected from the group consisting of hydrogen, C₁-C₁₈ alkyl and (C₀-C₄ alkyl)(C₆-C₁₀ aryl)R₇;

R₃ is C₁-C₁₈ alkyl;

R₄ and R₈ are each hydrogen;

R₅ is NHR₆ or OH;

R₆ is H or C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that, if R₁ and R₂ are both independently an alkyl or (C₀-C₄ alkyl)(C₆-C₁₀ aryl)R₇, either R₁ or R₂ is linked through (CH₂)ₚ to R₅, wherein p is 2-9.

In one embodiment the dipeptide element is linked to a side chain amine of an internal amino acid of a bioactive peptide wherein the internal amino acid comprises the structure of Formula IV:

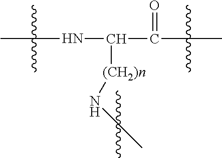

wherein

n is an integer selected from 1 to 4. In one embodiment n is 3 or 4 and in one embodiment the internal amino acid is lysine.

In a further embodiment the dipeptide element is linked to the bioactive peptide via an amine substituent of an aryl group present in the bioactive peptide. In one embodiment the amino group substituent is a primary amine. In those embodiments where the dipeptide element is linked to the medicinal agent via an amine substituent of an aryl group present in the medicinal agent, prodrugs having a t_{1/2} of about 1 hour have a dipeptide structure of:

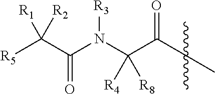

wherein R₁ and R₂ are independently C₁-C₁₈ alkyl or aryl;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₁₈ alkyl and aryl; and R₅ is an amine or a hydroxyl.

In some embodiments where the dipeptide element is linked to the medicinal agent via an amine substituent of an aryl group present in the medicinal agent, prodrugs having a t_{1/2} of about 1 hour have a dipeptide structure of:

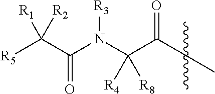

wherein R₁ and R₂ are independently C₁-C₁₈ alkyl or (C₀-C₄ alkyl)(C₆-C₁₀ aryl)R₇;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₁₈ alkyl and (C₀-C₄ alkyl)(C₆-C₁₀ aryl)R₇;

R₅ is NH₂ or OH; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

Furthermore, prodrugs having the dipeptide element linked to the medicinal agent via an amine substituent of an aryl group present in the medicinal agent, and having a t½ of about 6 to about 24 hours are provided wherein the dipeptide comprises a structure of:

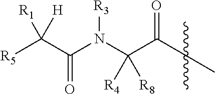

wherein

R₁ is selected from the group consisting of hydrogen, C₁-C₁₈ alkyl and aryl, or R₁ and R₂ are linked through —(CH₂)ₚ, wherein p is 2-9;

R₃ is C₁-C₁₈ alkyl or R₃ and R₄ together with the atoms to which they are attached form a 4-6 heterocyclic ring;

R₄ and R₈ are independently selected from the group consisting of hydrogen, C₁-C₁₈ alkyl and aryl; and R₅ is an amine or N-substituted amine.

In some embodiments, prodrugs having the dipeptide prodrug element linked via an aromatic amino acid and having a t_{1/2}, e.g., of about 6 to about 24 hours are provided wherein the dipeptide comprises a structure of:

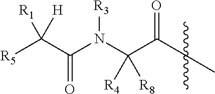

wherein

R₁ is selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇;

R₃ is C₁-C₁₈ alkyl or R₃ and R₄ together with the atoms to which they are attached form a 4-6 heterocyclic ring;

R₄ and R₈ are independently selected from the group consisting of hydrogen, C₁-C₁₈ alkyl and (C₀-C₄ alkyl)(C₆-C₁₀ aryl)R₇;

R₅ is NHR₆;

R₆ is H, C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In addition, prodrugs having the dipeptide element linked to the medicinal agent via an amine substituent of an aryl group present in the medicinal agent, and having a t½ of about 72 to about 168 hours are provided wherein the dipeptide comprises a structure of:

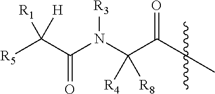

wherein R₁ and R₂ are independently selected from the group consisting of hydrogen, C₁-C₈ alkyl and aryl;

R₃ is C₁-C₁₈;

R₄ and R₈ are each hydrogen; and

R₅ is selected from the group consisting of amine, N-substituted amine and hydroxyl.

In some embodiments, prodrugs having the dipeptide prodrug element linked via an aromatic amino acid and having a t_{1/2}, e.g., of about 72 to about 168 hours are provided wherein the dipeptide comprises a structure of:

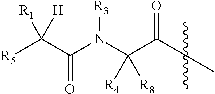

wherein R₁ and R₂ are independently selected from the group consisting of hydrogen, C₁-C₈ alkyl, (C₁-C₄ alkyl)COOH, and (C₀-C₄ alkyl)(C₆-C₁₀ aryl)R₇, or R₁ and R₅ together with the atoms to which they are attached form a 4-11 heterocyclic ring;

R₃ is C₁-C₁₈ alkyl or R₃ and R₄ together with the atoms to which they are attached form a 4-6 heterocyclic ring;

R₄ is hydrogen or forms a 4-6 heterocyclic ring with R₃;

R₈ is hydrogen;

R₅ is NHR₆ or OH;

R₆ is H or C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In one embodiment the dipeptide element is linked to a bioactive peptide via an amine present on an aryl group of an aromatic amino acid present in the bioactive peptide. In one embodiment the aromatic amino acid is an internal amino acid of the medicinal agent, however the aromatic amino acid can also be the N-terminal amino acid. In one embodiment the aromatic amino acid is selected from the group consisting of amino-Phe, amino-napthyl alanine, amino tryptophan, amino-phenyl-glycine, amino-homo-Phe, and amino tyrosine. In one embodiment the primary amine that forms an amide bond with the dipeptide element is in the para-position on the aryl group. In one embodiment the aromatic amine comprises the structure of Formula III:

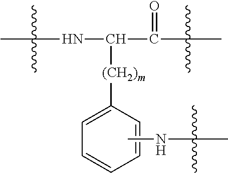

wherein m is an integer from 1 to 3.

In accordance with one embodiment the dipeptide element comprises the structure:

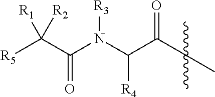

wherein R₁ is selected from the group consisting of H and C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, CH₂(C₅-C₉ heteroaryl), or R₁ and R₂ together with the atoms to which they are attached form a C₃-C₆ cycloalkyl;

R₃ is selected from the group consisting of C₁-C₈ alkyl, (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring; and

R₇ is selected from the group consisting of H and OH. In one embodiment R₁ is H or C₁-C₈ alkyl, R₂ is selected from the group consisting of H, C₁-C₆ alkyl, CH₂OH, (C₁-C₄ alkyl)NH₂, (C₃-C₆ cycloalkyl) and CH₂(C₆ aryl)R₇ or R₆ and R₂ together with the atoms to which they are attached form a 5 member heterocyclic ring, R₃ is C₁-C₆ alkyl, and R₄ is selected from the group consisting of H, C₁-C₄ alkyl, (C₃-C₆)cycloalkyl, (C₁-C₄ alkyl)OH, (C₁-C₄ alkyl)SH and (C₀-C₄ alkyl)(C₆ aryl)R₇, or R₃ and R₄ together with the atoms to which they are attached form a 5 member heterocyclic ring. In a further embodiment R₃ is CH₃, R₅ is NHR₆, and in an alternative further embodiment R₃ and R₄ together with the atoms to which they are attached form a 5 member heterocyclic ring and R₅ is NHR₆.

In accordance with other embodiments the dipeptide prodrug element comprises the structure:

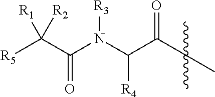

wherein R₁ is selected from the group consisting of H and C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, CH₂(C₅-C₉ heteroaryl), or R₁ and R₂ together with the atoms to which they are attached form a C₃-C₆ cycloalkyl;

R₃ is selected from the group consisting of C₁-C₈ alkyl, (C₃-C₆)cycloalkyl or R₄ and R₃ together with the atoms to which they are attached form a 5 or 6 member heterocyclic ring;

R₅ is NHR₆ or OH;

R₆ is H, or R₆ and R₂ together with the atoms to which they are attached form a 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo. In some embodiments R₁ is H or C₁-C₈ alkyl, R₂ is selected from the group consisting of H, C₁-C₆ alkyl, CH₂OH, (C₁-C₄ alkyl)NH₂, (C₃-C₆ cycloalkyl) and CH₂(C₆ aryl)R₇ or R₆ and R₂ together with the atoms to which they are attached form a 5 member heterocyclic ring, R₃ is C₁-C₆ alkyl, and R₄ is selected from the group consisting of H, C₁-C₄ alkyl, (C₃-C₆)cycloalkyl, (C₁-C₄ alkyl)OH, (C₁-C₄ alkyl)SH and (C₀-C₄ alkyl)(C₆ aryl)R₇, or R₃ and R₄ together with the atoms to which they are attached form a 5 member heterocyclic ring. In further embodiments R₃ is CH₃, R₅ is NHR₆, and in alternative further embodiments R₃ and R₄ together with the atoms to which they are attached form a 5 member heterocyclic ring and R₅ is NHR₆.

The following compounds are provided as examples of compounds that can be combined with the prodrug elements disclosed herein to form prodrug derivatives or sequestered complexes of the known drugs and bioactive peptides.

I. Glucocorticoids

Glucocorticoids, a class of corticosteroids, are endogenous hormones with profound effects on the immune system and multiple organ systems. They suppress a variety of immune and inflammatory functions by inhibition of inflammatory cytokines such as IL-1, IL-2, IL-6, and TNF, inhibition of arachidonic acid metabolites including pro staglandins and leukotrienes, depletion of T-lymphocytes, and reduction of the expression of adhesion molecules on endothelial cells (P. J. Barnes, Clin. Sci., 1998, 94, pp. 557-572; P. J. Barnes et al., Trends Pharmacol. Sci., 1993, 14, pp. 436-441). In addition to these effects, glucocorticoids stimulate glucose production in the liver and catabolism of proteins, play a role in electrolyte and water balance, reduce calcium absorption, and inhibit osteoblast function.

The effects of glucocorticoids are mediated at the cellular level by the glucocorticoid receptor (R. H. Oakley and J. Cidlowski, Glucocorticoids, N. J. Goulding and R. J. Flowers (eds.), Boston: Birkhauser, 2001, pp. 55-80). The glucocorticoid receptor is a member of a class of structurally related intracellular receptors that when coupled with a ligand can function as a transcription factor that affects gene expression (R. M. Evans, Science, 1988, 240, pp. 889-895). Other members of the family of steroid receptors include the mineralocorticoid, progesterone, estrogen, and androgen receptors.

The anti-inflammatory and immune suppressive activities of endogenous glucocorticoids have stimulated the development of synthetic glucocorticoid derivatives including dexamethasone, prednisone, and prednisolone (L. Parente, Glucocorticoids, N. J. Goulding and R. J. Flowers (eds.), Boston: Birkhauser, 2001, pp. 35-54). These have found wide use in the treatment of inflammatory, immune, and allergic disorders including rheumatic diseases such as rheumatoid arthritis, juvenile arthritis, and ankylosing spondylitis, dermatological diseases including psoriasis and pemphigus, allergic disorders including allergic rhinitis, atopic dermatitis, and contact dermatitis, pulmonary conditions including asthma and chronic obstructive pulmonary disease (COPD), and other immune and inflammatory diseases including Crohn's disease, ulcerative colitis, systemic lupus erythematosus, autoimmune chronic active hepatitis, osteoarthritis, tendonitis, and bursitis (J. Toogood, Glucocorticoids, N. J. Goulding and R. J. Flowers (eds.), Boston: Birkhauser, 2001, pp. 161-174). They have also been used to help prevent rejection in organ transplantation.

Novel ligands for the glucocorticoid receptor have been described in the scientific and patent literature. For example, PCT International Publication No. WO 99/33786 discloses triphenylpropanamide compounds with potential use in treating inflammatory diseases. PCT International Publication No. WO 00/66522 describes non-steroidal compounds as selective modulators of the glucocorticoid receptor potentially useful in treating metabolic and inflammatory diseases. PCT International Publication No. WO 99/41256 describes tetracyclic modulators of the glucocorticoid receptor potentially useful in treating immune, autoimmune, and inflammatory diseases. U.S. Pat. No. 5,688,810 describes various non-steroidal compounds as modulators of glucocorticoid and other steroid receptors. PCT International Publication No. WO 99/63976 describes a non-steroidal, liver-selective glucocorticoid antagonist potentially useful in the treatment of diabetes. PCT International Publication No. WO 00/32584 discloses non-steroidal compounds having anti-inflammatory activity with dissociation between anti-inflammatory and metabolic effects. PCT International Publication No. WO 98/54159 describes non-steroidal cyclically substituted acylanilides with mixed gestagen and androgen activity. U.S. Pat. No. 4,880,839 describes acylanilides having progestational activity and EP 253503 discloses acylanilides with antiandrogenic properties. PCT International Publication No. WO 97/27852 describes amides that are inhibitors of farnesyl-protein transferase.

In accordance with one embodiment a derivative of a glucocorticoid receptor agonist or antagonist is provided comprising the structure A-B-Q. In this embodiment, Q is the glucocorticoid receptor agonist or antagonist, A is an amino acid or a hydroxyl acid and B is an N-alkylated amino acid. A and B together represent the dipeptide element that is linked to Q through formation of an amide bond between A-B and an amine of Q. In one embodiment at least one of A or B is a non-coded amino acid. In accordance with one embodiment Q is selected from the group consisting of dexamethasone, prednisone, and prednisolone. Furthermore, in one embodiment the dipeptide element is selected wherein chemical cleavage of A-B from Q is at least about 90% complete within about 1 to about 720 hours in PBS under physiological conditions. In a further embodiment the amino acids of the dipeptide are selected wherein the cleavage half-life of A-B from Q in PBS under physiological conditions is not more than two to five fold the cleavage half-life of A-B from Q in a solution comprising a DPP-IV protease (including for example, human serum).

II. Thyroid Hormone

Thyroxine (T₄) is a thyroid hormone involved in the control of cellular metabolism. Chemically, thyroxine is an iodinated derivative of the amino acid tyrosine. The maintenance of a normal level of thyroxine is important for normal growth and development of children as well as for proper bodily function in the adult. Its absence leads to delayed or arrested development. Hypothyroidism, a condition in which the thyroid gland fails to produce enough thyroxine, leads to a decrease in the general metabolism of all cells, most characteristically measured as a decrease in nucleic acid and protein synthesis, and a slowing down of all major metabolic processes. Conversely, hyperthyroidism is an imbalance of metabolism caused by overproduction of thyroxine.

During metabolism, T4 is converted to T3 or to rT3 via removal of an iodine atom from one of the hormonal rings. T3 is the biologically active thyroid hormone, whereas rT3 has no biological activity. Both T3 and T4 are used to treat thyroid hormone deficiency (hypothyroidism). They are both absorbed well by the gut, so can be given orally.

In accordance with one embodiment a thyroid hormone derivative is provided comprising the structure A-B-Q. In this embodiment, Q is the thyroid hormone, A is an amino acid or a hydroxyl acid and B is an N-alkylated amino acid. A and B together represent the dipeptide element that is linked to Q through formation of an amide bond between A-B and an amine of Q. In one embodiment at least one of A, B, or the amino acid of Q to which A-B is linked, is a non-coded amino acid. In accordance with one embodiment Q is selected from the group consisting of thyroxine T4 (3,5,3′,5′-tetraiodothyronine), 3,5,3′-triiodo L-thyronine and 3,3′,5′-triiodo L-thyronine. In one embodiment the dipeptide element is linked via an amide bond through the primary amine of 3,5,3′,5′-tetraiodothyronine or 3,5,3′-triiodo L-thyronine. Furthermore, in one embodiment the dipeptide element is selected wherein chemical cleavage of A-B from Q is at least about 90% complete within about 1 to about 720 hours in PBS under physiological conditions. In a further embodiment the amino acids of the dipeptide are selected wherein the cleavage half-life of A-B from Q in PBS under physiological conditions is not more than two to five fold the cleavage half-life of A-B from Q in a solution comprising a DPP-IV protease (including for example, human serum).

III. Anti-Cancer Agents

Numerous antitumor drugs possess a limited bioavailability due to low chemical stability, a limited oral absorption, or a rapid breakdown in vivo (i.e., by first-pass metabolism). To overcome these problems, various prodrugs that can be activated into antitumor drugs have been designed. In this case it is preferred if prodrugs are activated relatively slowly in the blood or liver, for example, thereby preventing acute toxic effects due to high peak concentrations of the antitumor drug. An ideal prodrug designed to increase the bioavailability of an antitumor drug is slowly released. In one embodiment the prodrug is targeted to tumor cells by complexing the prodrug with a tumor specific ligand or antibody. In one embodiment the anti-cancer drugs is selected from the group consisting of taxanes, such as paclitaxel or taxotere; camptothecins, such as camptothecin, CPT 11, irinotecan, topotecan or HCl; podophyllotoxins, such as teniposide; vinblastine sulfate; vincristine sulfate; vinorelbine tartrate; procarbazine HCl; cladribine, leustatin; hydroxyurea; gemcitabine HCl; leuprolide acetate; thioguanine; purinethol; florouricil; anthracyclines, such as daunorubicin or doxorubicin (adriamycin); methotrexates; p-aminoaniline mustard; cytarabine (ara-C or cytosine arabinoside); etoposide; bleomycin sulfate; actinomycin D; idarubicin HCl; mitomycin; plicamycin; mitoxantrone HCl; pentostatin; streptozocin; L-phenylalanine mustard; carboplatin derivatives; platinol; busulfan; fluconazole; amifostine; leucovorin calcium and octreotide acetate.

In accordance with one embodiment a known anti-cancer agent derivative is provided comprising the structure A-B-Q. In this embodiment, Q is the anti-cancer agent, A is an amino acid or a hydroxyl acid and B is an N-alkylated amino acid. A and B together represent the dipeptide element that is linked to Q through formation of an amide bond between A-B and an amine of Q. In one embodiment at least one of A, B, or the amino acid of Q to which A-B is linked, is a non-coded amino acid.

IV. Antibiotics

The present invention also provides novel methods of administering compositions and formulations comprising derivatives of known antibiotics. The methods provide compositions of active compounds that, if presented in presently available forms, may result in toxicity to the treated mammal. Thus, the formulations and methods of the present invention enable one to administer compounds that previously have not been able to be widely used in particular species due to safety considerations. The methods also enable one to extend the release times of compounds and provide a controlled dose of active compound to the treated patient.

In accordance with one embodiment a prodrug derivative of a known antibiotic is provided. In accordance with one embodiment the antibiotic is selected from the group consisting of oxytetracycline, doxycycline, fluoxetime, roxithromycin, terbinarefine, or metoprolol.

Oxytetracycline is a widely used and useful antibiotic for treating various infections in mammals. In particular it is used for treating and preventing respiratory infections in domestic animals. There are significant costs associated with repeated administrations through conventional means. In accordance with one embodiment a dipeptide element A-B is covalently linked to an antibiotic, including for example, oxytetracycline, wherein the complex optionally further includes a depot polymer.

V. Additional Bioactive Compounds Suitable for Linkage to the Dipeptide Element

Additional compounds can be linked to the dipeptide element disclosed herein to form prodrug derivatives or depot derivatives of the compounds. These additional compounds include growth factors, both natural and recombinant, as well as peptide fractions of growth factors that bind to receptors on the cell surface (EGF, VEGF, FGF, ILGF-I, ILGF-II, TGF). Prodrug derivatives of interferons both natural or recombinant (including IFN-alpha, beta, and gamma) and interferon agonists; and prodrug derivatives of cytokines, either natural or recombinant, including for example (IL-1, IL-2, IL-3, IL-4, IL-5, IL-6, IL-7, IL-8, IL-9, IL-10, IL-12, IL-15, TNF, etc) are also encompassed within the scope of the present invention. In accordance with one embodiment any peptide, natural, recombinant, or synthetic that binds to a cell surface receptor can be modified to by linking the dipeptide element disclosed herein to form a prodrug or depot derivative of that peptide.

In accordance with one embodiment the dipeptide element can be attached via an amide linkage to any of the bioactive compounds previously disclosed in International application no. PCT/US2008/053857 (filed on Feb. 13, 2008), the disclosure of which is hereby expressly incorporated by reference into the present application. The dipeptide element disclosed herein can be linked to the bioactive peptides disclosed in PCT/US2008/053857 either through the N-terminal amine or to the side chain amino group of a lysine at position 20 or the aromatic amino group of a 4-amino phenylalanine substituted for the amino acid at position 22 of any of the disclosed bioactive peptides. In one embodiment the dipeptide element disclosed herein is linked via an amide bond to the N-terminal amine of a bioactive peptide disclosed in PCT/US2008/053857.

In accordance with one embodiment a complex comprising a medicinal agent and a dipeptide element, A-B, is provided. In one embodiment the dipeptide A-B comprises the structure:

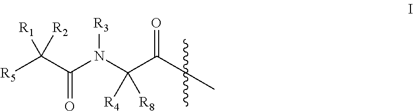

wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl or aryl;

R₅ is NHR₆; and

R₆ is H or C₁-C₈ alkyl.

In some embodiments the dipeptide A-B comprises the structure:

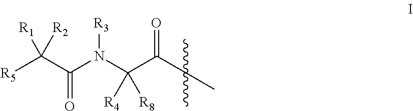

wherein

R₁ and R₈ are independently H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl;

R₃ is C₁-C₁₈ alkyl;

R₅ is NHR₆;

R₆ is H or C₁-C₈ alkyl; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In one embodiment the dipeptide A-B is linked via an amide bond to an aliphatic amino acid of a compound “Q” as defined herein.

In accordance with one embodiment the dipeptide of formula I is provided wherein

R₁ and R₂ are independently C₁-C₁₈ alkyl or aryl; or R₁ and R₂ are linked through —(CH₂)ₚ—, wherein p is 2-9;

R₃ is C₁-C₁₈ alkyl;

R₄ and R₈ are each hydrogen; and

R₅ is an amine.

In some embodiments, the dipeptide A-B comprises the structure:

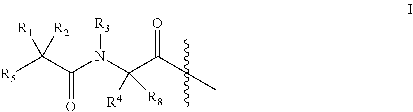

wherein

R₁ and R₂ are independently C₁-C₁₈ alkyl or (C₀-C₄ alkyl)(C₆-C₁₀ aryl)R₇; or R₁ and R₂ are linked through —(CH₂)ₚ, wherein p is 2-9;

R₃ is C₁-C₁₈ alkyl;

R₄ and R₈ are each hydrogen;

R₅ is NH₂; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In an alternative embodiment A-B comprises the structure of formula I wherein

R₁ and R₂ are independently selected from the group consisting of hydrogen, C₁-C₁₈ alkyl and aryl, or R₁ and R₂ are linked through —(CH₂)ₚ—, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and aryl; and

R₅ is an amine; with the proviso that both R₁ and R₂ are not hydrogen and provided that one of R₄ or R₈ is hydrogen.

In some embodiments, the dipeptide A-B comprises the structure:

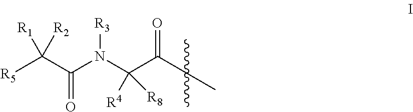

wherein R₁ and R₂ are independently selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇, or R₁ and R₂ are linked through (CH₂)ₚ, wherein p is 2-9;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₈ alkyl and (C₀-C₄ alkyl)(C₆-C₁₀ aryl)R₇;

R₅ is NH₂; and

R₇ is selected from the group consisting of H, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that both R₁ and R₂ are not hydrogen and provided that at least one of R₄ or R₈ is hydrogen.

In another embodiment a dipeptide element of formula I is provided, wherein

R₁ is selected from the group consisting of hydrogen, C₁-C₈ alkyl and aryl;

R₃ is C₁-C₁₈ alkyl;

R₄ and R₈ are each hydrogen; and

R₅ is an amine or N-substituted amine or a hydroxyl;

with the proviso that, if R₁ is alkyl or aryl, then R₁ and R₅ together with the atoms to which they are attached form a 4-11 heterocyclic ring.

In some embodiments, a dipeptide element is provided:

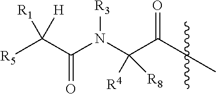

wherein R₁ is selected from the group consisting of hydrogen, C₁-C₁₈ alkyl and (C₀-C₄ alkyl)(C₆-C₁₀ aryl)R₇;

R₃ is C₁-C₁₈ alkyl;

R₄ and R₈ are each hydrogen;

R₅ is NHR₆ or OH;

R₆ is H or C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo;

with the proviso that, if R₁ is alkyl or (C₀-C₄ alkyl)(C₆-C₁₀ aryl)R₇, R₁ is linked through (CH₂)ₚ to R₅, wherein p is 2-9.

In some embodiments, a dipeptide element is provided:

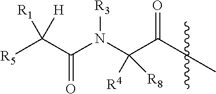

wherein R₁ and R₂ are independently selected from the group consisting of hydrogen, C₁-C₈ alkyl and (C₁-C₄ alkyl)NH₂, or R₁ and R₂ are linked through (CH₂)ₚ, wherein p is 2-9;

R₃ is C₁-C₈ alkyl or R₃ and R₄ together with the atoms to which they are attached form a 4-6 heterocyclic ring;

R₄ is selected from the group consisting of hydrogen and C₁-C₈ alkyl; and

R₅ is NH₂;

with the proviso that both R₁ and R₂ are not hydrogen.

In some embodiments, a dipeptide element is provided:

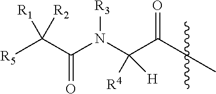

wherein R₁ and R₂ are independently selected from the group consisting of hydrogen, C₁-C₈ alkyl and (C₁-C₄ alkyl)NH₂;

R₃ is C₁-C₆ alkyl;

R₄ is hydrogen; and

R₅ is NH₂;

with the proviso that both R₁ and R₂ are not hydrogen.

In some embodiments, a dipeptide element is provided:

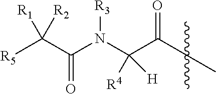

wherein

R₁ and R₂ are independently selected from the group consisting of hydrogen and C₁-C₈ alkyl, (C₁-C₄ alkyl)NH₂, or R₁ and R₂ are linked through (CH₂)ₚ, wherein p is 2-9;

R₃ is C₁-C₈ alkyl;

R₄ is (C₀-C₄ alkyl)(C₆-C₁₀ aryl)R₇;

R₅ is NH₂; and

R₇ is selected from the group consisting of hydrogen, C₁-C₈ alkyl and (C₀-C₄ alkyl)OH;

with the proviso that both R₁ and R₂ are not hydrogen.

In another embodiment the dipeptide element (A-B) is linked via an amide bond to an amine substituent on an aryl group of Q of the complex A-B-Q. In one embodiment where the dipeptide element comprises the structure of formula I linked via an amide bond to an amine substituent on an aryl,

R₁ and R₂ are independently C₁-C₁₈ alkyl or aryl;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₁₈ alkyl and aryl; and

R₅ is an amine or a hydroxyl.

In other embodiments, the dipeptide element comprises the structure of formula I linked via an amide bond to an amine substituent on an aryl,

wherein R₁ and R₂ are independently C₁-C₁₈ alkyl or (C₀-C₄ alkyl)(C₆-C₁₀ aryl)R₇;

R₃ is C₁-C₁₈ alkyl or R₃ and R₄ together with the atoms to which they are attached form a 4-12 heterocyclic ring;

R₄ and R₈ are independently selected from the group consisting of hydrogen, C₁-C₁₈ alkyl and (C₀-C₄ alkyl)(C₆-C₁₀ aryl)R₇;

R₅ is NH₂ or OH; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In another embodiment A-B comprises the structure of formula I linked via an amide bond to an amine substituent on an aryl of Q of the complex A-B-Q, wherein

R₁ is selected from the group consisting of hydrogen, C₁-C₁₈ alkyl and aryl, or R₁ and R₂ are linked through —(CH₂)ₚ—, wherein p is 2-9;

R₃ is C₁-C₁₈ alkyl or R₃ and R₄ together with the atoms to which they are attached form a 4-6 heterocyclic ring;

R₄ and R₈ are independently selected from the group consisting of hydrogen, C₁-C₁₈ alkyl and aryl; and

R₅ is an amine or N-substituted amine.

In other embodiments, the dipeptide element comprises the structure of formula I linked via an amide bond to an amine substituent on an aryl of Q of the complex A-B-Q, wherein

R₁ is selected from the group consisting of hydrogen, C₁-C₁₈ alkyl, (C₁-C₁₈ alkyl)OH, (C₁-C₄ alkyl)NH₂, and (C₀-C₄ alkyl)(C₆-C₁₀ aryl)R₇;

R₃ is C₁-C₁₈ alkyl or R₃ and R₄ together with the atoms to which they are attached form a 4-6 heterocyclic ring;

R₄ and R₈ are independently selected from the group consisting of hydrogen, C₁-C₁₈ alkyl and (C₀-C₄ alkyl)(C₆-C₁₀ aryl)R₇;

R₅ is NHR₆;

R₆ is H, C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In another embodiment the dipeptide element (A-B) comprises the structure of formula I linked via an amide bond to an amine substituent on an aryl of Q of the complex A-B-Q, wherein

R₁ and R₂ are independently selected from the group consisting of hydrogen, C₁-C₈ alkyl and aryl;

R₃ is C₁-C₁₈ alkyl or R₃ and R₄ together with the atoms to which they are attached form a 4-6 heterocyclic ring;

R₄ and R₈ are each hydrogen; and

R₅ is selected from the group consisting of amine, N-substituted amine and hydroxyl.

In other embodiments, the dipeptide element is linked via an amide bond to an amine substituent on an aryl and comprises the structure:

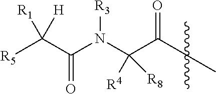

wherein R₁ and R₂ are independently selected from the group consisting of hydrogen, C₁-C₈ alkyl, (C₁-C₄ alkyl)COOH, and (C₀-C₄ alkyl)(C₆-C₁₀ aryl)R₇, or R₁ and R₅ together with the atoms to which they are attached form a 4-11 heterocyclic ring;

R₃ is C₁-C₁₈ alkyl or R₃ and R₄ together with the atoms to which they are attached form a 4-6 heterocyclic ring;

R₄ and R₈ are each hydrogen;

R₅ is NHR₆ or OH;

R₆ is H or C₁-C₈ alkyl, or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In accordance with one embodiment Q is a medicinal agent and in one embodiment Q is a compound selected from the group consisting of thyroxine T4 (3,5,3′,5′-tetraiodothyronine), 3,5,3′-triiodo L-thyronine and 3,3′,5′-triiodo L-thyronine. In one embodiment the dipeptide/drug complex comprises the structure of Formula II;

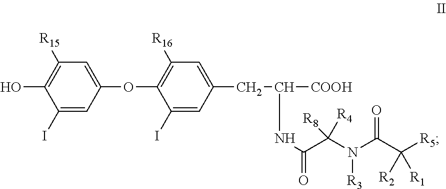

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring;

R₇ is selected from the group consisting of H and OH;

R₁₅ and R₁₆ are independently selected from hydrogen and iodine.

In other embodiments the dipeptide/drug complex comprises the structure of Formula II;

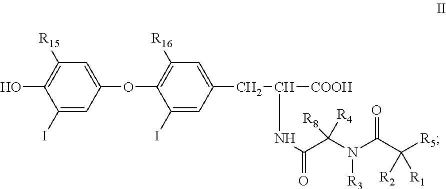

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring;

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo; and

R₁₅ and R₁₆ are independently selected from hydrogen and iodine.

In accordance with one embodiment a compound of Formula II is provided wherein
R₁ is selected from the group consisting of H and C₁-C₈ alkyl;
R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₆-C₁₀ aryl)R₇, CH₂(C₅-C₉ heteroaryl), or R₁ and R₂ together with the atoms to which they are attached form a C₃-C₆ cycloalkyl;
R₃ is selected from the group consisting of C₁-C₈ alkyl, (C₁-C₄ alkyl)OH, (C₁-C₄ alkyl)NH₂, (C₁-C₄ alkyl)SH, (C₃-C₆)cycloalkyl or R₄ and R₃ together with the atoms to which they are attached form a 5 or 6 member heterocyclic ring;
R₅ is NHR₆ or OH;
R₆ is H, or R₆ and R₂ together with the atoms to which they are attached form a 5 or 6 member heterocyclic ring; and
R₇ is selected from the group consisting of H and OH; and
R₈ is H, with the proviso that when R₄ and R₃ together with the atoms to which they are attached form a 5 or 6 member heterocyclic ring, at least one of R₁ and R₂ are not H, and in one embodiment both R₁ and R₂ are other than H.

In accordance with other embodiments a compound of Formula II is provided wherein

R₁ is H or C₁-C₈ alkyl;

R₂ and R₄ are independently selected from the group consisting of H, C₁-C₈ alkyl, C₂-C₈ alkenyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+) NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, and CH₂(C₃-C₉ heteroaryl), or R₁ and R₂ together with the atoms to which they are attached form a C₃-C₁₂ cycloalkyl;

R₃ is C₁-C₁₈ alkyl; (C₁-C₄ alkyl)OH, (C₁-C₄ alkyl)NH₂, (C₁-C₄ alkyl)SH, (C₃-C₆)cycloalkyl or R₄ and R₃ together with the atoms to which they are attached form a 5 or 6 member heterocyclic ring;

R₅ is NHR₆ or OH;

R₆ is H or R₆ and R₂ together with the atoms to which they are attached form a 5 or 6 member heterocyclic ring;

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo; and

R₈ is H, with the proviso that when R₄ and R₃ together with the atoms to which they are attached form a 5 or 6 member heterocyclic ring, at least one of R₁ and R₂ are not H, and in one embodiment both R₁ and R₂ are other than H.

Any of the complexes disclosed herein can be further modified to improve the peptide's solubility in aqueous solutions at physiological pH, while enhancing the effective duration of the peptide by preventing renal clearance of the peptide. Increasing the molecular weight of a medicinal agent above 40 kDa exceeds the renal threshold and significantly extends duration in the plasma. Accordingly, in one embodiment the peptide prodrugs are further modified to comprise a covalently linked hydrophilic moiety. In one embodiment the hydrophilic moiety is a plasma protein, polyethylene glycol chain or the Fc portion of an immunoglobin. Therefore, in one embodiment the presently disclosed complexes are further modified to comprise one or more hydrophilic groups covalently linked to the side chain of the dipeptide element A-B, or optional to other amino acid side chains when the medicinal agent is a bioactive peptide.

In accordance with some embodiments, the dipeptide/drug complexes are modified to comprise an acyl group or alkyl group. Acylation or alkylation can increase the half-life of the drug in circulation. Acylation or alkylation can advantageously delay the onset of action and/or extend the duration of action at the drugs target receptor and/or improve resistance to proteases such as DPP-IV. Acylation may also enhance solubility of the dipeptide/drug complex at neutral pH. In one embodiment an amino acid of the dipeptide element A-B is acylated.

The acyl group can be covalently linked directly to the medicinal agent, or indirectly to the medicinal agent via a spacer, wherein the spacer is positioned between the medicinal agent and the acyl group. In some embodiments wherein the medicinal agent comprises an amino acid, the medicinal agent is acylated through the side chain amine, hydroxyl, or thiol of an amino acid of the medicinal agent. Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmaceutical Res. “Synthesis of Palmitoyl Derivatives of Insulin and their Biological Activity” Vol. 6, No: 2 pp. 171-176 (1989).

The acyl group of the acylated medicinal agent can be of any size, e.g., any length carbon chain, and can be linear or branched. In some specific embodiments of the invention, the acyl group is a C4 to C28 fatty acid. For example, the acyl group can be any of a C4 fatty acid, C6 fatty acid, C8 fatty acid, C10 fatty acid, C12 fatty acid, C14 fatty acid, C16 fatty acid, C18 fatty acid, C20 fatty acid, C22 fatty acid, C24 fatty acid, C26 fatty acid, or a C28 fatty acid. In some embodiments, the acyl group is a C8 to C20 fatty acid, e.g., a C14 fatty acid or a C16 fatty acid. In some embodiments, the acyl group is a fatty acid or bile acid, or salt thereof, e.g. a C4 to C30 fatty acid, a C8 to C24 fatty acid, cholic acid, a C4 to C30 alkyl, a C8 to C24 alkyl, or an alkyl comprising a steroid moiety of a bile acid.

In one embodiment the amino acid at the position of the dipeptide element A-B where the hydrophilic moiety is to be linked is selected to allow for ease in attaching the hydrophilic moiety. For example, the dipeptide element may comprise a lysine or cysteine residue to allow for the covalent attachment of a polyethylene glycol chain.

In one embodiment the dipeptide/drug complex has a single cysteine residue, present in the dipeptide element A-B, wherein the side chain of the cysteine residue is further modified with a thiol reactive reagent, including for example, maleimido, vinyl sulfone, 2-pyridylthio, haloalkyl, and haloacyl. These thiol reactive reagents may contain carboxy, keto, hydroxyl, and ether groups as well as other hydrophilic moieties such as polyethylene glycol units. In an alternative embodiment, the complex has a single lysine residue, present in the dipeptide element A-B, and the side chain of the substituting lysine residue is further modified using amine reactive reagents such as active esters (succinimido, anhydride, etc) of carboxylic acids or aldehydes of hydrophilic moieties such as polyethylene glycol.

In those embodiments wherein the dipeptide/drug complex comprises a polyethylene glycol chain, the polyethylene glycol chain may be in the form of a straight chain or it may be branched. In accordance with one embodiment the polyethylene glycol chain has an average molecular weight selected from the range of about 20,000 to about 60,000 Daltons. Multiple polyethylene glycol chains can be linked to the prodrugs to provide a prodrug with optimal solubility and blood clearance properties. In one embodiment the dipeptide/drug complex is linked to a single polyethylene glycol chain that has an average molecular weight selected from the range of about 20,000 to about 60,000 Daltons. In another embodiment the dipeptide/drug complex is linked to a two polyethylene glycol chains wherein the combined average molecular weight of the two chains is selected from the range of about 40,000 to about 80,000 Daltons. In one embodiment a single polyethylene glycol chain having an average molecular weight of 20,000 or 60,000 Daltons is linked to the dipeptide/drug complex. In another embodiment a single polyethylene glycol chain is linked to the dipeptide/drug complex and has an average molecular weight selected from the range of about 40,000 to about 50,000 Daltons. In one embodiment two polyethylene glycol chains are linked to the dipeptide/drug complex wherein the first and second polyethylene glycol chains each have an average molecular weight of 20,000 Daltons. In another embodiment two polyethylene glycol chains are linked to the dipeptide/drug complex wherein the first and second polyethylene glycol chains each have an average molecular weight of 40,000 Daltons.

In accordance with one embodiment, a medicinal prodrug analog is provided wherein a plasma protein has been covalently linked to an amino acid side chain of the dipeptide element, or optionally to another amino acid side chain when the medicinal agent is a bioactive peptide, to improve the solubility, stability and/or pharmacokinetics of the prodrug. For example, one or more serum albumins can be covalently bound, or non-covalently bound via a high affinity association (e.g. via a C16-C18 acylated amino acid side chain) to the dipeptide/medicinal agent complex.

In accordance with one embodiment, a dipeptide/medicinal agent complex is provided wherein a linear amino acid sequence representing the Fc portion of an immunoglobin molecule has been covalently linked to an amino acid side chain of the dipeptide element, or optionally to another amino acid side chain when the medicinal agent is a bioactive peptide, to improve the solubility, stability and/or pharmacokinetics of the prodrug. The Fc portion is typically one isolated from IgG, but the Fc peptide fragment from any immunoglobin should function equivalently.

The present disclosure also encompasses other conjugates in which prodrugs of the invention are linked, optionally via covalent bonding and optionally via a linker, to a conjugate moiety. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

Exemplary conjugates include but are not limited to a heterologous peptide or polypeptide (including for example, a plasma protein), a targeting agent, an immunoglobulin or portion thereof (e.g. variable region, CDR, or Fc region), a diagnostic label such as a radioisotope, fluorophore or enzymatic label, a polymer including water soluble polymers, or other therapeutic or diagnostic agents. In one embodiment a conjugate is provided comprising a prodrug of the present invention and a plasma protein, wherein the plasma protein is selected form the group consisting of albumin, transferin and fibrinogen. In one embodiment the plasma protein moiety of the conjugate is albumin or transferin. In embodiments comprising a linker, the linker may comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary linkers are from about 1 to 50 amino acids in length, 5 to 50, 3 to 5, 5 to 10, 5 to 15, or 10 to 30 amino acids in length. Such fusion proteins may alternatively be produced by recombinant genetic engineering methods known to one of ordinary skill in the art.

In accordance with some embodiments the dipeptide prodrug element can be further modified to comprise a hydrophilic moiety. In some embodiments the hydrophilic moiety is a polyethylene glycol chain. In accordance with some embodiments a polyethylene glycol chain of 40 k or higher is covalently bound to the side chain of the A or B amino acid of the dipeptide prodrug element. In another embodiment the dipeptide prodrug element is additionally or alternatively acylated or alkylated with a fatty acid or bile acid, or salt thereof, e.g. a C4 to C30 fatty acid, a C8 to C24 fatty acid, cholic acid, a C4 to C30 alkyl, a C8 to C24 alkyl, or an alkyl comprising a steroid moiety of a bile acid. The ‘A’ amino acid of the dipeptide prodrug element can include, for example, d-lysine covalently bound to an acyl or alkyl group through its side chain amino group, or d-cysteine covalently bound to a PEG molecule through its side chain sulfhydryl group. The dipeptide prodrug element can be directly bound to the hydrophilic moiety, acyl group, or alkyl group, or bound to the hydrophilic moiety, acyl group, or alkyl group through a spacer, as described herein. Alternatively, the dipeptide prodrug element can be linked to a depot protein such as dextran or a large PEG molecule (greater or equal to 80,000 daltons) that serves to sequester the prodrug at an injection site until cleavage of the dipeptide releases the active bioactive peptide

### Effect of Dipeptide Prodrug Element Structure on Cleavage Rate

As previously described herein, the rate of cleavage of the dipeptide prodrug element A-B from the bioactive peptide, and thus activation of the prodrug, depends on the structure (including N-alkylation, number of substituents, length or bulkiness), and stereochemistry of the amino acids of the dipeptide prodrug element. The rate of cleavage of the dipeptide prodrug element A-B from the bioactive peptide also depends on the steric hindrance, nucleophilicity, and stability of the leaving group of Q during diketopiperazine formation. Some of these structural features are described in Category I, Category I I, and Category III below, which form part of the invention. Explicitly excluded from any of these categories are peptide sequences disclosed in Int'l Application No. PCT/US2009/68745, filed Dec. 18, 2009 or its sequence listing, and sub-categories of (1) dipeptide prodrug elements, (2) A amino acids, and/or (3) B amino acids disclosed in Int'l Application No. PCT/US2009/68745, filed Dec. 18, 2009, to the extent they fall completely within and/or overlap with a portion of any of the sub-categories described herein, and only to the extent necessary to confer novelty on claimed subject matter.

### Category I. Composition of Amino Acid B of the Dipeptide Prodrug Element

In some embodiments, the half-life of the prodrug, e.g., the chemical cleavage half-life (t_{1/2}) of A-B from Q of at least about 1 hour to about 1 week in PBS, under physiological conditions, is dependent on the presence and length of the N-alkyl substituent on the B amino acid. For example, a prodrug that has a shorter N-alkyl substituent on the B amino acid (e.g. Gly(N-methyl)), will undergo a slower rate of cleavage of A-B, and have a longer half-life, than a prodrug that has a longer N-alkyl substituent on the B amino acid (e.g., Gly(N-hexyl)).

In some embodiments, the half-life of the prodrug is dependent on the presence or absence of an alkyl side chain, and the degree of substitution at the beta position of the alkyl side chain, of the B amino acid of the dipeptide prodrug element. For example, a prodrug that has an N-alkylated B amino acid that is disubstituted at the beta position (e.g., N-alkylated isoleucine) will undergo slower cleavage of A-B, and have a longer half-life, than a prodrug that has an N-alkylated B amino acid that is monosubstituted at the beta position (e.g., N-alkylated leucine). Further, a prodrug that has an N-alkylated B amino acid that is monosubstituted at the beta position (e.g., N-alkylated leucine) will undergo slower cleavage of A-B, and have a longer half-life, than a prodrug that has an N-alkylated B amino acid that is unsubstituted at the beta position (e.g., N-alkylated alanine). Further still, a prodrug with an N-alkylated B amino acid that has an unsubstituted beta position (e.g., N-alkylated alanine) will undergo slower cleavage of A-B, and have a longer half-life, than a prodrug that has glycine or N-alkylated glycine as the B amino acid.

In some embodiments, the half-life of the prodrug is dependent on the bulkiness of the side chain of the B amino acid. For example, a prodrug that has a bulkier side chain on the B amino acid (e.g., N-alkylated phenylalanine), will undergo slower cleavage of A-B, and have a longer half-life, than a prodrug that has a less bulky side chain on the B amino acid (e.g., N-alkylated alanine). Cleavage rates of dipeptides can be further differentiated by the amine of the drug (e.g., insulin) to which they are attached. More particularly the same dipeptide will cleave at a faster rate when linked to an aromatic amine relative to an N-terminal amine, where the dipeptide linked to an N-terminal amine will cleave at a faster rate relative to when the dipeptide is linked to the side chain amine of a lysine residue.

The composition of the B amino acid of the dipeptide prodrug element can be classified into the below sub-categories IA, IB, and IC. Generally, the dipeptide prodrug elements in sub-category IA undergo cleavage the fastest and the dipeptide prodrug elements in sub-category IC undergo cleavage the slowest.

Sub-Category IA: Amino Acid B of the Dipeptide Prodrug Element is N-Alkylated Glycine

In some embodiments, the prodrug comprises the structure:

A-B-Q;

wherein Q is a bioactive peptide (e.g., an insulin peptide);

wherein A-B comprises the structure:

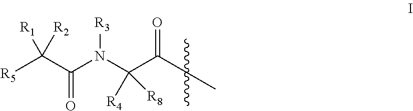

wherein

R₁ and R₂ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl;

R₃ is C₁-C₁₈ alkyl;

R₄ and R₈ are each H;

R₅ is NHR₆;

R₆ is H or C₁-C₄ alkyl, or R₅ and R₂ together with the atoms to which they are attached form a 4, 5 or 6 member heterocyclic ring; and,

R₇ is selected from the group consisting of H and OH.

In some embodiments, the B amino acid is selected from the group consisting of glycine(N-methyl), glycine(N-ethyl), glycine(N-propyl), glycine(N-butyl), glycine(N-pentyl), glycine(N-hexyl), glycine(N-heptyl), and glycine(N-octyl). For example, the B amino acid can be glycine(N-methyl) or glycine(N-hexyl).

In some embodiments when R₁ and R₂ are both hydrogen, R₃ is C₁-C₄ alkyl. In some embodiments when one of R₁ or R₂ is other than hydrogen, R₃ is C₁-C₄ alkyl.

Sub-Category IB: Amino Acid B of the Dipeptide Prodrug Element is Unsubstituted or Monosubstituted at the Beta Position

In some embodiments, the prodrug comprises the structure:

A-B-Q;

wherein Q is a bioactive peptide (e.g., an insulin peptide);

wherein A-B comprises the structure:

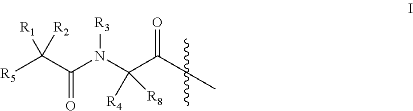

wherein

R₁ and R₂ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl;

R₃ is C₁-C₁₈ alkyl;

R₄ is selected from the group consisting of CH₃, CH₂(C₁-C₁₀ alkyl), CH₂(C₂-C₁₀ alkenyl), CH₂(C₀-C₁₀ alkyl)OH, CH₂(C₀-C₁₀ alkyl)SH, CH₂(C₀-C₃ alkyl)SCH₃, CH₂(C₀-C₃ alkyl)CONH₂, CH₂(C₀-C₃ alkyl)COOH, CH₂(C₀-C₃ alkyl)NH₂, CH₂(C₀-C₃ alkyl)NHC(NH₂⁺)NH₂, CH₂(C₀-C₃ alkyl)(C₃-C₆ cycloalkyl), CH₂(C₀-C₃ alkyl)(C₂-C₅ heterocyclic), CH₂(C₀-C₃ alkyl)(C₆-C₁₀ aryl)R₇, CH₂(C₁-C₃ alkyl)(C₃-C₉ heteroaryl), and CH₂(C₀-C₁₂ alkyl)(W₁)C₁-C₁₂ alkyl, wherein W₁ is a heteroatom selected from the group consisting of N, S and O; or R₄ and R₃ together with the atoms to which they are attached form a 4, 5 or 6 member heterocyclic ring;

R₈ is H,

R₅ is NHR₆, or R₅ and R₂ together with the atoms to which they are attached form a 4, 5 or 6 member heterocyclic ring;

R₆ is H or C₁-C₄ alkyl; and,

R₇ is selected from the group consisting of H and OH.

In some embodiments, R₄ is selected from the group consisting of CH₃, CH₂(C₁-C₄ alkyl), CH₂(C₁-C₄) alkenyl, CH₂(C₀-C₄ alkyl)OH, CH₂(C₀-C₄ alkyl)SH, CH₂(C₀-C₃ alkyl)SCH₃, CH₂(C₀-C₃ alkyl)CONH₂, CH₂(C₀-C₃ alkyl)COOH, CH₂(C₀-C₄ alkyl)NH₂, and CH₂(C₀-C₃ alkyl)NHC(NH₂⁺)NH₂.

Nonlimiting examples of the B amino acid in these embodiments include alanine(N—C₁-C₁₀alkyl), leucine(N—C₁-C₁₀alkyl), methionine(N—C₁-C₁₀alkyl), asparagine(N—C₁-C₁₀alkyl), glutamic acid(N—C₁-C₁₀alkyl), aspartic acid(N—C₁-C₁₀ alkyl), glutamine(N—C₁-C₁₀alkyl), histidine(N—C₁-C₁₀alkyl), lysine(N—C₁-C₁₀alkyl), arginine(N—C₁-C₁₀alkyl), serine(N—C₁-C₁₀alkyl), and cysteine(N—C₁-C₁₀alkyl).

In some embodiments, the B amino acid is selected from the group consisting of alanine(N—C₁-C₆alkyl), leucine(N—C₁-C₆alkyl), methionine(N—C₁-C₆alkyl), asparagine(N—C₁-C₆alkyl), glutamic acid(N—C₁-C₆alkyl), aspartic acid(N—C₁-C₆ alkyl), glutamine(N—C₁-C₆alkyl), histidine(N—C₁-C₆alkyl), lysine(N—C₁-C₆alkyl), arginine(N—C₁-C₆alkyl), serine(N—C₁-C₆alkyl), and cysteine(N—C₁-C₆alkyl).

For example, the B amino acid can include alanine(N-methyl), leucine(N-methyl), methionine(N-methyl), asparagine(N-methyl), glutamic acid(N-methyl), aspartic acid(N-methyl), glutamine(N-methyl), histidine(N-methyl), lysine(N-methyl), arginine(N-methyl), serine(N-methyl), and cysteine(N-methyl).

In some embodiments, R₄ is selected from the group consisting of CH₂(C₀-C₃ alkyl)(C₃-C₆ cycloalkyl), CH₂(C₀-C₃ alkyl)(C₂-C₅ heterocyclic), CH₂(C₀-C₃ alkyl)(C₆-C₁₀ aryl)R₇, CH₂(C₁-C₃ alkyl)(C₃-C₉ heteroaryl), and CH₂(C₀-C₁₂ alkyl)(W₁)C₁-C₁₂ alkyl, wherein W₁ is a heteroatom selected from the group consisting of N, S and O, and wherein R₇ is selected from the group consisting of H and OH.

Nonlimiting examples of the B amino acid in these embodiments include phenylalanine(N—C₁-C₁₀alkyl), tyrosine(N—C₁-C₁₀alkyl), and tryptophan(N—C₁-C₁₀alkyl). In some embodiments, the B amino acid is selected from the group consisting of phenylalanine(N—C₁-C₆alkyl), tyrosine(N—C₁-C₆alkyl), and tryptophan(N—C₁-C₆alkyl). For example, the B amino acid can include phenylalanine(N-methyl), tyrosine(N-methyl), and tryptophan(N-methyl).

In some embodiments, the B amino acid is proline. In some embodiments, proline is excluded from Sub-Category IB.

Sub-Category IC: Amino Acid B of the Dipeptide Prodrug Element Disubstituted at the Beta Position

In some embodiments, the prodrug comprises the structure:

A-B-Q;

wherein Q is a bioactive peptide (e.g., an insulin peptide);

wherein A-B comprises the structure:

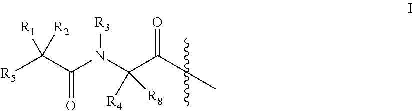

wherein

R₁ and R₂ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₁ and R₂ together with the atoms to which they are attached form a C₃-C₁₂ cycloalkyl;

R₃ is C₁-C₁₈ alkyl;

R₄ is independently selected from the group consisting of CH(C₁-C₈ alkyl)₂, CH(C₂-C₈ alkenyl)₂, CH(C₁-C₈ alkyl)(OH), CH(C₁-C₈ alkyl)((C₁-C₈ alkyl)SH), CH(C₁-C₃ alkyl)((C₁-C₈ alkyl)(NH₂));

R₈ is H;

R₅ is NHR₆, or R₅ and R₂ together with the atoms to which they are attached form a 4, 5 or 6 member heterocyclic ring;

R₆ is H or C₁-C₄ alkyl; and,

R₇ is selected from the group consisting of H and OH.

In some embodiments, R₄ is CH(C₁-C₈ alkyl)₂ or CH(C₁-C₈ alkyl)OH.

Nonlimiting examples of the B amino acid include isoleucine(N—C₁-C₁₀alkyl), valine(N—C₁-C₁₀alkyl), and threonine(N—C₁-C₁₀alkyl). In some embodiments, the B amino acid is selected from the group consisting of isoleucine(N—C₁-C₆alkyl), valine(N—C₁-C₆alkyl), and threonine(N—C₁-C₆alkyl). For example, the B amino acid can include isoleucine(N-methyl), valine(N-methyl), and threonine(N-methyl).

### Category II: Composition of Amino Acid A of the Dipeptide Prodrug Element

In some embodiments, the half-life of the prodrug is dependent on the number of substituents at the alpha position of the A amino acid. For example, a prodrug comprising an A amino acid that is an a-monosubstituted amino acid (e.g., Ala) will undergo cleavage more slowly, and have a longer half-life than, a prodrug comprising an A amino acid that is an α,α-disubstituted amino acid (e.g., Aib).

In some embodiments, the half-life of the prodrug is dependent on the degree of alkylation on the alpha amino group of the A amino acid. Generally, the greater the degree of alkylation, the slower the rate of cleavage and the longer the half-life of the prodrug. For example, a dipeptide prodrug element having N-alkylated Ala will cleave at a slower rate, and have a longer half-life, than Ala.

The composition of the A amino acid of the dipeptide prodrug element can be classified into the below sub-categories IIA and IIB. Generally, the dipeptide prodrug elements in sub-category IIA cleave faster than dipeptide prodrug elements in sub-category IIB.

Sub-Category IIA: Amino Acid A of the Dipeptide Prodrug Element is Disubstituted at the Alpha Position

In some embodiments, the A amino acid of the dipeptide prodrug element is disubstituted at the alpha position. In these embodiments, R₁ and R₂ of the structures described in sub-categories IA, IB, and IC are independently selected from the group consisting of C₁-C₁₀ alkyl, C₂-C₁₀ alkenyl, (C₁-C₁₀ alkyl)OH, (C₁-C₁₀ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl, and wherein R₇ is selected from the group consisting of H and OH.

For example, the A amino acid can include aminoisobutyric acid (Aib).

Sub-Category IIB: Amino Acid A of the Dipeptide Prodrug Element is Unsubstituted or Monosubstituted at the Alpha Position

In some embodiments, the A amino acid of the dipeptide prodrug element is unsubstituted or monosubstituted at the alpha position. In these embodiments, R₁ of the structures described in sub-categories IA, IB, and IC is H, and R₂ of the structures described in sub-categories IA, IB, and IC is selected from the group consisting of H, C₁-C₁₀ alkyl, C₂-C₁₀ alkenyl, (C₁-C₁₀ alkyl)OH, (C₁-C₁₀ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein R₇ is selected from the group consisting of H and OH, wherein W₁ is a heteroatom selected from the group consisting of N, S and O, or R₁ and R₂ together with the atoms to which they are attached form a C₃-C₁₂ cycloalkyl, or R₂ and R₅ together with the atoms to which they are attached form a 4, 5 or 6 member heterocyclic ring.

In some embodiments, the A amino acid of the dipeptide prodrug element has ‘d’ stereochemistry. Nonlimiting examples of the A amino acid in these embodiments include lysine, cysteine, and alanine. For example, d-lysine, d-cysteine, and d-alanine. In some embodiments, d-stereochemistry may enhance half-life through reducing proteolytic degradation of the prodrug peptide.

In some embodiments, the A amino acid is N-alkylated with a group that has 1 to 4 carbon atoms such as Ala(N—C₁-C₄alkyl), Lys(N—C₁-C₄alkyl), and Cys(N—C₁-C₄alkyl). For example, the A amino acid can be Ala(N-methyl), Lys(N-methyl), and Cys(N-methyl). N-alkylation of the A amino acid decreases the rate of cleavage of the dipeptide prodrug element from Q and provides a longer half-life.

Category III: Conjugation Site of the Dipeptide Prodrug Element (A-B) to the Peptide Drug (Q)

In some embodiments, the half-life of the prodrug depends on the steric hindrance, nucleophilicity, and stability of the leaving group on Q during diketopiperazine formation. The less sterically hindered the leaving group, the less nucleophilic the leaving group, or the more stable the leaving group after cleavage, the shorter the half life of the prodrug. The type of leaving group on Q can be determined by the type of the linkage between A-B and an amino group of Q, as described in sub-categories IIIA and IIIB below. Generally, dipeptide prodrug elements in sub-category IIIB cleave faster from Q and have a shorter half-life than dipeptide prodrug elements in subcategory IIIA.

Sub-Category IIIA: A-B Linked to an Aliphatic Amino Group of Q

In some embodiments, A-B is linked to Q through an amide bond between A-B and an aliphatic amino group of Q to result in a prodrug with a chemical cleavage half-life (t_{1/2}) of A-B from Q of at least about 1 hour to about 1 week in PBS, under physiological conditions, as previously described herein.

In some embodiments, A-B is linked to Q through an amide bond between A-B and the alpha amino group of the N-terminal amino acid of Q. For example, a dipeptide prodrug element having a B amino acid from any of sub-categories IA, IB, and IC and an A amino acid from any of sub-categories IIA and IIB can be linked to the N-terminal amino acid of Q to result in a prodrug with a chemical cleavage half-life (t_{1/2}) of A-B from Q of at least about 1 hour to about 1 week in PBS, under physiological conditions.

In some embodiments, A-B is linked to Q through an amide bond between A-B and an aliphatic amino group on a side chain of an amino acid of Q. For example, a dipeptide prodrug element having a B amino acid from any of sub-categories IA, IB, and IC and an A amino acid from any of sub-categories IIA and IIB can be linked to an aliphatic amino group of a side chain of an amino acid of Q to result in a prodrug with a chemical cleavage half-life (t_{1/2}) of A-B from Q of at least about 1 hour to about 1 week in PBS, under physiological conditions.

In some embodiments, when A-B is linked to Q through an amide bond between A-B and an aliphatic amino group of Q, either A should be an α,α-disubstituted amino acid (Sub-category IIA) or B should be N-alkylated (any of Sub-categories IA, IB or IC), or both. For example, when A is an a-monosubstituted amino acid (e.g., Ala), B is not N-alkylated, and A-B is attached to Q through an aliphatic amino group of Q, then there will not be significant cleavage of A-B.

In other embodiments, when A-B is linked to Q through an amide bond between A-B and an aliphatic amino group of Q, and A is an amino acid that is unsubstituted at the alpha position (e.g. glycine) and B is an amino acid from Sub-category IA (N-alkylated glycine), the N-alkyl substitutent of the B amino acid has a length of at least five carbon atoms (for example, N—C₅-C₈alkyl).

In yet other embodiments, when A-B is linked to Q through an amide bond between A-B and an aliphatic amino group of Q, and the A amino acid is unsubstituted or monosubstituted at the alpha position (Sub-category IIB), the B amino acid is not proline.

Sub-Category IIIB: A-B Linked to an Aromatic Amino Group of Q

In some embodiments, A-B is linked to Q through an amide bond between A-B and an aromatic amino group of a side chain of an amino acid of Q to result in a prodrug with a chemical cleavage half-life (t_{1/2}) of A-B from Q of at least about 1 hour to about 1 week in PBS, under physiological conditions, as previously described herein. For example, a dipeptide prodrug element having a B amino acid from any of sub-categories IA, IB, and IC and an A amino acid from any of sub-categories IIA and IIB can be linked to an aromatic amino group of a side chain of an amino acid of Q to result in a prodrug with a chemical cleavage half-life (t_{1/2}) of A-B from Q of at least about 1 hour to about 1 week in PBS, under physiological conditions.

Any of the B amino acids defined by Category I can be combined with any of the A amino acids defined by Category II to form a dipeptide prodrug element. This dipeptide prodrug element can be linked to any of the positions described in Category III. The half life of the prodrug can be tuned through the selection of:

(i) the number of substituents on the alpha position of the A amino acid;
(ii) the degree of N-alkylation of the A and the B amino acids;
(iii) the number of substituents on the beta position of the B amino acid;
(iv) the bulkiness of the side chain of the B amino acid; and,
(iii) the steric hindrance, nucleophilicity, and stability of the leaving group on Q during diketopiperazine formation.

### Modification of Dipeptide Prodrug Element A-B

The dipeptide prodrug elements described above can be further modified to comprise a hydrophilic moiety, an acyl group, or an alkyl group, as previously described herein. In some embodiments, the dipeptide prodrug element includes lysine that is conjugated to an acyl group or an alkyl group through its side chain amino group. In some embodiments, the dipeptide prodrug element includes cysteine that is conjugated to a hydrophilic moiety (e.g., 40 kD PEG) through the side chain sulfhydryl group. The hydrophilic moiety, acyl group, or alkyl group can be conjugated directly to the dipeptide prodrug element or through a spacer. In some exemplary embodiments, the hydrophilic group, the alkyl group and/or the acyl group are conjugated to the A amino acid of the dipeptide prodrug element.

In some embodiments, the following dipeptide prodrug elements are PEGylated: dCys-Gly(N-Hexyl) dCys-Gly(N-Methyl), and dCys-Phe(N-Methyl). In some embodiments, the following dipeptide prodrug elements include an acyl group: dLys-Gly(N-Hexyl), dLys-Gly(N-Methyl), and dLys-Phe(N-Methyl). In some embodiments, the following dipeptide prodrug elements include an alkyl group: dLys-Gly(N-Hexyl), dLys-Gly(N-Methyl), and dLys-Phe(N-Methyl).

### EXEMPLARY EMBODIMENTS

The dipeptide prodrug element of the invention can include combinations of any of the B amino acids from Category I with any of the A amino acids from Category II. Nonlimiting examples of amino acids suitable for the A amino acid and for the B amino acid of the dipeptide prodrug element are listed in the below Table.

| Amino | Amino | Amino |
|---|---|---|
| Acid # | Acid ‘A’ | Acid ‘B’ |
| 1 | Aib | Gly(N-C₁-C₈alkyl) |
| 2 | Gly | Ala(N-C₁-C₈alkyl) |
| 3 | Ala | Leu(N-C₁-C₈alkyl) |
| 4 | Leu | Met(N-C₁-C₈alkyl) |
| 5 | Met | Asn(N-C₁-C₈alkyl) |
| 6 | Asn | Glu(N-C₁-C₈alkyl) |
| 7 | Glu | Asp(N-C₁-C₈alkyl) |
| 8 | Asp | Gln(N-C₁-C₈alkyl) |
| 9 | Gln | His(N-C₁-C₈alkyl) |
| 10 | His | Lys(N-C₁-C₈alkyl) |
| 11 | Lys | Arg(N-C₁-C₈alkyl) |
| 12 | Arg | Ser(N-C₁-C₈alkyl) |
| 13 | Ser | Cys(N-C₁-C₈alkyl) |
| 14 | Cys | Pro |
| 15 | Pro | Phe(N-C₁-C₈alkyl) |
| 16 | Phe | Tyr(N-C₁-C₈alkyl) |
| 17 | Tyr | Trp(N-C₁-C₈alkyl) |
| 18 | Trp | Ile(N-C₁-C₈alkyl) |
| 19 | Ile | Val(N-C₁-C₈alkyl) |
| 20 | Val | Thr(N-C₁-C₈alkyl) |
| 21 | Thr | d-Ala(N-C₁-C₈alkyl) |
| 22 | d-Ala | d-Leu(N-C₁-C₈alkyl) |
| 23 | d-Leu | d-Met(N-C₁-C₈alkyl) |
| 24 | d-Met | d-Asn(N-C₁-C₈alkyl) |
| 25 | d-Asn | d-Glu(N-C₁-C₈alkyl) |
| 26 | d-Glu | d-Asp(N-C₁-C₈alkyl) |
| 27 | d-Asp | d-Gln(N-C₁-C₈alkyl) |
| 28 | d-Gln | d-His(N-C₁-C₈alkyl) |
| 29 | d-His | d-Lys(N-C₁-C₈alkyl) |
| 30 | d-Lys | d-Arg(N-C₁-C₈alkyl) |
| 31 | d-Arg | d-Ser(N-C₁-C₈alkyl) |
| 32 | d-Ser | d-Cys(N-C₁-C₈alkyl) |
| 33 | d-Cys | d-Pro |
| 34 | d-Pro | d-Phe(N-C₁-C₈alkyl) |
| 35 | d-Phe | d-Tyr(N-C₁-C₈alkyl) |
| 36 | d-Tyr | d-Trp(N-C₁-C₈alkyl) |
| 37 | d-Trp | d-Ile(N-C₁-C₈alkyl) |
| 38 | d-Ile | d-Val(N-C₁-C₈alkyl) |
| 39 | d-Val | d-Thr(N-C₁-C₈alkyl) |
| 40 | d-Thr | Gly(N-methyl) |
| 41 | Gly(N-methyl) | Ala(N-methyl) |
| 42 | Ala(N-methyl) | Leu(N-methyl) |
| 43 | Leu(N-methyl) | Met(N-methyl) |
| 44 | Met(N-methyl) | Asn(N-methyl) |
| 45 | Asn(N-methyl) | Glu(N-methyl) |
| 46 | Glu(N-methyl) | Asp(N-methyl) |
| 47 | Asp(N-methyl) | Gln(N-methyl) |
| 48 | Gln(N-methyl) | His(N-methyl) |
| 49 | His(N-methyl) | Lys(N-methyl) |
| 50 | Lys(N-methyl) | Arg(N-methyl) |
| 51 | Arg(N-methyl) | Ser(N-methyl) |
| 52 | Ser(N-methyl) | Cys(N-methyl) |
| 53 | Cys(N-methyl) | Phe(N-methyl) |
| 54 | Phe(N-methyl) | Tyr(N-methyl) |
| 55 | Tyr(N-methyl) | Trp(N-methyl) |
| 56 | Trp(N-methyl) | Ile(N-methyl) |
| 57 | Ile(N-methyl) | Val(N-methyl) |
| 58 | Val(N-methyl) | Thr(N-methyl) |
| 59 | Thr(N-methyl) | d-Ala(N-methyl) |
| 60 | d-Ala(N-methyl) | d-Leu(N-methyl) |
| 61 | d-Leu(N-methyl) | d-Met(N-methyl) |
| 62 | d-Met(N-methyl) | d-Asn(N-methyl) |
| 63 | d-Asn(N-methyl) | d-Glu(N-methyl) |
| 64 | d-Glu(N-methyl) | d-Asp(N-methyl) |
| 65 | d-Asp(N-methyl) | d-Gln(N-methyl) |
| 66 | d-Gln(N-methyl) | d-His(N-methyl) |
| 67 | d-His(N-methyl) | d-Lys(N-methyl) |
| 68 | d-Lys(N-methyl) | d-Arg(N-methyl) |
| 69 | d-Arg(N-methyl) | d-Ser(N-methyl) |
| 70 | d-Ser(N-methyl) | d-Cys(N-methyl) |
| 71 | d-Cys(N-methyl) | d-Phe(N-methyl) |
| 72 | d-Phe(N-methyl) | d-Tyr(N-methyl) |
| 73 | d-Tyr(N-methyl) | d-Trp(N-methyl) |
| 74 | d-Trp(N-methyl) | d-Ile(N-methyl) |
| 75 | d-Ile(N-methyl) | d-Val(N-methyl) |
| 76 | d-Val(N-methyl) | d-Thr(N-methyl) |
| 77 | d-Thr(N-methyl) | Gly(N-hexyl) |
| 78 | | Ala(N-hexyl) |
| 79 | | Leu(N-hexyl) |
| 80 | | Met(N-hexyl) |
| 81 | | Asn(N-hexyl) |
| 82 | | Glu(N-hexyl) |
| 83 | | Asp(N-hexyl) |
| 84 | | Gln(N-hexyl) |
| 85 | | His(N-hexyl) |
| 86 | | Lys(N-hexyl) |
| 87 | | Arg(N-hexyl) |
| 88 | | Ser(N-hexyl) |
| 89 | | Cys(N-hexyl) |
| 90 | | Phe(N-hexyl) |
| 91 | | Tyr(N-hexyl) |
| 92 | | Trp(N-hexyl) |
| 93 | | Ile(N-hexyl) |
| 94 | | Val(N-hexyl) |
| 95 | | Thr(N-hexyl) |
| 96 | | d-Ala(N-hexyl) |
| 97 | | d-Leu(N-hexyl) |
| 98 | | d-Met(N-hexyl) |
| 99 | | d-Asn(N-hexyl) |
| 100 | | d-Glu(N-hexyl) |
| 101 | | d-Asp(N-hexyl) |
| 102 | | d-Gln(N-hexyl) |
| 103 | | d-His(N-hexyl) |
| 104 | | d-Lys(N-hexyl) |
| 105 | | d-Arg(N-hexyl) |
| 106 | | d-Ser(N-hexyl) |
| 107 | | d-Cys(N-hexyl) |
| 108 | | d-Phe(N-hexyl) |
| 109 | | d-Tyr(N-hexyl) |
| 110 | | d-Trp(N-hexyl) |
| 111 | | d-Ile(N-hexyl) |
| 112 | | d-Val(N-hexyl) |
| 113 | | d-Thr(N-hexyl) |

In some embodiments, the dipeptide prodrug element includes the combination of any one of A1-A77 with any one of B1-B113. For example, combinations of the A amino acid and the B amino acid of the dipeptide prodrug element can include: A1-B1; A1-B2; A1-B3; A1-B4; A1-B5; A1-B6; A1-B7; A1-B8; A1-B9; A1-B10; A1-B11; A1-B12; A1-B13; A1-B14; A1-B15; A1-B16; A1-B17; A1-B18; A1-B19; A1-B20; A1-B21; A1-B22; A1-B23; A1-B24; A1-B25; A1-B26; A1-B27; A1-B28; A1-B29; A1-B30; A1-B31; A1-B32; A1-B33; A1-B34; A1-B35; A1-B36; A1-B37; A1-B38; A1-B39; A1-B40; A1-B41; A1-B42; A1-B43; A1-B44; A1-B45; A1-B46; A1-B47; A1-B48; A1-B49; A1-B50; A1-B51; A1-B52; A1-B53; A1-B54; A1-B55; A1-B56; A1-B57; A1-B58; A1-B59; A1-B60; A1-B61; A1-B62; A1-B63; A1-B64; A1-B65; A1-B66; A1-B67; A1-B68; A1-B69; A1-B70; A1-B71; A1-B72; A1-B73; A1-B74; A1-B75; A1-B76; A1-B77; A1-B78; A1-B79; A1-B80; A1-B81; A1-B82; A1-B83; A1-B84; A1-B85; A1-B86; A1-B87; A1-B88; A1-B89; A1-B90; A1-B91; A1-B92; A1-B93; A1-B94; A1-B95; A1-B96; A1-B97; A1-B98; A1-B99; A1-B100; A1-B101; A1-B102; A1-B103; A1-B104; A1-B105; A1-B106; A1-B107; A1-B108; A1-B109; A1-B110; A1-B111; A1-B112; A1-B113;

In some embodiments, the dipeptide prodrug element includes the combination of any one of A1-A154 with any one of B1-B113. For example, combinations of the A amino acid and the B amino acid of the dipeptide prodrug element can include: A1-B1; A1-B2; A1-B3; A1-B4; A1-B5; A1-B6; A1-B7; A1-B8; A1-B9; A1-B10; A1-B11; A1-B12; A1-B13; A1-B14; A1-B15; A1-B16; A1-B17; A1-B18; A1-B19; A1-B20; A1-B21; A1-B22; A1-B23; A1-B24; A1-B25; A1-B26; A1-B27; A1-B28; A1-B29; A1-B30; A1-B31; A1-B32; A1-B33; A1-B34; A1-B35; A1-B36; A1-B37; A1-B38; A1-B39; A1-B40; A1-B41; A1-B42; A1-B43; A1-B44; A1-B45; A1-B46; A1-B47; A1-B48; A1-B49; A1-B50; A1-B51; A1-B52; A1-B53; A1-B54; A1-B55; A1-B56; A1-B57; A1-B58; A1-B59; A1-B60; A1-B61; A1-B62; A1-B63; A1-B64; A1-B65; A1-B66; A1-B67; A1-B68; A1-B69; A1-B70; A1-B71; A1-B72; A1-B73; A1-B74; A1-B75; A1-B76; A1-B77; A1-B78; A1-B79; A1-B80; A1-B81; A1-B82; A1-B83; A1-B84; A1-B85; A1-B86; A1-B87; A1-B88; A1-B89; A1-B90; A1-B91; A1-B92; A1-B93; A1-B94; A1-B95; A1-B96; A1-B97; A1-B98; A1-B99; A1-B100; A1-B101; A1-B102; A1-B103; A1-B104; A1-B105; A1-B106; A1-B107; A1-B108; A1-B109; A1-B110; A1-B111; A1-B112; A1-B113;

A2-B1; A2-B2; A2-B3; A2-B4; A2-B5; A2-B6; A2-B7; A2-B8; A2-B9; A2-B10; A2-B11; A2-B12; A2-B13; A2-B14; A2-B15; A2-B16; A2-B17; A2-B18; A2-B19; A2-B20; A2-B21; A2-B22; A2-B23; A2-B24; A2-B25; A2-B26; A2-B27; A2-B28; A2-B29; A2-B30; A2-B31; A2-B32; A2-B33; A2-B34; A2-B35; A2-B36; A2-B37; A2-B38; A2-B39; A2-B40; A2-B41; A2-B42; A2-B43; A2-B44; A2-B45; A2-B46; A2-B47; A2-B48; A2-B49; A2-B50; A2-B51; A2-B52; A2-B53; A2-B54; A2-B55; A2-B56; A2-B57; A2-B58; A2-B59; A2-B60; A2-B61; A2-B62; A2-B63; A2-B64; A2-B65; A2-B66; A2-B67; A2-B68; A2-B69; A2-B70; A2-B71; A2-B72; A2-B73; A2-B74; A2-B75; A2-B76; A2-B77; A2-B78; A2-B79; A2-B80; A2-B81; A2-B82; A2-B83; A2-B84; A2-B85; A2-B86; A2-B87; A2-B88; A2-B89; A2-B90; A2-B91; A2-B92; A2-B93; A2-B94; A2-B95; A2-B96; A2-B97; A2-B98; A2-B99; A2-B100; A2-B101; A2-B102; A2-B103; A2-B104; A2-B105; A2-B106; A2-B107; A2-B108; A2-B109; A2-B110; A2-B111; A2-B112; A2-B113;

A3-B1; A3-B2; A3-B3; A3-B4; A3-B5; A3-B6; A3-B7; A3-B8; A3-B9; A3-B10; A3-B11; A3-B12; A3-B13; A3-B14; A3-B15; A3-B16; A3-B17; A3-B18; A3-B19; A3-B20; A3-B21; A3-B22; A3-B23; A3-B24; A3-B25; A3-B26; A3-B27; A3-B28; A3-B29; A3-B30; A3-B31; A3-B32; A3-B33; A3-B34; A3-B35; A3-B36; A3-B37; A3-B38; A3-B39; A3-B40; A3-B41; A3-B42; A3-B43; A3-B44; A3-B45; A3-B46; A3-B47; A3-B48; A3-B49; A3-B50; A3-B51; A3-B52; A3-B53; A3-B54; A3-B55; A3-B56; A3-B57; A3-B58; A3-B59; A3-B60; A3-B61; A3-B62; A3-B63; A3-B64; A3-B65; A3-B66; A3-B67; A3-B68; A3-B69; A3-B70; A3-B71; A3-B72; A3-B73; A3-B74; A3-B75; A3-B76; A3-B77; A3-B78; A3-B79; A3-B80; A3-B81; A3-B82; A3-B83; A3-B84; A3-B85; A3-B86; A3-B87; A3-B88; A3-B89; A3-B90; A3-B91; A3-B92; A3-B93; A3-B94; A3-B95; A3-B96; A3-B97; A3-B98; A3-B99; A3-B100; A3-B101; A3-B102; A3-B103; A3-B104; A3-B105; A3-B106; A3-B107; A3-B108; A3-B109; A3-B110; A3-B111; A3-B112; A3-B113;

A4-B1; A4-B2; A4-B3; A4-B4; A4-B5; A4-B6; A4-B7; A4-B8; A4-B9; A4-B10; A4-B11; A4-B12; A4-B13; A4-B14; A4-B15; A4-B16; A4-B17; A4-B18; A4-B19; A4-B20; A4-B21; A4-B22; A4-B23; A4-B24; A4-B25; A4-B26; A4-B27; A4-B28; A4-B29; A4-B30; A4-B31; A4-B32; A4-B33; A4-B34; A4-B35; A4-B36; A4-B37; A4-B38; A4-B39; A4-B40; A4-B41; A4-B42; A4-B43; A4-B44; A4-B45; A4-B46; A4-B47; A4-B48; A4-B49; A4-B50; A4-B51; A4-B52; A4-B53; A4-B54; A4-B55; A4-B56; A4-B57; A4-B58; A4-B59; A4-B60; A4-B61; A4-B62; A4-B63; A4-B64; A4-B65; A4-B66; A4-B67; A4-B68; A4-B69; A4-B70; A4-B71; A4-B72; A4-B73; A4-B74; A4-B75; A4-B76; A4-B77; A4-B78; A4-B79; A4-B80; A4-B81; A4-B82; A4-B83; A4-B84; A4-B85; A4-B86; A4-B87; A4-B88; A4-B89; A4-B90; A4-B91; A4-B92; A4-B93; A4-B94; A4-B95; A4-B96; A4-B97; A4-B98; A4-B99; A4-B100; A4-B101; A4-B102; A4-B103; A4-B104; A4-B105; A4-B106; A4-B107; A4-B108; A4-B109; A4-B110; A4-B111; A4-B112; A4-B113;

A5-B1; A5-B2; A5-B3; A5-B4; A5-B5; A5-B6; A5-B7; A5-B8; A5-B9; A5-B10; A5-B11; A5-B12; A5-B13; A5-B14; A5-B15; A5-B16; A5-B17; A5-B18; A5-B19; A5-B20; A5-B21; A5-B22; A5-B23; A5-B24; A5-B25; A5-B26; A5-B27; A5-B28; A5-B29; A5-B30; A5-B31; A5-B32; A5-B33; A5-B34; A5-B35; A5-B36; A5-B37; A5-B38; A5-B39; A5-B40; A5-B41; A5-B42; A5-B43; A5-B44; A5-B45; A5-B46; A5-B47; A5-B48; A5-B49; A5-B50; A5-B51; A5-B52; A5-B53; A5-B54; A5-B55; A5-B56; A5-B57; A5-B58; A5-B59; A5-B60; A5-B61; A5-B62; A5-B63; A5-B64; A5-B65; A5-B66; A5-B67; A5-B68; A5-B69; A5-B70; A5-B71; A5-B72; A5-B73; A5-B74; A5-B75; A5-B76; A5-B77; A5-B78; A5-B79; A5-B80; A5-B81; A5-B82; A5-B83; A5-B84; A5-B85; A5-B86; A5-B87; A5-B88; A5-B89; A5-B90; A5-B91; A5-B92; A5-B93; A5-B94; A5-B95; A5-B96; A5-B97; A5-B98; A5-B99; A5-B100; A5-B101; A5-B102; A5-B103; A5-B104; A5-B105; A5-B106; A5-B107; A5-B108; A5-B109; A5-B110; A5-B111; A5-B112; A5-B113;

A6-B1; A6-B2; A6-B3; A6-B4; A6-B5; A6-B6; A6-B7; A6-B8; A6-B9; A6-B10; A6-B11; A6-B12; A6-B13; A6-B14; A6-B15; A6-B16; A6-B17; A6-B18; A6-B19; A6-B20; A6-B21; A6-B22; A6-B23; A6-B24; A6-B25; A6-B26; A6-B27; A6-B28; A6-B29; A6-B30; A6-B31; A6-B32; A6-B33; A6-B34; A6-B35; A6-B36; A6-B37; A6-B38; A6-B39; A6-B40; A6-B41; A6-B42; A6-B43; A6-B44; A6-B45; A6-B46; A6-B47; A6-B48; A6-B49; A6-B50; A6-B51; A6-B52; A6-B53; A6-B54; A6-B55; A6-B56; A6-B57; A6-B58; A6-B59; A6-B60; A6-B61; A6-B62; A6-B63; A6-B64; A6-B65; A6-B66; A6-B67; A6-B68; A6-B69; A6-B70; A6-B71; A6-B72; A6-B73; A6-B74; A6-B75; A6-B76; A6-B77; A6-B78; A6-B79; A6-B80; A6-B81; A6-B82; A6-B83; A6-B84; A6-B85; A6-B86; A6-B87; A6-B88; A6-B89; A6-B90; A6-B91; A6-B92; A6-B93; A6-B94; A6-B95; A6-B96; A6-B97; A6-B98; A6-B99; A6-B100; A6-B101; A6-B102; A6-B103; A6-B104; A6-B105; A6-B106; A6-B107; A6-B108; A6-B109; A6-B110; A6-B111; A6-B112; A6-B113;

A7-B1; A7-B2; A7-B3; A7-B4; A7-B5; A7-B6; A7-B7; A7-B8; A7-B9; A7-B10; A7-B11; A7-B12; A7-B13; A7-B14; A7-B15; A7-B16; A7-B17; A7-B18; A7-B19; A7-B20; A7-B21; A7-B22; A7-B23; A7-B24; A7-B25; A7-B26; A7-B27; A7-B28; A7-B29; A7-B30; A7-B31; A7-B32; A7-B33; A7-B34; A7-B35; A7-B36; A7-B37; A7-B38; A7-B39; A7-B40; A7-B41; A7-B42; A7-B43; A7-B44; A7-B45; A7-B46; A7-B47; A7-B48; A7-B49; A7-B50; A7-B51; A7-B52; A7-B53; A7-B54; A7-B55; A7-B56; A7-B57; A7-B58; A7-B59; A7-B60; A7-B61; A7-B62; A7-B63; A7-B64; A7-B65; A7-B66; A7-B67; A7-B68; A7-B69; A7-B70; A7-B71; A7-B72; A7-B73; A7-B74; A7-B75; A7-B76; A7-B77; A7-B78; A7-B79; A7-B80; A7-B81; A7-B82; A7-B83; A7-B84; A7-B85; A7-B86; A7-B87; A7-B88; A7-B89; A7-B90; A7-B91; A7-B92; A7-B93; A7-B94; A7-B95; A7-B96; A7-B97; A7-B98; A7-B99; A7-B100; A7-B101; A7-B102; A7-B103; A7-B104; A7-B105; A7-B106; A7-B107; A7-B108; A7-B109; A7-B110; A7-B111; A7-B112; A7-B113;

A8-B1; A8-B2; A8-B3; A8-B4; A8-B5; A8-B6; A8-B7; A8-B8; A8-B9; A8-B10; A8-B11; A8-B12; A8-B13; A8-B14; A8-B15; A8-B16; A8-B17; A8-B18; A8-B19; A8-B20; A8-B21; A8-B22; A8-B23; A8-B24; A8-B25; A8-B26; A8-B27; A8-B28; A8-B29; A8-B30; A8-B31; A8-B32; A8-B33; A8-B34; A8-B35; A8-B36; A8-B37; A8-B38; A8-B39; A8-B40; A8-B41; A8-B42; A8-B43; A8-B44; A8-B45; A8-B46; A8-B47; A8-B48; A8-B49; A8-B50; A8-B51; A8-B52; A8-B53; A8-B54; A8-B55; A8-B56; A8-B57; A8-B58; A8-B59; A8-B60; A8-B61; A8-B62; A8-B63; A8-B64; A8-B65; A8-B66; A8-B67; A8-B68; A8-B69; A8-B70; A8-B71; A8-B72; A8-B73; A8-B74; A8-B75; A8-B76; A8-B77; A8-B78; A8-B79; A8-B80; A8-B81; A8-B82; A8-B83; A8-B84; A8-B85; A8-B86; A8-B87; A8-B88; A8-B89; A8-B90; A8-B91; A8-B92; A8-B93; A8-B94; A8-B95; A8-B96; A8-B97; A8-B98; A8-B99; A8-B100; A8-B101; A8-B102; A8-B103; A8-B104; A8-B105; A8-B106; A8-B107; A8-B108; A8-B109; A8-B110; A8-B111; A8-B112; A8-B113;

A9-B1; A9-B2; A9-B3; A9-B4; A9-B5; A9-B6; A9-B7; A9-B8; A9-B9; A9-B10; A9-B11; A9-B12; A9-B13; A9-B14; A9-B15; A9-B16; A9-B17; A9-B18; A9-B19; A9-B20; A9-B21; A9-B22; A9-B23; A9-B24; A9-B25; A9-B26; A9-B27; A9-B28; A9-B29; A9-B30; A9-B31; A9-B32; A9-B33; A9-B34; A9-B35; A9-B36; A9-B37; A9-B38; A9-B39; A9-B40; A9-B41; A9-B42; A9-B43; A9-B44; A9-B45; A9-B46; A9-B47; A9-B48; A9-B49; A9-B50; A9-B51; A9-B52; A9-B53; A9-B54; A9-B55; A9-B56; A9-B57; A9-B58; A9-B59; A9-B60; A9-B61; A9-B62; A9-B63; A9-B64; A9-B65; A9-B66; A9-B67; A9-B68; A9-B69; A9-B70; A9-B71; A9-B72; A9-B73; A9-B74; A9-B75; A9-B76; A9-B77; A9-B78; A9-B79; A9-B80; A9-B81; A9-B82; A9-B83; A9-B84; A9-B85; A9-B86; A9-B87; A9-B88; A9-B89; A9-B90; A9-B91; A9-B92; A9-B93; A9-B94; A9-B95; A9-B96; A9-B97; A9-B98; A9-B99; A9-B100; A9-B101; A9-B102; A9-B103; A9-B104; A9-B105; A9-B106; A9-B107; A9-B108; A9-B109; A9-B110; A9-B111; A9-B112; A9-B113;

A10-B1; A10-B2; A10-B3; A10-B4; A10-B5; A10-B6; A10-B7; A10-B8; A10-B9; A10-B10; A10-B11; A10-B12; A10-B13; A10-B14; A10-B15; A10-B16; A10-B17; A10-B18; A10-B19; A10-B20; A10-B21; A10-B22; A10-B23; A10-B24; A10-B25; A10-B26; A10-B27; A10-B28; A10-B29; A10-B30; A10-B31; A10-B32; A10-B33; A10-B34; A10-B35; A10-B36; A10-B37; A10-B38; A10-B39; A10-B40; A10-B41; A10-B42; A10-B43; A10-B44; A10-B45; A10-B46; A10-B47; A10-B48; A10-B49; A10-B50; A10-B51; A10-B52; A10-B53; A10-B54; A10-B55; A10-B56; A10-B57; A10-B58; A10-B59; A10-B60; A10-B61; A10-B62; A10-B63; A10-B64; A10-B65; A10-B66; A10-B67; A10-B68; A10-B69; A10-B70; A10-B71; A10-B72; A10-B73; A10-B74; A10-B75; A10-B76; A10-B77; A10-B78; A10-B79; A10-B80; A10-B81; A10-B82; A10-B83; A10-B84; A10-B85; A10-B86; A10-B87; A10-B88; A10-B89; A10-B90; A10-B91; A10-B92; A10-B93; A10-B94; A10-B95; A10-B96; A10-B97; A10-B98; A10-B99; A10-B100; A10-B101; A10-B102; A10-B103; A10-B104; A10-B105; A10-B106; A10-B107; A10-B108; A10-B109; A10-B110; A10-B111; A10-B112; A10-B113;

A11-B1; A11-B2; A11-B3; A11-B4; A11-B5; A11-B6; A11-B7; A11-B8; A11-B9; A11-B10; A11-B11; A11-B12; A11-B13; A11-B14; A11-B15; A11-B16; A11-B17; A11-B18; A11-B19; A11-B20; A11-B21; A11-B22; A11-B23; A11-B24; A11-B25; A11-B26; A11-B27; A11-B28; A11-B29; A11-B30; A11-B31; A11-B32; A11-B33; A11-B34; A11-B35; A11-B36; A11-B37; A11-B38; A11-B39; A11-B40; A11-B41; A11-B42; A11-B43; A11-B44; A11-B45; A11-B46; A11-B47; A11-B48; A11-B49; A11-B50; A11-B51; A11-B52; A11-B53; A11-B54; A11-B55; A11-B56; A11-B57; A11-B58; A11-B59; A11-B60; A11-B61; A11-B62; A11-B63; A11-B64; A11-B65; A11-B66; A11-B67; A11-B68; A11-B69; A11-B70; A11-B71; A11-B72; A11-B73; A11-B74; A11-B75; A11-B76; A11-B77; A11-B78; A11-B79; A11-B80; A11-B81; A11-B82; A11-B83; A11-B84; A11-B85; A11-B86; A11-B87; A11-B88; A11-B89; A11-B90; A11-B91; A11-B92; A11-B93; A11-B94; A11-B95; A11-B96; A11-B97; A11-B98; A11-B99; A11-B100; A11-B101; A11-B102; A11-B103; A11-B104; A11-B105; A11-B106; A11-B107; A11-B108; A11-B109; A11-B110; A11-B111; A11-B112; A11-B113;

A12-B1; A12-B2; A12-B3; A12-B4; A12-B5; A12-B6; A12-B7; A12-B8; A12-B9; A12-B10; A12-B11; A12-B12; A12-B13; A12-B14; A12-B15; A12-B16; A12-B17; A12-B18; A12-B19; A12-B20; A12-B21; A12-B22; A12-B23; A12-B24; A12-B25; A12-B26; A12-B27; A12-B28; A12-B29; A12-B30; A12-B31; A12-B32; A12-B33; A12-B34; A12-B35; A12-B36; A12-B37; A12-B38; A12-B39; A12-B40; A12-B41; A12-B42; A12-B43; A12-B44; A12-B45; A12-B46; A12-B47; A12-B48; A12-B49; A12-B50; A12-B51; A12-B52; A12-B53; A12-B54; A12-B55; A12-B56; A12-B57; A12-B58; A12-B59; A12-B60; A12-B61; A12-B62; A12-B63; A12-B64; A12-B65; A12-B66; A12-B67; A12-B68; A12-B69; A12-B70; A12-B71; A12-B72; A12-B73; A12-B74; A12-B75; A12-B76; A12-B77; A12-B78; A12-B79; A12-B80; A12-B81; A12-B82; A12-B83; A12-B84; A12-B85; A12-B86; A12-B87; A12-B88; A12-B89; A12-B90; A12-B91; A12-B92; A12-B93; A12-B94; A12-B95; A12-B96; A12-B97; A12-B98; A12-B99; A12-B100; A12-B101; A12-B102; A12-B103; A12-B104; A12-B105; A12-B106; A12-B107; A12-B108; A12-B109; A12-B110; A12-B111; A12-B112; A12-B113;

A13-B1; A13-B2; A13-B3; A13-B4; A13-B5; A13-B6; A13-B7; A13-B8; A13-B9; A13-B10; A13-B11; A13-B12; A13-B13; A13-B14; A13-B15; A13-B16; A13-B17; A13-B18; A13-B19; A13-B20; A13-B21; A13-B22; A13-B23; A13-B24; A13-B25; A13-B26; A13-B27; A13-B28; A13-B29; A13-B30; A13-B31; A13-B32; A13-B33; A13-B34; A13-B35; A13-B36; A13-B37; A13-B38; A13-B39; A13-B40; A13-B41; A13-B42; A13-B43; A13-B44; A13-B45; A13-B46; A13-B47; A13-B48; A13-B49; A13-B50; A13-B51; A13-B52; A13-B53; A13-B54; A13-B55; A13-B56; A13-B57; A13-B58; A13-B59; A13-B60; A13-B61; A13-B62; A13-B63; A13-B64; A13-B65; A13-B66; A13-B67; A13-B68; A13-B69; A13-B70; A13-B71; A13-B72; A13-B73; A13-B74; A13-B75; A13-B76; A13-B77; A13-B78; A13-B79; A13-B80; A13-B81; A13-B82; A13-B83; A13-B84; A13-B85; A13-B86; A13-B87; A13-B88; A13-B89; A13-B90; A13-B91; A13-B92; A13-B93; A13-B94; A13-B95; A13-B96; A13-B97; A13-B98; A13-B99; A13-B100; A13-B101; A13-B102; A13-B103; A13-B104; A13-B105; A13-B106; A13-B107; A13-B108; A13-B109; A13-B110; A13-B111; A13-B112; A13-B113;

A14-B1; A14-B2; A14-B3; A14-B4; A14-B5; A14-B6; A14-B7; A14-B8; A14-B9; A14-B10; A14-B11; A14-B12; A14-B13; A14-B14; A14-B15; A14-B16; A14-B17; A14-B18; A14-B19; A14-B20; A14-B21; A14-B22; A14-B23; A14-B24; A14-B25; A14-B26; A14-B27; A14-B28; A14-B29; A14-B30; A14-B31; A14-B32; A14-B33; A14-B34; A14-B35; A14-B36; A14-B37; A14-B38; A14-B39; A14-B40; A14-B41; A14-B42; A14-B43; A14-B44; A14-B45; A14-B46; A14-B47; A14-B48; A14-B49; A14-B50; A14-B51; A14-B52; A14-B53; A14-B54; A14-B55; A14-B56; A14-B57; A14-B58; A14-B59; A14-B60; A14-B61; A14-B62; A14-B63; A14-B64; A14-B65; A14-B66; A14-B67; A14-B68; A14-B69; A14-B70; A14-B71; A14-B72; A14-B73; A14-B74; A14-B75; A14-B76; A14-B77; A14-B78; A14-B79; A14-B80; A14-B81; A14-B82; A14-B83; A14-B84; A14-B85; A14-B86; A14-B87; A14-B88; A14-B89; A14-B90; A14-B91; A14-B92; A14-B93; A14-B94; A14-B95; A14-B96; A14-B97; A14-B98; A14-B99; A14-B100; A14-B101; A14-B102; A14-B103; A14-B104; A14-B105; A14-B106; A14-B107; A14-B108; A14-B109; A14-B110; A14-B111; A14-B112; A14-B113;

A15-B1; A15-B2; A15-B3; A15-B4; A15-B5; A15-B6; A15-B7; A15-B8; A15-B9; A15-B10; A15-B11; A15-B12; A15-B13; A15-B14; A15-B15; A15-B16; A15-B17; A15-B18; A15-B19; A15-B20; A15-B21; A15-B22; A15-B23; A15-B24; A15-B25; A15-B26; A15-B27; A15-B28; A15-B29; A15-B30; A15-B31; A15-B32; A15-B33; A15-B34; A15-B35; A15-B36; A15-B37; A15-B38; A15-B39; A15-B40; A15-B41; A15-B42; A15-B43; A15-B44; A15-B45; A15-B46; A15-B47; A15-B48; A15-B49; A15-B50; A15-B51; A15-B52; A15-B53; A15-B54; A15-B55; A15-B56; A15-B57; A15-B58; A15-B59; A15-B60; A15-B61; A15-B62; A15-B63; A15-B64; A15-B65; A15-B66; A15-B67; A15-B68; A15-B69; A15-B70; A15-B71; A15-B72; A15-B73; A15-B74; A15-B75; A15-B76; A15-B77; A15-B78; A15-B79; A15-B80; A15-B81; A15-B82; A15-B83; A15-B84; A15-B85; A15-B86; A15-B87; A15-B88; A15-B89; A15-B90; A15-B91; A15-B92; A15-B93; A15-B94; A15-B95; A15-B96; A15-B97; A15-B98; A15-B99; A15-B100; A15-B101; A15-B102; A15-B103; A15-B104; A15-B105; A15-B106; A15-B107; A15-B108; A15-B109; A15-B110; A15-B111; A15-B112; A15-B113;

A16-B1; A16-B2; A16-B3; A16-B4; A16-B5; A16-B6; A16-B7; A16-B8; A16-B9; A16-B10; A16-B11; A16-B12; A16-B13; A16-B14; A16-B15; A16-B16; A16-B17; A16-B18; A16-B19; A16-B20; A16-B21; A16-B22; A16-B23; A16-B24; A16-B25; A16-B26; A16-B27; A16-B28; A16-B29; A16-B30; A16-B31; A16-B32; A16-B33; A16-B34; A16-B35; A16-B36; A16-B37; A16-B38; A16-B39; A16-B40; A16-B41; A16-B42; A16-B43; A16-B44; A16-B45; A16-B46; A16-B47; A16-B48; A16-B49; A16-B50; A16-B51; A16-B52; A16-B53; A16-B54; A16-B55; A16-B56; A16-B57; A16-B58; A16-B59; A16-B60; A16-B61; A16-B62; A16-B63; A16-B64; A16-B65; A16-B66; A16-B67; A16-B68; A16-B69; A16-B70; A16-B71; A16-B72; A16-B73; A16-B74; A16-B75; A16-B76; A16-B77; A16-B78; A16-B79; A16-B80; A16-B81; A16-B82; A16-B83; A16-B84; A16-B85; A16-B86; A16-B87; A16-B88; A16-B89; A16-B90; A16-B91; A16-B92; A16-B93; A16-B94; A16-B95; A16-B96; A16-B97; A16-B98; A16-B99; A16-B100; A16-B101; A16-B102; A16-B103; A16-B104; A16-B105; A16-B106; A16-B107; A16-B108; A16-B109; A16-B110; A16-B111; A16-B112; A16-B113;

A17-B1; A17-B2; A17-B3; A17-B4; A17-B5; A17-B6; A17-B7; A17-B8; A17-B9; A17-B10; A17-B11; A17-B12; A17-B13; A17-B14; A17-B15; A17-B16; A17-B17; A17-B18; A17-B19; A17-B20; A17-B21; A17-B22; A17-B23; A17-B24; A17-B25; A17-B26; A17-B27; A17-B28; A17-B29; A17-B30; A17-B31; A17-B32; A17-B33; A17-B34; A17-B35; A17-B36; A17-B37; A17-B38; A17-B39; A17-B40; A17-B41; A17-B42; A17-B43; A17-B44; A17-B45; A17-B46; A17-B47; A17-B48; A17-B49; A17-B50; A17-B51; A17-B52; A17-B53; A17-B54; A17-B55; A17-B56; A17-B57; A17-B58; A17-B59; A17-B60; A17-B61; A17-B62; A17-B63; A17-B64; A17-B65; A17-B66; A17-B67; A17-B68; A17-B69; A17-B70; A17-B71; A17-B72; A17-B73; A17-B74; A17-B75; A17-B76; A17-B77; A17-B78; A17-B79; A17-B80; A17-B81; A17-B82; A17-B83; A17-B84; A17-B85; A17-B86; A17-B87; A17-B88; A17-B89; A17-B90; A17-B91; A17-B92; A17-B93; A17-B94; A17-B95; A17-B96; A17-B97; A17-B98; A17-B99; A17-B100; A17-B101; A17-B102; A17-B103; A17-B104; A17-B105; A17-B106; A17-B107; A17-B108; A17-B109; A17-B110; A17-B111; A17-B112; A17-B113;

A18-B1; A18-B2; A18-B3; A18-B4; A18-B5; A18-B6; A18-B7; A18-B8; A18-B9; A18-B10; A18-B11; A18-B12; A18-B13; A18-B14; A18-B15; A18-B16; A18-B17; A18-B18; A18-B19; A18-B20; A18-B21; A18-B22; A18-B23; A18-B24; A18-B25; A18-B26; A18-B27; A18-B28; A18-B29; A18-B30; A18-B31; A18-B32; A18-B33; A18-B34; A18-B35; A18-B36; A18-B37; A18-B38; A18-B39; A18-B40; A18-B41; A18-B42; A18-B43; A18-B44; A18-B45; A18-B46; A18-B47; A18-B48; A18-B49; A18-B50; A18-B51; A18-B52; A18-B53; A18-B54; A18-B55; A18-B56; A18-B57; A18-B58; A18-B59; A18-B60; A18-B61; A18-B62; A18-B63; A18-B64; A18-B65; A18-B66; A18-B67; A18-B68; A18-B69; A18-B70; A18-B71; A18-B72; A18-B73; A18-B74; A18-B75; A18-B76; A18-B77; A18-B78; A18-B79; A18-B80; A18-B81; A18-B82; A18-B83; A18-B84; A18-B85; A18-B86; A18-B87; A18-B88; A18-B89; A18-B90; A18-B91; A18-B92; A18-B93; A18-B94; A18-B95; A18-B96; A18-B97; A18-B98; A18-B99; A18-B100; A18-B101; A18-B102; A18-B103; A18-B104; A18-B105; A18-B106; A18-B107; A18-B108; A18-B109; A18-B110; A18-B111; A18-B112; A18-B113;

A19-B1; A19-B2; A19-B3; A19-B4; A19-B5; A19-B6; A19-B7; A19-B8; A19-B9; A19-B10; A19-B11; A19-B12; A19-B13; A19-B14; A19-B15; A19-B16; A19-B17; A19-B18; A19-B19; A19-B20; A19-B21; A19-B22; A19-B23; A19-B24; A19-B25; A19-B26; A19-B27; A19-B28; A19-B29; A19-B30; A19-B31; A19-B32; A19-B33; A19-B34; A19-B35; A19-B36; A19-B37; A19-B38; A19-B39; A19-B40; A19-B41; A19-B42; A19-B43; A19-B44; A19-B45; A19-B46; A19-B47; A19-B48; A19-B49; A19-B50; A19-B51; A19-B52; A19-B53; A19-B54; A19-B55; A19-B56; A19-B57; A19-B58; A19-B59; A19-B60; A19-B61; A19-B62; A19-B63; A19-B64; A19-B65; A19-B66; A19-B67; A19-B68; A19-B69; A19-B70; A19-B71; A19-B72; A19-B73; A19-B74; A19-B75; A19-B76; A19-B77; A19-B78; A19-B79; A19-B80; A19-B81; A19-B82; A19-B83; A19-B84; A19-B85; A19-B86; A19-B87; A19-B88; A19-B89; A19-B90; A19-B91; A19-B92; A19-B93; A19-B94; A19-B95; A19-B96; A19-B97; A19-B98; A19-B99; A19-B100; A19-B101; A19-B102; A19-B103; A19-B104; A19-B105; A19-B106; A19-B107; A19-B108; A19-B109; A19-B110; A19-B111; A19-B112; A19-B113;

A20-B1; A20-B2; A20-B3; A20-B4; A20-B5; A20-B6; A20-B7; A20-B8; A20-B9; A20-B10; A20-B11; A20-B12; A20-B13; A20-B14; A20-B15; A20-B16; A20-B17; A20-B18; A20-B19; A20-B20; A20-B21; A20-B22; A20-B23; A20-B24; A20-B25; A20-B26; A20-B27; A20-B28; A20-B29; A20-B30; A20-B31; A20-B32; A20-B33; A20-B34; A20-B35; A20-B36; A20-B37; A20-B38; A20-B39; A20-B40; A20-B41; A20-B42; A20-B43; A20-B44; A20-B45; A20-B46; A20-B47; A20-B48; A20-B49; A20-B50; A20-B51; A20-B52; A20-B53; A20-B54; A20-B55; A20-B56; A20-B57; A20-B58; A20-B59; A20-B60; A20-B61; A20-B62; A20-B63; A20-B64; A20-B65; A20-B66; A20-B67; A20-B68; A20-B69; A20-B70; A20-B71; A20-B72; A20-B73; A20-B74; A20-B75; A20-B76; A20-B77; A20-B78; A20-B79; A20-B80; A20-B81; A20-B82; A20-B83; A20-B84; A20-B85; A20-B86; A20-B87; A20-B88; A20-B89; A20-B90; A20-B91; A20-B92; A20-B93; A20-B94; A20-B95; A20-B96; A20-B97; A20-B98; A20-B99; A20-B100; A20-B101; A20-B102; A20-B103; A20-B104; A20-B105; A20-B106; A20-B107; A20-B108; A20-B109; A20-B110; A20-B111; A20-B112; A20-B113;

A21-B1; A21-B2; A21-B3; A21-B4; A21-B5; A21-B6; A21-B7; A21-B8; A21-B9; A21-B10; A21-B11; A21-B12; A21-B13; A21-B14; A21-B15; A21-B16; A21-B17; A21-B18; A21-B19; A21-B20; A21-B21; A21-B22; A21-B23; A21-B24; A21-B25; A21-B26; A21-B27; A21-B28; A21-B29; A21-B30; A21-B31; A21-B32; A21-B33; A21-B34; A21-B35; A21-B36; A21-B37; A21-B38; A21-B39; A21-B40; A21-B41; A21-B42; A21-B43; A21-B44; A21-B45; A21-B46; A21-B47; A21-B48; A21-B49; A21-B50; A21-B51; A21-B52; A21-B53; A21-B54; A21-B55; A21-B56; A21-B57; A21-B58; A21-B59; A21-B60; A21-B61; A21-B62; A21-B63; A21-B64; A21-B65; A21-B66; A21-B67; A21-B68; A21-B69; A21-B70; A21-B71; A21-B72; A21-B73; A21-B74; A21-B75; A21-B76; A21-B77; A21-B78; A21-B79; A21-B80; A21-B81; A21-B82; A21-B83; A21-B84; A21-B85; A21-B86; A21-B87; A21-B88; A21-B89; A21-B90; A21-B91; A21-B92; A21-B93; A21-B94; A21-B95; A21-B96; A21-B97; A21-B98; A21-B99; A21-B100; A21-B101; A21-B102; A21-B103; A21-B104; A21-B105; A21-B106; A21-B107; A21-B108; A21-B109; A21-B110; A21-B111; A21-B112; A21-B113;

A22-B1; A22-B2; A22-B3; A22-B4; A22-B5; A22-B6; A22-B7; A22-B8; A22-B9; A22-B10; A22-B11; A22-B12; A22-B13; A22-B14; A22-B15; A22-B16; A22-B17; A22-B18; A22-B19; A22-B20; A22-B21; A22-B22; A22-B23; A22-B24; A22-B25; A22-B26; A22-B27; A22-B28; A22-B29; A22-B30; A22-B31; A22-B32; A22-B33; A22-B34; A22-B35; A22-B36; A22-B37; A22-B38; A22-B39; A22-B40; A22-B41; A22-B42; A22-B43; A22-B44; A22-B45; A22-B46; A22-B47; A22-B48; A22-B49; A22-B50; A22-B51; A22-B52; A22-B53; A22-B54; A22-B55; A22-B56; A22-B57; A22-B58; A22-B59; A22-B60; A22-B61; A22-B62; A22-B63; A22-B64; A22-B65; A22-B66; A22-B67; A22-B68; A22-B69; A22-B70; A22-B71; A22-B72; A22-B73; A22-B74; A22-B75; A22-B76; A22-B77; A22-B78; A22-B79; A22-B80; A22-B81; A22-B82; A22-B83; A22-B84; A22-B85; A22-B86; A22-B87; A22-B88; A22-B89; A22-B90; A22-B91; A22-B92; A22-B93; A22-B94; A22-B95; A22-B96; A22-B97; A22-B98; A22-B99; A22-B100; A22-B101; A22-B102; A22-B103; A22-B104; A22-B105; A22-B106; A22-B107; A22-B108; A22-B109; A22-B110; A22-B111; A22-B112; A22-B113;

A23-B1; A23-B2; A23-B3; A23-B4; A23-B5; A23-B6; A23-B7; A23-B8; A23-B9; A23-B10; A23-B11; A23-B12; A23-B13; A23-B14; A23-B15; A23-B16; A23-B17; A23-B18; A23-B19; A23-B20; A23-B21; A23-B22; A23-B23; A23-B24; A23-B25; A23-B26; A23-B27; A23-B28; A23-B29; A23-B30; A23-B31; A23-B32; A23-B33; A23-B34; A23-B35; A23-B36; A23-B37; A23-B38; A23-B39; A23-B40; A23-B41; A23-B42; A23-B43; A23-B44; A23-B45; A23-B46; A23-B47; A23-B48; A23-B49; A23-B50; A23-B51; A23-B52; A23-B53; A23-B54; A23-B55; A23-B56; A23-B57; A23-B58; A23-B59; A23-B60; A23-B61; A23-B62; A23-B63; A23-B64; A23-B65; A23-B66; A23-B67; A23-B68; A23-B69; A23-B70; A23-B71; A23-B72; A23-B73; A23-B74; A23-B75; A23-B76; A23-B77; A23-B78; A23-B79; A23-B80; A23-B81; A23-B82; A23-B83; A23-B84; A23-B85; A23-B86; A23-B87; A23-B88; A23-B89; A23-B90; A23-B91; A23-B92; A23-B93; A23-B94; A23-B95; A23-B96; A23-B97; A23-B98; A23-B99; A23-B100; A23-B101; A23-B102; A23-B103; A23-B104; A23-B105; A23-B106; A23-B107; A23-B108; A23-B109; A23-B110; A23-B111; A23-B112; A23-B113;

A24-B1; A24-B2; A24-B3; A24-B4; A24-B5; A24-B6; A24-B7; A24-B8; A24-B9; A24-B10; A24-B11; A24-B12; A24-B13; A24-B14; A24-B15; A24-B16; A24-B17; A24-B18; A24-B19; A24-B20; A24-B21; A24-B22; A24-B23; A24-B24; A24-B25; A24-B26; A24-B27; A24-B28; A24-B29; A24-B30; A24-B31; A24-B32; A24-B33; A24-B34; A24-B35; A24-B36; A24-B37; A24-B38; A24-B39; A24-B40; A24-B41; A24-B42; A24-B43; A24-B44; A24-B45; A24-B46; A24-B47; A24-B48; A24-B49; A24-B50; A24-B51; A24-B52; A24-B53; A24-B54; A24-B55; A24-B56; A24-B57; A24-B58; A24-B59; A24-B60; A24-B61; A24-B62; A24-B63; A24-B64; A24-B65; A24-B66; A24-B67; A24-B68; A24-B69; A24-B70; A24-B71; A24-B72; A24-B73; A24-B74; A24-B75; A24-B76; A24-B77; A24-B78; A24-B79; A24-B80; A24-B81; A24-B82; A24-B83; A24-B84; A24-B85; A24-B86; A24-B87; A24-B88; A24-B89; A24-B90; A24-B91; A24-B92; A24-B93; A24-B94; A24-B95; A24-B96; A24-B97; A24-B98; A24-B99; A24-B100; A24-B101; A24-B102; A24-B103; A24-B104; A24-B105; A24-B106; A24-B107; A24-B108; A24-B109; A24-B110; A24-B111; A24-B112; A24-B113;

A25-B1; A25-B2; A25-B3; A25-B4; A25-B5; A25-B6; A25-B7; A25-B8; A25-B9; A25-B10; A25-B11; A25-B12; A25-B13; A25-B14; A25-B15; A25-B16; A25-B17; A25-B18; A25-B19; A25-B20; A25-B21; A25-B22; A25-B23; A25-B24; A25-B25; A25-B26; A25-B27; A25-B28; A25-B29; A25-B30; A25-B31; A25-B32; A25-B33; A25-B34; A25-B35; A25-B36; A25-B37; A25-B38; A25-B39; A25-B40; A25-B41; A25-B42; A25-B43; A25-B44; A25-B45; A25-B46; A25-B47; A25-B48; A25-B49; A25-B50; A25-B51; A25-B52; A25-B53; A25-B54; A25-B55; A25-B56; A25-B57; A25-B58; A25-B59; A25-B60; A25-B61; A25-B62; A25-B63; A25-B64; A25-B65; A25-B66; A25-B67; A25-B68; A25-B69; A25-B70; A25-B71; A25-B72; A25-B73; A25-B74; A25-B75; A25-B76; A25-B77; A25-B78; A25-B79; A25-B80; A25-B81; A25-B82; A25-B83; A25-B84; A25-B85; A25-B86; A25-B87; A25-B88; A25-B89; A25-B90; A25-B91; A25-B92; A25-B93; A25-B94; A25-B95; A25-B96; A25-B97; A25-B98; A25-B99; A25-B100; A25-B101; A25-B102; A25-B103; A25-B104; A25-B105; A25-B106; A25-B107; A25-B108; A25-B109; A25-B110; A25-B111; A25-B112; A25-B113;

A26-B1; A26-B2; A26-B3; A26-B4; A26-B5; A26-B6; A26-B7; A26-B8; A26-B9; A26-B10; A26-B11; A26-B12; A26-B13; A26-B14; A26-B15; A26-B16; A26-B17; A26-B18; A26-B19; A26-B20; A26-B21; A26-B22; A26-B23; A26-B24; A26-B25; A26-B26; A26-B27; A26-B28; A26-B29; A26-B30; A26-B31; A26-B32; A26-B33; A26-B34; A26-B35; A26-B36; A26-B37; A26-B38; A26-B39; A26-B40; A26-B41; A26-B42; A26-B43; A26-B44; A26-B45; A26-B46; A26-B47; A26-B48; A26-B49; A26-B50; A26-B51; A26-B52; A26-B53; A26-B54; A26-B55; A26-B56; A26-B57; A26-B58; A26-B59; A26-B60; A26-B61; A26-B62; A26-B63; A26-B64; A26-B65; A26-B66; A26-B67; A26-B68; A26-B69; A26-B70; A26-B71; A26-B72; A26-B73; A26-B74; A26-B75; A26-B76; A26-B77; A26-B78; A26-B79; A26-B80; A26-B81; A26-B82; A26-B83; A26-B84; A26-B85; A26-B86; A26-B87; A26-B88; A26-B89; A26-B90; A26-B91; A26-B92; A26-B93; A26-B94; A26-B95; A26-B96; A26-B97; A26-B98; A26-B99; A26-B100; A26-B101; A26-B102; A26-B103; A26-B104; A26-B105; A26-B106; A26-B107; A26-B108; A26-B109; A26-B110; A26-B111; A26-B112; A26-B113;

A27-B1; A27-B2; A27-B3; A27-B4; A27-B5; A27-B6; A27-B7; A27-B8; A27-B9; A27-B10; A27-B11; A27-B12; A27-B13; A27-B14; A27-B15; A27-B16; A27-B17; A27-B18; A27-B19; A27-B20; A27-B21; A27-B22; A27-B23; A27-B24; A27-B25; A27-B26; A27-B27; A27-B28; A27-B29; A27-B30; A27-B31; A27-B32; A27-B33; A27-B34; A27-B35; A27-B36; A27-B37; A27-B38; A27-B39; A27-B40; A27-B41; A27-B42; A27-B43; A27-B44; A27-B45; A27-B46; A27-B47; A27-B48; A27-B49; A27-B50; A27-B51; A27-B52; A27-B53; A27-B54; A27-B55; A27-B56; A27-B57; A27-B58; A27-B59; A27-B60; A27-B61; A27-B62; A27-B63; A27-B64; A27-B65; A27-B66; A27-B67; A27-B68; A27-B69; A27-B70; A27-B71; A27-B72; A27-B73; A27-B74; A27-B75; A27-B76; A27-B77; A27-B78; A27-B79; A27-B80; A27-B81; A27-B82; A27-B83; A27-B84; A27-B85; A27-B86; A27-B87; A27-B88; A27-B89; A27-B90; A27-B91; A27-B92; A27-B93; A27-B94; A27-B95; A27-B96; A27-B97; A27-B98; A27-B99; A27-B100; A27-B101; A27-B102; A27-B103; A27-B104; A27-B105; A27-B106; A27-B107; A27-B108; A27-B109; A27-B110; A27-B111; A27-B112; A27-B113;

A28-B1; A28-B2; A28-B3; A28-B4; A28-B5; A28-B6; A28-B7; A28-B8; A28-B9; A28-B10; A28-B11; A28-B12; A28-B13; A28-B14; A28-B15; A28-B16; A28-B17; A28-B18; A28-B19; A28-B20; A28-B21; A28-B22; A28-B23; A28-B24; A28-B25; A28-B26; A28-B27; A28-B28; A28-B29; A28-B30; A28-B31; A28-B32; A28-B33; A28-B34; A28-B35; A28-B36; A28-B37; A28-B38; A28-B39; A28-B40; A28-B41; A28-B42; A28-B43; A28-B44; A28-B45; A28-B46; A28-B47; A28-B48; A28-B49; A28-B50; A28-B51; A28-B52; A28-B53; A28-B54; A28-B55; A28-B56; A28-B57; A28-B58; A28-B59; A28-B60; A28-B61; A28-B62; A28-B63; A28-B64; A28-B65; A28-B66; A28-B67; A28-B68; A28-B69; A28-B70; A28-B71; A28-B72; A28-B73; A28-B74; A28-B75; A28-B76; A28-B77; A28-B78; A28-B79; A28-B80; A28-B81; A28-B82; A28-B83; A28-B84; A28-B85; A28-B86; A28-B87; A28-B88; A28-B89; A28-B90; A28-B91; A28-B92; A28-B93; A28-B94; A28-B95; A28-B96; A28-B97; A28-B98; A28-B99; A28-B100; A28-B101; A28-B102; A28-B103; A28-B104; A28-B105; A28-B106; A28-B107; A28-B108; A28-B109; A28-B110; A28-B111; A28-B112; A28-B113;

A29-B1; A29-B2; A29-B3; A29-B4; A29-B5; A29-B6; A29-B7; A29-B8; A29-B9; A29-B10; A29-B11; A29-B12; A29-B13; A29-B14; A29-B15; A29-B16; A29-B17; A29-B18; A29-B19; A29-B20; A29-B21; A29-B22; A29-B23; A29-B24; A29-B25; A29-B26; A29-B27; A29-B28; A29-B29; A29-B30; A29-B31; A29-B32; A29-B33; A29-B34; A29-B35; A29-B36; A29-B37; A29-B38; A29-B39; A29-B40; A29-B41; A29-B42; A29-B43; A29-B44; A29-B45; A29-B46; A29-B47; A29-B48; A29-B49; A29-B50; A29-B51; A29-B52; A29-B53; A29-B54; A29-B55; A29-B56; A29-B57; A29-B58; A29-B59; A29-B60; A29-B61; A29-B62; A29-B63; A29-B64; A29-B65; A29-B66; A29-B67; A29-B68; A29-B69; A29-B70; A29-B71; A29-B72; A29-B73; A29-B74; A29-B75; A29-B76; A29-B77; A29-B78; A29-B79; A29-B80; A29-B81; A29-B82; A29-B83; A29-B84; A29-B85; A29-B86; A29-B87; A29-B88; A29-B89; A29-B90; A29-B91; A29-B92; A29-B93; A29-B94; A29-B95; A29-B96; A29-B97; A29-B98; A29-B99; A29-B100; A29-B101; A29-B102; A29-B103; A29-B104; A29-B105; A29-B106; A29-B107; A29-B108; A29-B109; A29-B110; A29-B111; A29-B112; A29-B113;

A30-B1; A30-B2; A30-B3; A30-B4; A30-B5; A30-B6; A30-B7; A30-B8; A30-B9; A30-B10; A30-B11; A30-B12; A30-B13; A30-B14; A30-B15; A30-B16; A30-B17; A30-B18; A30-B19; A30-B20; A30-B21; A30-B22; A30-B23; A30-B24; A30-B25; A30-B26; A30-B27; A30-B28; A30-B29; A30-B30; A30-B31; A30-B32; A30-B33; A30-B34; A30-B35; A30-B36; A30-B37; A30-B38; A30-B39; A30-B40; A30-B41; A30-B42; A30-B43; A30-B44; A30-B45; A30-B46; A30-B47; A30-B48; A30-B49; A30-B50; A30-B51; A30-B52; A30-B53; A30-B54; A30-B55; A30-B56; A30-B57; A30-B58; A30-B59; A30-B60; A30-B61; A30-B62; A30-B63; A30-B64; A30-B65; A30-B66; A30-B67; A30-B68; A30-B69; A30-B70; A30-B71; A30-B72; A30-B73; A30-B74; A30-B75; A30-B76; A30-B77; A30-B78; A30-B79; A30-B80; A30-B81; A30-B82; A30-B83; A30-B84; A30-B85; A30-B86; A30-B87; A30-B88; A30-B89; A30-B90; A30-B91; A30-B92; A30-B93; A30-B94; A30-B95; A30-B96; A30-B97; A30-B98; A30-B99; A30-B100; A30-B101; A30-B102; A30-B103; A30-B104; A30-B105; A30-B106; A30-B107; A30-B108; A30-B109; A30-B110; A30-B111; A30-B112; A30-B113;

A31-B1; A31-B2; A31-B3; A31-B4; A31-B5; A31-B6; A31-B7; A31-B8; A31-B9; A31-B10; A31-B11; A31-B12; A31-B13; A31-B14; A31-B15; A31-B16; A31-B17; A31-B18; A31-B19; A31-B20; A31-B21; A31-B22; A31-B23; A31-B24; A31-B25; A31-B26; A31-B27; A31-B28; A31-B29; A31-B30; A31-B31; A31-B32; A31-B33; A31-B34; A31-B35; A31-B36; A31-B37; A31-B38; A31-B39; A31-B40; A31-B41; A31-B42; A31-B43; A31-B44; A31-B45; A31-B46; A31-B47; A31-B48; A31-B49; A31-B50; A31-B51; A31-B52; A31-B53; A31-B54; A31-B55; A31-B56; A31-B57; A31-B58; A31-B59; A31-B60; A31-B61; A31-B62; A31-B63; A31-B64; A31-B65; A31-B66; A31-B67; A31-B68; A31-B69; A31-B70; A31-B71; A31-B72; A31-B73; A31-B74; A31-B75; A31-B76; A31-B77; A31-B78; A31-B79; A31-B80; A31-B81; A31-B82; A31-B83; A31-B84; A31-B85; A31-B86; A31-B87; A31-B88; A31-B89; A31-B90; A31-B91; A31-B92; A31-B93; A31-B94; A31-B95; A31-B96; A31-B97; A31-B98; A31-B99; A31-B100; A31-B101; A31-B102; A31-B103; A31-B104; A31-B105; A31-B106; A31-B107; A31-B108; A31-B109; A31-B110; A31-B111; A31-B112; A31-B113;

A32-B1; A32-B2; A32-B3; A32-B4; A32-B5; A32-B6; A32-B7; A32-B8; A32-B9; A32-B10; A32-B11; A32-B12; A32-B13; A32-B14; A32-B15; A32-B16; A32-B17; A32-B18; A32-B19; A32-B20; A32-B21; A32-B22; A32-B23; A32-B24; A32-B25; A32-B26; A32-B27; A32-B28; A32-B29; A32-B30; A32-B31; A32-B32; A32-B33; A32-B34; A32-B35; A32-B36; A32-B37; A32-B38; A32-B39; A32-B40; A32-B41; A32-B42; A32-B43; A32-B44; A32-B45; A32-B46; A32-B47; A32-B48; A32-B49; A32-B50; A32-B51; A32-B52; A32-B53; A32-B54; A32-B55; A32-B56; A32-B57; A32-B58; A32-B59; A32-B60; A32-B61; A32-B62; A32-B63; A32-B64; A32-B65; A32-B66; A32-B67; A32-B68; A32-B69; A32-B70; A32-B71; A32-B72; A32-B73; A32-B74; A32-B75; A32-B76; A32-B77; A32-B78; A32-B79; A32-B80; A32-B81; A32-B82; A32-B83; A32-B84; A32-B85; A32-B86; A32-B87; A32-B88; A32-B89; A32-B90; A32-B91; A32-B92; A32-B93; A32-B94; A32-B95; A32-B96; A32-B97; A32-B98; A32-B99; A32-B100; A32-B101; A32-B102; A32-B103; A32-B104; A32-B105; A32-B106; A32-B107; A32-B108; A32-B109; A32-B110; A32-B111; A32-B112; A32-B113;

A33-B1; A33-B2; A33-B3; A33-B4; A33-B5; A33-B6; A33-B7; A33-B8; A33-B9; A33-B10; A33-B11; A33-B12; A33-B13; A33-B14; A33-B15; A33-B16; A33-B17; A33-B18; A33-B19; A33-B20; A33-B21; A33-B22; A33-B23; A33-B24; A33-B25; A33-B26; A33-B27; A33-B28; A33-B29; A33-B30; A33-B31; A33-B32; A33-B33; A33-B34; A33-B35; A33-B36; A33-B37; A33-B38; A33-B39; A33-B40; A33-B41; A33-B42; A33-B43; A33-B44; A33-B45; A33-B46; A33-B47; A33-B48; A33-B49; A33-B50; A33-B51; A33-B52; A33-B53; A33-B54; A33-B55; A33-B56; A33-B57; A33-B58; A33-B59; A33-B60; A33-B61; A33-B62; A33-B63; A33-B64; A33-B65; A33-B66; A33-B67; A33-B68; A33-B69; A33-B70; A33-B71; A33-B72; A33-B73; A33-B74; A33-B75; A33-B76; A33-B77; A33-B78; A33-B79; A33-B80; A33-B81; A33-B82; A33-B83; A33-B84; A33-B85; A33-B86; A33-B87; A33-B88; A33-B89; A33-B90; A33-B91; A33-B92; A33-B93; A33-B94; A33-B95; A33-B96; A33-B97; A33-B98; A33-B99; A33-B100; A33-B101; A33-B102; A33-B103; A33-B104; A33-B105; A33-B106; A33-B107; A33-B108; A33-B109; A33-B110; A33-B111; A33-B112; A33-B113;

A34-B1; A34-B2; A34-B3; A34-B4; A34-B5; A34-B6; A34-B7; A34-B8; A34-B9; A34-B10; A34-B11; A34-B12; A34-B13; A34-B14; A34-B15; A34-B16; A34-B17; A34-B18; A34-B19; A34-B20; A34-B21; A34-B22; A34-B23; A34-B24; A34-B25; A34-B26; A34-B27; A34-B28; A34-B29; A34-B30; A34-B31; A34-B32; A34-B33; A34-B34; A34-B35; A34-B36; A34-B37; A34-B38; A34-B39; A34-B40; A34-B41; A34-B42; A34-B43; A34-B44; A34-B45; A34-B46; A34-B47; A34-B48; A34-B49; A34-B50; A34-B51; A34-B52; A34-B53; A34-B54; A34-B55; A34-B56; A34-B57; A34-B58; A34-B59; A34-B60; A34-B61; A34-B62; A34-B63; A34-B64; A34-B65; A34-B66; A34-B67; A34-B68; A34-B69; A34-B70; A34-B71; A34-B72; A34-B73; A34-B74; A34-B75; A34-B76; A34-B77; A34-B78; A34-B79; A34-B80; A34-B81; A34-B82; A34-B83; A34-B84; A34-B85; A34-B86; A34-B87; A34-B88; A34-B89; A34-B90; A34-B91; A34-B92; A34-B93; A34-B94; A34-B95; A34-B96; A34-B97; A34-B98; A34-B99; A34-B100; A34-B101; A34-B102; A34-B103; A34-B104; A34-B105; A34-B106; A34-B107; A34-B108; A34-B109; A34-B110; A34-B111; A34-B112; A34-B113;

A35-B1; A35-B2; A35-B3; A35-B4; A35-B5; A35-B6; A35-B7; A35-B8; A35-B9; A35-B10; A35-B11; A35-B12; A35-B13; A35-B14; A35-B15; A35-B16; A35-B17; A35-B18; A35-B19; A35-B20; A35-B21; A35-B22; A35-B23; A35-B24; A35-B25; A35-B26; A35-B27; A35-B28; A35-B29; A35-B30; A35-B31; A35-B32; A35-B33; A35-B34; A35-B35; A35-B36; A35-B37; A35-B38; A35-B39; A35-B40; A35-B41; A35-B42; A35-B43; A35-B44; A35-B45; A35-B46; A35-B47; A35-B48; A35-B49; A35-B50; A35-B51; A35-B52; A35-B53; A35-B54; A35-B55; A35-B56; A35-B57; A35-B58; A35-B59; A35-B60; A35-B61; A35-B62; A35-B63; A35-B64; A35-B65; A35-B66; A35-B67; A35-B68; A35-B69; A35-B70; A35-B71; A35-B72; A35-B73; A35-B74; A35-B75; A35-B76; A35-B77; A35-B78; A35-B79; A35-B80; A35-B81; A35-B82; A35-B83; A35-B84; A35-B85; A35-B86; A35-B87; A35-B88; A35-B89; A35-B90; A35-B91; A35-B92; A35-B93; A35-B94; A35-B95; A35-B96; A35-B97; A35-B98; A35-B99; A35-B100; A35-B101; A35-B102; A35-B103; A35-B104; A35-B105; A35-B106; A35-B107; A35-B108; A35-B109; A35-B110; A35-B111; A35-B112; A35-B113;

A36-B1; A36-B2; A36-B3; A36-B4; A36-B5; A36-B6; A36-B7; A36-B8; A36-B9; A36-B10; A36-B11; A36-B12; A36-B13; A36-B14; A36-B15; A36-B16; A36-B17; A36-B18; A36-B19; A36-B20; A36-B21; A36-B22; A36-B23; A36-B24; A36-B25; A36-B26; A36-B27; A36-B28; A36-B29; A36-B30; A36-B31; A36-B32; A36-B33; A36-B34; A36-B35; A36-B36; A36-B37; A36-B38; A36-B39; A36-B40; A36-B41; A36-B42; A36-B43; A36-B44; A36-B45; A36-B46; A36-B47; A36-B48; A36-B49; A36-B50; A36-B51; A36-B52; A36-B53; A36-B54; A36-B55; A36-B56; A36-B57; A36-B58; A36-B59; A36-B60; A36-B61; A36-B62; A36-B63; A36-B64; A36-B65; A36-B66; A36-B67; A36-B68; A36-B69; A36-B70; A36-B71; A36-B72; A36-B73; A36-B74; A36-B75; A36-B76; A36-B77; A36-B78; A36-B79; A36-B80; A36-B81; A36-B82; A36-B83; A36-B84; A36-B85; A36-B86; A36-B87; A36-B88; A36-B89; A36-B90; A36-B91; A36-B92; A36-B93; A36-B94; A36-B95; A36-B96; A36-B97; A36-B98; A36-B99; A36-B100; A36-B101; A36-B102; A36-B103; A36-B104; A36-B105; A36-B106; A36-B107; A36-B108; A36-B109; A36-B110; A36-B111; A36-B112; A36-B113;

A37-B1; A37-B2; A37-B3; A37-B4; A37-B5; A37-B6; A37-B7; A37-B8; A37-B9; A37-B10; A37-B11; A37-B12; A37-B13; A37-B14; A37-B15; A37-B16; A37-B17; A37-B18; A37-B19; A37-B20; A37-B21; A37-B22; A37-B23; A37-B24; A37-B25; A37-B26; A37-B27; A37-B28; A37-B29; A37-B30; A37-B31; A37-B32; A37-B33; A37-B34; A37-B35; A37-B36; A37-B37; A37-B38; A37-B39; A37-B40; A37-B41; A37-B42; A37-B43; A37-B44; A37-B45; A37-B46; A37-B47; A37-B48; A37-B49; A37-B50; A37-B51; A37-B52; A37-B53; A37-B54; A37-B55; A37-B56; A37-B57; A37-B58; A37-B59; A37-B60; A37-B61; A37-B62; A37-B63; A37-B64; A37-B65; A37-B66; A37-B67; A37-B68; A37-B69; A37-B70; A37-B71; A37-B72; A37-B73; A37-B74; A37-B75; A37-B76; A37-B77; A37-B78; A37-B79; A37-B80; A37-B81; A37-B82; A37-B83; A37-B84; A37-B85; A37-B86; A37-B87; A37-B88; A37-B89; A37-B90; A37-B91; A37-B92; A37-B93; A37-B94; A37-B95; A37-B96; A37-B97; A37-B98; A37-B99; A37-B100; A37-B101; A37-B102; A37-B103; A37-B104; A37-B105; A37-B106; A37-B107; A37-B108; A37-B109; A37-B110; A37-B111; A37-B112; A37-B113;

A38-B1; A38-B2; A38-B3; A38-B4; A38-B5; A38-B6; A38-B7; A38-B8; A38-B9; A38-B10; A38-B11; A38-B12; A38-B13; A38-B14; A38-B15; A38-B16; A38-B17; A38-B18; A38-B19; A38-B20; A38-B21; A38-B22; A38-B23; A38-B24; A38-B25; A38-B26; A38-B27; A38-B28; A38-B29; A38-B30; A38-B31; A38-B32; A38-B33; A38-B34; A38-B35; A38-B36; A38-B37; A38-B38; A38-B39; A38-B40; A38-B41; A38-B42; A38-B43; A38-B44; A38-B45; A38-B46; A38-B47; A38-B48; A38-B49; A38-B50; A38-B51; A38-B52; A38-B53; A38-B54; A38-B55; A38-B56; A38-B57; A38-B58; A38-B59; A38-B60; A38-B61; A38-B62; A38-B63; A38-B64; A38-B65; A38-B66; A38-B67; A38-B68; A38-B69; A38-B70; A38-B71; A38-B72; A38-B73; A38-B74; A38-B75; A38-B76; A38-B77; A38-B78; A38-B79; A38-B80; A38-B81; A38-B82; A38-B83; A38-B84; A38-B85; A38-B86; A38-B87; A38-B88; A38-B89; A38-B90; A38-B91; A38-B92; A38-B93; A38-B94; A38-B95; A38-B96; A38-B97; A38-B98; A38-B99; A38-B100; A38-B101; A38-B102; A38-B103; A38-B104; A38-B105; A38-B106; A38-B107; A38-B108; A38-B109; A38-B110; A38-B111; A38-B112; A38-B113;

A39-B1; A39-B2; A39-B3; A39-B4; A39-B5; A39-B6; A39-B7; A39-B8; A39-B9; A39-B10; A39-B11; A39-B12; A39-B13; A39-B14; A39-B15; A39-B16; A39-B17; A39-B18; A39-B19; A39-B20; A39-B21; A39-B22; A39-B23; A39-B24; A39-B25; A39-B26; A39-B27; A39-B28; A39-B29; A39-B30; A39-B31; A39-B32; A39-B33; A39-B34; A39-B35; A39-B36; A39-B37; A39-B38; A39-B39; A39-B40; A39-B41; A39-B42; A39-B43; A39-B44; A39-B45; A39-B46; A39-B47; A39-B48; A39-B49; A39-B50; A39-B51; A39-B52; A39-B53; A39-B54; A39-B55; A39-B56; A39-B57; A39-B58; A39-B59; A39-B60; A39-B61; A39-B62; A39-B63; A39-B64; A39-B65; A39-B66; A39-B67; A39-B68; A39-B69; A39-B70; A39-B71; A39-B72; A39-B73; A39-B74; A39-B75; A39-B76; A39-B77; A39-B78; A39-B79; A39-B80; A39-B81; A39-B82; A39-B83; A39-B84; A39-B85; A39-B86; A39-B87; A39-B88; A39-B89; A39-B90; A39-B91; A39-B92; A39-B93; A39-B94; A39-B95; A39-B96; A39-B97; A39-B98; A39-B99; A39-B100; A39-B101; A39-B102; A39-B103; A39-B104; A39-B105; A39-B106; A39-B107; A39-B108; A39-B109; A39-B110; A39-B111; A39-B112; A39-B113;

A40-B1; A40-B2; A40-B3; A40-B4; A40-B5; A40-B6; A40-B7; A40-B8; A40-B9; A40-B10; A40-B11; A40-B12; A40-B13; A40-B14; A40-B15; A40-B16; A40-B17; A40-B18; A40-B19; A40-B20; A40-B21; A40-B22; A40-B23; A40-B24; A40-B25; A40-B26; A40-B27; A40-B28; A40-B29; A40-B30; A40-B31; A40-B32; A40-B33; A40-B34; A40-B35; A40-B36; A40-B37; A40-B38; A40-B39; A40-B40; A40-B41; A40-B42; A40-B43; A40-B44; A40-B45; A40-B46; A40-B47; A40-B48; A40-B49; A40-B50; A40-B51; A40-B52; A40-B53; A40-B54; A40-B55; A40-B56; A40-B57; A40-B58; A40-B59; A40-B60; A40-B61; A40-B62; A40-B63; A40-B64; A40-B65; A40-B66; A40-B67; A40-B68; A40-B69; A40-B70; A40-B71; A40-B72; A40-B73; A40-B74; A40-B75; A40-B76; A40-B77; A40-B78; A40-B79; A40-B80; A40-B81; A40-B82; A40-B83; A40-B84; A40-B85; A40-B86; A40-B87; A40-B88; A40-B89; A40-B90; A40-B91; A40-B92; A40-B93; A40-B94; A40-B95; A40-B96; A40-B97; A40-B98; A40-B99; A40-B100; A40-B101; A40-B102; A40-B103; A40-B104; A40-B105; A40-B106; A40-B107; A40-B108; A40-B109; A40-B110; A40-B111; A40-B112; A40-B113;

A41-B1; A41-B2; A41-B3; A41-B4; A41-B5; A41-B6; A41-B7; A41-B8; A41-B9; A41-B10; A41-B11; A41-B12; A41-B13; A41-B14; A41-B15; A41-B16; A41-B17; A41-B18; A41-B19; A41-B20; A41-B21; A41-B22; A41-B23; A41-B24; A41-B25; A41-B26; A41-B27; A41-B28; A41-B29; A41-B30; A41-B31; A41-B32; A41-B33; A41-B34; A41-B35; A41-B36; A41-B37; A41-B38; A41-B39; A41-B40; A41-B41; A41-B42; A41-B43; A41-B44; A41-B45; A41-B46; A41-B47; A41-B48; A41-B49; A41-B50; A41-B51; A41-B52; A41-B53; A41-B54; A41-B55; A41-B56; A41-B57; A41-B58; A41-B59; A41-B60; A41-B61; A41-B62; A41-B63; A41-B64; A41-B65; A41-B66; A41-B67; A41-B68; A41-B69; A41-B70; A41-B71; A41-B72; A41-B73; A41-B74; A41-B75; A41-B76; A41-B77; A41-B78; A41-B79; A41-B80; A41-B81; A41-B82; A41-B83; A41-B84; A41-B85; A41-B86; A41-B87; A41-B88; A41-B89; A41-B90; A41-B91; A41-B92; A41-B93; A41-B94; A41-B95; A41-B96; A41-B97; A41-B98; A41-B99; A41-B100; A41-B101; A41-B102; A41-B103; A41-B104; A41-B105; A41-B106; A41-B107; A41-B108; A41-B109; A41-B110; A41-B111; A41-B112; A41-B113;

A42-B1; A42-B2; A42-B3; A42-B4; A42-B5; A42-B6; A42-B7; A42-B8; A42-B9; A42-B10; A42-B11; A42-B12; A42-B13; A42-B14; A42-B15; A42-B16; A42-B17; A42-B18; A42-B19; A42-B20; A42-B21; A42-B22; A42-B23; A42-B24; A42-B25; A42-B26; A42-B27; A42-B28; A42-B29; A42-B30; A42-B31; A42-B32; A42-B33; A42-B34; A42-B35; A42-B36; A42-B37; A42-B38; A42-B39; A42-B40; A42-B41; A42-B42; A42-B43; A42-B44; A42-B45; A42-B46; A42-B47; A42-B48; A42-B49; A42-B50; A42-B51; A42-B52; A42-B53; A42-B54; A42-B55; A42-B56; A42-B57; A42-B58; A42-B59; A42-B60; A42-B61; A42-B62; A42-B63; A42-B64; A42-B65; A42-B66; A42-B67; A42-B68; A42-B69; A42-B70; A42-B71; A42-B72; A42-B73; A42-B74; A42-B75; A42-B76; A42-B77; A42-B78; A42-B79; A42-B80; A42-B81; A42-B82; A42-B83; A42-B84; A42-B85; A42-B86; A42-B87; A42-B88; A42-B89; A42-B90; A42-B91; A42-B92; A42-B93; A42-B94; A42-B95; A42-B96; A42-B97; A42-B98; A42-B99; A42-B100; A42-B101; A42-B102; A42-B103; A42-B104; A42-B105; A42-B106; A42-B107; A42-B108; A42-B109; A42-B110; A42-B111; A42-B112; A42-B113;

A43-B1; A43-B2; A43-B3; A43-B4; A43-B5; A43-B6; A43-B7; A43-B8; A43-B9; A43-B10; A43-B11; A43-B12; A43-B13; A43-B14; A43-B15; A43-B16; A43-B17; A43-B18; A43-B19; A43-B20; A43-B21; A43-B22; A43-B23; A43-B24; A43-B25; A43-B26; A43-B27; A43-B28; A43-B29; A43-B30; A43-B31; A43-B32; A43-B33; A43-B34; A43-B35; A43-B36; A43-B37; A43-B38; A43-B39; A43-B40; A43-B41; A43-B42; A43-B43; A43-B44; A43-B45; A43-B46; A43-B47; A43-B48; A43-B49; A43-B50; A43-B51; A43-B52; A43-B53; A43-B54; A43-B55; A43-B56; A43-B57; A43-B58; A43-B59; A43-B60; A43-B61; A43-B62; A43-B63; A43-B64; A43-B65; A43-B66; A43-B67; A43-B68; A43-B69; A43-B70; A43-B71; A43-B72; A43-B73; A43-B74; A43-B75; A43-B76; A43-B77; A43-B78; A43-B79; A43-B80; A43-B81; A43-B82; A43-B83; A43-B84; A43-B85; A43-B86; A43-B87; A43-B88; A43-B89; A43-B90; A43-B91; A43-B92; A43-B93; A43-B94; A43-B95; A43-B96; A43-B97; A43-B98; A43-B99; A43-B100; A43-B101; A43-B102; A43-B103; A43-B104; A43-B105; A43-B106; A43-B107; A43-B108; A43-B109; A43-B110; A43-B111; A43-B112; A43-B113;

A44-B1; A44-B2; A44-B3; A44-B4; A44-B5; A44-B6; A44-B7; A44-B8; A44-B9; A44-B10; A44-B11; A44-B12; A44-B13; A44-B14; A44-B15; A44-B16; A44-B17; A44-B18; A44-B19; A44-B20; A44-B21; A44-B22; A44-B23; A44-B24; A44-B25; A44-B26; A44-B27; A44-B28; A44-B29; A44-B30; A44-B31; A44-B32; A44-B33; A44-B34; A44-B35; A44-B36; A44-B37; A44-B38; A44-B39; A44-B40; A44-B41; A44-B42; A44-B43; A44-B44; A44-B45; A44-B46; A44-B47; A44-B48; A44-B49; A44-B50; A44-B51; A44-B52; A44-B53; A44-B54; A44-B55; A44-B56; A44-B57; A44-B58; A44-B59; A44-B60; A44-B61; A44-B62; A44-B63; A44-B64; A44-B65; A44-B66; A44-B67; A44-B68; A44-B69; A44-B70; A44-B71; A44-B72; A44-B73; A44-B74; A44-B75; A44-B76; A44-B77; A44-B78; A44-B79; A44-B80; A44-B81; A44-B82; A44-B83; A44-B84; A44-B85; A44-B86; A44-B87; A44-B88; A44-B89; A44-B90; A44-B91; A44-B92; A44-B93; A44-B94; A44-B95; A44-B96; A44-B97; A44-B98; A44-B99; A44-B100; A44-B101; A44-B102; A44-B103; A44-B104; A44-B105; A44-B106; A44-B107; A44-B108; A44-B109; A44-B110; A44-B111; A44-B112; A44-B113;

A45-B1; A45-B2; A45-B3; A45-B4; A45-B5; A45-B6; A45-B7; A45-B8; A45-B9; A45-B10; A45-B11; A45-B12; A45-B13; A45-B14; A45-B15; A45-B16; A45-B17; A45-B18; A45-B19; A45-B20; A45-B21; A45-B22; A45-B23; A45-B24; A45-B25; A45-B26; A45-B27; A45-B28; A45-B29; A45-B30; A45-B31; A45-B32; A45-B33; A45-B34; A45-B35; A45-B36; A45-B37; A45-B38; A45-B39; A45-B40; A45-B41; A45-B42; A45-B43; A45-B44; A45-B45; A45-B46; A45-B47; A45-B48; A45-B49; A45-B50; A45-B51; A45-B52; A45-B53; A45-B54; A45-B55; A45-B56; A45-B57; A45-B58; A45-B59; A45-B60; A45-B61; A45-B62; A45-B63; A45-B64; A45-B65; A45-B66; A45-B67; A45-B68; A45-B69; A45-B70; A45-B71; A45-B72; A45-B73; A45-B74; A45-B75; A45-B76; A45-B77; A45-B78; A45-B79; A45-B80; A45-B81; A45-B82; A45-B83; A45-B84; A45-B85; A45-B86; A45-B87; A45-B88; A45-B89; A45-B90; A45-B91; A45-B92; A45-B93; A45-B94; A45-B95; A45-B96; A45-B97; A45-B98; A45-B99; A45-B100; A45-B101; A45-B102; A45-B103; A45-B104; A45-B105; A45-B106; A45-B107; A45-B108; A45-B109; A45-B110; A45-B111; A45-B112; A45-B113;

A46-B1; A46-B2; A46-B3; A46-B4; A46-B5; A46-B6; A46-B7; A46-B8; A46-B9; A46-B10; A46-B11; A46-B12; A46-B13; A46-B14; A46-B15; A46-B16; A46-B17; A46-B18; A46-B19; A46-B20; A46-B21; A46-B22; A46-B23; A46-B24; A46-B25; A46-B26; A46-B27; A46-B28; A46-B29; A46-B30; A46-B31; A46-B32; A46-B33; A46-B34; A46-B35; A46-B36; A46-B37; A46-B38; A46-B39; A46-B40; A46-B41; A46-B42; A46-B43; A46-B44; A46-B45; A46-B46; A46-B47; A46-B48; A46-B49; A46-B50; A46-B51; A46-B52; A46-B53; A46-B54; A46-B55; A46-B56; A46-B57; A46-B58; A46-B59; A46-B60; A46-B61; A46-B62; A46-B63; A46-B64; A46-B65; A46-B66; A46-B67; A46-B68; A46-B69; A46-B70; A46-B71; A46-B72; A46-B73; A46-B74; A46-B75; A46-B76; A46-B77; A46-B78; A46-B79; A46-B80; A46-B81; A46-B82; A46-B83; A46-B84; A46-B85; A46-B86; A46-B87; A46-B88; A46-B89; A46-B90; A46-B91; A46-B92; A46-B93; A46-B94; A46-B95; A46-B96; A46-B97; A46-B98; A46-B99; A46-B100; A46-B101; A46-B102; A46-B103; A46-B104; A46-B105; A46-B106; A46-B107; A46-B108; A46-B109; A46-B110; A46-B111; A46-B112; A46-B113;

A47-B1; A47-B2; A47-B3; A47-B4; A47-B5; A47-B6; A47-B7; A47-B8; A47-B9; A47-B10; A47-B11; A47-B12; A47-B13; A47-B14; A47-B15; A47-B16; A47-B17; A47-B18; A47-B19; A47-B20; A47-B21; A47-B22; A47-B23; A47-B24; A47-B25; A47-B26; A47-B27; A47-B28; A47-B29; A47-B30; A47-B31; A47-B32; A47-B33; A47-B34; A47-B35; A47-B36; A47-B37; A47-B38; A47-B39; A47-B40; A47-B41; A47-B42; A47-B43; A47-B44; A47-B45; A47-B46; A47-B47; A47-B48; A47-B49; A47-B50; A47-B51; A47-B52; A47-B53; A47-B54; A47-B55; A47-B56; A47-B57; A47-B58; A47-B59; A47-B60; A47-B61; A47-B62; A47-B63; A47-B64; A47-B65; A47-B66; A47-B67; A47-B68; A47-B69; A47-B70; A47-B71; A47-B72; A47-B73; A47-B74; A47-B75; A47-B76; A47-B77; A47-B78; A47-B79; A47-B80; A47-B81; A47-B82; A47-B83; A47-B84; A47-B85; A47-B86; A47-B87; A47-B88; A47-B89; A47-B90; A47-B91; A47-B92; A47-B93; A47-B94; A47-B95; A47-B96; A47-B97; A47-B98; A47-B99; A47-B100; A47-B101; A47-B102; A47-B103; A47-B104; A47-B105; A47-B106; A47-B107; A47-B108; A47-B109; A47-B110; A47-B111; A47-B112; A47-B113;

A48-B1; A48-B2; A48-B3; A48-B4; A48-B5; A48-B6; A48-B7; A48-B8; A48-B9; A48-B10; A48-B11; A48-B12; A48-B13; A48-B14; A48-B15; A48-B16; A48-B17; A48-B18; A48-B19; A48-B20; A48-B21; A48-B22; A48-B23; A48-B24; A48-B25; A48-B26; A48-B27; A48-B28; A48-B29; A48-B30; A48-B31; A48-B32; A48-B33; A48-B34; A48-B35; A48-B36; A48-B37; A48-B38; A48-B39; A48-B40; A48-B41; A48-B42; A48-B43; A48-B44; A48-B45; A48-B46; A48-B47; A48-B48; A48-B49; A48-B50; A48-B51; A48-B52; A48-B53; A48-B54; A48-B55; A48-B56; A48-B57; A48-B58; A48-B59; A48-B60; A48-B61; A48-B62; A48-B63; A48-B64; A48-B65; A48-B66; A48-B67; A48-B68; A48-B69; A48-B70; A48-B71; A48-B72; A48-B73; A48-B74; A48-B75; A48-B76; A48-B77; A48-B78; A48-B79; A48-B80; A48-B81; A48-B82; A48-B83; A48-B84; A48-B85; A48-B86; A48-B87; A48-B88; A48-B89; A48-B90; A48-B91; A48-B92; A48-B93; A48-B94; A48-B95; A48-B96; A48-B97; A48-B98; A48-B99; A48-B100; A48-B101; A48-B102; A48-B103; A48-B104; A48-B105; A48-B106; A48-B107; A48-B108; A48-B109; A48-B110; A48-B111; A48-B112; A48-B113;

A49-B1; A49-B2; A49-B3; A49-B4; A49-B5; A49-B6; A49-B7; A49-B8; A49-B9; A49-B10; A49-B11; A49-B12; A49-B13; A49-B14; A49-B15; A49-B16; A49-B17; A49-B18; A49-B19; A49-B20; A49-B21; A49-B22; A49-B23; A49-B24; A49-B25; A49-B26; A49-B27; A49-B28; A49-B29; A49-B30; A49-B31; A49-B32; A49-B33; A49-B34; A49-B35; A49-B36; A49-B37; A49-B38; A49-B39; A49-B40; A49-B41; A49-B42; A49-B43; A49-B44; A49-B45; A49-B46; A49-B47; A49-B48; A49-B49; A49-B50; A49-B51; A49-B52; A49-B53; A49-B54; A49-B55; A49-B56; A49-B57; A49-B58; A49-B59; A49-B60; A49-B61; A49-B62; A49-B63; A49-B64; A49-B65; A49-B66; A49-B67; A49-B68; A49-B69; A49-B70; A49-B71; A49-B72; A49-B73; A49-B74; A49-B75; A49-B76; A49-B77; A49-B78; A49-B79; A49-B80; A49-B81; A49-B82; A49-B83; A49-B84; A49-B85; A49-B86; A49-B87; A49-B88; A49-B89; A49-B90; A49-B91; A49-B92; A49-B93; A49-B94; A49-B95; A49-B96; A49-B97; A49-B98; A49-B99; A49-B100; A49-B101; A49-B102; A49-B103; A49-B104; A49-B105; A49-B106; A49-B107; A49-B108; A49-B109; A49-B110; A49-B111; A49-B112; A49-B113;

A50-B1; A50-B2; A50-B3; A50-B4; A50-B5; A50-B6; A50-B7; A50-B8; A50-B9; A50-B10; A50-B11; A50-B12; A50-B13; A50-B14; A50-B15; A50-B16; A50-B17; A50-B18; A50-B19; A50-B20; A50-B21; A50-B22; A50-B23; A50-B24; A50-B25; A50-B26; A50-B27; A50-B28; A50-B29; A50-B30; A50-B31; A50-B32; A50-B33; A50-B34; A50-B35; A50-B36; A50-B37; A50-B38; A50-B39; A50-B40; A50-B41; A50-B42; A50-B43; A50-B44; A50-B45; A50-B46; A50-B47; A50-B48; A50-B49; A50-B50; A50-B51; A50-B52; A50-B53; A50-B54; A50-B55; A50-B56; A50-B57; A50-B58; A50-B59; A50-B60; A50-B61; A50-B62; A50-B63; A50-B64; A50-B65; A50-B66; A50-B67; A50-B68; A50-B69; A50-B70; A50-B71; A50-B72; A50-B73; A50-B74; A50-B75; A50-B76; A50-B77; A50-B78; A50-B79; A50-B80; A50-B81; A50-B82; A50-B83; A50-B84; A50-B85; A50-B86; A50-B87; A50-B88; A50-B89; A50-B90; A50-B91; A50-B92; A50-B93; A50-B94; A50-B95; A50-B96; A50-B97; A50-B98; A50-B99; A50-B100; A50-B101; A50-B102; A50-B103; A50-B104; A50-B105; A50-B106; A50-B107; A50-B108; A50-B109; A50-B110; A50-B111; A50-B112; A50-B113;

A51-B1; A51-B2; A51-B3; A51-B4; A51-B5; A51-B6; A51-B7; A51-B8; A51-B9; A51-B10; A51-B11; A51-B12; A51-B13; A51-B14; A51-B15; A51-B16; A51-B17; A51-B18; A51-B19; A51-B20; A51-B21; A51-B22; A51-B23; A51-B24; A51-B25; A51-B26; A51-B27; A51-B28; A51-B29; A51-B30; A51-B31; A51-B32; A51-B33; A51-B34; A51-B35; A51-B36; A51-B37; A51-B38; A51-B39; A51-B40; A51-B41; A51-B42; A51-B43; A51-B44; A51-B45; A51-B46; A51-B47; A51-B48; A51-B49; A51-B50; A51-B51; A51-B52; A51-B53; A51-B54; A51-B55; A51-B56; A51-B57; A51-B58; A51-B59; A51-B60; A51-B61; A51-B62; A51-B63; A51-B64; A51-B65; A51-B66; A51-B67; A51-B68; A51-B69; A51-B70; A51-B71; A51-B72; A51-B73; A51-B74; A51-B75; A51-B76; A51-B77; A51-B78; A51-B79; A51-B80; A51-B81; A51-B82; A51-B83; A51-B84; A51-B85; A51-B86; A51-B87; A51-B88; A51-B89; A51-B90; A51-B91; A51-B92; A51-B93; A51-B94; A51-B95; A51-B96; A51-B97; A51-B98; A51-B99; A51-B100; A51-B101; A51-B102; A51-B103; A51-B104; A51-B105; A51-B106; A51-B107; A51-B108; A51-B109; A51-B110; A51-B111; A51-B112; A51-B113;

A52-B1; A52-B2; A52-B3; A52-B4; A52-B5; A52-B6; A52-B7; A52-B8; A52-B9; A52-B10; A52-B11; A52-B12; A52-B13; A52-B14; A52-B15; A52-B16; A52-B17; A52-B18; A52-B19; A52-B20; A52-B21; A52-B22; A52-B23; A52-B24; A52-B25; A52-B26; A52-B27; A52-B28; A52-B29; A52-B30; A52-B31; A52-B32; A52-B33; A52-B34; A52-B35; A52-B36; A52-B37; A52-B38; A52-B39; A52-B40; A52-B41; A52-B42; A52-B43; A52-B44; A52-B45; A52-B46; A52-B47; A52-B48; A52-B49; A52-B50; A52-B51; A52-B52; A52-B53; A52-B54; A52-B55; A52-B56; A52-B57; A52-B58; A52-B59; A52-B60; A52-B61; A52-B62; A52-B63; A52-B64; A52-B65; A52-B66; A52-B67; A52-B68; A52-B69; A52-B70; A52-B71; A52-B72; A52-B73; A52-B74; A52-B75; A52-B76; A52-B77; A52-B78; A52-B79; A52-B80; A52-B81; A52-B82; A52-B83; A52-B84; A52-B85; A52-B86; A52-B87; A52-B88; A52-B89; A52-B90; A52-B91; A52-B92; A52-B93; A52-B94; A52-B95; A52-B96; A52-B97; A52-B98; A52-B99; A52-B100; A52-B101; A52-B102; A52-B103; A52-B104; A52-B105; A52-B106; A52-B107; A52-B108; A52-B109; A52-B110; A52-B111; A52-B112; A52-B113;

A53-B1; A53-B2; A53-B3; A53-B4; A53-B5; A53-B6; A53-B7; A53-B8; A53-B9; A53-B10; A53-B11; A53-B12; A53-B13; A53-B14; A53-B15; A53-B16; A53-B17; A53-B18; A53-B19; A53-B20; A53-B21; A53-B22; A53-B23; A53-B24; A53-B25; A53-B26; A53-B27; A53-B28; A53-B29; A53-B30; A53-B31; A53-B32; A53-B33; A53-B34; A53-B35; A53-B36; A53-B37; A53-B38; A53-B39; A53-B40; A53-B41; A53-B42; A53-B43; A53-B44; A53-B45; A53-B46; A53-B47; A53-B48; A53-B49; A53-B50; A53-B51; A53-B52; A53-B53; A53-B54; A53-B55; A53-B56; A53-B57; A53-B58; A53-B59; A53-B60; A53-B61; A53-B62; A53-B63; A53-B64; A53-B65; A53-B66; A53-B67; A53-B68; A53-B69; A53-B70; A53-B71; A53-B72; A53-B73; A53-B74; A53-B75; A53-B76; A53-B77; A53-B78; A53-B79; A53-B80; A53-B81; A53-B82; A53-B83; A53-B84; A53-B85; A53-B86; A53-B87; A53-B88; A53-B89; A53-B90; A53-B91; A53-B92; A53-B93; A53-B94; A53-B95; A53-B96; A53-B97; A53-B98; A53-B99; A53-B100; A53-B101; A53-B102; A53-B103; A53-B104; A53-B105; A53-B106; A53-B107; A53-B108; A53-B109; A53-B110; A53-B111; A53-B112; A53-B113;

A54-B1; A54-B2; A54-B3; A54-B4; A54-B5; A54-B6; A54-B7; A54-B8; A54-B9; A54-B10; A54-B11; A54-B12; A54-B13; A54-B14; A54-B15; A54-B16; A54-B17; A54-B18; A54-B19; A54-B20; A54-B21; A54-B22; A54-B23; A54-B24; A54-B25; A54-B26; A54-B27; A54-B28; A54-B29; A54-B30; A54-B31; A54-B32; A54-B33; A54-B34; A54-B35; A54-B36; A54-B37; A54-B38; A54-B39; A54-B40; A54-B41; A54-B42; A54-B43; A54-B44; A54-B45; A54-B46; A54-B47; A54-B48; A54-B49; A54-B50; A54-B51; A54-B52; A54-B53; A54-B54; A54-B55; A54-B56; A54-B57; A54-B58; A54-B59; A54-B60; A54-B61; A54-B62; A54-B63; A54-B64; A54-B65; A54-B66; A54-B67; A54-B68; A54-B69; A54-B70; A54-B71; A54-B72; A54-B73; A54-B74; A54-B75; A54-B76; A54-B77; A54-B78; A54-B79; A54-B80; A54-B81; A54-B82; A54-B83; A54-B84; A54-B85; A54-B86; A54-B87; A54-B88; A54-B89; A54-B90; A54-B91; A54-B92; A54-B93; A54-B94; A54-B95; A54-B96; A54-B97; A54-B98; A54-B99; A54-B100; A54-B101; A54-B102; A54-B103; A54-B104; A54-B105; A54-B106; A54-B107; A54-B108; A54-B109; A54-B110; A54-B111; A54-B112; A54-B113;

A55-B1; A55-B2; A55-B3; A55-B4; A55-B5; A55-B6; A55-B7; A55-B8; A55-B9; A55-B10; A55-B11; A55-B12; A55-B13; A55-B14; A55-B15; A55-B16; A55-B17; A55-B18; A55-B19; A55-B20; A55-B21; A55-B22; A55-B23; A55-B24; A55-B25; A55-B26; A55-B27; A55-B28; A55-B29; A55-B30; A55-B31; A55-B32; A55-B33; A55-B34; A55-B35; A55-B36; A55-B37; A55-B38; A55-B39; A55-B40; A55-B41; A55-B42; A55-B43; A55-B44; A55-B45; A55-B46; A55-B47; A55-B48; A55-B49; A55-B50; A55-B51; A55-B52; A55-B53; A55-B54; A55-B55; A55-B56; A55-B57; A55-B58; A55-B59; A55-B60; A55-B61; A55-B62; A55-B63; A55-B64; A55-B65; A55-B66; A55-B67; A55-B68; A55-B69; A55-B70; A55-B71; A55-B72; A55-B73; A55-B74; A55-B75; A55-B76; A55-B77; A55-B78; A55-B79; A55-B80; A55-B81; A55-B82; A55-B83; A55-B84; A55-B85; A55-B86; A55-B87; A55-B88; A55-B89; A55-B90; A55-B91; A55-B92; A55-B93; A55-B94; A55-B95; A55-B96; A55-B97; A55-B98; A55-B99; A55-B100; A55-B101; A55-B102; A55-B103; A55-B104; A55-B105; A55-B106; A55-B107; A55-B108; A55-B109; A55-B110; A55-B111; A55-B112; A55-B113;

A56-B1; A56-B2; A56-B3; A56-B4; A56-B5; A56-B6; A56-B7; A56-B8; A56-B9; A56-B10; A56-B11; A56-B12; A56-B13; A56-B14; A56-B15; A56-B16; A56-B17; A56-B18; A56-B19; A56-B20; A56-B21; A56-B22; A56-B23; A56-B24; A56-B25; A56-B26; A56-B27; A56-B28; A56-B29; A56-B30; A56-B31; A56-B32; A56-B33; A56-B34; A56-B35; A56-B36; A56-B37; A56-B38; A56-B39; A56-B40; A56-B41; A56-B42; A56-B43; A56-B44; A56-B45; A56-B46; A56-B47; A56-B48; A56-B49; A56-B50; A56-B51; A56-B52; A56-B53; A56-B54; A56-B55; A56-B56; A56-B57; A56-B58; A56-B59; A56-B60; A56-B61; A56-B62; A56-B63; A56-B64; A56-B65; A56-B66; A56-B67; A56-B68; A56-B69; A56-B70; A56-B71; A56-B72; A56-B73; A56-B74; A56-B75; A56-B76; A56-B77; A56-B78; A56-B79; A56-B80; A56-B81; A56-B82; A56-B83; A56-B84; A56-B85; A56-B86; A56-B87; A56-B88; A56-B89; A56-B90; A56-B91; A56-B92; A56-B93; A56-B94; A56-B95; A56-B96; A56-B97; A56-B98; A56-B99; A56-B100; A56-B101; A56-B102; A56-B103; A56-B104; A56-B105; A56-B106; A56-B107; A56-B108; A56-B109; A56-B110; A56-B111; A56-B112; A56-B113;

A57-B1; A57-B2; A57-B3; A57-B4; A57-B5; A57-B6; A57-B7; A57-B8; A57-B9; A57-B10; A57-B11; A57-B12; A57-B13; A57-B14; A57-B15; A57-B16; A57-B17; A57-B18; A57-B19; A57-B20; A57-B21; A57-B22; A57-B23; A57-B24; A57-B25; A57-B26; A57-B27; A57-B28; A57-B29; A57-B30; A57-B31; A57-B32; A57-B33; A57-B34; A57-B35; A57-B36; A57-B37; A57-B38; A57-B39; A57-B40; A57-B41; A57-B42; A57-B43; A57-B44; A57-B45; A57-B46; A57-B47; A57-B48; A57-B49; A57-B50; A57-B51; A57-B52; A57-B53; A57-B54; A57-B55; A57-B56; A57-B57; A57-B58; A57-B59; A57-B60; A57-B61; A57-B62; A57-B63; A57-B64; A57-B65; A57-B66; A57-B67; A57-B68; A57-B69; A57-B70; A57-B71; A57-B72; A57-B73; A57-B74; A57-B75; A57-B76; A57-B77; A57-B78; A57-B79; A57-B80; A57-B81; A57-B82; A57-B83; A57-B84; A57-B85; A57-B86; A57-B87; A57-B88; A57-B89; A57-B90; A57-B91; A57-B92; A57-B93; A57-B94; A57-B95; A57-B96; A57-B97; A57-B98; A57-B99; A57-B100; A57-B101; A57-B102; A57-B103; A57-B104; A57-B105; A57-B106; A57-B107; A57-B108; A57-B109; A57-B110; A57-B111; A57-B112; A57-B113;

A58-B1; A58-B2; A58-B3; A58-B4; A58-B5; A58-B6; A58-B7; A58-B8; A58-B9; A58-B10; A58-B11; A58-B12; A58-B13; A58-B14; A58-B15; A58-B16; A58-B17; A58-B18; A58-B19; A58-B20; A58-B21; A58-B22; A58-B23; A58-B24; A58-B25; A58-B26; A58-B27; A58-B28; A58-B29; A58-B30; A58-B31; A58-B32; A58-B33; A58-B34; A58-B35; A58-B36; A58-B37; A58-B38; A58-B39; A58-B40; A58-B41; A58-B42; A58-B43; A58-B44; A58-B45; A58-B46; A58-B47; A58-B48; A58-B49; A58-B50; A58-B51; A58-B52; A58-B53; A58-B54; A58-B55; A58-B56; A58-B57; A58-B58; A58-B59; A58-B60; A58-B61; A58-B62; A58-B63; A58-B64; A58-B65; A58-B66; A58-B67; A58-B68; A58-B69; A58-B70; A58-B71; A58-B72; A58-B73; A58-B74; A58-B75; A58-B76; A58-B77; A58-B78; A58-B79; A58-B80; A58-B81; A58-B82; A58-B83; A58-B84; A58-B85; A58-B86; A58-B87; A58-B88; A58-B89; A58-B90; A58-B91; A58-B92; A58-B93; A58-B94; A58-B95; A58-B96; A58-B97; A58-B98; A58-B99; A58-B100; A58-B101; A58-B102; A58-B103; A58-B104; A58-B105; A58-B106; A58-B107; A58-B108; A58-B109; A58-B110; A58-B111; A58-B112; A58-B113;

A59-B1; A59-B2; A59-B3; A59-B4; A59-B5; A59-B6; A59-B7; A59-B8; A59-B9; A59-B10; A59-B11; A59-B12; A59-B13; A59-B14; A59-B15; A59-B16; A59-B17; A59-B18; A59-B19; A59-B20; A59-B21; A59-B22; A59-B23; A59-B24; A59-B25; A59-B26; A59-B27; A59-B28; A59-B29; A59-B30; A59-B31; A59-B32; A59-B33; A59-B34; A59-B35; A59-B36; A59-B37; A59-B38; A59-B39; A59-B40; A59-B41; A59-B42; A59-B43; A59-B44; A59-B45; A59-B46; A59-B47; A59-B48; A59-B49; A59-B50; A59-B51; A59-B52; A59-B53; A59-B54; A59-B55; A59-B56; A59-B57; A59-B58; A59-B59; A59-B60; A59-B61; A59-B62; A59-B63; A59-B64; A59-B65; A59-B66; A59-B67; A59-B68; A59-B69; A59-B70; A59-B71; A59-B72; A59-B73; A59-B74; A59-B75; A59-B76; A59-B77; A59-B78; A59-B79; A59-B80; A59-B81; A59-B82; A59-B83; A59-B84; A59-B85; A59-B86; A59-B87; A59-B88; A59-B89; A59-B90; A59-B91; A59-B92; A59-B93; A59-B94; A59-B95; A59-B96; A59-B97; A59-B98; A59-B99; A59-B100; A59-B101; A59-B102; A59-B103; A59-B104; A59-B105; A59-B106; A59-B107; A59-B108; A59-B109; A59-B110; A59-B111; A59-B112; A59-B113;

A60-B1; A60-B2; A60-B3; A60-B4; A60-B5; A60-B6; A60-B7; A60-B8; A60-B9; A60-B10; A60-B11; A60-B12; A60-B13; A60-B14; A60-B15; A60-B16; A60-B17; A60-B18; A60-B19; A60-B20; A60-B21; A60-B22; A60-B23; A60-B24; A60-B25; A60-B26; A60-B27; A60-B28; A60-B29; A60-B30; A60-B31; A60-B32; A60-B33; A60-B34; A60-B35; A60-B36; A60-B37; A60-B38; A60-B39; A60-B40; A60-B41; A60-B42; A60-B43; A60-B44; A60-B45; A60-B46; A60-B47; A60-B48; A60-B49; A60-B50; A60-B51; A60-B52; A60-B53; A60-B54; A60-B55; A60-B56; A60-B57; A60-B58; A60-B59; A60-B60; A60-B61; A60-B62; A60-B63; A60-B64; A60-B65; A60-B66; A60-B67; A60-B68; A60-B69; A60-B70; A60-B71; A60-B72; A60-B73; A60-B74; A60-B75; A60-B76; A60-B77; A60-B78; A60-B79; A60-B80; A60-B81; A60-B82; A60-B83; A60-B84; A60-B85; A60-B86; A60-B87; A60-B88; A60-B89; A60-B90; A60-B91; A60-B92; A60-B93; A60-B94; A60-B95; A60-B96; A60-B97; A60-B98; A60-B99; A60-B100; A60-B101; A60-B102; A60-B103; A60-B104; A60-B105; A60-B106; A60-B107; A60-B108; A60-B109; A60-B110; A60-B111; A60-B112; A60-B113;

A61-B1; A61-B2; A61-B3; A61-B4; A61-B5; A61-B6; A61-B7; A61-B8; A61-B9; A61-B10; A61-B11; A61-B12; A61-B13; A61-B14; A61-B15; A61-B16; A61-B17; A61-B18; A61-B19; A61-B20; A61-B21; A61-B22; A61-B23; A61-B24; A61-B25; A61-B26; A61-B27; A61-B28; A61-B29; A61-B30; A61-B31; A61-B32; A61-B33; A61-B34; A61-B35; A61-B36; A61-B37; A61-B38; A61-B39; A61-B40; A61-B41; A61-B42; A61-B43; A61-B44; A61-B45; A61-B46; A61-B47; A61-B48; A61-B49; A61-B50; A61-B51; A61-B52; A61-B53; A61-B54; A61-B55; A61-B56; A61-B57; A61-B58; A61-B59; A61-B60; A61-B61; A61-B62; A61-B63; A61-B64; A61-B65; A61-B66; A61-B67; A61-B68; A61-B69; A61-B70; A61-B71; A61-B72; A61-B73; A61-B74; A61-B75; A61-B76; A61-B77; A61-B78; A61-B79; A61-B80; A61-B81; A61-B82; A61-B83; A61-B84; A61-B85; A61-B86; A61-B87; A61-B88; A61-B89; A61-B90; A61-B91; A61-B92; A61-B93; A61-B94; A61-B95; A61-B96; A61-B97; A61-B98; A61-B99; A61-B100; A61-B101; A61-B102; A61-B103; A61-B104; A61-B105; A61-B106; A61-B107; A61-B108; A61-B109; A61-B110; A61-B111; A61-B112; A61-B113;

A62-B1; A62-B2; A62-B3; A62-B4; A62-B5; A62-B6; A62-B7; A62-B8; A62-B9; A62-B10; A62-B11; A62-B12; A62-B13; A62-B14; A62-B15; A62-B16; A62-B17; A62-B18; A62-B19; A62-B20; A62-B21; A62-B22; A62-B23; A62-B24; A62-B25; A62-B26; A62-B27; A62-B28; A62-B29; A62-B30; A62-B31; A62-B32; A62-B33; A62-B34; A62-B35; A62-B36; A62-B37; A62-B38; A62-B39; A62-B40; A62-B41; A62-B42; A62-B43; A62-B44; A62-B45; A62-B46; A62-B47; A62-B48; A62-B49; A62-B50; A62-B51; A62-B52; A62-B53; A62-B54; A62-B55; A62-B56; A62-B57; A62-B58; A62-B59; A62-B60; A62-B61; A62-B62; A62-B63; A62-B64; A62-B65; A62-B66; A62-B67; A62-B68; A62-B69; A62-B70; A62-B71; A62-B72; A62-B73; A62-B74; A62-B75; A62-B76; A62-B77; A62-B78; A62-B79; A62-B80; A62-B81; A62-B82; A62-B83; A62-B84; A62-B85; A62-B86; A62-B87; A62-B88; A62-B89; A62-B90; A62-B91; A62-B92; A62-B93; A62-B94; A62-B95; A62-B96; A62-B97; A62-B98; A62-B99; A62-B100; A62-B101; A62-B102; A62-B103; A62-B104; A62-B105; A62-B106; A62-B107; A62-B108; A62-B109; A62-B110; A62-B111; A62-B112; A62-B113;

A63-B1; A63-B2; A63-B3; A63-B4; A63-B5; A63-B6; A63-B7; A63-B8; A63-B9; A63-B10; A63-B11; A63-B12; A63-B13; A63-B14; A63-B15; A63-B16; A63-B17; A63-B18; A63-B19; A63-B20; A63-B21; A63-B22; A63-B23; A63-B24; A63-B25; A63-B26; A63-B27; A63-B28; A63-B29; A63-B30; A63-B31; A63-B32; A63-B33; A63-B34; A63-B35; A63-B36; A63-B37; A63-B38; A63-B39; A63-B40; A63-B41; A63-B42; A63-B43; A63-B44; A63-B45; A63-B46; A63-B47; A63-B48; A63-B49; A63-B50; A63-B51; A63-B52; A63-B53; A63-B54; A63-B55; A63-B56; A63-B57; A63-B58; A63-B59; A63-B60; A63-B61; A63-B62; A63-B63; A63-B64; A63-B65; A63-B66; A63-B67; A63-B68; A63-B69; A63-B70; A63-B71; A63-B72; A63-B73; A63-B74; A63-B75; A63-B76; A63-B77; A63-B78; A63-B79; A63-B80; A63-B81; A63-B82; A63-B83; A63-B84; A63-B85; A63-B86; A63-B87; A63-B88; A63-B89; A63-B90; A63-B91; A63-B92; A63-B93; A63-B94; A63-B95; A63-B96; A63-B97; A63-B98; A63-B99; A63-B100; A63-B101; A63-B102; A63-B103; A63-B104; A63-B105; A63-B106; A63-B107; A63-B108; A63-B109; A63-B110; A63-B111; A63-B112; A63-B113;

A64-B1; A64-B2; A64-B3; A64-B4; A64-B5; A64-B6; A64-B7; A64-B8; A64-B9; A64-B10; A64-B11; A64-B12; A64-B13; A64-B14; A64-B15; A64-B16; A64-B17; A64-B18; A64-B19; A64-B20; A64-B21; A64-B22; A64-B23; A64-B24; A64-B25; A64-B26; A64-B27; A64-B28; A64-B29; A64-B30; A64-B31; A64-B32; A64-B33; A64-B34; A64-B35; A64-B36; A64-B37; A64-B38; A64-B39; A64-B40; A64-B41; A64-B42; A64-B43; A64-B44; A64-B45; A64-B46; A64-B47; A64-B48; A64-B49; A64-B50; A64-B51; A64-B52; A64-B53; A64-B54; A64-B55; A64-B56; A64-B57; A64-B58; A64-B59; A64-B60; A64-B61; A64-B62; A64-B63; A64-B64; A64-B65; A64-B66; A64-B67; A64-B68; A64-B69; A64-B70; A64-B71; A64-B72; A64-B73; A64-B74; A64-B75; A64-B76; A64-B77; A64-B78; A64-B79; A64-B80; A64-B81; A64-B82; A64-B83; A64-B84; A64-B85; A64-B86; A64-B87; A64-B88; A64-B89; A64-B90; A64-B91; A64-B92; A64-B93; A64-B94; A64-B95; A64-B96; A64-B97; A64-B98; A64-B99; A64-B100; A64-B101; A64-B102; A64-B103; A64-B104; A64-B105; A64-B106; A64-B107; A64-B108; A64-B109; A64-B110; A64-B111; A64-B112; A64-B113;

A65-B1; A65-B2; A65-B3; A65-B4; A65-B5; A65-B6; A65-B7; A65-B8; A65-B9; A65-B10; A65-B11; A65-B12; A65-B13; A65-B14; A65-B15; A65-B16; A65-B17; A65-B18; A65-B19; A65-B20; A65-B21; A65-B22; A65-B23; A65-B24; A65-B25; A65-B26; A65-B27; A65-B28; A65-B29; A65-B30; A65-B31; A65-B32; A65-B33; A65-B34; A65-B35; A65-B36; A65-B37; A65-B38; A65-B39; A65-B40; A65-B41; A65-B42; A65-B43; A65-B44; A65-B45; A65-B46; A65-B47; A65-B48; A65-B49; A65-B50; A65-B51; A65-B52; A65-B53; A65-B54; A65-B55; A65-B56; A65-B57; A65-B58; A65-B59; A65-B60; A65-B61; A65-B62; A65-B63; A65-B64; A65-B65; A65-B66; A65-B67; A65-B68; A65-B69; A65-B70; A65-B71; A65-B72; A65-B73; A65-B74; A65-B75; A65-B76; A65-B77; A65-B78; A65-B79; A65-B80; A65-B81; A65-B82; A65-B83; A65-B84; A65-B85; A65-B86; A65-B87; A65-B88; A65-B89; A65-B90; A65-B91; A65-B92; A65-B93; A65-B94; A65-B95; A65-B96; A65-B97; A65-B98; A65-B99; A65-B100; A65-B101; A65-B102; A65-B103; A65-B104; A65-B105; A65-B106; A65-B107; A65-B108; A65-B109; A65-B110; A65-B111; A65-B112; A65-B113;

A66-B1; A66-B2; A66-B3; A66-B4; A66-B5; A66-B6; A66-B7; A66-B8; A66-B9; A66-B10; A66-B11; A66-B12; A66-B13; A66-B14; A66-B15; A66-B16; A66-B17; A66-B18; A66-B19; A66-B20; A66-B21; A66-B22; A66-B23; A66-B24; A66-B25; A66-B26; A66-B27; A66-B28; A66-B29; A66-B30; A66-B31; A66-B32; A66-B33; A66-B34; A66-B35; A66-B36; A66-B37; A66-B38; A66-B39; A66-B40; A66-B41; A66-B42; A66-B43; A66-B44; A66-B45; A66-B46; A66-B47; A66-B48; A66-B49; A66-B50; A66-B51; A66-B52; A66-B53; A66-B54; A66-B55; A66-B56; A66-B57; A66-B58; A66-B59; A66-B60; A66-B61; A66-B62; A66-B63; A66-B64; A66-B65; A66-B66; A66-B67; A66-B68; A66-B69; A66-B70; A66-B71; A66-B72; A66-B73; A66-B74; A66-B75; A66-B76; A66-B77; A66-B78; A66-B79; A66-B80; A66-B81; A66-B82; A66-B83; A66-B84; A66-B85; A66-B86; A66-B87; A66-B88; A66-B89; A66-B90; A66-B91; A66-B92; A66-B93; A66-B94; A66-B95; A66-B96; A66-B97; A66-B98; A66-B99; A66-B100; A66-B101; A66-B102; A66-B103; A66-B104; A66-B105; A66-B106; A66-B107; A66-B108; A66-B109; A66-B110; A66-B111; A66-B112; A66-B113;

A67-B1; A67-B2; A67-B3; A67-B4; A67-B5; A67-B6; A67-B7; A67-B8; A67-B9; A67-B10; A67-B11; A67-B12; A67-B13; A67-B14; A67-B15; A67-B16; A67-B17; A67-B18; A67-B19; A67-B20; A67-B21; A67-B22; A67-B23; A67-B24; A67-B25; A67-B26; A67-B27; A67-B28; A67-B29; A67-B30; A67-B31; A67-B32; A67-B33; A67-B34; A67-B35; A67-B36; A67-B37; A67-B38; A67-B39; A67-B40; A67-B41; A67-B42; A67-B43; A67-B44; A67-B45; A67-B46; A67-B47; A67-B48; A67-B49; A67-B50; A67-B51; A67-B52; A67-B53; A67-B54; A67-B55; A67-B56; A67-B57; A67-B58; A67-B59; A67-B60; A67-B61; A67-B62; A67-B63; A67-B64; A67-B65; A67-B66; A67-B67; A67-B68; A67-B69; A67-B70; A67-B71; A67-B72; A67-B73; A67-B74; A67-B75; A67-B76; A67-B77; A67-B78; A67-B79; A67-B80; A67-B81; A67-B82; A67-B83; A67-B84; A67-B85; A67-B86; A67-B87; A67-B88; A67-B89; A67-B90; A67-B91; A67-B92; A67-B93; A67-B94; A67-B95; A67-B96; A67-B97; A67-B98; A67-B99; A67-B100; A67-B101; A67-B102; A67-B103; A67-B104; A67-B105; A67-B106; A67-B107; A67-B108; A67-B109; A67-B110; A67-B111; A67-B112; A67-B113;

A68-B1; A68-B2; A68-B3; A68-B4; A68-B5; A68-B6; A68-B7; A68-B8; A68-B9; A68-B10; A68-B11; A68-B12; A68-B13; A68-B14; A68-B15; A68-B16; A68-B17; A68-B18; A68-B19; A68-B20; A68-B21; A68-B22; A68-B23; A68-B24; A68-B25; A68-B26; A68-B27; A68-B28; A68-B29; A68-B30; A68-B31; A68-B32; A68-B33; A68-B34; A68-B35; A68-B36; A68-B37; A68-B38; A68-B39; A68-B40; A68-B41; A68-B42; A68-B43; A68-B44; A68-B45; A68-B46; A68-B47; A68-B48; A68-B49; A68-B50; A68-B51; A68-B52; A68-B53; A68-B54; A68-B55; A68-B56; A68-B57; A68-B58; A68-B59; A68-B60; A68-B61; A68-B62; A68-B63; A68-B64; A68-B65; A68-B66; A68-B67; A68-B68; A68-B69; A68-B70; A68-B71; A68-B72; A68-B73; A68-B74; A68-B75; A68-B76; A68-B77; A68-B78; A68-B79; A68-B80; A68-B81; A68-B82; A68-B83; A68-B84; A68-B85; A68-B86; A68-B87; A68-B88; A68-B89; A68-B90; A68-B91; A68-B92; A68-B93; A68-B94; A68-B95; A68-B96; A68-B97; A68-B98; A68-B99; A68-B100; A68-B101; A68-B102; A68-B103; A68-B104; A68-B105; A68-B106; A68-B107; A68-B108; A68-B109; A68-B110; A68-B111; A68-B112; A68-B113;

A69-B1; A69-B2; A69-B3; A69-B4; A69-B5; A69-B6; A69-B7; A69-B8; A69-B9; A69-B10; A69-B11; A69-B12; A69-B13; A69-B14; A69-B15; A69-B16; A69-B17; A69-B18; A69-B19; A69-B20; A69-B21; A69-B22; A69-B23; A69-B24; A69-B25; A69-B26; A69-B27; A69-B28; A69-B29; A69-B30; A69-B31; A69-B32; A69-B33; A69-B34; A69-B35; A69-B36; A69-B37; A69-B38; A69-B39; A69-B40; A69-B41; A69-B42; A69-B43; A69-B44; A69-B45; A69-B46; A69-B47; A69-B48; A69-B49; A69-B50; A69-B51; A69-B52; A69-B53; A69-B54; A69-B55; A69-B56; A69-B57; A69-B58; A69-B59; A69-B60; A69-B61; A69-B62; A69-B63; A69-B64; A69-B65; A69-B66; A69-B67; A69-B68; A69-B69; A69-B70; A69-B71; A69-B72; A69-B73; A69-B74; A69-B75; A69-B76; A69-B77; A69-B78; A69-B79; A69-B80; A69-B81; A69-B82; A69-B83; A69-B84; A69-B85; A69-B86; A69-B87; A69-B88; A69-B89; A69-B90; A69-B91; A69-B92; A69-B93; A69-B94; A69-B95; A69-B96; A69-B97; A69-B98; A69-B99; A69-B100; A69-B101; A69-B102; A69-B103; A69-B104; A69-B105; A69-B106; A69-B107; A69-B108; A69-B109; A69-B110; A69-B111; A69-B112; A69-B113;

A70-B1; A70-B2; A70-B3; A70-B4; A70-B5; A70-B6; A70-B7; A70-B8; A70-B9; A70-B10; A70-B11; A70-B12; A70-B13; A70-B14; A70-B15; A70-B16; A70-B17; A70-B18; A70-B19; A70-B20; A70-B21; A70-B22; A70-B23; A70-B24; A70-B25; A70-B26; A70-B27; A70-B28; A70-B29; A70-B30; A70-B31; A70-B32; A70-B33; A70-B34; A70-B35; A70-B36; A70-B37; A70-B38; A70-B39; A70-B40; A70-B41; A70-B42; A70-B43; A70-B44; A70-B45; A70-B46; A70-B47; A70-B48; A70-B49; A70-B50; A70-B51; A70-B52; A70-B53; A70-B54; A70-B55; A70-B56; A70-B57; A70-B58; A70-B59; A70-B60; A70-B61; A70-B62; A70-B63; A70-B64; A70-B65; A70-B66; A70-B67; A70-B68; A70-B69; A70-B70; A70-B71; A70-B72; A70-B73; A70-B74; A70-B75; A70-B76; A70-B77; A70-B78; A70-B79; A70-B80; A70-B81; A70-B82; A70-B83; A70-B84; A70-B85; A70-B86; A70-B87; A70-B88; A70-B89; A70-B90; A70-B91; A70-B92; A70-B93; A70-B94; A70-B95; A70-B96; A70-B97; A70-B98; A70-B99; A70-B100; A70-B101; A70-B102; A70-B103; A70-B104; A70-B105; A70-B106; A70-B107; A70-B108; A70-B109; A70-B110; A70-B111; A70-B112; A70-B113;

A71-B1; A71-B2; A71-B3; A71-B4; A71-B5; A71-B6; A71-B7; A71-B8; A71-B9; A71-B10; A71-B11; A71-B12; A71-B13; A71-B14; A71-B15; A71-B16; A71-B17; A71-B18; A71-B19; A71-B20; A71-B21; A71-B22; A71-B23; A71-B24; A71-B25; A71-B26; A71-B27; A71-B28; A71-B29; A71-B30; A71-B31; A71-B32; A71-B33; A71-B34; A71-B35; A71-B36; A71-B37; A71-B38; A71-B39; A71-B40; A71-B41; A71-B42; A71-B43; A71-B44; A71-B45; A71-B46; A71-B47; A71-B48; A71-B49; A71-B50; A71-B51; A71-B52; A71-B53; A71-B54; A71-B55; A71-B56; A71-B57; A71-B58; A71-B59; A71-B60; A71-B61; A71-B62; A71-B63; A71-B64; A71-B65; A71-B66; A71-B67; A71-B68; A71-B69; A71-B70; A71-B71; A71-B72; A71-B73; A71-B74; A71-B75; A71-B76; A71-B77; A71-B78; A71-B79; A71-B80; A71-B81; A71-B82; A71-B83; A71-B84; A71-B85; A71-B86; A71-B87; A71-B88; A71-B89; A71-B90; A71-B91; A71-B92; A71-B93; A71-B94; A71-B95; A71-B96; A71-B97; A71-B98; A71-B99; A71-B100; A71-B101; A71-B102; A71-B103; A71-B104; A71-B105; A71-B106; A71-B107; A71-B108; A71-B109; A71-B110; A71-B111; A71-B112; A71-B113;

A72-B1; A72-B2; A72-B3; A72-B4; A72-B5; A72-B6; A72-B7; A72-B8; A72-B9; A72-B10; A72-B11; A72-B12; A72-B13; A72-B14; A72-B15; A72-B16; A72-B17; A72-B18; A72-B19; A72-B20; A72-B21; A72-B22; A72-B23; A72-B24; A72-B25; A72-B26; A72-B27; A72-B28; A72-B29; A72-B30; A72-B31; A72-B32; A72-B33; A72-B34; A72-B35; A72-B36; A72-B37; A72-B38; A72-B39; A72-B40; A72-B41; A72-B42; A72-B43; A72-B44; A72-B45; A72-B46; A72-B47; A72-B48; A72-B49; A72-B50; A72-B51; A72-B52; A72-B53; A72-B54; A72-B55; A72-B56; A72-B57; A72-B58; A72-B59; A72-B60; A72-B61; A72-B62; A72-B63; A72-B64; A72-B65; A72-B66; A72-B67; A72-B68; A72-B69; A72-B70; A72-B71; A72-B72; A72-B73; A72-B74; A72-B75; A72-B76; A72-B77; A72-B78; A72-B79; A72-B80; A72-B81; A72-B82; A72-B83; A72-B84; A72-B85; A72-B86; A72-B87; A72-B88; A72-B89; A72-B90; A72-B91; A72-B92; A72-B93; A72-B94; A72-B95; A72-B96; A72-B97; A72-B98; A72-B99; A72-B100; A72-B101; A72-B102; A72-B103; A72-B104; A72-B105; A72-B106; A72-B107; A72-B108; A72-B109; A72-B110; A72-B111; A72-B112; A72-B113;

A73-B1; A73-B2; A73-B3; A73-B4; A73-B5; A73-B6; A73-B7; A73-B8; A73-B9; A73-B10; A73-B11; A73-B12; A73-B13; A73-B14; A73-B15; A73-B16; A73-B17; A73-B18; A73-B19; A73-B20; A73-B21; A73-B22; A73-B23; A73-B24; A73-B25; A73-B26; A73-B27; A73-B28; A73-B29; A73-B30; A73-B31; A73-B32; A73-B33; A73-B34; A73-B35; A73-B36; A73-B37; A73-B38; A73-B39; A73-B40; A73-B41; A73-B42; A73-B43; A73-B44; A73-B45; A73-B46; A73-B47; A73-B48; A73-B49; A73-B50; A73-B51; A73-B52; A73-B53; A73-B54; A73-B55; A73-B56; A73-B57; A73-B58; A73-B59; A73-B60; A73-B61; A73-B62; A73-B63; A73-B64; A73-B65; A73-B66; A73-B67; A73-B68; A73-B69; A73-B70; A73-B71; A73-B72; A73-B73; A73-B74; A73-B75; A73-B76; A73-B77; A73-B78; A73-B79; A73-B80; A73-B81; A73-B82; A73-B83; A73-B84; A73-B85; A73-B86; A73-B87; A73-B88; A73-B89; A73-B90; A73-B91; A73-B92; A73-B93; A73-B94; A73-B95; A73-B96; A73-B97; A73-B98; A73-B99; A73-B100; A73-B101; A73-B102; A73-B103; A73-B104; A73-B105; A73-B106; A73-B107; A73-B108; A73-B109; A73-B110; A73-B111; A73-B112; A73-B113;

A74-B1; A74-B2; A74-B3; A74-B4; A74-B5; A74-B6; A74-B7; A74-B8; A74-B9; A74-B10; A74-B11; A74-B12; A74-B13; A74-B14; A74-B15; A74-B16; A74-B17; A74-B18; A74-B19; A74-B20; A74-B21; A74-B22; A74-B23; A74-B24; A74-B25; A74-B26; A74-B27; A74-B28; A74-B29; A74-B30; A74-B31; A74-B32; A74-B33; A74-B34; A74-B35; A74-B36; A74-B37; A74-B38; A74-B39; A74-B40; A74-B41; A74-B42; A74-B43; A74-B44; A74-B45; A74-B46; A74-B47; A74-B48; A74-B49; A74-B50; A74-B51; A74-B52; A74-B53; A74-B54; A74-B55; A74-B56; A74-B57; A74-B58; A74-B59; A74-B60; A74-B61; A74-B62; A74-B63; A74-B64; A74-B65; A74-B66; A74-B67; A74-B68; A74-B69; A74-B70; A74-B71; A74-B72; A74-B73; A74-B74; A74-B75; A74-B76; A74-B77; A74-B78; A74-B79; A74-B80; A74-B81; A74-B82; A74-B83; A74-B84; A74-B85; A74-B86; A74-B87; A74-B88; A74-B89; A74-B90; A74-B91; A74-B92; A74-B93; A74-B94; A74-B95; A74-B96; A74-B97; A74-B98; A74-B99; A74-B100; A74-B101; A74-B102; A74-B103; A74-B104; A74-B105; A74-B106; A74-B107; A74-B108; A74-B109; A74-B110; A74-B111; A74-B112; A74-B113;

A75-B1; A75-B2; A75-B3; A75-B4; A75-B5; A75-B6; A75-B7; A75-B8; A75-B9; A75-B10; A75-B11; A75-B12; A75-B13; A75-B14; A75-B15; A75-B16; A75-B17; A75-B18; A75-B19; A75-B20; A75-B21; A75-B22; A75-B23; A75-B24; A75-B25; A75-B26; A75-B27; A75-B28; A75-B29; A75-B30; A75-B31; A75-B32; A75-B33; A75-B34; A75-B35; A75-B36; A75-B37; A75-B38; A75-B39; A75-B40; A75-B41; A75-B42; A75-B43; A75-B44; A75-B45; A75-B46; A75-B47; A75-B48; A75-B49; A75-B50; A75-B51; A75-B52; A75-B53; A75-B54; A75-B55; A75-B56; A75-B57; A75-B58; A75-B59; A75-B60; A75-B61; A75-B62; A75-B63; A75-B64; A75-B65; A75-B66; A75-B67; A75-B68; A75-B69; A75-B70; A75-B71; A75-B72; A75-B73; A75-B74; A75-B75; A75-B76; A75-B77; A75-B78; A75-B79; A75-B80; A75-B81; A75-B82; A75-B83; A75-B84; A75-B85; A75-B86; A75-B87; A75-B88; A75-B89; A75-B90; A75-B91; A75-B92; A75-B93; A75-B94; A75-B95; A75-B96; A75-B97; A75-B98; A75-B99; A75-B100; A75-B101; A75-B102; A75-B103; A75-B104; A75-B105; A75-B106; A75-B107; A75-B108; A75-B109; A75-B110; A75-B111; A75-B112; A75-B113;

A76-B1; A76-B2; A76-B3; A76-B4; A76-B5; A76-B6; A76-B7; A76-B8; A76-B9; A76-B10; A76-B11; A76-B12; A76-B13; A76-B14; A76-B15; A76-B16; A76-B17; A76-B18; A76-B19; A76-B20; A76-B21; A76-B22; A76-B23; A76-B24; A76-B25; A76-B26; A76-B27; A76-B28; A76-B29; A76-B30; A76-B31; A76-B32; A76-B33; A76-B34; A76-B35; A76-B36; A76-B37; A76-B38; A76-B39; A76-B40; A76-B41; A76-B42; A76-B43; A76-B44; A76-B45; A76-B46; A76-B47; A76-B48; A76-B49; A76-B50; A76-B51; A76-B52; A76-B53; A76-B54; A76-B55; A76-B56; A76-B57; A76-B58; A76-B59; A76-B60; A76-B61; A76-B62; A76-B63; A76-B64; A76-B65; A76-B66; A76-B67; A76-B68; A76-B69; A76-B70; A76-B71; A76-B72; A76-B73; A76-B74; A76-B75; A76-B76; A76-B77; A76-B78; A76-B79; A76-B80; A76-B81; A76-B82; A76-B83; A76-B84; A76-B85; A76-B86; A76-B87; A76-B88; A76-B89; A76-B90; A76-B91; A76-B92; A76-B93; A76-B94; A76-B95; A76-B96; A76-B97; A76-B98; A76-B99; A76-B100; A76-B101; A76-B102; A76-B103; A76-B104; A76-B105; A76-B106; A76-B107; A76-B108; A76-B109; A76-B110; A76-B111; A76-B112; A76-B113;

A77-B1; A77-B2; A77-B3; A77-B4; A77-B5; A77-B6; A77-B7; A77-B8; A77-B9; A77-B10; A77-B11; A77-B12; A77-B13; A77-B14; A77-B15; A77-B16; A77-B17; A77-B18; A77-B19; A77-B20; A77-B21; A77-B22; A77-B23; A77-B24; A77-B25; A77-B26; A77-B27; A77-B28; A77-B29; A77-B30; A77-B31; A77-B32; A77-B33; A77-B34; A77-B35; A77-B36; A77-B37; A77-B38; A77-B39; A77-B40; A77-B41; A77-B42; A77-B43; A77-B44; A77-B45; A77-B46; A77-B47; A77-B48; A77-B49; A77-B50; A77-B51; A77-B52; A77-B53; A77-B54; A77-B55; A77-B56; A77-B57; A77-B58; A77-B59; A77-B60; A77-B61; A77-B62; A77-B63; A77-B64; A77-B65; A77-B66; A77-B67; A77-B68; A77-B69; A77-B70; A77-B71; A77-B72; A77-B73; A77-B74; A77-B75; A77-B76; A77-B77; A77-B78; A77-B79; A77-B80; A77-B81; A77-B82; A77-B83; A77-B84; A77-B85; A77-B86; A77-B87; A77-B88; A77-B89; A77-B90; A77-B91; A77-B92; A77-B93; A77-B94; A77-B95; A77-B96; A77-B97; A77-B98; A77-B99; A77-B100; A77-B101; A77-B102; A77-B103; A77-B104; A77-B105; A77-B106; A77-B107; A77-B108; A77-B109; A77-B110; A77-B111; A77-B112; A77-B113;

A78-B1; A78-B2; A78-B3; A78-B4; A78-B5; A78-B6; A78-B7; A78-B8; A78-B9; A78-B10; A78-B11; A78-B12; A78-B13; A78-B14; A78-B15; A78-B16; A78-B17; A78-B18; A78-B19; A78-B20; A78-B21; A78-B22; A78-B23; A78-B24; A78-B25; A78-B26; A78-B27; A78-B28; A78-B29; A78-B30; A78-B31; A78-B32; A78-B33; A78-B34; A78-B35; A78-B36; A78-B37; A78-B38; A78-B39; A78-B40; A78-B41; A78-B42; A78-B43; A78-B44; A78-B45; A78-B46; A78-B47; A78-B48; A78-B49; A78-B50; A78-B51; A78-B52; A78-B53; A78-B54; A78-B55; A78-B56; A78-B57; A78-B58; A78-B59; A78-B60; A78-B61; A78-B62; A78-B63; A78-B64; A78-B65; A78-B66; A78-B67; A78-B68; A78-B69; A78-B70; A78-B71; A78-B72; A78-B73; A78-B74; A78-B75; A78-B76; A78-B77; A78-B78; A78-B79; A78-B80; A78-B81; A78-B82; A78-B83; A78-B84; A78-B85; A78-B86; A78-B87; A78-B88; A78-B89; A78-B90; A78-B91; A78-B92; A78-B93; A78-B94; A78-B95; A78-B96; A78-B97; A78-B98; A78-B99; A78-B100; A78-B101; A78-B102; A78-B103; A78-B104; A78-B105; A78-B106; A78-B107; A78-B108; A78-B109; A78-B110; A78-B111; A78-B112; A78-B113;

A79-B1; A79-B2; A79-B3; A79-B4; A79-B5; A79-B6; A79-B7; A79-B8; A79-B9; A79-B10; A79-B11; A79-B12; A79-B13; A79-B14; A79-B15; A79-B16; A79-B17; A79-B18; A79-B19; A79-B20; A79-B21; A79-B22; A79-B23; A79-B24; A79-B25; A79-B26; A79-B27; A79-B28; A79-B29; A79-B30; A79-B31; A79-B32; A79-B33; A79-B34; A79-B35; A79-B36; A79-B37; A79-B38; A79-B39; A79-B40; A79-B41; A79-B42; A79-B43; A79-B44; A79-B45; A79-B46; A79-B47; A79-B48; A79-B49; A79-B50; A79-B51; A79-B52; A79-B53; A79-B54; A79-B55; A79-B56; A79-B57; A79-B58; A79-B59; A79-B60; A79-B61; A79-B62; A79-B63; A79-B64; A79-B65; A79-B66; A79-B67; A79-B68; A79-B69; A79-B70; A79-B71; A79-B72; A79-B73; A79-B74; A79-B75; A79-B76; A79-B77; A79-B78; A79-B79; A79-B80; A79-B81; A79-B82; A79-B83; A79-B84; A79-B85; A79-B86; A79-B87; A79-B88; A79-B89; A79-B90; A79-B91; A79-B92; A79-B93; A79-B94; A79-B95; A79-B96; A79-B97; A79-B98; A79-B99; A79-B100; A79-B101; A79-B102; A79-B103; A79-B104; A79-B105; A79-B106; A79-B107; A79-B108; A79-B109; A79-B110; A79-B111; A79-B112; A79-B113;

A80-B1; A80-B2; A80-B3; A80-B4; A80-B5; A80-B6; A80-B7; A80-B8; A80-B9; A80-B10; A80-B11; A80-B12; A80-B13; A80-B14; A80-B15; A80-B16; A80-B17; A80-B18; A80-B19; A80-B20; A80-B21; A80-B22; A80-B23; A80-B24; A80-B25; A80-B26; A80-B27; A80-B28; A80-B29; A80-B30; A80-B31; A80-B32; A80-B33; A80-B34; A80-B35; A80-B36; A80-B37; A80-B38; A80-B39; A80-B40; A80-B41; A80-B42; A80-B43; A80-B44; A80-B45; A80-B46; A80-B47; A80-B48; A80-B49; A80-B50; A80-B51; A80-B52; A80-B53; A80-B54; A80-B55; A80-B56; A80-B57; A80-B58; A80-B59; A80-B60; A80-B61; A80-B62; A80-B63; A80-B64; A80-B65; A80-B66; A80-B67; A80-B68; A80-B69; A80-B70; A80-B71; A80-B72; A80-B73; A80-B74; A80-B75; A80-B76; A80-B77; A80-B78; A80-B79; A80-B80; A80-B81; A80-B82; A80-B83; A80-B84; A80-B85; A80-B86; A80-B87; A80-B88; A80-B89; A80-B90; A80-B91; A80-B92; A80-B93; A80-B94; A80-B95; A80-B96; A80-B97; A80-B98; A80-B99; A80-B100; A80-B101; A80-B102; A80-B103; A80-B104; A80-B105; A80-B106; A80-B107; A80-B108; A80-B109; A80-B110; A80-B111; A80-B112; A80-B113;

A81-B1; A81-B2; A81-B3; A81-B4; A81-B5; A81-B6; A81-B7; A81-B8; A81-B9; A81-B10; A81-B11; A81-B12; A81-B13; A81-B14; A81-B15; A81-B16; A81-B17; A81-B18; A81-B19; A81-B20; A81-B21; A81-B22; A81-B23; A81-B24; A81-B25; A81-B26; A81-B27; A81-B28; A81-B29; A81-B30; A81-B31; A81-B32; A81-B33; A81-B34; A81-B35; A81-B36; A81-B37; A81-B38; A81-B39; A81-B40; A81-B41; A81-B42; A81-B43; A81-B44; A81-B45; A81-B46; A81-B47; A81-B48; A81-B49; A81-B50; A81-B51; A81-B52; A81-B53; A81-B54; A81-B55; A81-B56; A81-B57; A81-B58; A81-B59; A81-B60; A81-B61; A81-B62; A81-B63; A81-B64; A81-B65; A81-B66; A81-B67; A81-B68; A81-B69; A81-B70; A81-B71; A81-B72; A81-B73; A81-B74; A81-B75; A81-B76; A81-B77; A81-B78; A81-B79; A81-B80; A81-B81; A81-B82; A81-B83; A81-B84; A81-B85; A81-B86; A81-B87; A81-B88; A81-B89; A81-B90; A81-B91; A81-B92; A81-B93; A81-B94; A81-B95; A81-B96; A81-B97; A81-B98; A81-B99; A81-B100; A81-B101; A81-B102; A81-B103; A81-B104; A81-B105; A81-B106; A81-B107; A81-B108; A81-B109; A81-B110; A81-B111; A81-B112; A81-B113;

A82-B1; A82-B2; A82-B3; A82-B4; A82-B5; A82-B6; A82-B7; A82-B8; A82-B9; A82-B10; A82-B11; A82-B12; A82-B13; A82-B14; A82-B15; A82-B16; A82-B17; A82-B18; A82-B19; A82-B20; A82-B21; A82-B22; A82-B23; A82-B24; A82-B25; A82-B26; A82-B27; A82-B28; A82-B29; A82-B30; A82-B31; A82-B32; A82-B33; A82-B34; A82-B35; A82-B36; A82-B37; A82-B38; A82-B39; A82-B40; A82-B41; A82-B42; A82-B43; A82-B44; A82-B45; A82-B46; A82-B47; A82-B48; A82-B49; A82-B50; A82-B51; A82-B52; A82-B53; A82-B54; A82-B55; A82-B56; A82-B57; A82-B58; A82-B59; A82-B60; A82-B61; A82-B62; A82-B63; A82-B64; A82-B65; A82-B66; A82-B67; A82-B68; A82-B69; A82-B70; A82-B71; A82-B72; A82-B73; A82-B74; A82-B75; A82-B76; A82-B77; A82-B78; A82-B79; A82-B80; A82-B81; A82-B82; A82-B83; A82-B84; A82-B85; A82-B86; A82-B87; A82-B88; A82-B89; A82-B90; A82-B91; A82-B92; A82-B93; A82-B94; A82-B95; A82-B96; A82-B97; A82-B98; A82-B99; A82-B100; A82-B101; A82-B102; A82-B103; A82-B104; A82-B105; A82-B106; A82-B107; A82-B108; A82-B109; A82-B110; A82-B111; A82-B112; A82-B113;

A83-B1; A83-B2; A83-B3; A83-B4; A83-B5; A83-B6; A83-B7; A83-B8; A83-B9; A83-B10; A83-B11; A83-B12; A83-B13; A83-B14; A83-B15; A83-B16; A83-B17; A83-B18; A83-B19; A83-B20; A83-B21; A83-B22; A83-B23; A83-B24; A83-B25; A83-B26; A83-B27; A83-B28; A83-B29; A83-B30; A83-B31; A83-B32; A83-B33; A83-B34; A83-B35; A83-B36; A83-B37; A83-B38; A83-B39; A83-B40; A83-B41; A83-B42; A83-B43; A83-B44; A83-B45; A83-B46; A83-B47; A83-B48; A83-B49; A83-B50; A83-B51; A83-B52; A83-B53; A83-B54; A83-B55; A83-B56; A83-B57; A83-B58; A83-B59; A83-B60; A83-B61; A83-B62; A83-B63; A83-B64; A83-B65; A83-B66; A83-B67; A83-B68; A83-B69; A83-B70; A83-B71; A83-B72; A83-B73; A83-B74; A83-B75; A83-B76; A83-B77; A83-B78; A83-B79; A83-B80; A83-B81; A83-B82; A83-B83; A83-B84; A83-B85; A83-B86; A83-B87; A83-B88; A83-B89; A83-B90; A83-B91; A83-B92; A83-B93; A83-B94; A83-B95; A83-B96; A83-B97; A83-B98; A83-B99; A83-B100; A83-B101; A83-B102; A83-B103; A83-B104; A83-B105; A83-B106; A83-B107; A83-B108; A83-B109; A83-B110; A83-B111; A83-B112; A83-B113;

A84-B1; A84-B2; A84-B3; A84-B4; A84-B5; A84-B6; A84-B7; A84-B8; A84-B9; A84-B10; A84-B11; A84-B12; A84-B13; A84-B14; A84-B15; A84-B16; A84-B17; A84-B18; A84-B19; A84-B20; A84-B21; A84-B22; A84-B23; A84-B24; A84-B25; A84-B26; A84-B27; A84-B28; A84-B29; A84-B30; A84-B31; A84-B32; A84-B33; A84-B34; A84-B35; A84-B36; A84-B37; A84-B38; A84-B39; A84-B40; A84-B41; A84-B42; A84-B43; A84-B44; A84-B45; A84-B46; A84-B47; A84-B48; A84-B49; A84-B50; A84-B51; A84-B52; A84-B53; A84-B54; A84-B55; A84-B56; A84-B57; A84-B58; A84-B59; A84-B60; A84-B61; A84-B62; A84-B63; A84-B64; A84-B65; A84-B66; A84-B67; A84-B68; A84-B69; A84-B70; A84-B71; A84-B72; A84-B73; A84-B74; A84-B75; A84-B76; A84-B77; A84-B78; A84-B79; A84-B80; A84-B81; A84-B82; A84-B83; A84-B84; A84-B85; A84-B86; A84-B87; A84-B88; A84-B89; A84-B90; A84-B91; A84-B92; A84-B93; A84-B94; A84-B95; A84-B96; A84-B97; A84-B98; A84-B99; A84-B100; A84-B101; A84-B102; A84-B103; A84-B104; A84-B105; A84-B106; A84-B107; A84-B108; A84-B109; A84-B110; A84-B111; A84-B112; A84-B113;

A85-B1; A85-B2; A85-B3; A85-B4; A85-B5; A85-B6; A85-B7; A85-B8; A85-B9; A85-B10; A85-B11; A85-B12; A85-B13; A85-B14; A85-B15; A85-B16; A85-B17; A85-B18; A85-B19; A85-B20; A85-B21; A85-B22; A85-B23; A85-B24; A85-B25; A85-B26; A85-B27; A85-B28; A85-B29; A85-B30; A85-B31; A85-B32; A85-B33; A85-B34; A85-B35; A85-B36; A85-B37; A85-B38; A85-B39; A85-B40; A85-B41; A85-B42; A85-B43; A85-B44; A85-B45; A85-B46; A85-B47; A85-B48; A85-B49; A85-B50; A85-B51; A85-B52; A85-B53; A85-B54; A85-B55; A85-B56; A85-B57; A85-B58; A85-B59; A85-B60; A85-B61; A85-B62; A85-B63; A85-B64; A85-B65; A85-B66; A85-B67; A85-B68; A85-B69; A85-B70; A85-B71; A85-B72; A85-B73; A85-B74; A85-B75; A85-B76; A85-B77; A85-B78; A85-B79; A85-B80; A85-B81; A85-B82; A85-B83; A85-B84; A85-B85; A85-B86; A85-B87; A85-B88; A85-B89; A85-B90; A85-B91; A85-B92; A85-B93; A85-B94; A85-B95; A85-B96; A85-B97; A85-B98; A85-B99; A85-B100; A85-B101; A85-B102; A85-B103; A85-B104; A85-B105; A85-B106; A85-B107; A85-B108; A85-B109; A85-B110; A85-B111; A85-B112; A85-B113;

A86-B1; A86-B2; A86-B3; A86-B4; A86-B5; A86-B6; A86-B7; A86-B8; A86-B9; A86-B10; A86-B11; A86-B12; A86-B13; A86-B14; A86-B15; A86-B16; A86-B17; A86-B18; A86-B19; A86-B20; A86-B21; A86-B22; A86-B23; A86-B24; A86-B25; A86-B26; A86-B27; A86-B28; A86-B29; A86-B30; A86-B31; A86-B32; A86-B33; A86-B34; A86-B35; A86-B36; A86-B37; A86-B38; A86-B39; A86-B40; A86-B41; A86-B42; A86-B43; A86-B44; A86-B45; A86-B46; A86-B47; A86-B48; A86-B49; A86-B50; A86-B51; A86-B52; A86-B53; A86-B54; A86-B55; A86-B56; A86-B57; A86-B58; A86-B59; A86-B60; A86-B61; A86-B62; A86-B63; A86-B64; A86-B65; A86-B66; A86-B67; A86-B68; A86-B69; A86-B70; A86-B71; A86-B72; A86-B73; A86-B74; A86-B75; A86-B76; A86-B77; A86-B78; A86-B79; A86-B80; A86-B81; A86-B82; A86-B83; A86-B84; A86-B85; A86-B86; A86-B87; A86-B88; A86-B89; A86-B90; A86-B91; A86-B92; A86-B93; A86-B94; A86-B95; A86-B96; A86-B97; A86-B98; A86-B99; A86-B100; A86-B101; A86-B102; A86-B103; A86-B104; A86-B105; A86-B106; A86-B107; A86-B108; A86-B109; A86-B110; A86-B111; A86-B112; A86-B113;

A87-B1; A87-B2; A87-B3; A87-B4; A87-B5; A87-B6; A87-B7; A87-B8; A87-B9; A87-B10; A87-B11; A87-B12; A87-B13; A87-B14; A87-B15; A87-B16; A87-B17; A87-B18; A87-B19; A87-B20; A87-B21; A87-B22; A87-B23; A87-B24; A87-B25; A87-B26; A87-B27; A87-B28; A87-B29; A87-B30; A87-B31; A87-B32; A87-B33; A87-B34; A87-B35; A87-B36; A87-B37; A87-B38; A87-B39; A87-B40; A87-B41; A87-B42; A87-B43; A87-B44; A87-B45; A87-B46; A87-B47; A87-B48; A87-B49; A87-B50; A87-B51; A87-B52; A87-B53; A87-B54; A87-B55; A87-B56; A87-B57; A87-B58; A87-B59; A87-B60; A87-B61; A87-B62; A87-B63; A87-B64; A87-B65; A87-B66; A87-B67; A87-B68; A87-B69; A87-B70; A87-B71; A87-B72; A87-B73; A87-B74; A87-B75; A87-B76; A87-B77; A87-B78; A87-B79; A87-B80; A87-B81; A87-B82; A87-B83; A87-B84; A87-B85; A87-B86; A87-B87; A87-B88; A87-B89; A87-B90; A87-B91; A87-B92; A87-B93; A87-B94; A87-B95; A87-B96; A87-B97; A87-B98; A87-B99; A87-B100; A87-B101; A87-B102; A87-B103; A87-B104; A87-B105; A87-B106; A87-B107; A87-B108; A87-B109; A87-B110; A87-B111; A87-B112; A87-B113;

A88-B1; A88-B2; A88-B3; A88-B4; A88-B5; A88-B6; A88-B7; A88-B8; A88-B9; A88-B10; A88-B11; A88-B12; A88-B13; A88-B14; A88-B15; A88-B16; A88-B17; A88-B18; A88-B19; A88-B20; A88-B21; A88-B22; A88-B23; A88-B24; A88-B25; A88-B26; A88-B27; A88-B28; A88-B29; A88-B30; A88-B31; A88-B32; A88-B33; A88-B34; A88-B35; A88-B36; A88-B37; A88-B38; A88-B39; A88-B40; A88-B41; A88-B42; A88-B43; A88-B44; A88-B45; A88-B46; A88-B47; A88-B48; A88-B49; A88-B50; A88-B51; A88-B52; A88-B53; A88-B54; A88-B55; A88-B56; A88-B57; A88-B58; A88-B59; A88-B60; A88-B61; A88-B62; A88-B63; A88-B64; A88-B65; A88-B66; A88-B67; A88-B68; A88-B69; A88-B70; A88-B71; A88-B72; A88-B73; A88-B74; A88-B75; A88-B76; A88-B77; A88-B78; A88-B79; A88-B80; A88-B81; A88-B82; A88-B83; A88-B84; A88-B85; A88-B86; A88-B87; A88-B88; A88-B89; A88-B90; A88-B91; A88-B92; A88-B93; A88-B94; A88-B95; A88-B96; A88-B97; A88-B98; A88-B99; A88-B100; A88-B101; A88-B102; A88-B103; A88-B104; A88-B105; A88-B106; A88-B107; A88-B108; A88-B109; A88-B110; A88-B111; A88-B112; A88-B113;

A89-B1; A89-B2; A89-B3; A89-B4; A89-B5; A89-B6; A89-B7; A89-B8; A89-B9; A89-B10; A89-B11; A89-B12; A89-B13; A89-B14; A89-B15; A89-B16; A89-B17; A89-B18; A89-B19; A89-B20; A89-B21; A89-B22; A89-B23; A89-B24; A89-B25; A89-B26; A89-B27; A89-B28; A89-B29; A89-B30; A89-B31; A89-B32; A89-B33; A89-B34; A89-B35; A89-B36; A89-B37; A89-B38; A89-B39; A89-B40; A89-B41; A89-B42; A89-B43; A89-B44; A89-B45; A89-B46; A89-B47; A89-B48; A89-B49; A89-B50; A89-B51; A89-B52; A89-B53; A89-B54; A89-B55; A89-B56; A89-B57; A89-B58; A89-B59; A89-B60; A89-B61; A89-B62; A89-B63; A89-B64; A89-B65; A89-B66; A89-B67; A89-B68; A89-B69; A89-B70; A89-B71; A89-B72; A89-B73; A89-B74; A89-B75; A89-B76; A89-B77; A89-B78; A89-B79; A89-B80; A89-B81; A89-B82; A89-B83; A89-B84; A89-B85; A89-B86; A89-B87; A89-B88; A89-B89; A89-B90; A89-B91; A89-B92; A89-B93; A89-B94; A89-B95; A89-B96; A89-B97; A89-B98; A89-B99; A89-B100; A89-B101; A89-B102; A89-B103; A89-B104; A89-B105; A89-B106; A89-B107; A89-B108; A89-B109; A89-B110; A89-B111; A89-B112; A89-B113;

A90-B1; A90-B2; A90-B3; A90-B4; A90-B5; A90-B6; A90-B7; A90-B8; A90-B9; A90-B10; A90-B11; A90-B12; A90-B13; A90-B14; A90-B15; A90-B16; A90-B17; A90-B18; A90-B19; A90-B20; A90-B21; A90-B22; A90-B23; A90-B24; A90-B25; A90-B26; A90-B27; A90-B28; A90-B29; A90-B30; A90-B31; A90-B32; A90-B33; A90-B34; A90-B35; A90-B36; A90-B37; A90-B38; A90-B39; A90-B40; A90-B41; A90-B42; A90-B43; A90-B44; A90-B45; A90-B46; A90-B47; A90-B48; A90-B49; A90-B50; A90-B51; A90-B52; A90-B53; A90-B54; A90-B55; A90-B56; A90-B57; A90-B58; A90-B59; A90-B60; A90-B61; A90-B62; A90-B63; A90-B64; A90-B65; A90-B66; A90-B67; A90-B68; A90-B69; A90-B70; A90-B71; A90-B72; A90-B73; A90-B74; A90-B75; A90-B76; A90-B77; A90-B78; A90-B79; A90-B80; A90-B81; A90-B82; A90-B83; A90-B84; A90-B85; A90-B86; A90-B87; A90-B88; A90-B89; A90-B90; A90-B91; A90-B92; A90-B93; A90-B94; A90-B95; A90-B96; A90-B97; A90-B98; A90-B99; A90-B100; A90-B101; A90-B102; A90-B103; A90-B104; A90-B105; A90-B106; A90-B107; A90-B108; A90-B109; A90-B110; A90-B111; A90-B112; A90-B113;

A91-B1; A91-B2; A91-B3; A91-B4; A91-B5; A91-B6; A91-B7; A91-B8; A91-B9; A91-B10; A91-B11; A91-B12; A91-B13; A91-B14; A91-B15; A91-B16; A91-B17; A91-B18; A91-B19; A91-B20; A91-B21; A91-B22; A91-B23; A91-B24; A91-B25; A91-B26; A91-B27; A91-B28; A91-B29; A91-B30; A91-B31; A91-B32; A91-B33; A91-B34; A91-B35; A91-B36; A91-B37; A91-B38; A91-B39; A91-B40; A91-B41; A91-B42; A91-B43; A91-B44; A91-B45; A91-B46; A91-B47; A91-B48; A91-B49; A91-B50; A91-B51; A91-B52; A91-B53; A91-B54; A91-B55; A91-B56; A91-B57; A91-B58; A91-B59; A91-B60; A91-B61; A91-B62; A91-B63; A91-B64; A91-B65; A91-B66; A91-B67; A91-B68; A91-B69; A91-B70; A91-B71; A91-B72; A91-B73; A91-B74; A91-B75; A91-B76; A91-B77; A91-B78; A91-B79; A91-B80; A91-B81; A91-B82; A91-B83; A91-B84; A91-B85; A91-B86; A91-B87; A91-B88; A91-B89; A91-B90; A91-B91; A91-B92; A91-B93; A91-B94; A91-B95; A91-B96; A91-B97; A91-B98; A91-B99; A91-B100; A91-B101; A91-B102; A91-B103; A91-B104; A91-B105; A91-B106; A91-B107; A91-B108; A91-B109; A91-B110; A91-B111; A91-B112; A91-B113;

A92-B1; A92-B2; A92-B3; A92-B4; A92-B5; A92-B6; A92-B7; A92-B8; A92-B9; A92-B10; A92-B11; A92-B12; A92-B13; A92-B14; A92-B15; A92-B16; A92-B17; A92-B18; A92-B19; A92-B20; A92-B21; A92-B22; A92-B23; A92-B24; A92-B25; A92-B26; A92-B27; A92-B28; A92-B29; A92-B30; A92-B31; A92-B32; A92-B33; A92-B34; A92-B35; A92-B36; A92-B37; A92-B38; A92-B39; A92-B40; A92-B41; A92-B42; A92-B43; A92-B44; A92-B45; A92-B46; A92-B47; A92-B48; A92-B49; A92-B50; A92-B51; A92-B52; A92-B53; A92-B54; A92-B55; A92-B56; A92-B57; A92-B58; A92-B59; A92-B60; A92-B61; A92-B62; A92-B63; A92-B64; A92-B65; A92-B66; A92-B67; A92-B68; A92-B69; A92-B70; A92-B71; A92-B72; A92-B73; A92-B74; A92-B75; A92-B76; A92-B77; A92-B78; A92-B79; A92-B80; A92-B81; A92-B82; A92-B83; A92-B84; A92-B85; A92-B86; A92-B87; A92-B88; A92-B89; A92-B90; A92-B91; A92-B92; A92-B93; A92-B94; A92-B95; A92-B96; A92-B97; A92-B98; A92-B99; A92-B100; A92-B101; A92-B102; A92-B103; A92-B104; A92-B105; A92-B106; A92-B107; A92-B108; A92-B109; A92-B110; A92-B111; A92-B112; A92-B113;

A93-B1; A93-B2; A93-B3; A93-B4; A93-B5; A93-B6; A93-B7; A93-B8; A93-B9; A93-B10; A93-B11; A93-B12; A93-B13; A93-B14; A93-B15; A93-B16; A93-B17; A93-B18; A93-B19; A93-B20; A93-B21; A93-B22; A93-B23; A93-B24; A93-B25; A93-B26; A93-B27; A93-B28; A93-B29; A93-B30; A93-B31; A93-B32; A93-B33; A93-B34; A93-B35; A93-B36; A93-B37; A93-B38; A93-B39; A93-B40; A93-B41; A93-B42; A93-B43; A93-B44; A93-B45; A93-B46; A93-B47; A93-B48; A93-B49; A93-B50; A93-B51; A93-B52; A93-B53; A93-B54; A93-B55; A93-B56; A93-B57; A93-B58; A93-B59; A93-B60; A93-B61; A93-B62; A93-B63; A93-B64; A93-B65; A93-B66; A93-B67; A93-B68; A93-B69; A93-B70; A93-B71; A93-B72; A93-B73; A93-B74; A93-B75; A93-B76; A93-B77; A93-B78; A93-B79; A93-B80; A93-B81; A93-B82; A93-B83; A93-B84; A93-B85; A93-B86; A93-B87; A93-B88; A93-B89; A93-B90; A93-B91; A93-B92; A93-B93; A93-B94; A93-B95; A93-B96; A93-B97; A93-B98; A93-B99; A93-B100; A93-B101; A93-B102; A93-B103; A93-B104; A93-B105; A93-B106; A93-B107; A93-B108; A93-B109; A93-B110; A93-B111; A93-B112; A93-B113;

A94-B1; A94-B2; A94-B3; A94-B4; A94-B5; A94-B6; A94-B7; A94-B8; A94-B9; A94-B10; A94-B11; A94-B12; A94-B13; A94-B14; A94-B15; A94-B16; A94-B17; A94-B18; A94-B19; A94-B20; A94-B21; A94-B22; A94-B23; A94-B24; A94-B25; A94-B26; A94-B27; A94-B28; A94-B29; A94-B30; A94-B31; A94-B32; A94-B33; A94-B34; A94-B35; A94-B36; A94-B37; A94-B38; A94-B39; A94-B40; A94-B41; A94-B42; A94-B43; A94-B44; A94-B45; A94-B46; A94-B47; A94-B48; A94-B49; A94-B50; A94-B51; A94-B52; A94-B53; A94-B54; A94-B55; A94-B56; A94-B57; A94-B58; A94-B59; A94-B60; A94-B61; A94-B62; A94-B63; A94-B64; A94-B65; A94-B66; A94-B67; A94-B68; A94-B69; A94-B70; A94-B71; A94-B72; A94-B73; A94-B74; A94-B75; A94-B76; A94-B77; A94-B78; A94-B79; A94-B80; A94-B81; A94-B82; A94-B83; A94-B84; A94-B85; A94-B86; A94-B87; A94-B88; A94-B89; A94-B90; A94-B91; A94-B92; A94-B93; A94-B94; A94-B95; A94-B96; A94-B97; A94-B98; A94-B99; A94-B100; A94-B101; A94-B102; A94-B103; A94-B104; A94-B105; A94-B106; A94-B107; A94-B108; A94-B109; A94-B110; A94-B111; A94-B112; A94-B113;

A95-B1; A95-B2; A95-B3; A95-B4; A95-B5; A95-B6; A95-B7; A95-B8; A95-B9; A95-B10; A95-B11; A95-B12; A95-B13; A95-B14; A95-B15; A95-B16; A95-B17; A95-B18; A95-B19; A95-B20; A95-B21; A95-B22; A95-B23; A95-B24; A95-B25; A95-B26; A95-B27; A95-B28; A95-B29; A95-B30; A95-B31; A95-B32; A95-B33; A95-B34; A95-B35; A95-B36; A95-B37; A95-B38; A95-B39; A95-B40; A95-B41; A95-B42; A95-B43; A95-B44; A95-B45; A95-B46; A95-B47; A95-B48; A95-B49; A95-B50; A95-B51; A95-B52; A95-B53; A95-B54; A95-B55; A95-B56; A95-B57; A95-B58; A95-B59; A95-B60; A95-B61; A95-B62; A95-B63; A95-B64; A95-B65; A95-B66; A95-B67; A95-B68; A95-B69; A95-B70; A95-B71; A95-B72; A95-B73; A95-B74; A95-B75; A95-B76; A95-B77; A95-B78; A95-B79; A95-B80; A95-B81; A95-B82; A95-B83; A95-B84; A95-B85; A95-B86; A95-B87; A95-B88; A95-B89; A95-B90; A95-B91; A95-B92; A95-B93; A95-B94; A95-B95; A95-B96; A95-B97; A95-B98; A95-B99; A95-B100; A95-B101; A95-B102; A95-B103; A95-B104; A95-B105; A95-B106; A95-B107; A95-B108; A95-B109; A95-B110; A95-B111; A95-B112; A95-B113;

A96-B1; A96-B2; A96-B3; A96-B4; A96-B5; A96-B6; A96-B7; A96-B8; A96-B9; A96-B10; A96-B11; A96-B12; A96-B13; A96-B14; A96-B15; A96-B16; A96-B17; A96-B18; A96-B19; A96-B20; A96-B21; A96-B22; A96-B23; A96-B24; A96-B25; A96-B26; A96-B27; A96-B28; A96-B29; A96-B30; A96-B31; A96-B32; A96-B33; A96-B34; A96-B35; A96-B36; A96-B37; A96-B38; A96-B39; A96-B40; A96-B41; A96-B42; A96-B43; A96-B44; A96-B45; A96-B46; A96-B47; A96-B48; A96-B49; A96-B50; A96-B51; A96-B52; A96-B53; A96-B54; A96-B55; A96-B56; A96-B57; A96-B58; A96-B59; A96-B60; A96-B61; A96-B62; A96-B63; A96-B64; A96-B65; A96-B66; A96-B67; A96-B68; A96-B69; A96-B70; A96-B71; A96-B72; A96-B73; A96-B74; A96-B75; A96-B76; A96-B77; A96-B78; A96-B79; A96-B80; A96-B81; A96-B82; A96-B83; A96-B84; A96-B85; A96-B86; A96-B87; A96-B88; A96-B89; A96-B90; A96-B91; A96-B92; A96-B93; A96-B94; A96-B95; A96-B96; A96-B97; A96-B98; A96-B99; A96-B100; A96-B101; A96-B102; A96-B103; A96-B104; A96-B105; A96-B106; A96-B107; A96-B108; A96-B109; A96-B110; A96-B111; A96-B112; A96-B113;

A97-B1; A97-B2; A97-B3; A97-B4; A97-B5; A97-B6; A97-B7; A97-B8; A97-B9; A97-B10; A97-B11; A97-B12; A97-B13; A97-B14; A97-B15; A97-B16; A97-B17; A97-B18; A97-B19; A97-B20; A97-B21; A97-B22; A97-B23; A97-B24; A97-B25; A97-B26; A97-B27; A97-B28; A97-B29; A97-B30; A97-B31; A97-B32; A97-B33; A97-B34; A97-B35; A97-B36; A97-B37; A97-B38; A97-B39; A97-B40; A97-B41; A97-B42; A97-B43; A97-B44; A97-B45; A97-B46; A97-B47; A97-B48; A97-B49; A97-B50; A97-B51; A97-B52; A97-B53; A97-B54; A97-B55; A97-B56; A97-B57; A97-B58; A97-B59; A97-B60; A97-B61; A97-B62; A97-B63; A97-B64; A97-B65; A97-B66; A97-B67; A97-B68; A97-B69; A97-B70; A97-B71; A97-B72; A97-B73; A97-B74; A97-B75; A97-B76; A97-B77; A97-B78; A97-B79; A97-B80; A97-B81; A97-B82; A97-B83; A97-B84; A97-B85; A97-B86; A97-B87; A97-B88; A97-B89; A97-B90; A97-B91; A97-B92; A97-B93; A97-B94; A97-B95; A97-B96; A97-B97; A97-B98; A97-B99; A97-B100; A97-B101; A97-B102; A97-B103; A97-B104; A97-B105; A97-B106; A97-B107; A97-B108; A97-B109; A97-B110; A97-B111; A97-B112; A97-B113;

A98-B1; A98-B2; A98-B3; A98-B4; A98-B5; A98-B6; A98-B7; A98-B8; A98-B9; A98-B10; A98-B11; A98-B12; A98-B13; A98-B14; A98-B15; A98-B16; A98-B17; A98-B18; A98-B19; A98-B20; A98-B21; A98-B22; A98-B23; A98-B24; A98-B25; A98-B26; A98-B27; A98-B28; A98-B29; A98-B30; A98-B31; A98-B32; A98-B33; A98-B34; A98-B35; A98-B36; A98-B37; A98-B38; A98-B39; A98-B40; A98-B41; A98-B42; A98-B43; A98-B44; A98-B45; A98-B46; A98-B47; A98-B48; A98-B49; A98-B50; A98-B51; A98-B52; A98-B53; A98-B54; A98-B55; A98-B56; A98-B57; A98-B58; A98-B59; A98-B60; A98-B61; A98-B62; A98-B63; A98-B64; A98-B65; A98-B66; A98-B67; A98-B68; A98-B69; A98-B70; A98-B71; A98-B72; A98-B73; A98-B74; A98-B75; A98-B76; A98-B77; A98-B78; A98-B79; A98-B80; A98-B81; A98-B82; A98-B83; A98-B84; A98-B85; A98-B86; A98-B87; A98-B88; A98-B89; A98-B90; A98-B91; A98-B92; A98-B93; A98-B94; A98-B95; A98-B96; A98-B97; A98-B98; A98-B99; A98-B100; A98-B101; A98-B102; A98-B103; A98-B104; A98-B105; A98-B106; A98-B107; A98-B108; A98-B109; A98-B110; A98-B111; A98-B112; A98-B113;

A99-B1; A99-B2; A99-B3; A99-B4; A99-B5; A99-B6; A99-B7; A99-B8; A99-B9; A99-B10; A99-B11; A99-B12; A99-B13; A99-B14; A99-B15; A99-B16; A99-B17; A99-B18; A99-B19; A99-B20; A99-B21; A99-B22; A99-B23; A99-B24; A99-B25; A99-B26; A99-B27; A99-B28; A99-B29; A99-B30; A99-B31; A99-B32; A99-B33; A99-B34; A99-B35; A99-B36; A99-B37; A99-B38; A99-B39; A99-B40; A99-B41; A99-B42; A99-B43; A99-B44; A99-B45; A99-B46; A99-B47; A99-B48; A99-B49; A99-B50; A99-B51; A99-B52; A99-B53; A99-B54; A99-B55; A99-B56; A99-B57; A99-B58; A99-B59; A99-B60; A99-B61; A99-B62; A99-B63; A99-B64; A99-B65; A99-B66; A99-B67; A99-B68; A99-B69; A99-B70; A99-B71; A99-B72; A99-B73; A99-B74; A99-B75; A99-B76; A99-B77; A99-B78; A99-B79; A99-B80; A99-B81; A99-B82; A99-B83; A99-B84; A99-B85; A99-B86; A99-B87; A99-B88; A99-B89; A99-B90; A99-B91; A99-B92; A99-B93; A99-B94; A99-B95; A99-B96; A99-B97; A99-B98; A99-B99; A99-B100; A99-B101; A99-B102; A99-B103; A99-B104; A99-B105; A99-B106; A99-B107; A99-B108; A99-B109; A99-B110; A99-B111; A99-B112; A99-B113;

A100-B1; A100-B2; A100-B3; A100-B4; A100-B5; A100-B6; A100-B7; A100-B8; A100-B9; A100-B10; A100-B11; A100-B12; A100-B13; A100-B14; A100-B15; A100-B16; A100-B17; A100-B18; A100-B19; A100-B20; A100-B21; A100-B22; A100-B23; A100-B24; A100-B25; A100-B26; A100-B27; A100-B28; A100-B29; A100-B30; A100-B31; A100-B32; A100-B33; A100-B34; A100-B35; A100-B36; A100-B37; A100-B38; A100-B39; A100-B40; A100-B41; A100-B42; A100-B43; A100-B44; A100-B45; A100-B46; A100-B47; A100-B48; A100-B49; A100-B50; A100-B51; A100-B52; A100-B53; A100-B54; A100-B55; A100-B56; A100-B57; A100-B58; A100-B59; A100-B60; A100-B61; A100-B62; A100-B63; A100-B64; A100-B65; A100-B66; A100-B67; A100-B68; A100-B69; A100-B70; A100-B71; A100-B72; A100-B73; A100-B74; A100-B75; A100-B76; A100-B77; A100-B78; A100-B79; A100-B80; A100-B81; A100-B82; A100-B83; A100-B84; A100-B85; A100-B86; A100-B87; A100-B88; A100-B89; A100-B90; A100-B91; A100-B92; A100-B93; A100-B94; A100-B95; A100-B96; A100-B97; A100-B98; A100-B99; A100-B100; A100-B101; A100-B102; A100-B103; A100-B104; A100-B105; A100-B106; A100-B107; A100-B108; A100-B109; A100-B110; A100-B111; A100-B112; A100-B113;

A101-B1; A101-B2; A101-B3; A101-B4; A101-B5; A101-B6; A101-B7; A101-B8; A101-B9; A101-B10; A101-B11; A101-B12; A101-B13; A101-B14; A101-B15; A101-B16; A101-B17; A101-B18; A101-B19; A101-B20; A101-B21; A101-B22; A101-B23; A101-B24; A101-B25; A101-B26; A101-B27; A101-B28; A101-B29; A101-B30; A101-B31; A101-B32; A101-B33; A101-B34; A101-B35; A101-B36; A101-B37; A101-B38; A101-B39; A101-B40; A101-B41; A101-B42; A101-B43; A101-B44; A101-B45; A101-B46; A101-B47; A101-B48; A101-B49; A101-B50; A101-B51; A101-B52; A101-B53; A101-B54; A101-B55; A101-B56; A101-B57; A101-B58; A101-B59; A101-B60; A101-B61; A101-B62; A101-B63; A101-B64; A101-B65; A101-B66; A101-B67; A101-B68; A101-B69; A101-B70; A101-B71; A101-B72; A101-B73; A101-B74; A101-B75; A101-B76; A101-B77; A101-B78; A101-B79; A101-B80; A101-B81; A101-B82; A101-B83; A101-B84; A101-B85; A101-B86; A101-B87; A101-B88; A101-B89; A101-B90; A101-B91; A101-B92; A101-B93; A101-B94; A101-B95; A101-B96; A101-B97; A101-B98; A101-B99; A101-B100; A101-B101; A101-B102; A101-B103; A101-B104; A101-B105; A101-B106; A101-B107; A101-B108; A101-B109; A101-B110; A101-B111; A101-B112; A101-B113;

A102-B1; A102-B2; A102-B3; A102-B4; A102-B5; A102-B6; A102-B7; A102-B8; A102-B9; A102-B10; A102-B11; A102-B12; A102-B13; A102-B14; A102-B15; A102-B16; A102-B17; A102-B18; A102-B19; A102-B20; A102-B21; A102-B22; A102-B23; A102-B24; A102-B25; A102-B26; A102-B27; A102-B28; A102-B29; A102-B30; A102-B31; A102-B32; A102-B33; A102-B34; A102-B35; A102-B36; A102-B37; A102-B38; A102-B39; A102-B40; A102-B41; A102-B42; A102-B43; A102-B44; A102-B45; A102-B46; A102-B47; A102-B48; A102-B49; A102-B50; A102-B51; A102-B52; A102-B53; A102-B54; A102-B55; A102-B56; A102-B57; A102-B58; A102-B59; A102-B60; A102-B61; A102-B62; A102-B63; A102-B64; A102-B65; A102-B66; A102-B67; A102-B68; A102-B69; A102-B70; A102-B71; A102-B72; A102-B73; A102-B74; A102-B75; A102-B76; A102-B77; A102-B78; A102-B79; A102-B80; A102-B81; A102-B82; A102-B83; A102-B84; A102-B85; A102-B86; A102-B87; A102-B88; A102-B89; A102-B90; A102-B91; A102-B92; A102-B93; A102-B94; A102-B95; A102-B96; A102-B97; A102-B98; A102-B99; A102-B100; A102-B101; A102-B102; A102-B103; A102-B104; A102-B105; A102-B106; A102-B107; A102-B108; A102-B109; A102-B110; A102-B111; A102-B112; A102-B113;

A103-B1; A103-B2; A103-B3; A103-B4; A103-B5; A103-B6; A103-B7; A103-B8; A103-B9; A103-B10; A103-B11; A103-B12; A103-B13; A103-B14; A103-B15; A103-B16; A103-B17; A103-B18; A103-B19; A103-B20; A103-B21; A103-B22; A103-B23; A103-B24; A103-B25; A103-B26; A103-B27; A103-B28; A103-B29; A103-B30; A103-B31; A103-B32; A103-B33; A103-B34; A103-B35; A103-B36; A103-B37; A103-B38; A103-B39; A103-B40; A103-B41; A103-B42; A103-B43; A103-B44; A103-B45; A103-B46; A103-B47; A103-B48; A103-B49; A103-B50; A103-B51; A103-B52; A103-B53; A103-B54; A103-B55; A103-B56; A103-B57; A103-B58; A103-B59; A103-B60; A103-B61; A103-B62; A103-B63; A103-B64; A103-B65; A103-B66; A103-B67; A103-B68; A103-B69; A103-B70; A103-B71; A103-B72; A103-B73; A103-B74; A103-B75; A103-B76; A103-B77; A103-B78; A103-B79; A103-B80; A103-B81; A103-B82; A103-B83; A103-B84; A103-B85; A103-B86; A103-B87; A103-B88; A103-B89; A103-B90; A103-B91; A103-B92; A103-B93; A103-B94; A103-B95; A103-B96; A103-B97; A103-B98; A103-B99; A103-B100; A103-B101; A103-B102; A103-B103; A103-B104; A103-B105; A103-B106; A103-B107; A103-B108; A103-B109; A103-B110; A103-B111; A103-B112; A103-B113;

A104-B1; A104-B2; A104-B3; A104-B4; A104-B5; A104-B6; A104-B7; A104-B8; A104-B9; A104-B10; A104-B11; A104-B12; A104-B13; A104-B14; A104-B15; A104-B16; A104-B17; A104-B18; A104-B19; A104-B20; A104-B21; A104-B22; A104-B23; A104-B24; A104-B25; A104-B26; A104-B27; A104-B28; A104-B29; A104-B30; A104-B31; A104-B32; A104-B33; A104-B34; A104-B35; A104-B36; A104-B37; A104-B38; A104-B39; A104-B40; A104-B41; A104-B42; A104-B43; A104-B44; A104-B45; A104-B46; A104-B47; A104-B48; A104-B49; A104-B50; A104-B51; A104-B52; A104-B53; A104-B54; A104-B55; A104-B56; A104-B57; A104-B58; A104-B59; A104-B60; A104-B61; A104-B62; A104-B63; A104-B64; A104-B65; A104-B66; A104-B67; A104-B68; A104-B69; A104-B70; A104-B71; A104-B72; A104-B73; A104-B74; A104-B75; A104-B76; A104-B77; A104-B78; A104-B79; A104-B80; A104-B81; A104-B82; A104-B83; A104-B84; A104-B85; A104-B86; A104-B87; A104-B88; A104-B89; A104-B90; A104-B91; A104-B92; A104-B93; A104-B94; A104-B95; A104-B96; A104-B97; A104-B98; A104-B99; A104-B100; A104-B101; A104-B102; A104-B103; A104-B104; A104-B105; A104-B106; A104-B107; A104-B108; A104-B109; A104-B110; A104-B111; A104-B112; A104-B113;

A105-B1; A105-B2; A105-B3; A105-B4; A105-B5; A105-B6; A105-B7; A105-B8; A105-B9; A105-B10; A105-B11; A105-B12; A105-B13; A105-B14; A105-B15; A105-B16; A105-B17; A105-B18; A105-B19; A105-B20; A105-B21; A105-B22; A105-B23; A105-B24; A105-B25; A105-B26; A105-B27; A105-B28; A105-B29; A105-B30; A105-B31; A105-B32; A105-B33; A105-B34; A105-B35; A105-B36; A105-B37; A105-B38; A105-B39; A105-B40; A105-B41; A105-B42; A105-B43; A105-B44; A105-B45; A105-B46; A105-B47; A105-B48; A105-B49; A105-B50; A105-B51; A105-B52; A105-B53; A105-B54; A105-B55; A105-B56; A105-B57; A105-B58; A105-B59; A105-B60; A105-B61; A105-B62; A105-B63; A105-B64; A105-B65; A105-B66; A105-B67; A105-B68; A105-B69; A105-B70; A105-B71; A105-B72; A105-B73; A105-B74; A105-B75; A105-B76; A105-B77; A105-B78; A105-B79; A105-B80; A105-B81; A105-B82; A105-B83; A105-B84; A105-B85; A105-B86; A105-B87; A105-B88; A105-B89; A105-B90; A105-B91; A105-B92; A105-B93; A105-B94; A105-B95; A105-B96; A105-B97; A105-B98; A105-B99; A105-B100; A105-B101; A105-B102; A105-B103; A105-B104; A105-B105; A105-B106; A105-B107; A105-B108; A105-B109; A105-B110; A105-B111; A105-B112; A105-B113;

A106-B1; A106-B2; A106-B3; A106-B4; A106-B5; A106-B6; A106-B7; A106-B8; A106-B9; A106-B10; A106-B11; A106-B12; A106-B13; A106-B14; A106-B15; A106-B16; A106-B17; A106-B18; A106-B19; A106-B20; A106-B21; A106-B22; A106-B23; A106-B24; A106-B25; A106-B26; A106-B27; A106-B28; A106-B29; A106-B30; A106-B31; A106-B32; A106-B33; A106-B34; A106-B35; A106-B36; A106-B37; A106-B38; A106-B39; A106-B40; A106-B41; A106-B42; A106-B43; A106-B44; A106-B45; A106-B46; A106-B47; A106-B48; A106-B49; A106-B50; A106-B51; A106-B52; A106-B53; A106-B54; A106-B55; A106-B56; A106-B57; A106-B58; A106-B59; A106-B60; A106-B61; A106-B62; A106-B63; A106-B64; A106-B65; A106-B66; A106-B67; A106-B68; A106-B69; A106-B70; A106-B71; A106-B72; A106-B73; A106-B74; A106-B75; A106-B76; A106-B77; A106-B78; A106-B79; A106-B80; A106-B81; A106-B82; A106-B83; A106-B84; A106-B85; A106-B86; A106-B87; A106-B88; A106-B89; A106-B90; A106-B91; A106-B92; A106-B93; A106-B94; A106-B95; A106-B96; A106-B97; A106-B98; A106-B99; A106-B100; A106-B101; A106-B102; A106-B103; A106-B104; A106-B105; A106-B106; A106-B107; A106-B108; A106-B109; A106-B110; A106-B111; A106-B112; A106-B113;

A107-B1; A107-B2; A107-B3; A107-B4; A107-B5; A107-B6; A107-B7; A107-B8; A107-B9; A107-B10; A107-B11; A107-B12; A107-B13; A107-B14; A107-B15; A107-B16; A107-B17; A107-B18; A107-B19; A107-B20; A107-B21; A107-B22; A107-B23; A107-B24; A107-B25; A107-B26; A107-B27; A107-B28; A107-B29; A107-B30; A107-B31; A107-B32; A107-B33; A107-B34; A107-B35; A107-B36; A107-B37; A107-B38; A107-B39; A107-B40; A107-B41; A107-B42; A107-B43; A107-B44; A107-B45; A107-B46; A107-B47; A107-B48; A107-B49; A107-B50; A107-B51; A107-B52; A107-B53; A107-B54; A107-B55; A107-B56; A107-B57; A107-B58; A107-B59; A107-B60; A107-B61; A107-B62; A107-B63; A107-B64; A107-B65; A107-B66; A107-B67; A107-B68; A107-B69; A107-B70; A107-B71; A107-B72; A107-B73; A107-B74; A107-B75; A107-B76; A107-B77; A107-B78; A107-B79; A107-B80; A107-B81; A107-B82; A107-B83; A107-B84; A107-B85; A107-B86; A107-B87; A107-B88; A107-B89; A107-B90; A107-B91; A107-B92; A107-B93; A107-B94; A107-B95; A107-B96; A107-B97; A107-B98; A107-B99; A107-B100; A107-B101; A107-B102; A107-B103; A107-B104; A107-B105; A107-B106; A107-B107; A107-B108; A107-B109; A107-B110; A107-B111; A107-B112; A107-B113;

A108-B1; A108-B2; A108-B3; A108-B4; A108-B5; A108-B6; A108-B7; A108-B8; A108-B9; A108-B10; A108-B11; A108-B12; A108-B13; A108-B14; A108-B15; A108-B16; A108-B17; A108-B18; A108-B19; A108-B20; A108-B21; A108-B22; A108-B23; A108-B24; A108-B25; A108-B26; A108-B27; A108-B28; A108-B29; A108-B30; A108-B31; A108-B32; A108-B33; A108-B34; A108-B35; A108-B36; A108-B37; A108-B38; A108-B39; A108-B40; A108-B41; A108-B42; A108-B43; A108-B44; A108-B45; A108-B46; A108-B47; A108-B48; A108-B49; A108-B50; A108-B51; A108-B52; A108-B53; A108-B54; A108-B55; A108-B56; A108-B57; A108-B58; A108-B59; A108-B60; A108-B61; A108-B62; A108-B63; A108-B64; A108-B65; A108-B66; A108-B67; A108-B68; A108-B69; A108-B70; A108-B71; A108-B72; A108-B73; A108-B74; A108-B75; A108-B76; A108-B77; A108-B78; A108-B79; A108-B80; A108-B81; A108-B82; A108-B83; A108-B84; A108-B85; A108-B86; A108-B87; A108-B88; A108-B89; A108-B90; A108-B91; A108-B92; A108-B93; A108-B94; A108-B95; A108-B96; A108-B97; A108-B98; A108-B99; A108-B100; A108-B101; A108-B102; A108-B103; A108-B104; A108-B105; A108-B106; A108-B107; A108-B108; A108-B109; A108-B110; A108-B111; A108-B112; A108-B113;

A109-B1; A109-B2; A109-B3; A109-B4; A109-B5; A109-B6; A109-B7; A109-B8; A109-B9; A109-B10; A109-B11; A109-B12; A109-B13; A109-B14; A109-B15; A109-B16; A109-B17; A109-B18; A109-B19; A109-B20; A109-B21; A109-B22; A109-B23; A109-B24; A109-B25; A109-B26; A109-B27; A109-B28; A109-B29; A109-B30; A109-B31; A109-B32; A109-B33; A109-B34; A109-B35; A109-B36; A109-B37; A109-B38; A109-B39; A109-B40; A109-B41; A109-B42; A109-B43; A109-B44; A109-B45; A109-B46; A109-B47; A109-B48; A109-B49; A109-B50; A109-B51; A109-B52; A109-B53; A109-B54; A109-B55; A109-B56; A109-B57; A109-B58; A109-B59; A109-B60; A109-B61; A109-B62; A109-B63; A109-B64; A109-B65; A109-B66; A109-B67; A109-B68; A109-B69; A109-B70; A109-B71; A109-B72; A109-B73; A109-B74; A109-B75; A109-B76; A109-B77; A109-B78; A109-B79; A109-B80; A109-B81; A109-B82; A109-B83; A109-B84; A109-B85; A109-B86; A109-B87; A109-B88; A109-B89; A109-B90; A109-B91; A109-B92; A109-B93; A109-B94; A109-B95; A109-B96; A109-B97; A109-B98; A109-B99; A109-B100; A109-B101; A109-B102; A109-B103; A109-B104; A109-B105; A109-B106; A109-B107; A109-B108; A109-B109; A109-B110; A109-B111; A109-B112; A109-B113;

A110-B1; A110-B2; A110-B3; A110-B4; A110-B5; A110-B6; A110-B7; A110-B8; A110-B9; A110-B10; A110-B11; A110-B12; A110-B13; A110-B14; A110-B15; A110-B16; A110-B17; A110-B18; A110-B19; A110-B20; A110-B21; A110-B22; A110-B23; A110-B24; A110-B25; A110-B26; A110-B27; A110-B28; A110-B29; A110-B30; A110-B31; A110-B32; A110-B33; A110-B34; A110-B35; A110-B36; A110-B37; A110-B38; A110-B39; A110-B40; A110-B41; A110-B42; A110-B43; A110-B44; A110-B45; A110-B46; A110-B47; A110-B48; A110-B49; A110-B50; A110-B51; A110-B52; A110-B53; A110-B54; A110-B55; A110-B56; A110-B57; A110-B58; A110-B59; A110-B60; A110-B61; A110-B62; A110-B63; A110-B64; A110-B65; A110-B66; A110-B67; A110-B68; A110-B69; A110-B70; A110-B71; A110-B72; A110-B73; A110-B74; A110-B75; A110-B76; A110-B77; A110-B78; A110-B79; A110-B80; A110-B81; A110-B82; A110-B83; A110-B84; A110-B85; A110-B86; A110-B87; A110-B88; A110-B89; A110-B90; A110-B91; A110-B92; A110-B93; A110-B94; A110-B95; A110-B96; A110-B97; A110-B98; A110-B99; A110-B100; A110-B101; A110-B102; A110-B103; A110-B104; A110-B105; A110-B106; A110-B107; A110-B108; A110-B109; A110-B110; A110-B111; A110-B112; A110-B113;

A111-B1; A111-B2; A111-B3; A111-B4; A111-B5; A111-B6; A111-B7; A111-B8; A111-B9; A111-B10; A111-B11; A111-B12; A111-B13; A111-B14; A111-B15; A111-B16; A111-B17; A111-B18; A111-B19; A111-B20; A111-B21; A111-B22; A111-B23; A111-B24; A111-B25; A111-B26; A111-B27; A111-B28; A111-B29; A111-B30; A111-B31; A111-B32; A111-B33; A111-B34; A111-B35; A111-B36; A111-B37; A111-B38; A111-B39; A111-B40; A111-B41; A111-B42; A111-B43; A111-B44; A111-B45; A111-B46; A111-B47; A111-B48; A111-B49; A111-B50; A111-B51; A111-B52; A111-B53; A111-B54; A111-B55; A111-B56; A111-B57; A111-B58; A111-B59; A111-B60; A111-B61; A111-B62; A111-B63; A111-B64; A111-B65; A111-B66; A111-B67; A111-B68; A111-B69; A111-B70; A111-B71; A111-B72; A111-B73; A111-B74; A111-B75; A111-B76; A111-B77; A111-B78; A111-B79; A111-B80; A111-B81; A111-B82; A111-B83; A111-B84; A111-B85; A111-B86; A111-B87; A111-B88; A111-B89; A111-B90; A111-B91; A111-B92; A111-B93; A111-B94; A111-B95; A111-B96; A111-B97; A111-B98; A111-B99; A111-B100; A111-B101; A111-B102; A111-B103; A111-B104; A111-B105; A111-B106; A111-B107; A111-B108; A111-B109; A111-B110; A111-B111; A111-B112; A111-B113;

A112-B1; A112-B2; A112-B3; A112-B4; A112-B5; A112-B6; A112-B7; A112-B8; A112-B9; A112-B10; A112-B11; A112-B12; A112-B13; A112-B14; A112-B15; A112-B16; A112-B17; A112-B18; A112-B19; A112-B20; A112-B21; A112-B22; A112-B23; A112-B24; A112-B25; A112-B26; A112-B27; A112-B28; A112-B29; A112-B30; A112-B31; A112-B32; A112-B33; A112-B34; A112-B35; A112-B36; A112-B37; A112-B38; A112-B39; A112-B40; A112-B41; A112-B42; A112-B43; A112-B44; A112-B45; A112-B46; A112-B47; A112-B48; A112-B49; A112-B50; A112-B51; A112-B52; A112-B53; A112-B54; A112-B55; A112-B56; A112-B57; A112-B58; A112-B59; A112-B60; A112-B61; A112-B62; A112-B63; A112-B64; A112-B65; A112-B66; A112-B67; A112-B68; A112-B69; A112-B70; A112-B71; A112-B72; A112-B73; A112-B74; A112-B75; A112-B76; A112-B77; A112-B78; A112-B79; A112-B80; A112-B81; A112-B82; A112-B83; A112-B84; A112-B85; A112-B86; A112-B87; A112-B88; A112-B89; A112-B90; A112-B91; A112-B92; A112-B93; A112-B94; A112-B95; A112-B96; A112-B97; A112-B98; A112-B99; A112-B100; A112-B101; A112-B102; A112-B103; A112-B104; A112-B105; A112-B106; A112-B107; A112-B108; A112-B109; A112-B110; A112-B111; A112-B112; A112-B113;

A113-B1; A113-B2; A113-B3; A113-B4; A113-B5; A113-B6; A113-B7; A113-B8; A113-B9; A113-B10; A113-B11; A113-B12; A113-B13; A113-B14; A113-B15; A113-B16; A113-B17; A113-B18; A113-B19; A113-B20; A113-B21; A113-B22; A113-B23; A113-B24; A113-B25; A113-B26; A113-B27; A113-B28; A113-B29; A113-B30; A113-B31; A113-B32; A113-B33; A113-B34; A113-B35; A113-B36; A113-B37; A113-B38; A113-B39; A113-B40; A113-B41; A113-B42; A113-B43; A113-B44; A113-B45; A113-B46; A113-B47; A113-B48; A113-B49; A113-B50; A113-B51; A113-B52; A113-B53; A113-B54; A113-B55; A113-B56; A113-B57; A113-B58; A113-B59; A113-B60; A113-B61; A113-B62; A113-B63; A113-B64; A113-B65; A113-B66; A113-B67; A113-B68; A113-B69; A113-B70; A113-B71; A113-B72; A113-B73; A113-B74; A113-B75; A113-B76; A113-B77; A113-B78; A113-B79; A113-B80; A113-B81; A113-B82; A113-B83; A113-B84; A113-B85; A113-B86; A113-B87; A113-B88; A113-B89; A113-B90; A113-B91; A113-B92; A113-B93; A113-B94; A113-B95; A113-B96; A113-B97; A113-B98; A113-B99; A113-B100; A113-B101; A113-B102; A113-B103; A113-B104; A113-B105; A113-B106; A113-B107; A113-B108; A113-B109; A113-B110; A113-B111; A113-B112; A113-B113;

A114-B1; A114-B2; A114-B3; A114-B4; A114-B5; A114-B6; A114-B7; A114-B8; A114-B9; A114-B10; A114-B11; A114-B12; A114-B13; A114-B14; A114-B15; A114-B16; A114-B17; A114-B18; A114-B19; A114-B20; A114-B21; A114-B22; A114-B23; A114-B24; A114-B25; A114-B26; A114-B27; A114-B28; A114-B29; A114-B30; A114-B31; A114-B32; A114-B33; A114-B34; A114-B35; A114-B36; A114-B37; A114-B38; A114-B39; A114-B40; A114-B41; A114-B42; A114-B43; A114-B44; A114-B45; A114-B46; A114-B47; A114-B48; A114-B49; A114-B50; A114-B51; A114-B52; A114-B53; A114-B54; A114-B55; A114-B56; A114-B57; A114-B58; A114-B59; A114-B60; A114-B61; A114-B62; A114-B63; A114-B64; A114-B65; A114-B66; A114-B67; A114-B68; A114-B69; A114-B70; A114-B71; A114-B72; A114-B73; A114-B74; A114-B75; A114-B76; A114-B77; A114-B78; A114-B79; A114-B80; A114-B81; A114-B82; A114-B83; A114-B84; A114-B85; A114-B86; A114-B87; A114-B88; A114-B89; A114-B90; A114-B91; A114-B92; A114-B93; A114-B94; A114-B95; A114-B96; A114-B97; A114-B98; A114-B99; A114-B100; A114-B101; A114-B102; A114-B103; A114-B104; A114-B105; A114-B106; A114-B107; A114-B108; A114-B109; A114-B110; A114-B111; A114-B112; A114-B113;

A115-B1; A115-B2; A115-B3; A115-B4; A115-B5; A115-B6; A115-B7; A115-B8; A115-B9; A115-B10; A115-B11; A115-B12; A115-B13; A115-B14; A115-B15; A115-B16; A115-B17; A115-B18; A115-B19; A115-B20; A115-B21; A115-B22; A115-B23; A115-B24; A115-B25; A115-B26; A115-B27; A115-B28; A115-B29; A115-B30; A115-B31; A115-B32; A115-B33; A115-B34; A115-B35; A115-B36; A115-B37; A115-B38; A115-B39; A115-B40; A115-B41; A115-B42; A115-B43; A115-B44; A115-B45; A115-B46; A115-B47; A115-B48; A115-B49; A115-B50; A115-B51; A115-B52; A115-B53; A115-B54; A115-B55; A115-B56; A115-B57; A115-B58; A115-B59; A115-B60; A115-B61; A115-B62; A115-B63; A115-B64; A115-B65; A115-B66; A115-B67; A115-B68; A115-B69; A115-B70; A115-B71; A115-B72; A115-B73; A115-B74; A115-B75; A115-B76; A115-B77; A115-B78; A115-B79; A115-B80; A115-B81; A115-B82; A115-B83; A115-B84; A115-B85; A115-B86; A115-B87; A115-B88; A115-B89; A115-B90; A115-B91; A115-B92; A115-B93; A115-B94; A115-B95; A115-B96; A115-B97; A115-B98; A115-B99; A115-B100; A115-B101; A115-B102; A115-B103; A115-B104; A115-B105; A115-B106; A115-B107; A115-B108; A115-B109; A115-B110; A115-B111; A115-B112; A115-B113;

A116-B1; A116-B2; A116-B3; A116-B4; A116-B5; A116-B6; A116-B7; A116-B8; A116-B9; A116-B10; A116-B11; A116-B12; A116-B13; A116-B14; A116-B15; A116-B16; A116-B17; A116-B18; A116-B19; A116-B20; A116-B21; A116-B22; A116-B23; A116-B24; A116-B25; A116-B26; A116-B27; A116-B28; A116-B29; A116-B30; A116-B31; A116-B32; A116-B33; A116-B34; A116-B35; A116-B36; A116-B37; A116-B38; A116-B39; A116-B40; A116-B41; A116-B42; A116-B43; A116-B44; A116-B45; A116-B46; A116-B47; A116-B48; A116-B49; A116-B50; A116-B51; A116-B52; A116-B53; A116-B54; A116-B55; A116-B56; A116-B57; A116-B58; A116-B59; A116-B60; A116-B61; A116-B62; A116-B63; A116-B64; A116-B65; A116-B66; A116-B67; A116-B68; A116-B69; A116-B70; A116-B71; A116-B72; A116-B73; A116-B74; A116-B75; A116-B76; A116-B77; A116-B78; A116-B79; A116-B80; A116-B81; A116-B82; A116-B83; A116-B84; A116-B85; A116-B86; A116-B87; A116-B88; A116-B89; A116-B90; A116-B91; A116-B92; A116-B93; A116-B94; A116-B95; A116-B96; A116-B97; A116-B98; A116-B99; A116-B100; A116-B101; A116-B102; A116-B103; A116-B104; A116-B105; A116-B106; A116-B107; A116-B108; A116-B109; A116-B110; A116-B111; A116-B112; A116-B113;

A117-B1; A117-B2; A117-B3; A117-B4; A117-B5; A117-B6; A117-B7; A117-B8; A117-B9; A117-B10; A117-B11; A117-B12; A117-B13; A117-B14; A117-B15; A117-B16; A117-B17; A117-B18; A117-B19; A117-B20; A117-B21; A117-B22; A117-B23; A117-B24; A117-B25; A117-B26; A117-B27; A117-B28; A117-B29; A117-B30; A117-B31; A117-B32; A117-B33; A117-B34; A117-B35; A117-B36; A117-B37; A117-B38; A117-B39; A117-B40; A117-B41; A117-B42; A117-B43; A117-B44; A117-B45; A117-B46; A117-B47; A117-B48; A117-B49; A117-B50; A117-B51; A117-B52; A117-B53; A117-B54; A117-B55; A117-B56; A117-B57; A117-B58; A117-B59; A117-B60; A117-B61; A117-B62; A117-B63; A117-B64; A117-B65; A117-B66; A117-B67; A117-B68; A117-B69; A117-B70; A117-B71; A117-B72; A117-B73; A117-B74; A117-B75; A117-B76; A117-B77; A117-B78; A117-B79; A117-B80; A117-B81; A117-B82; A117-B83; A117-B84; A117-B85; A117-B86; A117-B87; A117-B88; A117-B89; A117-B90; A117-B91; A117-B92; A117-B93; A117-B94; A117-B95; A117-B96; A117-B97; A117-B98; A117-B99; A117-B100; A117-B101; A117-B102; A117-B103; A117-B104; A117-B105; A117-B106; A117-B107; A117-B108; A117-B109; A117-B110; A117-B111; A117-B112; A117-B113;

A118-B1; A118-B2; A118-B3; A118-B4; A118-B5; A118-B6; A118-B7; A118-B8; A118-B9; A118-B10; A118-B11; A118-B12; A118-B13; A118-B14; A118-B15; A118-B16; A118-B17; A118-B18; A118-B19; A118-B20; A118-B21; A118-B22; A118-B23; A118-B24; A118-B25; A118-B26; A118-B27; A118-B28; A118-B29; A118-B30; A118-B31; A118-B32; A118-B33; A118-B34; A118-B35; A118-B36; A118-B37; A118-B38; A118-B39; A118-B40; A118-B41; A118-B42; A118-B43; A118-B44; A118-B45; A118-B46; A118-B47; A118-B48; A118-B49; A118-B50; A118-B51; A118-B52; A118-B53; A118-B54; A118-B55; A118-B56; A118-B57; A118-B58; A118-B59; A118-B60; A118-B61; A118-B62; A118-B63; A118-B64; A118-B65; A118-B66; A118-B67; A118-B68; A118-B69; A118-B70; A118-B71; A118-B72; A118-B73; A118-B74; A118-B75; A118-B76; A118-B77; A118-B78; A118-B79; A118-B80; A118-B81; A118-B82; A118-B83; A118-B84; A118-B85; A118-B86; A118-B87; A118-B88; A118-B89; A118-B90; A118-B91; A118-B92; A118-B93; A118-B94; A118-B95; A118-B96; A118-B97; A118-B98; A118-B99; A118-B100; A118-B101; A118-B102; A118-B103; A118-B104; A118-B105; A118-B106; A118-B107; A118-B108; A118-B109; A118-B110; A118-B111; A118-B112; A118-B113;

A119-B1; A119-B2; A119-B3; A119-B4; A119-B5; A119-B6; A119-B7; A119-B8; A119-B9; A119-B10; A119-B11; A119-B12; A119-B13; A119-B14; A119-B15; A119-B16; A119-B17; A119-B18; A119-B19; A119-B20; A119-B21; A119-B22; A119-B23; A119-B24; A119-B25; A119-B26; A119-B27; A119-B28; A119-B29; A119-B30; A119-B31; A119-B32; A119-B33; A119-B34; A119-B35; A119-B36; A119-B37; A119-B38; A119-B39; A119-B40; A119-B41; A119-B42; A119-B43; A119-B44; A119-B45; A119-B46; A119-B47; A119-B48; A119-B49; A119-B50; A119-B51; A119-B52; A119-B53; A119-B54; A119-B55; A119-B56; A119-B57; A119-B58; A119-B59; A119-B60; A119-B61; A119-B62; A119-B63; A119-B64; A119-B65; A119-B66; A119-B67; A119-B68; A119-B69; A119-B70; A119-B71; A119-B72; A119-B73; A119-B74; A119-B75; A119-B76; A119-B77; A119-B78; A119-B79; A119-B80; A119-B81; A119-B82; A119-B83; A119-B84; A119-B85; A119-B86; A119-B87; A119-B88; A119-B89; A119-B90; A119-B91; A119-B92; A119-B93; A119-B94; A119-B95; A119-B96; A119-B97; A119-B98; A119-B99; A119-B100; A119-B101; A119-B102; A119-B103; A119-B104; A119-B105; A119-B106; A119-B107; A119-B108; A119-B109; A119-B110; A119-B111; A119-B112; A119-B113;

A120-B1; A120-B2; A120-B3; A120-B4; A120-B5; A120-B6; A120-B7; A120-B8; A120-B9; A120-B10; A120-B11; A120-B12; A120-B13; A120-B14; A120-B15; A120-B16; A120-B17; A120-B18; A120-B19; A120-B20; A120-B21; A120-B22; A120-B23; A120-B24; A120-B25; A120-B26; A120-B27; A120-B28; A120-B29; A120-B30; A120-B31; A120-B32; A120-B33; A120-B34; A120-B35; A120-B36; A120-B37; A120-B38; A120-B39; A120-B40; A120-B41; A120-B42; A120-B43; A120-B44; A120-B45; A120-B46; A120-B47; A120-B48; A120-B49; A120-B50; A120-B51; A120-B52; A120-B53; A120-B54; A120-B55; A120-B56; A120-B57; A120-B58; A120-B59; A120-B60; A120-B61; A120-B62; A120-B63; A120-B64; A120-B65; A120-B66; A120-B67; A120-B68; A120-B69; A120-B70; A120-B71; A120-B72; A120-B73; A120-B74; A120-B75; A120-B76; A120-B77; A120-B78; A120-B79; A120-B80; A120-B81; A120-B82; A120-B83; A120-B84; A120-B85; A120-B86; A120-B87; A120-B88; A120-B89; A120-B90; A120-B91; A120-B92; A120-B93; A120-B94; A120-B95; A120-B96; A120-B97; A120-B98; A120-B99; A120-B100; A120-B101; A120-B102; A120-B103; A120-B104; A120-B105; A120-B106; A120-B107; A120-B108; A120-B109; A120-B110; A120-B111; A120-B112; A120-B113;

A121-B1; A121-B2; A121-B3; A121-B4; A121-B5; A121-B6; A121-B7; A121-B8; A121-B9; A121-B10; A121-B11; A121-B12; A121-B13; A121-B14; A121-B15; A121-B16; A121-B17; A121-B18; A121-B19; A121-B20; A121-B21; A121-B22; A121-B23; A121-B24; A121-B25; A121-B26; A121-B27; A121-B28; A121-B29; A121-B30; A121-B31; A121-B32; A121-B33; A121-B34; A121-B35; A121-B36; A121-B37; A121-B38; A121-B39; A121-B40; A121-B41; A121-B42; A121-B43; A121-B44; A121-B45; A121-B46; A121-B47; A121-B48; A121-B49; A121-B50; A121-B51; A121-B52; A121-B53; A121-B54; A121-B55; A121-B56; A121-B57; A121-B58; A121-B59; A121-B60; A121-B61; A121-B62; A121-B63; A121-B64; A121-B65; A121-B66; A121-B67; A121-B68; A121-B69; A121-B70; A121-B71; A121-B72; A121-B73; A121-B74; A121-B75; A121-B76; A121-B77; A121-B78; A121-B79; A121-B80; A121-B81; A121-B82; A121-B83; A121-B84; A121-B85; A121-B86; A121-B87; A121-B88; A121-B89; A121-B90; A121-B91; A121-B92; A121-B93; A121-B94; A121-B95; A121-B96; A121-B97; A121-B98; A121-B99; A121-B100; A121-B101; A121-B102; A121-B103; A121-B104; A121-B105; A121-B106; A121-B107; A121-B108; A121-B109; A121-B110; A121-B111; A121-B112; A121-B113;

A122-B1; A122-B2; A122-B3; A122-B4; A122-B5; A122-B6; A122-B7; A122-B8; A122-B9; A122-B10; A122-B11; A122-B12; A122-B13; A122-B14; A122-B15; A122-B16; A122-B17; A122-B18; A122-B19; A122-B20; A122-B21; A122-B22; A122-B23; A122-B24; A122-B25; A122-B26; A122-B27; A122-B28; A122-B29; A122-B30; A122-B31; A122-B32; A122-B33; A122-B34; A122-B35; A122-B36; A122-B37; A122-B38; A122-B39; A122-B40; A122-B41; A122-B42; A122-B43; A122-B44; A122-B45; A122-B46; A122-B47; A122-B48; A122-B49; A122-B50; A122-B51; A122-B52; A122-B53; A122-B54; A122-B55; A122-B56; A122-B57; A122-B58; A122-B59; A122-B60; A122-B61; A122-B62; A122-B63; A122-B64; A122-B65; A122-B66; A122-B67; A122-B68; A122-B69; A122-B70; A122-B71; A122-B72; A122-B73; A122-B74; A122-B75; A122-B76; A122-B77; A122-B78; A122-B79; A122-B80; A122-B81; A122-B82; A122-B83; A122-B84; A122-B85; A122-B86; A122-B87; A122-B88; A122-B89; A122-B90; A122-B91; A122-B92; A122-B93; A122-B94; A122-B95; A122-B96; A122-B97; A122-B98; A122-B99; A122-B100; A122-B101; A122-B102; A122-B103; A122-B104; A122-B105; A122-B106; A122-B107; A122-B108; A122-B109; A122-B110; A122-B111; A122-B112; A122-B113;

A123-B1; A123-B2; A123-B3; A123-B4; A123-B5; A123-B6; A123-B7; A123-B8; A123-B9; A123-B10; A123-B11; A123-B12; A123-B13; A123-B14; A123-B15; A123-B16; A123-B17; A123-B18; A123-B19; A123-B20; A123-B21; A123-B22; A123-B23; A123-B24; A123-B25; A123-B26; A123-B27; A123-B28; A123-B29; A123-B30; A123-B31; A123-B32; A123-B33; A123-B34; A123-B35; A123-B36; A123-B37; A123-B38; A123-B39; A123-B40; A123-B41; A123-B42; A123-B43; A123-B44; A123-B45; A123-B46; A123-B47; A123-B48; A123-B49; A123-B50; A123-B51; A123-B52; A123-B53; A123-B54; A123-B55; A123-B56; A123-B57; A123-B58; A123-B59; A123-B60; A123-B61; A123-B62; A123-B63; A123-B64; A123-B65; A123-B66; A123-B67; A123-B68; A123-B69; A123-B70; A123-B71; A123-B72; A123-B73; A123-B74; A123-B75; A123-B76; A123-B77; A123-B78; A123-B79; A123-B80; A123-B81; A123-B82; A123-B83; A123-B84; A123-B85; A123-B86; A123-B87; A123-B88; A123-B89; A123-B90; A123-B91; A123-B92; A123-B93; A123-B94; A123-B95; A123-B96; A123-B97; A123-B98; A123-B99; A123-B100; A123-B101; A123-B102; A123-B103; A123-B104; A123-B105; A123-B106; A123-B107; A123-B108; A123-B109; A123-B110; A123-B111; A123-B112; A123-B113;

A124-B1; A124-B2; A124-B3; A124-B4; A124-B5; A124-B6; A124-B7; A124-B8; A124-B9; A124-B10; A124-B11; A124-B12; A124-B13; A124-B14; A124-B15; A124-B16; A124-B17; A124-B18; A124-B19; A124-B20; A124-B21; A124-B22; A124-B23; A124-B24; A124-B25; A124-B26; A124-B27; A124-B28; A124-B29; A124-B30; A124-B31; A124-B32; A124-B33; A124-B34; A124-B35; A124-B36; A124-B37; A124-B38; A124-B39; A124-B40; A124-B41; A124-B42; A124-B43; A124-B44; A124-B45; A124-B46; A124-B47; A124-B48; A124-B49; A124-B50; A124-B51; A124-B52; A124-B53; A124-B54; A124-B55; A124-B56; A124-B57; A124-B58; A124-B59; A124-B60; A124-B61; A124-B62; A124-B63; A124-B64; A124-B65; A124-B66; A124-B67; A124-B68; A124-B69; A124-B70; A124-B71; A124-B72; A124-B73; A124-B74; A124-B75; A124-B76; A124-B77; A124-B78; A124-B79; A124-B80; A124-B81; A124-B82; A124-B83; A124-B84; A124-B85; A124-B86; A124-B87; A124-B88; A124-B89; A124-B90; A124-B91; A124-B92; A124-B93; A124-B94; A124-B95; A124-B96; A124-B97; A124-B98; A124-B99; A124-B100; A124-B101; A124-B102; A124-B103; A124-B104; A124-B105; A124-B106; A124-B107; A124-B108; A124-B109; A124-B110; A124-B111; A124-B112; A124-B113;

A125-B1; A125-B2; A125-B3; A125-B4; A125-B5; A125-B6; A125-B7; A125-B8; A125-B9; A125-B10; A125-B11; A125-B12; A125-B13; A125-B14; A125-B15; A125-B16; A125-B17; A125-B18; A125-B19; A125-B20; A125-B21; A125-B22; A125-B23; A125-B24; A125-B25; A125-B26; A125-B27; A125-B28; A125-B29; A125-B30; A125-B31; A125-B32; A125-B33; A125-B34; A125-B35; A125-B36; A125-B37; A125-B38; A125-B39; A125-B40; A125-B41; A125-B42; A125-B43; A125-B44; A125-B45; A125-B46; A125-B47; A125-B48; A125-B49; A125-B50; A125-B51; A125-B52; A125-B53; A125-B54; A125-B55; A125-B56; A125-B57; A125-B58; A125-B59; A125-B60; A125-B61; A125-B62; A125-B63; A125-B64; A125-B65; A125-B66; A125-B67; A125-B68; A125-B69; A125-B70; A125-B71; A125-B72; A125-B73; A125-B74; A125-B75; A125-B76; A125-B77; A125-B78; A125-B79; A125-B80; A125-B81; A125-B82; A125-B83; A125-B84; A125-B85; A125-B86; A125-B87; A125-B88; A125-B89; A125-B90; A125-B91; A125-B92; A125-B93; A125-B94; A125-B95; A125-B96; A125-B97; A125-B98; A125-B99; A125-B100; A125-B101; A125-B102; A125-B103; A125-B104; A125-B105; A125-B106; A125-B107; A125-B108; A125-B109; A125-B110; A125-B111; A125-B112; A125-B113;

A126-B1; A126-B2; A126-B3; A126-B4; A126-B5; A126-B6; A126-B7; A126-B8; A126-B9; A126-B10; A126-B11; A126-B12; A126-B13; A126-B14; A126-B15; A126-B16; A126-B17; A126-B18; A126-B19; A126-B20; A126-B21; A126-B22; A126-B23; A126-B24; A126-B25; A126-B26; A126-B27; A126-B28; A126-B29; A126-B30; A126-B31; A126-B32; A126-B33; A126-B34; A126-B35; A126-B36; A126-B37; A126-B38; A126-B39; A126-B40; A126-B41; A126-B42; A126-B43; A126-B44; A126-B45; A126-B46; A126-B47; A126-B48; A126-B49; A126-B50; A126-B51; A126-B52; A126-B53; A126-B54; A126-B55; A126-B56; A126-B57; A126-B58; A126-B59; A126-B60; A126-B61; A126-B62; A126-B63; A126-B64; A126-B65; A126-B66; A126-B67; A126-B68; A126-B69; A126-B70; A126-B71; A126-B72; A126-B73; A126-B74; A126-B75; A126-B76; A126-B77; A126-B78; A126-B79; A126-B80; A126-B81; A126-B82; A126-B83; A126-B84; A126-B85; A126-B86; A126-B87; A126-B88; A126-B89; A126-B90; A126-B91; A126-B92; A126-B93; A126-B94; A126-B95; A126-B96; A126-B97; A126-B98; A126-B99; A126-B100; A126-B101; A126-B102; A126-B103; A126-B104; A126-B105; A126-B106; A126-B107; A126-B108; A126-B109; A126-B110; A126-B111; A126-B112; A126-B113;

A127-B1; A127-B2; A127-B3; A127-B4; A127-B5; A127-B6; A127-B7; A127-B8; A127-B9; A127-B10; A127-B11; A127-B12; A127-B13; A127-B14; A127-B15; A127-B16; A127-B17; A127-B18; A127-B19; A127-B20; A127-B21; A127-B22; A127-B23; A127-B24; A127-B25; A127-B26; A127-B27; A127-B28; A127-B29; A127-B30; A127-B31; A127-B32; A127-B33; A127-B34; A127-B35; A127-B36; A127-B37; A127-B38; A127-B39; A127-B40; A127-B41; A127-B42; A127-B43; A127-B44; A127-B45; A127-B46; A127-B47; A127-B48; A127-B49; A127-B50; A127-B51; A127-B52; A127-B53; A127-B54; A127-B55; A127-B56; A127-B57; A127-B58; A127-B59; A127-B60; A127-B61; A127-B62; A127-B63; A127-B64; A127-B65; A127-B66; A127-B67; A127-B68; A127-B69; A127-B70; A127-B71; A127-B72; A127-B73; A127-B74; A127-B75; A127-B76; A127-B77; A127-B78; A127-B79; A127-B80; A127-B81; A127-B82; A127-B83; A127-B84; A127-B85; A127-B86; A127-B87; A127-B88; A127-B89; A127-B90; A127-B91; A127-B92; A127-B93; A127-B94; A127-B95; A127-B96; A127-B97; A127-B98; A127-B99; A127-B100; A127-B101; A127-B102; A127-B103; A127-B104; A127-B105; A127-B106; A127-B107; A127-B108; A127-B109; A127-B110; A127-B111; A127-B112; A127-B113;

A128-B1; A128-B2; A128-B3; A128-B4; A128-B5; A128-B6; A128-B7; A128-B8; A128-B9; A128-B10; A128-B11; A128-B12; A128-B13; A128-B14; A128-B15; A128-B16; A128-B17; A128-B18; A128-B19; A128-B20; A128-B21; A128-B22; A128-B23; A128-B24; A128-B25; A128-B26; A128-B27; A128-B28; A128-B29; A128-B30; A128-B31; A128-B32; A128-B33; A128-B34; A128-B35; A128-B36; A128-B37; A128-B38; A128-B39; A128-B40; A128-B41; A128-B42; A128-B43; A128-B44; A128-B45; A128-B46; A128-B47; A128-B48; A128-B49; A128-B50; A128-B51; A128-B52; A128-B53; A128-B54; A128-B55; A128-B56; A128-B57; A128-B58; A128-B59; A128-B60; A128-B61; A128-B62; A128-B63; A128-B64; A128-B65; A128-B66; A128-B67; A128-B68; A128-B69; A128-B70; A128-B71; A128-B72; A128-B73; A128-B74; A128-B75; A128-B76; A128-B77; A128-B78; A128-B79; A128-B80; A128-B81; A128-B82; A128-B83; A128-B84; A128-B85; A128-B86; A128-B87; A128-B88; A128-B89; A128-B90; A128-B91; A128-B92; A128-B93; A128-B94; A128-B95; A128-B96; A128-B97; A128-B98; A128-B99; A128-B100; A128-B101; A128-B102; A128-B103; A128-B104; A128-B105; A128-B106; A128-B107; A128-B108; A128-B109; A128-B110; A128-B111; A128-B112; A128-B113;

A129-B1; A129-B2; A129-B3; A129-B4; A129-B5; A129-B6; A129-B7; A129-B8; A129-B9; A129-B10; A129-B11; A129-B12; A129-B13; A129-B14; A129-B15; A129-B16; A129-B17; A129-B18; A129-B19; A129-B20; A129-B21; A129-B22; A129-B23; A129-B24; A129-B25; A129-B26; A129-B27; A129-B28; A129-B29; A129-B30; A129-B31; A129-B32; A129-B33; A129-B34; A129-B35; A129-B36; A129-B37; A129-B38; A129-B39; A129-B40; A129-B41; A129-B42; A129-B43; A129-B44; A129-B45; A129-B46; A129-B47; A129-B48; A129-B49; A129-B50; A129-B51; A129-B52; A129-B53; A129-B54; A129-B55; A129-B56; A129-B57; A129-B58; A129-B59; A129-B60; A129-B61; A129-B62; A129-B63; A129-B64; A129-B65; A129-B66; A129-B67; A129-B68; A129-B69; A129-B70; A129-B71; A129-B72; A129-B73; A129-B74; A129-B75; A129-B76; A129-B77; A129-B78; A129-B79; A129-B80; A129-B81; A129-B82; A129-B83; A129-B84; A129-B85; A129-B86; A129-B87; A129-B88; A129-B89; A129-B90; A129-B91; A129-B92; A129-B93; A129-B94; A129-B95; A129-B96; A129-B97; A129-B98; A129-B99; A129-B100; A129-B101; A129-B102; A129-B103; A129-B104; A129-B105; A129-B106; A129-B107; A129-B108; A129-B109; A129-B110; A129-B111; A129-B112; A129-B113;

A130-B1; A130-B2; A130-B3; A130-B4; A130-B5; A130-B6; A130-B7; A130-B8; A130-B9; A130-B10; A130-B11; A130-B12; A130-B13; A130-B14; A130-B15; A130-B16; A130-B17; A130-B18; A130-B19; A130-B20; A130-B21; A130-B22; A130-B23; A130-B24; A130-B25; A130-B26; A130-B27; A130-B28; A130-B29; A130-B30; A130-B31; A130-B32; A130-B33; A130-B34; A130-B35; A130-B36; A130-B37; A130-B38; A130-B39; A130-B40; A130-B41; A130-B42; A130-B43; A130-B44; A130-B45; A130-B46; A130-B47; A130-B48; A130-B49; A130-B50; A130-B51; A130-B52; A130-B53; A130-B54; A130-B55; A130-B56; A130-B57; A130-B58; A130-B59; A130-B60; A130-B61; A130-B62; A130-B63; A130-B64; A130-B65; A130-B66; A130-B67; A130-B68; A130-B69; A130-B70; A130-B71; A130-B72; A130-B73; A130-B74; A130-B75; A130-B76; A130-B77; A130-B78; A130-B79; A130-B80; A130-B81; A130-B82; A130-B83; A130-B84; A130-B85; A130-B86; A130-B87; A130-B88; A130-B89; A130-B90; A130-B91; A130-B92; A130-B93; A130-B94; A130-B95; A130-B96; A130-B97; A130-B98; A130-B99; A130-B100; A130-B101; A130-B102; A130-B103; A130-B104; A130-B105; A130-B106; A130-B107; A130-B108; A130-B109; A130-B110; A130-B111; A130-B112; A130-B113;

A131-B1; A131-B2; A131-B3; A131-B4; A131-B5; A131-B6; A131-B7; A131-B8; A131-B9; A131-B10; A131-B11; A131-B12; A131-B13; A131-B14; A131-B15; A131-B16; A131-B17; A131-B18; A131-B19; A131-B20; A131-B21; A131-B22; A131-B23; A131-B24; A131-B25; A131-B26; A131-B27; A131-B28; A131-B29; A131-B30; A131-B31; A131-B32; A131-B33; A131-B34; A131-B35; A131-B36; A131-B37; A131-B38; A131-B39; A131-B40; A131-B41; A131-B42; A131-B43; A131-B44; A131-B45; A131-B46; A131-B47; A131-B48; A131-B49; A131-B50; A131-B51; A131-B52; A131-B53; A131-B54; A131-B55; A131-B56; A131-B57; A131-B58; A131-B59; A131-B60; A131-B61; A131-B62; A131-B63; A131-B64; A131-B65; A131-B66; A131-B67; A131-B68; A131-B69; A131-B70; A131-B71; A131-B72; A131-B73; A131-B74; A131-B75; A131-B76; A131-B77; A131-B78; A131-B79; A131-B80; A131-B81; A131-B82; A131-B83; A131-B84; A131-B85; A131-B86; A131-B87; A131-B88; A131-B89; A131-B90; A131-B91; A131-B92; A131-B93; A131-B94; A131-B95; A131-B96; A131-B97; A131-B98; A131-B99; A131-B100; A131-B101; A131-B102; A131-B103; A131-B104; A131-B105; A131-B106; A131-B107; A131-B108; A131-B109; A131-B110; A131-B111; A131-B112; A131-B113;

A132-B1; A132-B2; A132-B3; A132-B4; A132-B5; A132-B6; A132-B7; A132-B8; A132-B9; A132-B10; A132-B11; A132-B12; A132-B13; A132-B14; A132-B15; A132-B16; A132-B17; A132-B18; A132-B19; A132-B20; A132-B21; A132-B22; A132-B23; A132-B24; A132-B25; A132-B26; A132-B27; A132-B28; A132-B29; A132-B30; A132-B31; A132-B32; A132-B33; A132-B34; A132-B35; A132-B36; A132-B37; A132-B38; A132-B39; A132-B40; A132-B41; A132-B42; A132-B43; A132-B44; A132-B45; A132-B46; A132-B47; A132-B48; A132-B49; A132-B50; A132-B51; A132-B52; A132-B53; A132-B54; A132-B55; A132-B56; A132-B57; A132-B58; A132-B59; A132-B60; A132-B61; A132-B62; A132-B63; A132-B64; A132-B65; A132-B66; A132-B67; A132-B68; A132-B69; A132-B70; A132-B71; A132-B72; A132-B73; A132-B74; A132-B75; A132-B76; A132-B77; A132-B78; A132-B79; A132-B80; A132-B81; A132-B82; A132-B83; A132-B84; A132-B85; A132-B86; A132-B87; A132-B88; A132-B89; A132-B90; A132-B91; A132-B92; A132-B93; A132-B94; A132-B95; A132-B96; A132-B97; A132-B98; A132-B99; A132-B100; A132-B101; A132-B102; A132-B103; A132-B104; A132-B105; A132-B106; A132-B107; A132-B108; A132-B109; A132-B110; A132-B111; A132-B112; A132-B113;

A133-B1; A133-B2; A133-B3; A133-B4; A133-B5; A133-B6; A133-B7; A133-B8; A133-B9; A133-B10; A133-B11; A133-B12; A133-B13; A133-B14; A133-B15; A133-B16; A133-B17; A133-B18; A133-B19; A133-B20; A133-B21; A133-B22; A133-B23; A133-B24; A133-B25; A133-B26; A133-B27; A133-B28; A133-B29; A133-B30; A133-B31; A133-B32; A133-B33; A133-B34; A133-B35; A133-B36; A133-B37; A133-B38; A133-B39; A133-B40; A133-B41; A133-B42; A133-B43; A133-B44; A133-B45; A133-B46; A133-B47; A133-B48; A133-B49; A133-B50; A133-B51; A133-B52; A133-B53; A133-B54; A133-B55; A133-B56; A133-B57; A133-B58; A133-B59; A133-B60; A133-B61; A133-B62; A133-B63; A133-B64; A133-B65; A133-B66; A133-B67; A133-B68; A133-B69; A133-B70; A133-B71; A133-B72; A133-B73; A133-B74; A133-B75; A133-B76; A133-B77; A133-B78; A133-B79; A133-B80; A133-B81; A133-B82; A133-B83; A133-B84; A133-B85; A133-B86; A133-B87; A133-B88; A133-B89; A133-B90; A133-B91; A133-B92; A133-B93; A133-B94; A133-B95; A133-B96; A133-B97; A133-B98; A133-B99; A133-B100; A133-B101; A133-B102; A133-B103; A133-B104; A133-B105; A133-B106; A133-B107; A133-B108; A133-B109; A133-B110; A133-B111; A133-B112; A133-B113;

A134-B1; A134-B2; A134-B3; A134-B4; A134-B5; A134-B6; A134-B7; A134-B8; A134-B9; A134-B10; A134-B11; A134-B12; A134-B13; A134-B14; A134-B15; A134-B16; A134-B17; A134-B18; A134-B19; A134-B20; A134-B21; A134-B22; A134-B23; A134-B24; A134-B25; A134-B26; A134-B27; A134-B28; A134-B29; A134-B30; A134-B31; A134-B32; A134-B33; A134-B34; A134-B35; A134-B36; A134-B37; A134-B38; A134-B39; A134-B40; A134-B41; A134-B42; A134-B43; A134-B44; A134-B45; A134-B46; A134-B47; A134-B48; A134-B49; A134-B50; A134-B51; A134-B52; A134-B53; A134-B54; A134-B55; A134-B56; A134-B57; A134-B58; A134-B59; A134-B60; A134-B61; A134-B62; A134-B63; A134-B64; A134-B65; A134-B66; A134-B67; A134-B68; A134-B69; A134-B70; A134-B71; A134-B72; A134-B73; A134-B74; A134-B75; A134-B76; A134-B77; A134-B78; A134-B79; A134-B80; A134-B81; A134-B82; A134-B83; A134-B84; A134-B85; A134-B86; A134-B87; A134-B88; A134-B89; A134-B90; A134-B91; A134-B92; A134-B93; A134-B94; A134-B95; A134-B96; A134-B97; A134-B98; A134-B99; A134-B100; A134-B101; A134-B102; A134-B103; A134-B104; A134-B105; A134-B106; A134-B107; A134-B108; A134-B109; A134-B110; A134-B111; A134-B112; A134-B113;

A135-B1; A135-B2; A135-B3; A135-B4; A135-B5; A135-B6; A135-B7; A135-B8; A135-B9; A135-B10; A135-B11; A135-B12; A135-B13; A135-B14; A135-B15; A135-B16; A135-B17; A135-B18; A135-B19; A135-B20; A135-B21; A135-B22; A135-B23; A135-B24; A135-B25; A135-B26; A135-B27; A135-B28; A135-B29; A135-B30; A135-B31; A135-B32; A135-B33; A135-B34; A135-B35; A135-B36; A135-B37; A135-B38; A135-B39; A135-B40; A135-B41; A135-B42; A135-B43; A135-B44; A135-B45; A135-B46; A135-B47; A135-B48; A135-B49; A135-B50; A135-B51; A135-B52; A135-B53; A135-B54; A135-B55; A135-B56; A135-B57; A135-B58; A135-B59; A135-B60; A135-B61; A135-B62; A135-B63; A135-B64; A135-B65; A135-B66; A135-B67; A135-B68; A135-B69; A135-B70; A135-B71; A135-B72; A135-B73; A135-B74; A135-B75; A135-B76; A135-B77; A135-B78; A135-B79; A135-B80; A135-B81; A135-B82; A135-B83; A135-B84; A135-B85; A135-B86; A135-B87; A135-B88; A135-B89; A135-B90; A135-B91; A135-B92; A135-B93; A135-B94; A135-B95; A135-B96; A135-B97; A135-B98; A135-B99; A135-B100; A135-B101; A135-B102; A135-B103; A135-B104; A135-B105; A135-B106; A135-B107; A135-B108; A135-B109; A135-B110; A135-B111; A135-B112; A135-B113;

A136-B1; A136-B2; A136-B3; A136-B4; A136-B5; A136-B6; A136-B7; A136-B8; A136-B9; A136-B10; A136-B11; A136-B12; A136-B13; A136-B14; A136-B15; A136-B16; A136-B17; A136-B18; A136-B19; A136-B20; A136-B21; A136-B22; A136-B23; A136-B24; A136-B25; A136-B26; A136-B27; A136-B28; A136-B29; A136-B30; A136-B31; A136-B32; A136-B33; A136-B34; A136-B35; A136-B36; A136-B37; A136-B38; A136-B39; A136-B40; A136-B41; A136-B42; A136-B43; A136-B44; A136-B45; A136-B46; A136-B47; A136-B48; A136-B49; A136-B50; A136-B51; A136-B52; A136-B53; A136-B54; A136-B55; A136-B56; A136-B57; A136-B58; A136-B59; A136-B60; A136-B61; A136-B62; A136-B63; A136-B64; A136-B65; A136-B66; A136-B67; A136-B68; A136-B69; A136-B70; A136-B71; A136-B72; A136-B73; A136-B74; A136-B75; A136-B76; A136-B77; A136-B78; A136-B79; A136-B80; A136-B81; A136-B82; A136-B83; A136-B84; A136-B85; A136-B86; A136-B87; A136-B88; A136-B89; A136-B90; A136-B91; A136-B92; A136-B93; A136-B94; A136-B95; A136-B96; A136-B97; A136-B98; A136-B99; A136-B100; A136-B101; A136-B102; A136-B103; A136-B104; A136-B105; A136-B106; A136-B107; A136-B108; A136-B109; A136-B110; A136-B111; A136-B112; A136-B113;

A137-B1; A137-B2; A137-B3; A137-B4; A137-B5; A137-B6; A137-B7; A137-B8; A137-B9; A137-B10; A137-B11; A137-B12; A137-B13; A137-B14; A137-B15; A137-B16; A137-B17; A137-B18; A137-B19; A137-B20; A137-B21; A137-B22; A137-B23; A137-B24; A137-B25; A137-B26; A137-B27; A137-B28; A137-B29; A137-B30; A137-B31; A137-B32; A137-B33; A137-B34; A137-B35; A137-B36; A137-B37; A137-B38; A137-B39; A137-B40; A137-B41; A137-B42; A137-B43; A137-B44; A137-B45; A137-B46; A137-B47; A137-B48; A137-B49; A137-B50; A137-B51; A137-B52; A137-B53; A137-B54; A137-B55; A137-B56; A137-B57; A137-B58; A137-B59; A137-B60; A137-B61; A137-B62; A137-B63; A137-B64; A137-B65; A137-B66; A137-B67; A137-B68; A137-B69; A137-B70; A137-B71; A137-B72; A137-B73; A137-B74; A137-B75; A137-B76; A137-B77; A137-B78; A137-B79; A137-B80; A137-B81; A137-B82; A137-B83; A137-B84; A137-B85; A137-B86; A137-B87; A137-B88; A137-B89; A137-B90; A137-B91; A137-B92; A137-B93; A137-B94; A137-B95; A137-B96; A137-B97; A137-B98; A137-B99; A137-B100; A137-B101; A137-B102; A137-B103; A137-B104; A137-B105; A137-B106; A137-B107; A137-B108; A137-B109; A137-B110; A137-B111; A137-B112; A137-B113;

A138-B1; A138-B2; A138-B3; A138-B4; A138-B5; A138-B6; A138-B7; A138-B8; A138-B9; A138-B10; A138-B11; A138-B12; A138-B13; A138-B14; A138-B15; A138-B16; A138-B17; A138-B18; A138-B19; A138-B20; A138-B21; A138-B22; A138-B23; A138-B24; A138-B25; A138-B26; A138-B27; A138-B28; A138-B29; A138-B30; A138-B31; A138-B32; A138-B33; A138-B34; A138-B35; A138-B36; A138-B37; A138-B38; A138-B39; A138-B40; A138-B41; A138-B42; A138-B43; A138-B44; A138-B45; A138-B46; A138-B47; A138-B48; A138-B49; A138-B50; A138-B51; A138-B52; A138-B53; A138-B54; A138-B55; A138-B56; A138-B57; A138-B58; A138-B59; A138-B60; A138-B61; A138-B62; A138-B63; A138-B64; A138-B65; A138-B66; A138-B67; A138-B68; A138-B69; A138-B70; A138-B71; A138-B72; A138-B73; A138-B74; A138-B75; A138-B76; A138-B77; A138-B78; A138-B79; A138-B80; A138-B81; A138-B82; A138-B83; A138-B84; A138-B85; A138-B86; A138-B87; A138-B88; A138-B89; A138-B90; A138-B91; A138-B92; A138-B93; A138-B94; A138-B95; A138-B96; A138-B97; A138-B98; A138-B99; A138-B100; A138-B101; A138-B102; A138-B103; A138-B104; A138-B105; A138-B106; A138-B107; A138-B108; A138-B109; A138-B110; A138-B111; A138-B112; A138-B113;

A139-B1; A139-B2; A139-B3; A139-B4; A139-B5; A139-B6; A139-B7; A139-B8; A139-B9; A139-B10; A139-B11; A139-B12; A139-B13; A139-B14; A139-B15; A139-B16; A139-B17; A139-B18; A139-B19; A139-B20; A139-B21; A139-B22; A139-B23; A139-B24; A139-B25; A139-B26; A139-B27; A139-B28; A139-B29; A139-B30; A139-B31; A139-B32; A139-B33; A139-B34; A139-B35; A139-B36; A139-B37; A139-B38; A139-B39; A139-B40; A139-B41; A139-B42; A139-B43; A139-B44; A139-B45; A139-B46; A139-B47; A139-B48; A139-B49; A139-B50; A139-B51; A139-B52; A139-B53; A139-B54; A139-B55; A139-B56; A139-B57; A139-B58; A139-B59; A139-B60; A139-B61; A139-B62; A139-B63; A139-B64; A139-B65; A139-B66; A139-B67; A139-B68; A139-B69; A139-B70; A139-B71; A139-B72; A139-B73; A139-B74; A139-B75; A139-B76; A139-B77; A139-B78; A139-B79; A139-B80; A139-B81; A139-B82; A139-B83; A139-B84; A139-B85; A139-B86; A139-B87; A139-B88; A139-B89; A139-B90; A139-B91; A139-B92; A139-B93; A139-B94; A139-B95; A139-B96; A139-B97; A139-B98; A139-B99; A139-B100; A139-B101; A139-B102; A139-B103; A139-B104; A139-B105; A139-B106; A139-B107; A139-B108; A139-B109; A139-B110; A139-B111; A139-B112; A139-B113;

A140-B1; A140-B2; A140-B3; A140-B4; A140-B5; A140-B6; A140-B7; A140-B8; A140-B9; A140-B10; A140-B11; A140-B12; A140-B13; A140-B14; A140-B15; A140-B16; A140-B17; A140-B18; A140-B19; A140-B20; A140-B21; A140-B22; A140-B23; A140-B24; A140-B25; A140-B26; A140-B27; A140-B28; A140-B29; A140-B30; A140-B31; A140-B32; A140-B33; A140-B34; A140-B35; A140-B36; A140-B37; A140-B38; A140-B39; A140-B40; A140-B41; A140-B42; A140-B43; A140-B44; A140-B45; A140-B46; A140-B47; A140-B48; A140-B49; A140-B50; A140-B51; A140-B52; A140-B53; A140-B54; A140-B55; A140-B56; A140-B57; A140-B58; A140-B59; A140-B60; A140-B61; A140-B62; A140-B63; A140-B64; A140-B65; A140-B66; A140-B67; A140-B68; A140-B69; A140-B70; A140-B71; A140-B72; A140-B73; A140-B74; A140-B75; A140-B76; A140-B77; A140-B78; A140-B79; A140-B80; A140-B81; A140-B82; A140-B83; A140-B84; A140-B85; A140-B86; A140-B87; A140-B88; A140-B89; A140-B90; A140-B91; A140-B92; A140-B93; A140-B94; A140-B95; A140-B96; A140-B97; A140-B98; A140-B99; A140-B100; A140-B101; A140-B102; A140-B103; A140-B104; A140-B105; A140-B106; A140-B107; A140-B108; A140-B109; A140-B110; A140-B111; A140-B112; A140-B113;

A141-B1; A141-B2; A141-B3; A141-B4; A141-B5; A141-B6; A141-B7; A141-B8; A141-B9; A141-B10; A141-B11; A141-B12; A141-B13; A141-B14; A141-B15; A141-B16; A141-B17; A141-B18; A141-B19; A141-B20; A141-B21; A141-B22; A141-B23; A141-B24; A141-B25; A141-B26; A141-B27; A141-B28; A141-B29; A141-B30; A141-B31; A141-B32; A141-B33; A141-B34; A141-B35; A141-B36; A141-B37; A141-B38; A141-B39; A141-B40; A141-B41; A141-B42; A141-B43; A141-B44; A141-B45; A141-B46; A141-B47; A141-B48; A141-B49; A141-B50; A141-B51; A141-B52; A141-B53; A141-B54; A141-B55; A141-B56; A141-B57; A141-B58; A141-B59; A141-B60; A141-B61; A141-B62; A141-B63; A141-B64; A141-B65; A141-B66; A141-B67; A141-B68; A141-B69; A141-B70; A141-B71; A141-B72; A141-B73; A141-B74; A141-B75; A141-B76; A141-B77; A141-B78; A141-B79; A141-B80; A141-B81; A141-B82; A141-B83; A141-B84; A141-B85; A141-B86; A141-B87; A141-B88; A141-B89; A141-B90; A141-B91; A141-B92; A141-B93; A141-B94; A141-B95; A141-B96; A141-B97; A141-B98; A141-B99; A141-B100; A141-B101; A141-B102; A141-B103; A141-B104; A141-B105; A141-B106; A141-B107; A141-B108; A141-B109; A141-B110; A141-B111; A141-B112; A141-B113;

A142-B1; A142-B2; A142-B3; A142-B4; A142-B5; A142-B6; A142-B7; A142-B8; A142-B9; A142-B10; A142-B11; A142-B12; A142-B13; A142-B14; A142-B15; A142-B16; A142-B17; A142-B18; A142-B19; A142-B20; A142-B21; A142-B22; A142-B23; A142-B24; A142-B25; A142-B26; A142-B27; A142-B28; A142-B29; A142-B30; A142-B31; A142-B32; A142-B33; A142-B34; A142-B35; A142-B36; A142-B37; A142-B38; A142-B39; A142-B40; A142-B41; A142-B42; A142-B43; A142-B44; A142-B45; A142-B46; A142-B47; A142-B48; A142-B49; A142-B50; A142-B51; A142-B52; A142-B53; A142-B54; A142-B55; A142-B56; A142-B57; A142-B58; A142-B59; A142-B60; A142-B61; A142-B62; A142-B63; A142-B64; A142-B65; A142-B66; A142-B67; A142-B68; A142-B69; A142-B70; A142-B71; A142-B72; A142-B73; A142-B74; A142-B75; A142-B76; A142-B77; A142-B78; A142-B79; A142-B80; A142-B81; A142-B82; A142-B83; A142-B84; A142-B85; A142-B86; A142-B87; A142-B88; A142-B89; A142-B90; A142-B91; A142-B92; A142-B93; A142-B94; A142-B95; A142-B96; A142-B97; A142-B98; A142-B99; A142-B100; A142-B101; A142-B102; A142-B103; A142-B104; A142-B105; A142-B106; A142-B107; A142-B108; A142-B109; A142-B110; A142-B111; A142-B112; A142-B113;

A143-B1; A143-B2; A143-B3; A143-B4; A143-B5; A143-B6; A143-B7; A143-B8; A143-B9; A143-B10; A143-B11; A143-B12; A143-B13; A143-B14; A143-B15; A143-B16; A143-B17; A143-B18; A143-B19; A143-B20; A143-B21; A143-B22; A143-B23; A143-B24; A143-B25; A143-B26; A143-B27; A143-B28; A143-B29; A143-B30; A143-B31; A143-B32; A143-B33; A143-B34; A143-B35; A143-B36; A143-B37; A143-B38; A143-B39; A143-B40; A143-B41; A143-B42; A143-B43; A143-B44; A143-B45; A143-B46; A143-B47; A143-B48; A143-B49; A143-B50; A143-B51; A143-B52; A143-B53; A143-B54; A143-B55; A143-B56; A143-B57; A143-B58; A143-B59; A143-B60; A143-B61; A143-B62; A143-B63; A143-B64; A143-B65; A143-B66; A143-B67; A143-B68; A143-B69; A143-B70; A143-B71; A143-B72; A143-B73; A143-B74; A143-B75; A143-B76; A143-B77; A143-B78; A143-B79; A143-B80; A143-B81; A143-B82; A143-B83; A143-B84; A143-B85; A143-B86; A143-B87; A143-B88; A143-B89; A143-B90; A143-B91; A143-B92; A143-B93; A143-B94; A143-B95; A143-B96; A143-B97; A143-B98; A143-B99; A143-B100; A143-B101; A143-B102; A143-B103; A143-B104; A143-B105; A143-B106; A143-B107; A143-B108; A143-B109; A143-B110; A143-B111; A143-B112; A143-B113;

A144-B1; A144-B2; A144-B3; A144-B4; A144-B5; A144-B6; A144-B7; A144-B8; A144-B9; A144-B10; A144-B11; A144-B12; A144-B13; A144-B14; A144-B15; A144-B16; A144-B17; A144-B18; A144-B19; A144-B20; A144-B21; A144-B22; A144-B23; A144-B24; A144-B25; A144-B26; A144-B27; A144-B28; A144-B29; A144-B30; A144-B31; A144-B32; A144-B33; A144-B34; A144-B35; A144-B36; A144-B37; A144-B38; A144-B39; A144-B40; A144-B41; A144-B42; A144-B43; A144-B44; A144-B45; A144-B46; A144-B47; A144-B48; A144-B49; A144-B50; A144-B51; A144-B52; A144-B53; A144-B54; A144-B55; A144-B56; A144-B57; A144-B58; A144-B59; A144-B60; A144-B61; A144-B62; A144-B63; A144-B64; A144-B65; A144-B66; A144-B67; A144-B68; A144-B69; A144-B70; A144-B71; A144-B72; A144-B73; A144-B74; A144-B75; A144-B76; A144-B77; A144-B78; A144-B79; A144-B80; A144-B81; A144-B82; A144-B83; A144-B84; A144-B85; A144-B86; A144-B87; A144-B88; A144-B89; A144-B90; A144-B91; A144-B92; A144-B93; A144-B94; A144-B95; A144-B96; A144-B97; A144-B98; A144-B99; A144-B100; A144-B101; A144-B102; A144-B103; A144-B104; A144-B105; A144-B106; A144-B107; A144-B108; A144-B109; A144-B110; A144-B111; A144-B112; A144-B113;

A145-B1; A145-B2; A145-B3; A145-B4; A145-B5; A145-B6; A145-B7; A145-B8; A145-B9; A145-B10; A145-B11; A145-B12; A145-B13; A145-B14; A145-B15; A145-B16; A145-B17; A145-B18; A145-B19; A145-B20; A145-B21; A145-B22; A145-B23; A145-B24; A145-B25; A145-B26; A145-B27; A145-B28; A145-B29; A145-B30; A145-B31; A145-B32; A145-B33; A145-B34; A145-B35; A145-B36; A145-B37; A145-B38; A145-B39; A145-B40; A145-B41; A145-B42; A145-B43; A145-B44; A145-B45; A145-B46; A145-B47; A145-B48; A145-B49; A145-B50; A145-B51; A145-B52; A145-B53; A145-B54; A145-B55; A145-B56; A145-B57; A145-B58; A145-B59; A145-B60; A145-B61; A145-B62; A145-B63; A145-B64; A145-B65; A145-B66; A145-B67; A145-B68; A145-B69; A145-B70; A145-B71; A145-B72; A145-B73; A145-B74; A145-B75; A145-B76; A145-B77; A145-B78; A145-B79; A145-B80; A145-B81; A145-B82; A145-B83; A145-B84; A145-B85; A145-B86; A145-B87; A145-B88; A145-B89; A145-B90; A145-B91; A145-B92; A145-B93; A145-B94; A145-B95; A145-B96; A145-B97; A145-B98; A145-B99; A145-B100; A145-B101; A145-B102; A145-B103; A145-B104; A145-B105; A145-B106; A145-B107; A145-B108; A145-B109; A145-B110; A145-B111; A145-B112; A145-B113;

A146-B1; A146-B2; A146-B3; A146-B4; A146-B5; A146-B6; A146-B7; A146-B8; A146-B9; A146-B10; A146-B11; A146-B12; A146-B13; A146-B14; A146-B15; A146-B16; A146-B17; A146-B18; A146-B19; A146-B20; A146-B21; A146-B22; A146-B23; A146-B24; A146-B25; A146-B26; A146-B27; A146-B28; A146-B29; A146-B30; A146-B31; A146-B32; A146-B33; A146-B34; A146-B35; A146-B36; A146-B37; A146-B38; A146-B39; A146-B40; A146-B41; A146-B42; A146-B43; A146-B44; A146-B45; A146-B46; A146-B47; A146-B48; A146-B49; A146-B50; A146-B51; A146-B52; A146-B53; A146-B54; A146-B55; A146-B56; A146-B57; A146-B58; A146-B59; A146-B60; A146-B61; A146-B62; A146-B63; A146-B64; A146-B65; A146-B66; A146-B67; A146-B68; A146-B69; A146-B70; A146-B71; A146-B72; A146-B73; A146-B74; A146-B75; A146-B76; A146-B77; A146-B78; A146-B79; A146-B80; A146-B81; A146-B82; A146-B83; A146-B84; A146-B85; A146-B86; A146-B87; A146-B88; A146-B89; A146-B90; A146-B91; A146-B92; A146-B93; A146-B94; A146-B95; A146-B96; A146-B97; A146-B98; A146-B99; A146-B100; A146-B101; A146-B102; A146-B103; A146-B104; A146-B105; A146-B106; A146-B107; A146-B108; A146-B109; A146-B110; A146-B111; A146-B112; A146-B113;

A147-B1; A147-B2; A147-B3; A147-B4; A147-B5; A147-B6; A147-B7; A147-B8; A147-B9; A147-B10; A147-B11; A147-B12; A147-B13; A147-B14; A147-B15; A147-B16; A147-B17; A147-B18; A147-B19; A147-B20; A147-B21; A147-B22; A147-B23; A147-B24; A147-B25; A147-B26; A147-B27; A147-B28; A147-B29; A147-B30; A147-B31; A147-B32; A147-B33; A147-B34; A147-B35; A147-B36; A147-B37; A147-B38; A147-B39; A147-B40; A147-B41; A147-B42; A147-B43; A147-B44; A147-B45; A147-B46; A147-B47; A147-B48; A147-B49; A147-B50; A147-B51; A147-B52; A147-B53; A147-B54; A147-B55; A147-B56; A147-B57; A147-B58; A147-B59; A147-B60; A147-B61; A147-B62; A147-B63; A147-B64; A147-B65; A147-B66; A147-B67; A147-B68; A147-B69; A147-B70; A147-B71; A147-B72; A147-B73; A147-B74; A147-B75; A147-B76; A147-B77; A147-B78; A147-B79; A147-B80; A147-B81; A147-B82; A147-B83; A147-B84; A147-B85; A147-B86; A147-B87; A147-B88; A147-B89; A147-B90; A147-B91; A147-B92; A147-B93; A147-B94; A147-B95; A147-B96; A147-B97; A147-B98; A147-B99; A147-B100; A147-B101; A147-B102; A147-B103; A147-B104; A147-B105; A147-B106; A147-B107; A147-B108; A147-B109; A147-B110; A147-B111; A147-B112; A147-B113;

A148-B1; A148-B2; A148-B3; A148-B4; A148-B5; A148-B6; A148-B7; A148-B8; A148-B9; A148-B10; A148-B11; A148-B12; A148-B13; A148-B14; A148-B15; A148-B16; A148-B17; A148-B18; A148-B19; A148-B20; A148-B21; A148-B22; A148-B23; A148-B24; A148-B25; A148-B26; A148-B27; A148-B28; A148-B29; A148-B30; A148-B31; A148-B32; A148-B33; A148-B34; A148-B35; A148-B36; A148-B37; A148-B38; A148-B39; A148-B40; A148-B41; A148-B42; A148-B43; A148-B44; A148-B45; A148-B46; A148-B47; A148-B48; A148-B49; A148-B50; A148-B51; A148-B52; A148-B53; A148-B54; A148-B55; A148-B56; A148-B57; A148-B58; A148-B59; A148-B60; A148-B61; A148-B62; A148-B63; A148-B64; A148-B65; A148-B66; A148-B67; A148-B68; A148-B69; A148-B70; A148-B71; A148-B72; A148-B73; A148-B74; A148-B75; A148-B76; A148-B77; A148-B78; A148-B79; A148-B80; A148-B81; A148-B82; A148-B83; A148-B84; A148-B85; A148-B86; A148-B87; A148-B88; A148-B89; A148-B90; A148-B91; A148-B92; A148-B93; A148-B94; A148-B95; A148-B96; A148-B97; A148-B98; A148-B99; A148-B100; A148-B101; A148-B102; A148-B103; A148-B104; A148-B105; A148-B106; A148-B107; A148-B108; A148-B109; A148-B110; A148-B111; A148-B112; A148-B113;

A149-B1; A149-B2; A149-B3; A149-B4; A149-B5; A149-B6; A149-B7; A149-B8; A149-B9; A149-B10; A149-B11; A149-B12; A149-B13; A149-B14; A149-B15; A149-B16; A149-B17; A149-B18; A149-B19; A149-B20; A149-B21; A149-B22; A149-B23; A149-B24; A149-B25; A149-B26; A149-B27; A149-B28; A149-B29; A149-B30; A149-B31; A149-B32; A149-B33; A149-B34; A149-B35; A149-B36; A149-B37; A149-B38; A149-B39; A149-B40; A149-B41; A149-B42; A149-B43; A149-B44; A149-B45; A149-B46; A149-B47; A149-B48; A149-B49; A149-B50; A149-B51; A149-B52; A149-B53; A149-B54; A149-B55; A149-B56; A149-B57; A149-B58; A149-B59; A149-B60; A149-B61; A149-B62; A149-B63; A149-B64; A149-B65; A149-B66; A149-B67; A149-B68; A149-B69; A149-B70; A149-B71; A149-B72; A149-B73; A149-B74; A149-B75; A149-B76; A149-B77; A149-B78; A149-B79; A149-B80; A149-B81; A149-B82; A149-B83; A149-B84; A149-B85; A149-B86; A149-B87; A149-B88; A149-B89; A149-B90; A149-B91; A149-B92; A149-B93; A149-B94; A149-B95; A149-B96; A149-B97; A149-B98; A149-B99; A149-B100; A149-B101; A149-B102; A149-B103; A149-B104; A149-B105; A149-B106; A149-B107; A149-B108; A149-B109; A149-B110; A149-B111; A149-B112; A149-B113;

A150-B1; A150-B2; A150-B3; A150-B4; A150-B5; A150-B6; A150-B7; A150-B8; A150-B9; A150-B10; A150-B11; A150-B12; A150-B13; A150-B14; A150-B15; A150-B16; A150-B17; A150-B18; A150-B19; A150-B20; A150-B21; A150-B22; A150-B23; A150-B24; A150-B25; A150-B26; A150-B27; A150-B28; A150-B29; A150-B30; A150-B31; A150-B32; A150-B33; A150-B34; A150-B35; A150-B36; A150-B37; A150-B38; A150-B39; A150-B40; A150-B41; A150-B42; A150-B43; A150-B44; A150-B45; A150-B46; A150-B47; A150-B48; A150-B49; A150-B50; A150-B51; A150-B52; A150-B53; A150-B54; A150-B55; A150-B56; A150-B57; A150-B58; A150-B59; A150-B60; A150-B61; A150-B62; A150-B63; A150-B64; A150-B65; A150-B66; A150-B67; A150-B68; A150-B69; A150-B70; A150-B71; A150-B72; A150-B73; A150-B74; A150-B75; A150-B76; A150-B77; A150-B78; A150-B79; A150-B80; A150-B81; A150-B82; A150-B83; A150-B84; A150-B85; A150-B86; A150-B87; A150-B88; A150-B89; A150-B90; A150-B91; A150-B92; A150-B93; A150-B94; A150-B95; A150-B96; A150-B97; A150-B98; A150-B99; A150-B100; A150-B101; A150-B102; A150-B103; A150-B104; A150-B105; A150-B106; A150-B107; A150-B108; A150-B109; A150-B110; A150-B111; A150-B112; A150-B113;

A151-B1; A151-B2; A151-B3; A151-B4; A151-B5; A151-B6; A151-B7; A151-B8; A151-B9; A151-B10; A151-B11; A151-B12; A151-B13; A151-B14; A151-B15; A151-B16; A151-B17; A151-B18; A151-B19; A151-B20; A151-B21; A151-B22; A151-B23; A151-B24; A151-B25; A151-B26; A151-B27; A151-B28; A151-B29; A151-B30; A151-B31; A151-B32; A151-B33; A151-B34; A151-B35; A151-B36; A151-B37; A151-B38; A151-B39; A151-B40; A151-B41; A151-B42; A151-B43; A151-B44; A151-B45; A151-B46; A151-B47; A151-B48; A151-B49; A151-B50; A151-B51; A151-B52; A151-B53; A151-B54; A151-B55; A151-B56; A151-B57; A151-B58; A151-B59; A151-B60; A151-B61; A151-B62; A151-B63; A151-B64; A151-B65; A151-B66; A151-B67; A151-B68; A151-B69; A151-B70; A151-B71; A151-B72; A151-B73; A151-B74; A151-B75; A151-B76; A151-B77; A151-B78; A151-B79; A151-B80; A151-B81; A151-B82; A151-B83; A151-B84; A151-B85; A151-B86; A151-B87; A151-B88; A151-B89; A151-B90; A151-B91; A151-B92; A151-B93; A151-B94; A151-B95; A151-B96; A151-B97; A151-B98; A151-B99; A151-B100; A151-B101; A151-B102; A151-B103; A151-B104; A151-B105; A151-B106; A151-B107; A151-B108; A151-B109; A151-B110; A151-B111; A151-B112; A151-B113;

A152-B1; A152-B2; A152-B3; A152-B4; A152-B5; A152-B6; A152-B7; A152-B8; A152-B9; A152-B10; A152-B11; A152-B12; A152-B13; A152-B14; A152-B15; A152-B16; A152-B17; A152-B18; A152-B19; A152-B20; A152-B21; A152-B22; A152-B23; A152-B24; A152-B25; A152-B26; A152-B27; A152-B28; A152-B29; A152-B30; A152-B31; A152-B32; A152-B33; A152-B34; A152-B35; A152-B36; A152-B37; A152-B38; A152-B39; A152-B40; A152-B41; A152-B42; A152-B43; A152-B44; A152-B45; A152-B46; A152-B47; A152-B48; A152-B49; A152-B50; A152-B51; A152-B52; A152-B53; A152-B54; A152-B55; A152-B56; A152-B57; A152-B58; A152-B59; A152-B60; A152-B61; A152-B62; A152-B63; A152-B64; A152-B65; A152-B66; A152-B67; A152-B68; A152-B69; A152-B70; A152-B71; A152-B72; A152-B73; A152-B74; A152-B75; A152-B76; A152-B77; A152-B78; A152-B79; A152-B80; A152-B81; A152-B82; A152-B83; A152-B84; A152-B85; A152-B86; A152-B87; A152-B88; A152-B89; A152-B90; A152-B91; A152-B92; A152-B93; A152-B94; A152-B95; A152-B96; A152-B97; A152-B98; A152-B99; A152-B100; A152-B101; A152-B102; A152-B103; A152-B104; A152-B105; A152-B106; A152-B107; A152-B108; A152-B109; A152-B110; A152-B111; A152-B112; A152-B113;

A153-B1; A153-B2; A153-B3; A153-B4; A153-B5; A153-B6; A153-B7; A153-B8; A153-B9; A153-B10; A153-B11; A153-B12; A153-B13; A153-B14; A153-B15; A153-B16; A153-B17; A153-B18; A153-B19; A153-B20; A153-B21; A153-B22; A153-B23; A153-B24; A153-B25; A153-B26; A153-B27; A153-B28; A153-B29; A153-B30; A153-B31; A153-B32; A153-B33; A153-B34; A153-B35; A153-B36; A153-B37; A153-B38; A153-B39; A153-B40; A153-B41; A153-B42; A153-B43; A153-B44; A153-B45; A153-B46; A153-B47; A153-B48; A153-B49; A153-B50; A153-B51; A153-B52; A153-B53; A153-B54; A153-B55; A153-B56; A153-B57; A153-B58; A153-B59; A153-B60; A153-B61; A153-B62; A153-B63; A153-B64; A153-B65; A153-B66; A153-B67; A153-B68; A153-B69; A153-B70; A153-B71; A153-B72; A153-B73; A153-B74; A153-B75; A153-B76; A153-B77; A153-B78; A153-B79; A153-B80; A153-B81; A153-B82; A153-B83; A153-B84; A153-B85; A153-B86; A153-B87; A153-B88; A153-B89; A153-B90; A153-B91; A153-B92; A153-B93; A153-B94; A153-B95; A153-B96; A153-B97; A153-B98; A153-B99; A153-B100; A153-B101; A153-B102; A153-B103; A153-B104; A153-B105; A153-B106; A153-B107; A153-B108; A153-B109; A153-B110; A153-B111; A153-B112; A153-B113;

A154-B1; A154-B2; A154-B3; A154-B4; A154-B5; A154-B6; A154-B7; A154-B8; A154-B9; A154-B10; A154-B11; A154-B12; A154-B13; A154-B14; A154-B15; A154-B16; A154-B17; A154-B18; A154-B19; A154-B20; A154-B21; A154-B22; A154-B23; A154-B24; A154-B25; A154-B26; A154-B27; A154-B28; A154-B29; A154-B30; A154-B31; A154-B32; A154-B33; A154-B34; A154-B35; A154-B36; A154-B37; A154-B38; A154-B39; A154-B40; A154-B41; A154-B42; A154-B43; A154-B44; A154-B45; A154-B46; A154-B47; A154-B48; A154-B49; A154-B50; A154-B51; A154-B52; A154-B53; A154-B54; A154-B55; A154-B56; A154-B57; A154-B58; A154-B59; A154-B60; A154-B61; A154-B62; A154-B63; A154-B64; A154-B65; A154-B66; A154-B67; A154-B68; A154-B69; A154-B70; A154-B71; A154-B72; A154-B73; A154-B74; A154-B75; A154-B76; A154-B77; A154-B78; A154-B79; A154-B80; A154-B81; A154-B82; A154-B83; A154-B84; A154-B85; A154-B86; A154-B87; A154-B88; A154-B89; A154-B90; A154-B91; A154-B92; A154-B93; A154-B94; A154-B95; A154-B96; A154-B97; A154-B98; A154-B99; A154-B100; A154-B101; A154-B102; A154-B103; A154-B104; A154-B105; A154-B106; A154-B107; A154-B108; A154-B109; A154-B110; A154-B111; A154-B112; A154-B113;

### Sub-Category IA: Amino Acid B of the Dipeptide Prodrug Element is N-Alkylated Glycine

In some embodiments, the B amino acid of the dipeptide prodrug element is N-alkylated glycine. Nonlimiting examples of dipeptide prodrug elements having N-alkylated glycine as the B amino acid are shown in the below Table.

| Dipeptide Prodrug | Amino | Amino |
|---|---|---|
| Element # | Acid ‘A’ | Acid ‘B’ |
| 1 | Aib | Gly(N-C₁-C₈alkyl) |
| 2 | d-Ala | Gly(N-C₁-C₈alkyl) |
| 3 | d-Lys | Gly(N-C₁-C₈alkyl) |
| 4 | d-Cys | Gly(N-C₁-C₈alkyl) |
| 5 | Aib | Gly(N-methyl) |
| 6 | d-Ala | Gly(N-methyl) |
| 7 | d-Lys | Gly(N-methyl) |
| 8 | d-Cys | Gly(N-methyl) |
| 9 | Aib | Gly(N-hexyl) |
| 10 | d-Ala | Gly(N-hexyl) |
| 11 | d-Lys | Gly(N-hexyl) |
| 12 | d-Cys | Gly(N-hexyl) |

### Sub-Category IB: Amino Acid B of the Dipeptide Prodrug Element is Unsubstituted or Monosubstituted at the Beta Position

In some embodiments, the B amino acid of the dipeptide prodrug element is unsubstituted or monosubstituted at the beta position and has a relatively non-bulky side chain. Nonlimiting examples of dipeptide prodrug elements having a B amino acid that is unsubstituted or monosubstituted at the beta position and a relatively non-bulky side chain are shown in the below Table.

| Dipeptide Prodrug | Amino | Amino |
|---|---|---|
| Element # | Acid ‘A’ | Acid ‘B’ |
| 13 | Aib | Ala(N-C₁-C₈alkyl) |
| 14 | d-Ala | Ala(N-C₁-C₈alkyl) |
| 15 | d-Lys | Ala(N-C₁-C₈alkyl) |
| 16 | d-Cys | Ala(N-C₁-C₈alkyl) |
| 17 | Aib | Leu(N-C₁-C₈alkyl) |
| 18 | d-Ala | Leu(N-C₁-C₈alkyl) |
| 19 | d-Lys | Leu(N-C₁-C₈alkyl) |
| 20 | d-Cys | Leu(N-C₁-C₈alkyl) |
| 21 | Aib | Met(N-C₁-C₈alkyl) |
| 22 | d-Ala | Met(N-C₁-C₈alkyl) |
| 23 | d-Lys | Met(N-C₁-C₈alkyl) |
| 24 | d-Cys | Met(N-C₁-C₈alkyl) |
| 25 | Aib | Asn(N-C₁-C₈alkyl) |
| 26 | d-Ala | Asn(N-C₁-C₈alkyl) |
| 27 | d-Lys | Asn(N-C₁-C₈alkyl) |
| 28 | d-Cys | Asn(N-C₁-C₈alkyl) |
| 29 | Aib | Glu(N-C₁-C₈alkyl) |
| 30 | d-Ala | Glu(N-C₁-C₈alkyl) |
| 31 | d-Lys | Glu(N-C₁-C₈alkyl) |
| 32 | d-Cys | Glu(N-C₁-C₈alkyl) |
| 33 | Aib | Asp(N-C₁-C₈alkyl) |
| 34 | d-Ala | Asp(N-C₁-C₈alkyl) |
| 35 | d-Lys | Asp(N-C₁-C₈alkyl) |
| 36 | d-Cys | Asp(N-C₁-C₈alkyl) |
| 37 | Aib | Gln(N-C₁-C₈alkyl) |
| 38 | d-Ala | Gln(N-C₁-C₈alkyl) |
| 39 | d-Lys | Gln(N-C₁-C₈alkyl) |
| 40 | d-Cys | Gln(N-C₁-C₈alkyl) |
| 41 | Aib | His(N-C₁-C₈alkyl) |
| 42 | d-Ala | His(N-C₁-C₈alkyl) |
| 43 | d-Lys | His(N-C₁-C₈alkyl) |
| 44 | d-Cys | His(N-C₁-C₈alkyl) |
| 45 | Aib | Lys(N-C₁-C₈alkyl) |
| 46 | d-Ala | Lys(N-C₁-C₈alkyl) |
| 47 | d-Lys | Lys(N-C₁-C₈alkyl) |
| 48 | d-Cys | Lys(N-C₁-C₈alkyl) |
| 49 | Aib | Arg(N-C₁-C₈alkyl) |
| 50 | d-Ala | Arg(N-C₁-C₈alkyl) |
| 51 | d-Lys | Arg(N-C₁-C₈alkyl) |
| 52 | d-Cys | Arg(N-C₁-C₈alkyl) |
| 53 | Aib | Ser(N-C₁-C₈alkyl) |
| 54 | d-Ala | Ser(N-C₁-C₈alkyl) |
| 55 | d-Lys | Ser(N-C₁-C₈alkyl) |
| 56 | d-Cys | Ser(N-C₁-C₈alkyl) |
| 57 | Aib | Cys(N-C₁-C₈alkyl) |
| 58 | d-Ala | Cys(N-C₁-C₈alkyl) |
| 59 | d-Lys | Cys(N-C₁-C₈alkyl) |
| 60 | d-Cys | Cys(N-C₁-C₈alkyl) |
| 61 | Aib | Pro |
| 62 | d-Ala | Pro |
| 63 | d-Lys | Pro |
| 64 | d-Cys | Pro |
| 65 | Aib | Ala(N-methyl) |
| 66 | d-Ala | Ala(N-methyl) |
| 67 | d-Lys | Ala(N-methyl) |
| 68 | d-Cys | Ala(N-methyl) |
| 69 | Aib | Leu(N-methyl) |
| 70 | d-Ala | Leu(N-methyl) |
| 71 | d-Lys | Leu(N-methyl) |
| 72 | d-Cys | Leu(N-methyl) |
| 73 | Aib | Met(N-methyl) |
| 74 | d-Ala | Met(N-methyl) |
| 75 | d-Lys | Met(N-methyl) |
| 76 | d-Cys | Met(N-methyl) |
| 77 | Aib | Asn(N-methyl) |
| 78 | d-Ala | Asn(N-methyl) |
| 79 | d-Lys | Asn(N-methyl) |
| 80 | d-Cys | Asn(N-methyl) |
| 81 | Aib | Glu(N-methyl) |
| 82 | d-Ala | Glu(N-methyl) |
| 83 | d-Lys | Glu(N-methyl) |
| 84 | d-Cys | Glu(N-methyl) |
| 85 | Aib | Asp(N-methyl) |
| 86 | d-Ala | Asp(N-methyl) |
| 87 | d-Lys | Asp(N-methyl) |
| 88 | d-Cys | Asp(N-methyl) |
| 89 | Aib | Gln(N-methyl) |
| 90 | d-Ala | Gln(N-methyl) |
| 91 | d-Lys | Gln(N-methyl) |
| 92 | d-Cys | Gln(N-methyl) |
| 93 | Aib | His(N-methyl) |
| 94 | d-Ala | His(N-methyl) |
| 95 | d-Lys | His(N-methyl) |
| 96 | d-Cys | His(N-methyl) |
| 97 | Aib | Lys(N-methyl) |
| 98 | d-Ala | Lys(N-methyl) |
| 99 | d-Lys | Lys(N-methyl) |
| 100 | d-Cys | Lys(N-methyl) |
| 101 | Aib | Arg(N-methyl) |
| 102 | d-Ala | Arg(N-methyl) |
| 103 | d-Lys | Arg(N-methyl) |
| 104 | d-Cys | Arg(N-methyl) |
| 105 | Aib | Ser(N-methyl) |
| 106 | d-Ala | Ser(N-methyl) |
| 107 | d-Lys | Ser(N-methyl) |
| 108 | d-Cys | Ser(N-methyl) |
| 109 | Aib | Cys(N-methyl) |
| 110 | d-Ala | Cys(N-methyl) |
| 111 | d-Lys | Cys(N-methyl) |
| 112 | d-Cys | Cys(N-methyl) |
| 113 | Aib | Ala(N-hexyl) |
| 114 | d-Ala | Ala(N-hexyl) |
| 115 | d-Lys | Ala(N-hexyl) |
| 116 | d-Cys | Ala(N-hexyl) |
| 117 | Aib | Leu(N-hexyl) |
| 118 | d-Ala | Leu(N-hexyl) |
| 119 | d-Lys | Leu(N-hexyl) |
| 120 | d-Cys | Leu(N-hexyl) |
| 121 | Aib | Met(N-hexyl) |
| 122 | d-Ala | Met(N-hexyl) |
| 123 | d-Lys | Met(N-hexyl) |
| 124 | d-Cys | Met(N-hexyl) |
| 125 | Aib | Asn(N-hexyl) |
| 126 | d-Ala | Asn(N-hexyl) |
| 127 | d-Lys | Asn(N-hexyl) |
| 128 | d-Cys | Asn(N-hexyl) |
| 129 | Aib | Glu(N-hexyl) |
| 130 | d-Ala | Glu(N-hexyl) |
| 131 | d-Lys | Glu(N-hexyl) |
| 132 | d-Cys | Glu(N-hexyl) |
| 133 | Aib | Asp(N-hexyl) |
| 134 | d-Ala | Asp(N-hexyl) |
| 135 | d-Lys | Asp(N-hexyl) |
| 136 | d-Cys | Asp(N-hexyl) |
| 137 | Aib | Gln(N-hexyl) |
| 138 | d-Ala | Gln(N-hexyl) |
| 139 | d-Lys | Gln(N-hexyl) |
| 140 | d-Cys | Gln(N-hexyl) |
| 141 | Aib | His(N-hexyl) |
| 142 | d-Ala | His(N-hexyl) |
| 143 | d-Lys | His(N-hexyl) |
| 144 | d-Cys | His(N-hexyl) |
| 145 | Aib | Lys(N-hexyl) |
| 146 | d-Ala | Lys(N-hexyl) |
| 147 | d-Lys | Lys(N-hexyl) |
| 148 | d-Cys | Lys(N-hexyl) |
| 149 | Aib | Arg(N-hexyl) |
| 150 | d-Ala | Arg(N-hexyl) |
| 151 | d-Lys | Arg(N-hexyl) |
| 152 | d-Cys | Arg(N-hexyl) |
| 153 | Aib | Ser(N-hexyl) |
| 154 | d-Ala | Ser(N-hexyl) |
| 155 | d-Lys | Ser(N-hexyl) |
| 156 | d-Cys | Ser(N-hexyl) |
| 157 | Aib | Cys(N-hexyl) |
| 158 | d-Ala | Cys(N-hexyl) |
| 159 | d-Lys | Cys(N-hexyl) |
| 160 | d-Cys | Cys(N-hexyl) |

In some embodiments, the B amino acid of the dipeptide prodrug element is monosubstituted at the beta position and has a relatively bulky side chain, as shown in the below Table.

| Dipeptide Prodrug | Amino | Amino |
|---|---|---|
| Element # | Acid ‘A’ | Acid ‘B’ |
| 161 | Aib | Phe(N-C₁-C₈alkyl) |
| 162 | d-Ala | Phe(N-C₁-C₈alkyl) |
| 163 | d-Lys | Phe(N-C₁-C₈alkyl) |
| 164 | d-Cys | Phe(N-C₁-C₈alkyl) |
| 165 | Aib | Tyr(N-C₁-C₈alkyl) |
| 166 | d-Ala | Tyr(N-C₁-C₈alkyl) |
| 167 | d-Lys | Tyr(N-C₁-C₈alkyl) |
| 168 | d-Cys | Tyr(N-C₁-C₈alkyl) |
| 169 | Aib | Trp(N-C₁-C₈alkyl) |
| 170 | d-Ala | Trp(N-C₁-C₈alkyl) |
| 171 | d-Lys | Trp(N-C₁-C₈alkyl) |
| 172 | d-Cys | Trp(N-C₁-C₈alkyl) |
| 173 | Aib | Phe(N-methyl) |
| 174 | d-Ala | Phe(N-methyl) |
| 175 | d-Lys | Phe(N-methyl) |
| 176 | d-Cys | Phe(N-methyl) |
| 177 | Aib | Tyr(N-methyl) |
| 178 | d-Ala | Tyr(N-methyl) |
| 179 | d-Lys | Tyr(N-methyl) |
| 180 | d-Cys | Tyr(N-methyl) |
| 181 | Aib | Trp(N-methyl) |
| 182 | d-Ala | Trp(N-methyl) |
| 183 | d-Lys | Trp(N-methyl) |
| 184 | d-Cys | Trp(N-methyl) |
| 185 | Aib | Phe(N-hexyl) |
| 186 | d-Ala | Phe(N-hexyl) |
| 187 | d-Lys | Phe(N-hexyl) |
| 188 | d-Cys | Phe(N-hexyl) |
| 189 | Aib | Tyr(N-hexyl) |
| 190 | d-Ala | Tyr(N-hexyl) |
| 191 | d-Lys | Tyr(N-hexyl) |
| 192 | d-Cys | Tyr(N-hexyl) |
| 193 | Aib | Trp(N-hexyl) |
| 194 | d-Ala | Trp(N-hexyl) |
| 195 | d-Lys | Trp(N-hexyl) |
| 196 | d-Cys | Trp(N-hexyl) |

### Sub-Category IC: Amino Acid B of the Dipeptide Prodrug Element Disubstituted at the Beta Position

In some embodiments, the B amino acid of the dipeptide prodrug element is disubstituted at the beta position. Nonlimiting examples of dipeptide prodrug elements having a B amino acid that is disubstituted at the beta position are shown in the below Table.

| Dipeptide Prodrug | Amino | Amino |
|---|---|---|
| Element # | Acid ‘A’ | Acid ‘B’ |
| 197 | Aib | Ile(N-C₁-C₈alkyl) |
| 198 | d-Ala | Ile(N-C₁-C₈alkyl) |
| 199 | d-Lys | Ile(N-C₁-C₈alkyl) |
| 200 | d-Cys | Ile(N-C₁-C₈alkyl)) |
| 201 | Aib | Val(N-C₁-C₈alkyl) |
| 202 | d-Ala | Val(N-C₁-C₈alkyl) |
| 203 | d-Lys | Val(N-C₁-C₈alkyl) |
| 204 | d-Cys | Val(N-C₁-C₈alkyl) |
| 205 | Aib | Thr(N-C₁-C₈alkyl) |
| 206 | d-Ala | Thr(N-C₁-C₈alkyl) |
| 207 | d-Lys | Thr(N-C₁-C₈alkyl) |
| 208 | d-Cys | Thr(N-C₁-C₈alkyl) |
| 209 | Aib | Ile(N-methyl) |
| 210 | d-Ala | Ile(N-methyl) |
| 211 | d-Lys | Ile(N-methyl) |
| 212 | d-Cys | Ile(N-methyl)) |
| 213 | Aib | Val(N-methyl) |
| 214 | d-Ala | Val(N-methyl) |
| 215 | d-Lys | Val(N-methyl) |
| 216 | d-Cys | Val(N-methyl) |
| 217 | Aib | Thr(N-methyl) |
| 218 | d-Ala | Thr(N-methyl) |
| 219 | d-Lys | Thr(N-methyl) |
| 220 | d-Cys | Thr(N-methyl) |
| 221 | Aib | Ile(N-hexyl) |
| 222 | d-Ala | Ile(N-hexyl) |
| 223 | d-Lys | Ile(N-hexyl) |
| 224 | d-Cys | Ile(N-hexyl) |
| 225 | Aib | Val(N-hexyl) |
| 226 | d-Ala | Val(N-hexyl) |
| 227 | d-Lys | Val(N-hexyl) |
| 228 | d-Cys | Val(N-hexyl) |
| 229 | Aib | Thr(N-hexyl) |
| 230 | d-Ala | Thr(N-hexyl) |
| 231 | d-Lys | Thr(N-hexyl) |
| 232 | d-Cys | Thr(N-hexyl)) |

In some exemplary embodiments, Aib-Gly(N-Hexyl), dLys-Gly(N-Hexyl), dCys-Gly(N-Hexyl), dAla-Gly(N-Hexyl), Aib-Gly(N-Methyl), dLys-Gly(N-Methyl), dCys-Gly(N-Methyl), dAla-Gly(N-Hexyl), Aib-Phe(N-Methyl), dLys-Phe(N-Methyl), dCys-Phe(N-Methyl), or dAla-Phe(N-Methyl) is conjugated to the N-terminal alpha amino group of a peptide drug.

In accordance with one embodiment the dipeptide element comprises one of three amino acids at the B of the A-B dipeptide: Gly(N-Hexyl), Gly(N-Methyl) or Phe(N-Methyl).

Dipeptides selected from one of these three groups of dipeptides have relative cleavage rates wherein Gly(N-Hexyl)>Gly(N-Methyl)>Phe(N-Methyl) all other factors being equal. In one embodiment Cys or Lys is provided in the first position (i.e., the A amino acid) to provides a location for acylation or pegylation. Ala is used as the A amino acid in one embodiment where no acylation or pegylation is desired. In one embodiment an Aib in first position (i.e., the A amino acid) increases speed of cleavage relative to natural amino acids such as Ala, Cys, & Lys.
Exemplary dipeptides include:
dAla-Phe(N-Methyl)
dCys-Phe(N-Methyl)
dLys-Phe(N-Methyl)

### Aib-Phe(N-Methyl)

dAla-Gly(N-Methyl)
dCys-Gly(N-Methyl)
dLys-Gly(N-Methyl)

### Aib-Gly(N-Methyl)

dAla-Gly(N-Hexyl)
dCys-Gly(N-Hexyl)
dLys-Gly(N-Hexyl)

### Aib-Gly(N-Hexyl)

The disclosed medicinal agent and bioactive peptide prodrug derivatives are believed to be suitable for any use that has previously been described for its corresponding parent medicinal agent or bioactive peptide. Pharmaceutical compositions comprising the prodrugs disclosed herein can be formulated and administered to patients using standard pharmaceutically acceptable carriers and routes of administration known to those skilled in the art. Accordingly, the present disclosure also encompasses pharmaceutical compositions comprising one or more of the prodrugs disclosed herein, or a pharmaceutically acceptable salt thereof, in combination with a pharmaceutically acceptable carrier. In one embodiment the pharmaceutical composition comprises a 1 mg/ml concentration of the prodrug at pH of about 4.0 to about 7.0 in a phosphate buffer system. The pharmaceutical compositions may comprise the prodrug as the sole pharmaceutically active component, or the prodrugs can be combined with one or more additional active agents, including for example the active medicinal agent.

In accordance with one embodiment a pharmaceutical composition is provided comprising any of the novel dipeptide/medicinal agent complexes disclosed herein, preferably sterile and preferably at a purity level of at least 90%, 91%, 92%, 93%, 94%, 95%, 96%, 97%, 98% or 99%, and a pharmaceutically acceptable diluent, carrier or excipient. Such compositions may contain a dipeptide/medicinal agent complex as disclosed herein, wherein the resulting active agent is present at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored within various containers. The compounds disclosed herein can be used in accordance with one embodiment to prepare pre-formulated solutions ready for injection. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

All therapeutic methods, pharmaceutical compositions, kits and other similar embodiments described herein contemplate that the dipeptide/medicinal agent complexes include all pharmaceutically acceptable salts thereof.

In one embodiment the kit is provided with a device for administering the dipeptide/medicinal agent complex composition to a patient. The kit may further include a variety of containers, e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the prodrug composition is prepackaged within the syringe.

### Example 1

### Determination of Rate of Model Dipeptide Cleavage (in PBS)

A specific hexapeptide (HSRGTF-NH₂; SEQ ID NO: 2) was used as a model peptide to determine the half life of various dipeptides linked to the hexapeptide through an amide bond. The hexapeptide was assembled on a peptide synthesizer and Boc-protected sarcosine and lysine were successively added to the model peptide-bound resin to produce peptide A (Lys-Sar-HSRGTF-NH₂; SEQ ID NO: 3). Peptide A was cleaved by HF and purified by preparative HPLC.

Preparative Purification Using HPLC:

Purification was performed using HPLC analysis on a silica based 1×25 cm Vydac C18 (5μ particle size, 300 A° pore size) column. The instruments used were: Waters Associates model 600 pump, Injector model 717, and UV detector model 486. A wavelength of 230 nm was used for all samples. Solvent A contained 10% CH₃CN/0.1% TFA in distilled water, and solvent B contained 0.1% TFA in CH₃CN. A linear gradient was employed (0 to 100% B in 2 hours). The flow rate was 10 ml/min and the fraction size was 4 ml. From ˜150 mgs of crude peptide, 30 mgs of the pure peptide was obtained.

Peptide A was dissolved at a concentration of 1 mg/ml in PBS buffer. The solution was incubated at 37° C. Samples were collected for analysis at 5 h, 8 h, 24 h, 31 h, and 47 h. The dipeptide cleavage was quenched by lowering the pH with an equal volume of 0.1% TFA. The rate of cleavage was qualitatively monitored by LC-MS and quantitatively studied by HPLC. The retention time and relative peak area for the prodrug and the parent model peptide were quantified using Peak Simple Chromatography software.

### Analysis Using Mass Spectrometry

The mass spectra were obtained using a Sciex API-III electrospray quadrapole mass spectrometer with a standard ESI ion source. Ionization conditions that were used are as follows: ESI in the positive-ion mode; ion spray voltage, 3.9 kV; orifice potential, 60 V. The nebulizing and curtain gas used was nitrogen flow rate of 0.9 L/min. Mass spectra were recorded from 600-1800 Thompsons at 0.5 Th per step and 2 msec dwell time. The sample (about 1 mg/mL) was dissolved in 50% aqueous acetonitrile with 1% acetic acid and introduced by an external syringe pump at the rate of 5 μL/min.

Peptides solubilized in PBS were desalted using a ZipTip solid phase extraction tip containing 0.6 μL, C4 resin, according to instructions provided by the manufacturer (Millipore Corporation, Billerica, Mass.) prior to analysis.

### Analysis Using HPLC

The HPLC analyses were performed using a Beckman System Gold

Chromatography system equipped with a UV detector at 214 nm and a 150 mm×4.6 mm C8 Vydac column. The flow rate was 1 ml/min. Solvent A contained 0.1% TFA in distilled water, and solvent B contained 0.1% TFA in 90% CH₃CN. A linear gradient was employed (0% to 30% B in 10 minutes). The data were collected and analyzed using Peak Simple Chromatography software.

The initial rates of cleavage were used to measure the rate constant for the dissociation of the dipeptides from the respective prodrugs. The concentrations of the prodrugs and the model parent peptide were determined by their respective peak areas, ‘a’ and ‘b’ for each of the different collection times (Table 1). The first order dissociation rate constants of the prodrugs were determined by plotting the logarithm of the concentration of the prodrug at various time intervals. The slope of this plot provides the rate constant 1′. The half lives for cleavage of the various prodrugs were calculated by using the formula t_{1/2}=0.693/k. The half life of the Lys-Sar extension to this model peptide HSRGTF-NH₂ (SEQ ID NO: 2) was determined to be 14.0 h.

TABLE 1
HPLC and LC-MS data of Cleavage of A peptide (lys-sar-HSRGTF-NH₂) in PBS

| | 5 h | | 8 h | | 24 h | | 31 h | | 47 h | |
|---|---|---|---|---|---|---|---|---|---|---|
| HPLC peaks | a | b | a | b | a | b | a | b | a | b |
| Retention | 4.3 | 4.8 | 4.2 | 4.7 | 4.3 | 4.8 | 4.3 | 4.8 | 4.3 | 4.8 |
| time (min) | | | | | | | | | | |
| Molecular | 702 | 902 | 702 | 902 | 702 | 902 | 702 | 902 | 702 | 902 |
| weight | | | | | | | | | | |
| Relative | 26.5 | 73.5 | 28.9 | 71.1 | 28.8 | 71.2 | 77.7 | 22.3 | 90.0 | 10.0 |
| peak | | | | | | | | | | |
| area (%) | | | | | | | | | | |

### Example 2

### Rate of Dipeptide Cleavage Half Time in Plasma as Determined with an all D-Isoform Model Peptide

An additional model hexapeptide (dHdTdRGdTdF-NH₂ SEQ ID NO: 4) was used as a model to determine the rate of dipeptide cleavage in plasma. The d-isomer of each amino acid was used to prevent enzymatic cleavage of the model peptide, with the exception of the prodrug extension. This model d-isomer hexapeptide was synthesized in an analogous fashion to the 1-isomer. The sarcosine and lysine were successively added to the N-terminus as reported previously for peptide A to prepare peptide B (Lys-Sar-dHdTdRGdTdF-NH₂ SEQ ID NO: 5)

The initial rates of cleavage were used to measure the rate constant for the dissociation of the dipeptides from the respective prodrugs. The concentrations of the prodrug and the model parent peptide were determined by their respective peak areas ‘a’ and ‘b’ (Table 2). The first order dissociation rate constants of the prodrugs were determined by plotting the logarithm of the concentration of the prodrug at various time intervals. The slope of this plot provides the rate constant 1′. The half life of the Lys-Sar extension to this model peptide dHdTdRGdTdF-NH₂ (SEQ ID NO: 4) was determined to be 18.6 h.

TABLE 2
HPLC and LC-MS data of Cleavage of B peptide (lys-sar-dHdTdRGdTdF-
NH₂) in plasma

| | 5 h | | 11 h | | 24 h | | 32 h | | 48 h | |
|---|---|---|---|---|---|---|---|---|---|---|
| HPLC peaks | a | b | a | b | a | B | a | b | a | b |
| Retention | 5.7 | 6.2 | 5.8 | 6.3 | 5.7 | 6.2 | 5.7 | 6.2 | 5.7 | 6.2 |
| time (min) | | | | | | | | | | |
| Molecular | 702 | 902 | 702 | 902 | 702 | 902 | 702 | 902 | 702 | 902 |
| weight | | | | | | | | | | |
| Relative peak | 17.0 | 83.0 | 29.2 | 70.8 | 60.2 | 39.8 | 54.0 | 46.0 | 27.6 | 72.4 |
| area (%) | | | | | | | | | | |

### Example 3

The rate of cleavage for additional dipeptides linked to the model hexapeptide (HSRGTF-NH₂; SEQ ID NO: 2) were determined using the procedures described in Example 1. The results generated in these experiments are presented in Tables 3 and 4.

TABLE 3
Cleavage of the Dipeptides A-B that are linked to the side chain of an N-
terminal para-amino-Phe in the Model Peptides (in PBS)

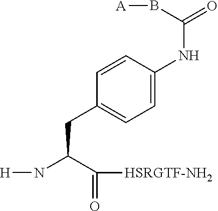

| Compounds | A (amino acid) | B (amino acid) | t_{1/2} | |
|---|---|---|---|---|
| 1 | F | P | 58 | h |
| 2 | Hydroxyl-F | P | 327 | h |
| 3 | d-F | P | 20 | h |
| 4 | d-F | d-P | 39 | h |
| 5 | G | P | 72 | h |
| 6 | Hydroxyl-G | P | 603 | h |
| 7 | L | P | 62 | h |
| 8 | tert-L | P | 200 | h |
| 9 | S | P | 34 | h |
| 10 | P | P | 97 | h |
| 11 | K | P | 33 | h |
| 12 | dK | P | 11 | h |
| 13 | E | P | 85 | h |
| 14 | Sar | P | about 1000 h | |
| 15 | Aib | P | 69 | min |
| 16 | Hydroxyl-Aib | P | 33 | h |
| 17 | cyclohexane | P | 6 | min |
| 18 | G | G | No cleavage | |
| 19 | Hydroxyl-G | G | No cleavage | |
| 20 | S | N-Methyl-Gly | 4.3 | h |
| 21 | K | N-Methyl-Gly | 5.2 | h |
| 22 | Aib | N-Methyl-Gly | 7.1 | min |
| 23 | Hydroxyl-Aib | N-Methyl-Gly | 1.0 | h |

TABLE 4
Cleavage of the Dipeptide A-B linked to histidine
(or a histidine derivative) at position1 (X) from the
Model Hexapeptide (XSRGTF-NH₂) in PBS
NH₂-A-B-XSRGTF-NH₂

| Com- | A | B | X₁ | | |
|---|---|---|---|---|---|
| pounds | (amino acid) | (amino acid) | (amino acid) | t_{1/2} | |
| 1 | F | P | H | No | |
| | | | | cleavage | |
| 2 | Hydroxyl-F | P | H | No | |
| | | | | cleavage | |
| 3 | G | P | H | No | |
| | | | | cleavage | |
| 4 | Hydroxyl-G | P | H | No | |
| | | | | cleavage | |
| 5 | A | P | H | No | |
| | | | | cleavage | |
| 6 | C | P | H | No | |
| | | | | cleavage | |
| 7 | S | P | H | No | |
| | | | | cleavage | |
| 8 | P | P | H | No | |
| | | | | cleavage | |
| 9 | K | P | H | No | |
| | | | | cleavage | |
| 10 | E | P | H | No | |
| | | | | cleavage | |
| 11 | Dehydro V | P | H | No | |
| | | | | cleavage | |
| 12 | P | d-P | H | No | |
| | | | | cleavage | |
| 13 | d-P | P | H | No | |
| | | | | cleavage | |
| 14 | Aib | P | H | 32 | h |
| 15 | Aib | d-P | H | 20 | h |
| 16 | Aib | P | d-H | 16 | h |
| 17 | Cyclohexyl- | P | H | 5 | h |
| 18 | Cyclopropyl- | P | H | 10 | h |
| 19 | N-Me-Aib | P | H | >500 | h |
| 20 | α,α-diethyl- | P | H | 46 | h |
| | Gly | | | | |
| 21 | Hydroxyl-Aib | P | H | 61 | |
| 22 | Aib | P | A | 58 | |
| 23 | Aib | P | N-Methyl-His | 30 | h |
| 24 | Aib | N-Methyl-Gly | H | 9 | min |
| 25 | Aib | N-Hexyl-Gly | H | 10 | min |
| 26 | Aib | Azetidine-2- | H | >500 | h |
| | | carboxylic acid | | | |
| 27 | G | N-Methyl-Gly | H | 104 | h |
| 28 | Hydroxyl-G | N-Methyl-Gly | H | 149 | h |
| 29 | G | N-Hexyl-Gly | H | 70 | h |
| 30 | dK | N-Methyl-Gly | H | 27 | h |
| 31 | dK | N-Methyl-Ala | H | 14 | h |
| 32 | dK | N-Methyl-Phe | H | 57 | h |
| 33 | K | N-Methyl-Gly | H | 14 | h |
| 34 | F | N-Methyl-Gly | H | 29 | h |
| 35 | S | N-Methyl-Gly | H | 17 | h |
| 36 | P | N-Methyl-Gly | H | 181 | h |

What is claimed:

1. A prodrug comprising the structure:
A-B-Q;
wherein Q is an amine bearing medicinal agent, A is an amino acid and B is an N-alkylated amino acid;
wherein A-B comprises the structure: 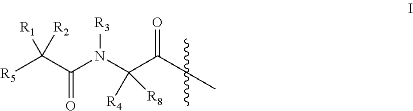
wherein
R₁ and R₂ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl;
R₃ is C₁-C₁₈ alkyl;
R₄ is H, C₁-C₄ alkyl, or (C₁-C₄ alkyl)NH₂;
R₅ is NH₂;
R₈ is H; and,
R₇ is selected from the group consisting of H and OH;
wherein A-B is linked to Q through an amide bond between A-B and an aliphatic amino group of Q;
wherein the chemical cleavage half-life (t_{1/2}) of A-B from Q is at least about 1 hour to about 1 week in PBS under physiological conditions;
with the proviso that when both R₁ and R₂ are H, R₃ is C₅-C₁₈ alkyl;

2. The prodrug of claim 1, wherein B is selected from the group consisting of glycine(N-methyl), glycine(N-ethyl), glycine(N-propyl), glycine(N-butyl), glycine(N-pentyl), glycine(N-hexyl), glycine(N-heptyl), and glycine(N-octyl).

3. The prodrug of claim 2 wherein
R₁ is H or C₁-C₈ alkyl; and
R₂ is selected from the group consisting of C₁-C₆ alkyl, CH₂OH, (C₁-C₄ alkyl)NH₂, and CH₂(C₆ aryl)R₇.

4. The prodrug of claim 2 wherein
R₁ is H;
R₂ is (C₁-C₄ alkyl)NH₂.

5. The prodrug of claim 1 wherein A-B is glycine-glycine(N-hexyl).

6. The prodrug of claim 1, wherein A has d-stereochemistry.

7. The prodrug of claim 1, wherein A-B is selected from the group consisting of dLys-Gly(N-Hexyl), dCys-Gly(N-Hexyl), dLys-Gly(N-Methyl), dCys-Gly(N-Methyl), dAla-Gly(N-Hexyl), dLys-Phe(N-Methyl), or dCys-Phe(N-Methyl).

8. A prodrug comprising the structure:
A-B-Q;
wherein Q is an amine bearing medicinal agent, A is an amino acid and B is an N-alkylated amino acid;
wherein A-B comprises the structure: 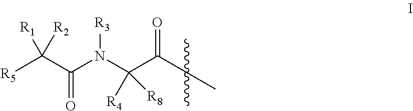
wherein
R₁ is C₁-C₈ alkyl;
R₂ is selected from the group consisting of C₁-C₆ alkyl, CH₂OH, (C₁-C₄ alkyl)NH₂, and CH₂(C₆ aryl)R₇,
R₃ and R₄ together with the atoms to which they are attached form a pyrrolidine ring;
R₅ is NH₂;
R₈ is H; and,
R₇ is selected from the group consisting of H and OH; wherein the chemical cleavage half-life (t_{1/2}) of A-B from Q is at least about 1 hour to about 1 week in PBS under physiological conditions.

9. The prodrug of claim 8, wherein A is aminoisobutyric acid.

10. The prodrug of claim 1, further comprising a hydrophilic moiety covalently linked to the prodrug.

11. The prodrug of claim 1, further comprising an acyl group or alkyl group covalently linked to the prodrug.

12. A prodrug comprising the structure of Formula II; 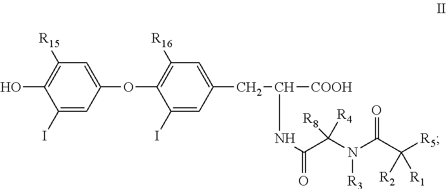
wherein
R₁ is selected from the group consisting of H and C₁-C₈ alkyl;
R₂ and R₄ are independently selected from the group consisting of H, C₁-C₈ alkyl, and (C₁-C₄ alkyl)NH₂;
R₃ is C₁-C₆ alkyl, or R₄ and R₃ together with the atoms to which they are attached form a pyrrolidine ring;
R₅ is NH₂;
R₇ is selected from the group consisting of H and OH; and
R₈ is H, with the proviso that when R₄ and R₃ together with the atoms to which they are attached form a pyrrolidine ring, both R₁ and R₂ are other than H, and
R₁₅ and R₁₆ are independently selected from hydrogen and iodine.

13. The complex of claim 12 wherein
R₁ is H;
R₂ is (C₁-C₄ alkyl)NH₂;
R₃ is C₁-C₈ alkyl;
R₄ and R₈ are each hydrogen; and
R₅ is an NH₂.

14. The complex of claim 12 wherein
R₁ and R₂ are each CH₃;
R₃ and R₄ together with the atoms to which they are attached form a pyrrolidine ring;
R₈ is H; and
R₅ is an NH₂.


SEQUENCE LISTING (58 sequences)

SEQ ID NO: 1 - protein, 29 aa, Homo sapiens
HSQGTFTSDYSKYLDSRRAQDFVQWLMNT

SEQ ID NO: 2 - protein, 6 aa, Artificial Sequence
  Glucagon analogue
HTRGTF

SEQ ID NO: 3 - protein, 8 aa, Artificial Sequence
  Glucagon analogue
KXHTRGTF

SEQ ID NO: 4 - protein, 6 aa, Artificial Sequence
  glucagon analogue
XXXGXX

SEQ ID NO: 5 - protein, 8 aa, Artificial Sequence
  glucagon analogue
KXXXXGXX

SEQ ID NO: 6 - protein, 11 aa, Homo sapiens
CYFQNCPRGGK

SEQ ID NO: 7 - protein, 28 aa, Homo sapiens
SLRRSSCFGGRMDRIGAQSGLGCNSFRY

SEQ ID NO: 8 - protein, 44 aa, Homo sapiens
YADAIFTNSYRKVLGQLSARKLLQDIMSRQQGESNQERGARARL

SEQ ID NO: 9 - protein, 36 aa, Homo sapiens
YPSKPDNPGEDAPAEDMARYYSALRHYINLITRQRY

SEQ ID NO: 10 - protein, 9 aa, Homo sapiens
CYIQNCPLG

SEQ ID NO: 11 - protein, 36 aa, Homo sapiens
APLEPVYPGDNATPEQMAQYAADLRRYINMLTRPRY

SEQ ID NO: 12 - protein, 28 aa, Homo sapiens
SANSNPAMAPRERKAGCKNFFWKTFTSC

SEQ ID NO: 13 - protein, 14 aa, Homo sapiens
AGCKNFFWKTFTSC

SEQ ID NO: 14 - protein, 32 aa, Homo sapiens
  DISULFID (1)..(7): Cys residues linked via disulfide bridge
CGNLSTCMLGTYTQDFNKFHTFPQTAIGVGAP

SEQ ID NO: 15 - protein, 48 aa, Mus musculus
  DISULFID (10)..(16): Cys residues linked via disulfide bridge
SLDSPRSKRCGNLSTCMLGTYTQDLNEFHTFPQTSIGVEAPGKKRDVA

SEQ ID NO: 16 - protein, 32 aa, Rattus norvegicus
  DISULFID (1)..(7): Cys residues linked via disulfide bridge
CGNLSTCMLGTYTQDLNKFHTFPQTSIGVGAP

SEQ ID NO: 17 - protein, 32 aa, Sus scrofa
  MOD_RES (32)..(32): C-terminal amidation
CSNLSTCVLSAYWRNLNNFHRFSGMGFGPETP

SEQ ID NO: 18 - protein, 32 aa, Canis lupus
  DISULFID (1)..(7): Cys residues linked via disulfide bridge
CSNLSTCVLGTYSKDLNNFHTFSGIGFGAETP

SEQ ID NO: 19 - protein, 32 aa, Bos taurus
  DISULFID (1)..(7): Cys residues linked via disulfide bridge
CSNLSTCVLSAYWKDLNNYHRFSGMGFGPETP

SEQ ID NO: 20 - protein, 126 aa, Danio rerio
  DISULFID (82)..(87): Cys residues linked via disulfide bridge
MVMLKISAFLVAYALIICQMYSSNAAPARPALESSPDRTTLSDYEARRLLQAIVKEFMQMTAEDMEQQATEENSVTTQKRACNTATCVTHRLADFLSRSGGIGSSKFVPTNVGSQAFGRRRRLSQE

SEQ ID NO: 21 - protein, 127 aa, Oryzias latipes
  DISULFID (82)..(113): Cys residues linked via disulfide bridge
MTMLKLWTLLLANALLLCQMYISEAAPSRTSKEFVTDGVPLLNNEAETLFRAIKDYIEMTSEEAAKEEAEEESLDRPLSKRCTGLSTCVLGRLSQDIHKLQTYPRTDVGAGTPGKKRSLFEQFENYS

SEQ ID NO: 22 - protein, 32 aa, Gallus gallus
  DISULFID (1)..(7): Cys residues linked via disulfide bridge
CASLSTCVLGKLSQELHKLQTYPRTDVGAGTP

SEQ ID NO: 23 - protein, 32 aa, Oncorhynchus gorbuscha
  DISULFID (1)..(7): Cys residues linked via disulfide bridge
CSNLSTCVLGKLSQELHKLQTYPRTNTGSGTP

SEQ ID NO: 24 - protein, 32 aa, Ovis aries
  DISULFID (1)..(7): Cys residues linked via disulfide bridge
CSNLSTCVLSAYWKDLNNYHRYSGMGFGPETP

SEQ ID NO: 25 - protein, 136 aa, Takifugu rubripes
  DISULFID (83)..(89): Cys residues linked via disulfide bridge
MVMLKISAFLLAYALVICQMYSSHAAPARPGLESMSDRVTLTDYEARRLLNAIVKEFVQMTAEELEQQATEGNSMDRPLTKRCSNLSTCVLGKLSQELHKLQTFPRTNVGAGTPGKKRSAAESDSYASYGETFGRI

SEQ ID NO: 26 - protein, 50 aa, Paralichthys olivaceus
  DISULFID (1)..(7): Cys residues linked via disulfide bridge
CTGLSTCVLGKLSQDIHKLQTYPRTNVGAGTPGKKRSLSEQYENHGSSYN

SEQ ID NO: 27 - protein, 32 aa, Sardinops melanostictus
  DISULFID (1)..(7): Cys residues linked via disulfide bridge
CSNLSTCALGKLSQELHKLQSYPRTNVGAGTP

SEQ ID NO: 28 - protein, 32 aa, Carassius auratus
  DISULFID (1)..(7): Cys residues linked via disulfide bridge
CSSLSTCVLGKLSQELHKLQTYPRTNVGAGTP

SEQ ID NO: 29 - protein, 32 aa, Salvelinus alpinus; identical to SEQ ID NO: 23
  DISULFID (1)..(7): Cys residues linked via disulfide bridge

SEQ ID NO: 30 - protein, 32 aa, Anguilliformes
  DISULFID (1)..(7): Cys residues linked via disulfide bridge
CSNLSTCVLGKLSQELHKLQTYPRTDVGAGTP

SEQ ID NO: 31 - protein, 32 aa, Oncorhynchus keta
  DISULFID (1)..(7): Cys residues linked via disulfide bridge
CSNLSTCVLGKLSQELHKLQTYPRTNTGNGTP

SEQ ID NO: 32 - protein, 32 aa, Oncorhynchus kisutch; identical to SEQ ID NO: 23
  DISULFID (1)..(7): Cys residues linked via disulfide bridge

SEQ ID NO: 33 - protein, 32 aa, Salmo salar; identical to SEQ ID NO: 23
  DISULFID (1)..(7): Cys residues linked via disulfide bridge

SEQ ID NO: 34 - protein, 31 aa, Dasyatis akajei
  DISULFID (1)..(7): Cys residues linked via disulfide bridge
CTSLSTCVVGKSQQLHKLQNIQRTDVGAATP

SEQ ID NO: 35 - protein, 37 aa, Homo sapiens [sequence omitted]

SEQ ID NO: 36 - protein, 93 aa, Mus musculus
  DISULFID (39)..(44): Cys residues linked via disulfide bridge
MMCISKLPAVLLILSVALNHLRATPVRSGSNPQMDKRKCNTATCATQRLANFLVRSSNNLGPVLPPTNVGSNTYGKRNAAGDPNRESLDFLLV

SEQ ID NO: 37 - protein, 93 aa, Rattus norvegicus
  DISULFID (39)..(44): Cys residues linked via disulfide bridge
MRCISRLPAVLLILSVALGHLRATPVGSGTNPQVDKRKCNTATCATQRLANFLVRSSNNLGPVLPPTNVGSNTYGKRNVAEDPNRESLDFLLL

SEQ ID NO: 38 - protein, 92 aa, Cavia porcellus
  DISULFID (38)..(43): Cys residues linked via disulfide bridge
MCLLRLPVTLLVLCVALNELKATSIASDTGHQVGKRKCNTATCATQRLTNFLVRSSHNLGAALLPTDVGSNTYGKRNAPQISDRELLHYLPL

SEQ ID NO: 39 - protein, 89 aa, Canis lupis
  DISULFID (35)..(40): Cys residues linked via disulfide bridge
MCLLKLPVVLIILSVALNHLKATPIKSHQMEKRKCNTATCATQRLANFLVRTSNNLGAILSPTNVGSNTYGKRNTIEILNRGPLNYLPL

SEQ ID NO: 40 - protein, 89 aa, Felis catus
  DISULFID (35)..(40): Cys residues linked via disulfide bridge
MCLLKLPVVLIVLLVALHHLKATPIESNQVEKRKCNTATCATQRLANFLIRSSNNLGAILSPTNVGSNTYGKRSTVDILNREPLNYLPF

SEQ ID NO: 41 - protein, 89 aa, Papio hamadrys
  DISULFID (35)..(40): Cys residues linked via disulfide bridge
MCILKLQVFLIVLFVALNHLKATPIESHQGEKRICNTATCATQRLANFLVRSSNNFGTILSSTNVGSNTYGKRNAVEVLKREPLNYLPL

SEQ ID NO: 42 - protein, 92 aa, Mesocricetus auratus
  DISULFID (38)..(43): Cys residues linked via disulfide bridge
MHISKLPAALLIFSVALNHLKATPVRSGTNHQMDKRKCNTATCATQRLANFLVHSNNNLGPVLSPTNVGSNTYGKRSAAEIPDGDSLDLFLL

SEQ ID NO: 43 - protein, 91 aa, Octodon degus
  DISULFID (38)..(43): Cys residues linked via disulfide bridge
MCLLQLPVVLLLLSAALNTLKATPIASDTDHRVDKRKCNTATCATQRLTNFLVRSSHNLGAALPPTKVGSNTYGRRNAEVVDVELLHYLPL

SEQ ID NO: 44 - protein, 89 aa, Monodelphis domestica
  DISULFID (35)..(40): Cys residues linked via disulfide bridge
MYNLKLPIVFIVLSVALSCLEATPIDSHHLEKRKCNTATCVTQRLADFLIRSSNNIGAVFSPTNVGSNTYGKREIAGILSREPLNQFPH

SEQ ID NO: 45 - protein, 126 aa, Carrassius auratus
  DISULFID (77)..(82): Cys residues linked via disulfide bridge
MYLPSQILIFLVMLQCVATVPYNRYSLSSNDKPDASREVNGWLVTDLSDNPFVSFTRPRPPWGLPAVNSHYMEKRKCNTATCVTQRLADFLVRSSNTRGTVYAPTNVGANTYGKRDLLQSPIYLPL

SEQ ID NO: 46 - protein, 130 aa, Osmerus mordax
  DISULFID (81)..(86): Cys residues linked via disulfide bridge
MYHLRLPMLLIVPLVLLPCVITAPSNRYFSPISSGQESAPPEREDWLLPEWVSNPFLSLVGARPQRGLPAVNSHHIEKRKCNTATCVTQRLADFLVRSSNTIGTVYAPTNVGSSTYGKRELLQPPSYFPL

SEQ ID NO: 47 - protein, 37 aa, Cricetulus griseus
  DISULFID (2)..(7): Cys residues linked via disulfide bridge
KCNTATCATQRLANFLVHSNNNLGPVLSPTNVGSNTY

SEQ ID NO: 48 - protein, 37 aa, Artificial Sequence
  Synthetic peptide
KCNTATCATQRLANFLVHSSNNFGPILPPTNVGSNTY

SEQ ID NO: 49 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 50 - protein, 31 aa, Homo sapiens
HAEGTFTSDVSSYLEGQAAKEFIAWLVKGRG

SEQ ID NO: 51 - protein, 37 aa, Homo sapiens
HSQGTFTSDYSKYLDSRRAQDFVQWLMDTKRNRNNIA

SEQ ID NO: 52 - protein, 42 aa, Homo sapiens
YAEGTFISDYSIAMDKIHQQDFVNWLLAQKGKKNDWKHNITQ

SEQ ID NO: 53 - protein, 33 aa, Homo sapiens
HADGSFSDEMNTILDNLAARDFINWLIQTKITD

SEQ ID NO: 54 - protein, 39 aa, Heloderma suspectum
HGEGTFTSDLSKQMEEEAVRLFIEWLKNGGPSSGAPPPS

SEQ ID NO: 55 - protein, 28 aa, Homo sapiens
  MOD_RES (28)..(28): AMIDATION
HSDAVFTDNYTRLRKQMAVKKYLNSILN

SEQ ID NO: 56 - protein, 27 aa, Homo sapiens
HSDGIFTDSYSRYRKQMAVKKYLAAVL

SEQ ID NO: 57 - protein, 27 aa, Homo sapiens
  MOD_RES (27)..(27): AMIDATION
HADGVFTSDFSKLLGQLSAKKYLESLM

SEQ ID NO: 58 - protein, 27 aa, Homo sapiens
  MOD_RES (27)..(27): AMIDATION
HSDGTFTSELSRLREGARLQRLLQGLV